(19) United States
(12) Patent Application Publication
(10) Pub. No.: US 2008/0058312 A1
(43) Pub. Date: Mar. 6, 2008

(54) Modulators of hepatocyte growth factor/c-Met activity

(75) Inventors: David E. Zembower, La Grange, IL (US); Jasbir Singh, Naperville, IL (US); Rama K. Mishra, Chicago, IL (US); Dawoon Jung, Tenafly, NJ (US); Xiaokang Zhu, Hempstead, NY (US)

Correspondence Address: FOLEY HOAG, LLP;PATENT GROUP, WORLD TRADE CENTER WEST, 155 SEAPORT BLVD, BOSTON, MA, 02110, US

(73) Assignee: Angion Biomedica Corporation, Manhasset, NY (US)

(21) Appl. No.: 11/651,926

(22) Filed: Jan. 10, 2007

Related U.S. Application Data
(60) Provisional application No. 60/758,478, filed on Jan. 11, 2006.
(60) Provisional application No. 60/837,655, filed on Aug. 15, 2006.
(60) Provisional application No. 60/853,654, filed on Oct. 23, 2006.

(51) Int. Cl. A61K 31/517; A61K 31/496; A61K 31/5377; A61K 31/541; A61K 31/55; A61P 19/02; A61P 35/00

(52) U.S. Cl. 514/217.06; 514/228.2; 514/234.5; 514/252.17; 514/266.22; 514/266.4

(57) ABSTRACT

This invention is directed to compounds and compositions that have biological properties useful for modulating HGF/SF activity. In certain embodiments, said compounds and compositions may be used in the treatment and prophylaxis of cancer or other dysproliferative diseases and inflammatory diseases such as rheumatoid arthritis.

### RELATED APPLICATIONS

This application claims the benefit of priority to U.S. provisional patent application Ser. Nos. 60/758,478, filed Jan. 11, 2006; 60/837,655, filed Aug. 15, 2006; and 60/853,654, filed Oct. 23, 2006.

### GOVERNMENT SUPPORT

The invention was made with government support under grant number 1R43CA096077-02 awarded by the National Institutes of Health. The government has certain rights in the invention.

### BACKGROUND OF THE INVENTION

Hepatocyte growth factor (HGF; also known as scatter factor, or SF, and hereinafter referred to and abbreviated as HGF/SF or HGF) is a pleiotropic growth factor that stimulates cell growth, cell motility, morphogenesis and angiogenesis. HGF/SF is produced as an inactive monomer (about 100 kDa) which is proteolytically converted to its active form. Active HGF/SF is a heparin-binding heterodimeric protein composed of a 62 kDa a chain and a 34 kDa β chain. HGF/SF is a potent mitogen for parenchymal liver, epithelial and endothelial cells. Matsumoto, K.; Nakamura, T. “Hepatocyte growth factor (HGF) as a tissue organizer for organogenesis and regeneration.” Biochem. Biophys. Res. Commun. 1997, 239, 639-44; Boros, P.; Miller, C. M. “Hepatocyte growth factor: a multifunctional cytokine.” Lancet 1995, 345, 293-5. It stimulates the growth of endothelial cells and also acts as a survival factor against endothelial cell death. Morishita, R.; Nakamura, S.; Nakamura, Y.; Aoki, M.; Moriguchi, A.; Kida, I.; Yo, Y.; Matsumoto, K.; Nakamura, T.; Higaki, J.; Ogihara, T. “Potential role of an endothelium-specific growth factor, hepatocyte growth factor, on endothelial damage in diabetes.” Diabetes 1997, 46, 138-42. HGF/SF synthesized and secreted by vascular smooth muscle cells stimulates endothelial cells to proliferate, migrate and differentiate into capillary-like tubes in vitro. Grant, D. S.; Kleinman, H. K.; Goldberg, I. D.; Bhargava, M. M.; Nickoloff, B. J.; Kinsella, J. L.; Polverini, P.; Rosen, E. M. “Scatter factor induces blood vessel formation in vivo.” Proc. Natl. Acad. Sci. USA 1993, 90, 1937-41; and Morishita, R.; Nakamura, S.; Hayashi, S.; Taniyama, Y.; Moriguchi, A.; Nagano, T.; Taiji, M.; Noguchi, H.; Takeshita, S.; Matsumoto, K.; Nakamura, T.; Higaki, J.; Ogihara, T. “Therapeutic angiogenesis induced by human recombinant hepatocyte growth factor in rabbit hind limb ischemia model as cytokine supplement therapy.” Hypertension 1999, 33, 1379-84. HGF/SF-containing implants in mouse subcutaneous tissue and rat cornea induce growth of new blood vessels from surrounding tissue. HGF/SF protein is expressed at sites of neovascularization including in tumors. Jeffers, M.; Rong, S.; Woude; G. F. “Hepatocyte growth factor/scatter factor-Met signaling in tumorigenicity and invasion/metastasis.” J. Mol. Med. 1996, 74, 505-13; and Moriyama, T.; Kataoka, H.; Koono, M.; Wakisaka, S. “Expression of hepatocyte growth factor/scatter factor and its receptor c-met in brain tumors: evidence for a role in progression of astrocytic tumors.” Int. J. Mol. Med. 1999, 3, 531-6). These findings suggest that HGF/SF plays a significant role in the formation and repair of blood vessels under physiologic and pathologic conditions. Further discussion of angiogenic proteins may be found in U.S. Pat. Nos. 6,011,009 and 5,997,868, both of which are incorporated herein by reference in their entireties.

Small-molecule modulators of HGF have been discussed in U.S. Pat. No. 6,589,997, U.S. Pat. No. 6,610,726, WO2006014420, and Christensen, J. G.; Burrows, J.; Salgia, R. “c-Met as a target for human cancer and characterization of inhibitors for therapeutic intervention.” Cancer Letters 2004, 225, 1-26; all of which are incorporated herein by reference in their entireties.

It is towards the identification of small organic molecules that inhibit HGF activity, antagonize c-Met, or exhibit at least one biological activity that is exhibited by a HGF inhibitor or c-Met antagonist, and are thus useful in the treatment or prevention of conditions or diseases in which inhibiting HGF/SF activity is desirable, such as cancers, inflammatory diseases, and other dysproliferative diseases, that the invention is directed.

All citations herein are incorporated by reference in their entireties. The citation of any reference herein is not an admission that such reference is available as “Prior Art” against the instant application.

### SUMMARY

This invention is directed to compounds and compositions that have biological properties useful for modulating, and preferably inhibiting HGF activity or antagonizing the HGF receptor, c-Met. Said compounds and compositions exhibit one, if not more, biological activities in common with HGF/SF inhibitors or c-Met antagonists. The use of such compounds and compositions include the treatment and prophylaxis of cancer, inflammatory diseases and other dysproliferative diseases. It should be pointed out that while in theory the compounds of the invention inhibit or antagonize such activity, the Applicants are by no means bound to this theory, and the compounds of the invention are useful for treating any of the various conditions indicated regardless of their activity related to HGF/SF per se.

One aspect of the invention relates to compounds, and pharmaceutically acceptable compositions thereof, represented by formula I: 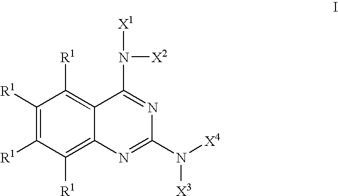
or a pharmaceutically acceptable salt thereof; wherein, independently for each occurrence: R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring; X¹, X², X³ and X⁴ are hydrogen or an optionally substituted aliphatic, alicyclic, heteroaliphatic, heterocyclic, aromatic, heteroaromatic or acyl group; or X¹ and X² taken together with the nitrogen to which they are bonded, or X³ and X⁴ taken together with the nitrogen to which they are bonded, are independently an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups; R^{R} is an optionally substituted aliphatic, alicyclic, heteroaliphatic, heterocyclic, aromatic, heteroaromatic or acyl moiety; R^{A} is hydrogen or an optionally substituted aliphatic, alicyclic, heteroaliphatic, heterocyclic, aromatic or heteroaromatic moiety; R^{B} is hydrogen, —OH, —SO₂R^{D}, or an optionally substituted aliphatic, alicyclic, heteroaliphatic, heterocyclic, aromatic, heteroaromatic or acyl moiety; R^{C} is hydrogen, —OH, —SO₂R^{D}, or an optionally substituted aliphatic, alicyclic, heteroaliphatic, heterocyclic, aromatic, heteroaromatic or acyl moiety; R^{D} is hydrogen, —N(R^{E})₂, or an optionally substituted aliphatic, alicyclic, heteroaliphatic, heterocyclic, aromatic or heteroaromatic moiety; and R^{E} is hydrogen or an optionally substituted aliphatic moiety.

Another aspect of the invention relates to compounds, and pharmaceutically acceptable compositions thereof, represented by formula II: 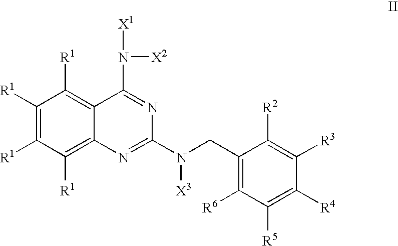
or a pharmaceutically acceptable salt thereof; wherein, independently for each occurrence: R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring; R²R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R}; X¹, X² and X³ are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups; R^{R} is hydrogen or an optionally substituted aliphatic, alicyclic, heteroaliphatic, heterocyclic, aromatic, heteroaromatic or acyl moiety; R^{A} is hydrogen or an optionally substituted aliphatic, alicyclic, heteroaliphatic, heterocyclic, aromatic or heteroaromatic moiety; R^{B} is hydrogen, —OH, —SO₂R^{D}, or an optionally substituted aliphatic, alicyclic, heteroaliphatic, heterocyclic, aromatic, heteroaromatic or acyl moiety; R^{C} is hydrogen, —OH, —SO₂R^{D}, or an optionally substituted aliphatic, alicyclic, heteroaliphatic, heterocyclic, aromatic, heteroaromatic or acyl moiety; R^{D} is hydrogen, —N(R^{E})₂, or an optionally substituted aliphatic, alicyclic, heteroaliphatic, heterocyclic, aromatic or heteroaromatic moiety; and R^{E} is hydrogen or an optionally substituted aliphatic moiety.

In another aspect, the invention is directed to compositions, including pharmaceutical compositions, comprising one or more compounds of formula I or II useful for various purposes including but not limited to prophylaxis and treatment of cancer, inflammatory diseases, and other dysproliferative diseases.

In another aspect, the invention is directed to a method for the prophylaxis or treatment of cancer, inflammatory, and other dysproliferative diseases by administering to a subject or patient in need thereof a therapeutically effective amount of a compound of formula I or II, or a pharmaceutical composition comprising a compound of formula I or II.

In another aspect, the invention is directed to the use of a compound of formula I or II, for the preparation of a medicament for administration to a subject or patient in need thereof for the treatment or prophylaxis of dysproliferative diseases, such as but not limited to cancer and inflammatory diseases.

### BRIEF DESCRIPTION OF THE FIGURE

FIG. 1 depicts compounds VII and IV of the invention.

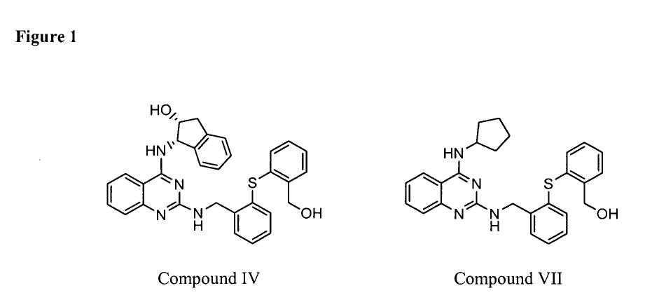

FIG. 2 shows the effects of compounds VII and IV of the invention (denoted with ∘ and ▪, respectively) on preventing growth of lung tumors in mice.

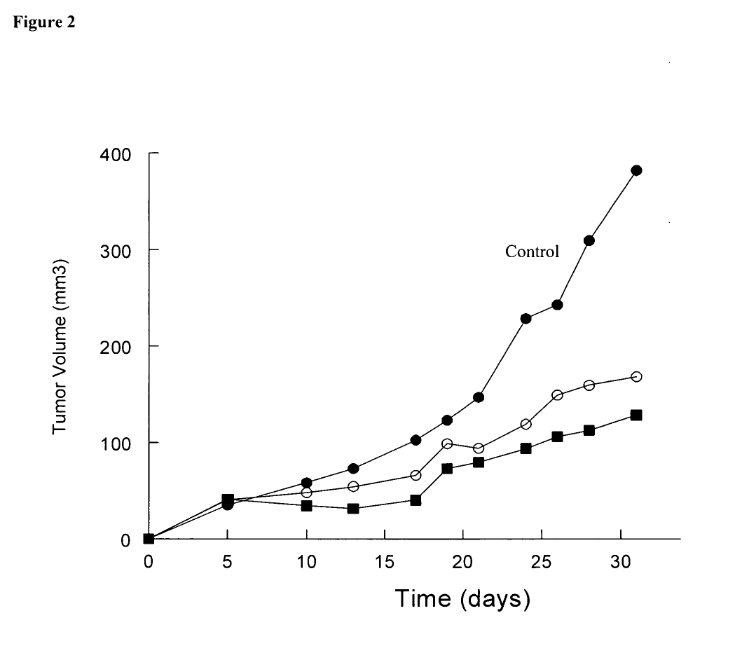

### DETAILED DESCRIPTION OF THE INVENTION

In certain embodiments, the present invention is directed to compounds and compositions useful for the treatment of cancer and other dysproliferative diseases as well as inflammatory disease in particular where inflammation, especially chronic inflammation, leads to inappropriate vascularization. Furthermore, the compounds of the invention have been identified as having biological properties useful for modulating, and preferably inhibiting or antagonizing, c-Met activity or Tie-2 activity, or at least exhibiting one, if not more, biological activities in common with a c-Met or Tie-2 inhibitor or antagonist. It should be pointed that while in theory the compounds of the invention inhibit or antagonize such at least one activity, Applicants are by no means bound to this theory, and the compounds of the invention are useful for treating any of the various conditions indicated regardless of their activity related to c-Met or Tie-2 per se.

Examples of cancers, tumors, malignancies, neoplasms, and other dysproliferative diseases that can be treated according to the invention include leukemias, such as myeloid and lymphocytic leukemias, lymphomas, myeloproliferative diseases, and solid tumors, such as but not limited to sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. In preferred but non-limiting embodiments, brain tumors, including glioma, and pancreatic cancers are amenable to treatment by the compounds of the present invention.

Examples of inflammatory diseases toward which compounds of the invention have benefit include rheumatoid arthritis, atherosclerosis, and neovascularization in the eye as a consequence of diabetic retinopathy.

The present invention is also directed to treatment of non-malignant tumors and other disorders involving inappropriate cell or tissue growth by administering a therapeutically effective amount of an agent of the invention. For example, the invention is useful for the treatment of arteriovenous (AV) malformations, particularly in intracranial sites. The invention can also be used to treat psoriasis, a dermatologic condition that is characterized by inflammation and vascular proliferation; benign prostatic hypertrophy, a condition associated with inflammation and possibly vascular proliferation; and cutaneous fungal infections. Treatment of other hyperproliferative disorders is also embraced herein. The agents may also be used topically to remove warts, birthmarks, moles, nevi, skin tags, lipomas, angiomas including hemangiomas, and other cutaneous lesions for cosmetic or other purposes.

Expression of HGF/SF, and its receptor, c-Met, is often associated with malignant progression (metastasis) of human tumors, including gliomas. Overexpression of HGF/SF in experimental gliomas enhances tumorigenicity and tumor-associated angiogenesis (i.e., growth of new blood vessels). More recent studies showed that human glioblastomas are HGF/SF-c-Met dependent and that a reduction in endogenous HGF/SF or c-Met expression can lead to inhibition of tumor growth and tumorigenicity. Thus, targeting the HGF/SF-c-Met signaling pathway using a compound as characterized above is an important approach in controlling tumor progression.

In addition, compounds of the invention have been found to also inhibit or antagonize Tie-2, a receptor tyrosine kinase involved in angiogenesis. The Tie-2 receptor is exclusively expressed on endothelial cells and plays an important role in the regulation of vascular remodeling. Inhibiting Tie-2 by compounds of the invention is another means for preventing the growth of tumors, by impairing formation of tumor vasculature and destabilizing existing vessels, and inhibiting the inappropriate vascularization that occurs in inflammatory processes. It is a further aspect of the present invention to provide compounds and pharmaceutical compositions that inhibit or antagonize Tie-2, and methods of using such compounds and pharmaceutical compositions for the treatment of dysproliferative diseases such as cancer, as well as inflammatory diseases and conditions.

In cases where abnormal or excessive cellular proliferation is the cause of pathology, such as in dysproliferative diseases including various cancers, inflammatory joint and skin diseases such as atherosclerosis, rheumatoid arthritis, and neovascularization in the eye as a consequence of diabetic retinopathy, suppression of cellular proliferation is a desired goal in treatment. Certain compounds of the invention are particularly beneficial for the treatment of cancer and other dysproliferative diseases and conditions. As compounds of the invention have been found to possess antiproliferative activity on cells, as well as antiangiogenic activity, both activities may be beneficial in the treatment of, for example, solid tumors, in which both the dysproliferative cells and the enhanced tumor vasculature elicited thereby are targets for inhibition by the agents of the invention. In either case, therapy to promote or suppress proliferation may be beneficial locally but not systemically, and for a particular duration, and proliferation modulating therapies must be appropriately applied. The invention embraces localized delivery of such compounds to the affected tissues and organs to achieve a particular effect.

As noted above, other uses of the compounds herein include intentional ablation or destruction of tissues or organs in a human or animal, for example, in the area of animal husbandry, and in the field of reproductive biology, to reduce the number of developing embryos; as an abortifacient, and as a means to achieve a biochemical castration, particularly for livestock and domesticated animals such as pets. Such animals are furthermore candidates for treatment of any of the dysproliferative diseases including cancers and other conditions described herein.

As mentioned above, vascularization of the vitreous humor of the eye as a consequence of diabetic retinopathy is a major cause of blindness, and inhibition of such vascularization is desirable. Other conditions in which vascularization is undesirable include certain chronic inflammatory diseases, in particular inflammatory joint and skin disease, but also other inflammatory diseases in which a proliferative response occurs and is responsible for part or all of the pathology. For example, psoriasis is a common inflammatory skin disease characterized by prominent epidermal hyperplasia and neovascularization in the dermal papillae. Proliferation of smooth muscle cells, perhaps as a consequence of growth factors, is a factor in the narrowing and occlusion of the macrovasculature in atherosclerosis, responsible for myocardial ischemia, angina, myocardial infarction, and stroke, to name a few examples. Peripheral vascular disease and arteriosclerosis obliterans comprise an inflammatory component as well, and thus amenable to therapeutic intervention with compounds of the invention.

### DEFINITIONS

For convenience, certain terms employed in the specification, examples, and appended claims are collected here.

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, or alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms. “Lower alkenyl” and “lower alkynyl” respectively include corresponding 1-6 carbon moieties.

In certain embodiments, the alkyl, and the unsaturated alkenyl and alkynyl groups employed in the invention contain 1-20; 2-20; 3-20; 4-20; 5-20; 6-20; 7-20 or 8-20 aliphatic carbon atoms. In certain other embodiments, the alkyl, alkenyl, and alkynyl groups employed in the invention contain 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl, alkenyl, and alkynyl groups employed in the invention contain 1-6; 2-6; 3-6; 4-6 or 5-6 aliphatic carbon atoms. In yet other embodiments, the alkyl, alkenyl, and alkynyl groups employed in the invention contain 1-4; 2-4 or 3-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl(propargyl), 1-propynyl and the like.

The term “alicyclic” or “cycloalkyl,” as used herein, refers to compounds which combine the properties of aliphatic and cyclic compounds and include but are not limited to monocyclic, or polycyclic aliphatic hydrocarbons and bridged cycloalkyl compounds, which are optionally substituted with one or more functional groups. As will be appreciated by one of ordinary skill in the art, “alicyclic” or “cycloalkyl” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In certain embodiments, the alkyl group contains 1-20; 2-20; 3-20; 4-20; 5-20; 6-20; 7-20 or 8-20 aliphatic carbon atoms. In certain other embodiments, the alkyl group contains 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl group contains 1-6; 2-6; 3-6; 4-6 or 5-6 aliphatic carbon atoms. In yet other embodiments, the alkyl group contains 1-4; 2-4 or 3-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “thioalkyl” or “—S-” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of thioalkyl include, but are not limited to, methylthio, ethylthio, propylthio, isopropylthio, n-butylthio, and the like. Moreover, this group of the invention may be substituted by an aromatic or heteroaromatic group, which may be even further substituted.

The term “alkylamino” refers to a group having the structure —NHR′ wherein R′ is aliphatic or alicyclic, as defined herein. The term “aminoalkyl” refers to a group having the structure NH₂R′—, wherein R′ is aliphatic or alicyclic, as defined herein. In certain embodiments, the aliphatic or alicyclic group contains 1-20 aliphatic carbon atoms. In certain other embodiments, the aliphatic or alicyclic group contains 1-10 aliphatic carbon atoms. In still other embodiments, the aliphatic or alicyclic group contains 1-6 aliphatic carbon atoms. In yet other embodiments, the aliphatic or alicyclic group contains 1-4 aliphatic carbon atoms. In yet other embodiments, R′ is an alkyl, alkenyl, or alkynyl group containing 1-8 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; —F; —Cl; —Br; —I; —OH; ═O; —SH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; and —S(O)₂N(Rₓ)₂; wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted. Additional examples of generally applicable substituents are illustrated by the specific embodiments shown in the Examples that are described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaromatic moiety”, as used herein, refers to a stable mono- or polycyclic, unsaturated moiety having preferably 3-14 carbon atoms, each of which may be substituted or unsubstituted; and comprising at least one heteroatom selected from the group consisting of O, S and N within the ring (i.e., in place of a ring carbon atom). In certain embodiments, the term “heteroaromatic moiety” refers to a planar ring comprising at least one heteroatom, having p-orbitals perpendicular to the plane of the ring at each ring atom, and satisfying the Huckel rule where the number of pi electrons in the ring is (4n+2) wherein n is an integer.

It will also be appreciated that aromatic and heteroaromatic moieties, as defined herein may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl and the like.

The term “heteroaryl” or “heteroaromatic”, as used herein, does not differ significantly from the common meaning of the term in the art, and refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, thiazinyl, isoquinolinyl, and the like.

It will be appreciated that aryl, heteroaromatic and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; —F; —Cl; —Br; —I; —OH; —SH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; and —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; —F; —Cl; —Br; —I; —OH; ═O; —SH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; and —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; —F; —Cl; —Br; —I; —OH; ═O; —SH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SR^{x}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; and —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from the group consisting of O, S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from the group consisting of O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, pyrazolyl, imidazolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; —F; —Cl; —Br; —I; —OH; ═O; —SH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; and —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto. Additional examples of generally applicable substituents are illustrated by the specific embodiments shown in the Examples that are described herein.

The terms “halo” and “halogen” as used herein refer to an atom or substituent selected from the group consisting of fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z}) amine, where Rₓ, R_{y} and R_{z} are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein, refers to a group having the general formula —C(═O)R, where R is an aliphatic, alicyclic, heteroaliphatic, heterocyclic, aromatic or heteroaromatic moiety, as defined herein.

The term “C₁₋₆ alkylidene”, as used herein, refers to a substituted or unsubstituted, linear or branched saturated divalent radical consisting solely of carbon and hydrogen atoms, having from two to six carbon atoms, having a free valence “-” at both ends of the radical.

The term “C₂₋₆ alkenylidene”, as used herein, refers to a substituted or unsubstituted, linear or branched unsaturated divalent radical consisting solely of carbon and hydrogen atoms, having from two to six carbon atoms, having a free valence “-” at both ends of the radical, and wherein the unsaturation is present only as double bonds and wherein a double bond can exist between the first carbon of the chain and the rest of the molecule.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aromatic”, “heteroaromatic”, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Another example is an N-methyl derivative of a compound, which is susceptible to oxidative metabolism resulting in N-demethylation. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

### Preparation of Compounds of the Invention

General Description of Synthetic Methods. The practitioner has a well-established literature of small molecule chemistry to draw upon, in combination with the information contained herein, for guidance on synthetic strategies, protecting groups, and other materials and methods useful for the synthesis of the compounds of this invention. The various references cited herein provide helpful background information on preparing compounds similar to the inventive compounds described herein or relevant intermediates, as well as information on formulation, uses, and administration of such compounds which may be of interest. Moreover, the practitioner is directed to the specific guidance and examples provided in this document relating to various exemplary compounds and intermediates thereof.

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

According to the present invention, any available techniques can be used to make or prepare the inventive compounds or compositions including them. For example, a variety of solution phase synthetic methods such as those discussed in detail below may be used. Alternatively or additionally, the inventive compounds may be prepared using any of a variety combinatorial techniques, parallel synthesis and/or solid phase synthetic methods known in the art.

It will be appreciated as described below, that a variety of inventive compounds can be synthesized according to the methods described herein. The starting materials and reagents used in preparing these compounds are either available from commercial suppliers such as Aldrich Chemical Company (Milwaukee, Wis.), Bachem (Torrance, Calif.), Sigma (St. Louis, Mo.), or are prepared by methods well known to a person of ordinary skill in the art following procedures described in such references as Fieser and Fieser 1991, “Reagents for Organic Synthesis”, vols 1-17, John Wiley and Sons, New York, N.Y., 1991; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; and Larock 1990, “Comprehensive Organic Transformations: A Guide to Functional Group Preparations”, 2nd ed. VCH Publishers. These schemes are merely illustrative of some methods by which the compounds of this invention can be synthesized, and various modifications to these schemes can be made and will be suggested to a person of ordinary skill in the art having regard to this disclosure.

The starting materials, intermediates, and compounds of this invention may be isolated and purified using conventional techniques, including filtration, distillation, crystallization, chromatography, and the like. They may be characterized using conventional methods, including physical constants and spectral data.

General Reaction Procedures. Unless mentioned specifically, reaction mixtures were stirred using a magnetically driven stirrer bar. An inert atmosphere refers to either dry argon or dry nitrogen. Reactions were monitored either by thin layer chromatography, by proton nuclear magnetic resonance (NMR) or by high-pressure liquid chromatography (HPLC), of a suitably worked up sample of the reaction mixture.

General Work Up Procedures. Unless mentioned specifically, reaction mixtures were cooled to room temperature or below then quenched, when necessary, with either water or a saturated aqueous solution of ammonium chloride. Desired products were extracted by partitioning between water and a suitable water-immiscible solvent (e.g., ethyl acetate, dichloromethane, diethyl ether). The desired product containing extracts were washed appropriately with water followed by a saturated solution of brine. On occasions where the product containing extract was deemed to contain residual oxidants, the extract was washed with a 10% solution of sodium sulphite in saturated aqueous sodium bicarbonate solution, prior to the aforementioned washing procedure. On occasions where the product containing extract was deemed to contain residual acids, the extract was washed with saturated aqueous sodium bicarbonate solution, prior to the aforementioned washing procedure (except in those cases where the desired product itself had acidic character). On occasions where the product containing extract was deemed to contain residual bases, the extract was washed with 10% aqueous citric acid solution, prior to the aforementioned washing procedure (except in those cases where the desired product itself had basic character). Post washing, the desired product containing extracts were dried over anhydrous magnesium sulphate, and then filtered. The crude products were then isolated by removal of solvent(s) by rotary evaporation under reduced pressure, at an appropriate temperature (generally less than 45° C.).

General Purification Procedures. Unless mentioned specifically, chromatographic purification refers to flash column chromatography on silica, using a single solvent or mixed solvent as eluent. Suitably purified desired product containing elutes were combined and concentrated under reduced pressure at an appropriate temperature (generally less than 45° C.) to constant mass. Final compounds were dissolved in 50% aqueous acetonitrile, filtered and transferred to vials, then freeze-dried under high vacuum before submission for biological testing.

Synthesis of Exemplary Compounds. Compounds of the invention are prepared as illustrated in Scheme 1 below. For example, for the preparation of one exemplary compound of the invention, treatment of 2,4-quinazolinedione (1) with POCl₃ will afford the dichloroquinazoline 2. Displacement of the 4-chloro moiety with cyclopentylamine for an extended period (2-4 days) will provide intermediate 3, and subsequent displacement of the 2-chloro moiety with reagent 4 at elevated temperatures will afford the desired compound. The final product is analyzed by ¹H NMR, ¹³C NMR, LC/MS, elemental analysis, and melting point. A purity level of greater than about 95% will be targeted.

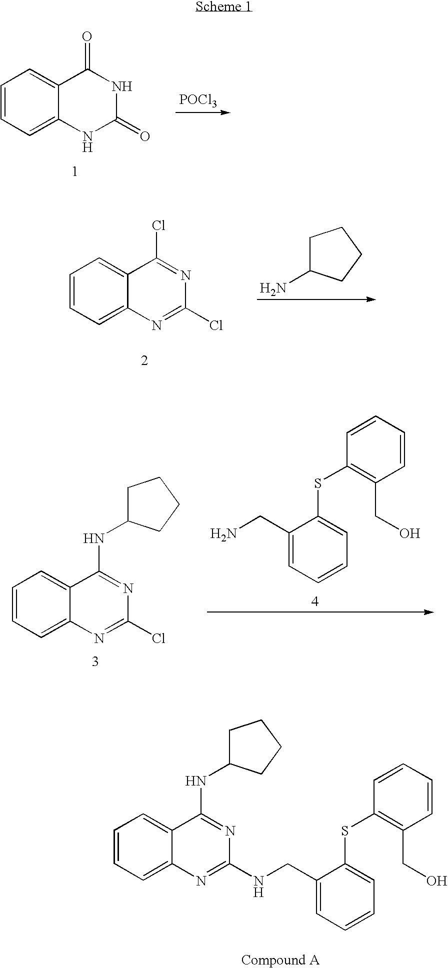

To prepare compounds of the invention with different substituents on the diaminoquinazoline core, the cyclopentylamine and reagent 4 are replaced with the corresponding reagents to afford the desired compound.

For example, to prepare the compound shown in the above scheme, the reagent used to prepare compound 3 from compound 2 is cyclopentylamine, available from Aldrich Chemical Co., Milwaukee Wis.; reagent 4 is 2-[2-(aminomethyl)phenylthio]benzyl alcohol, also available from Aldrich.

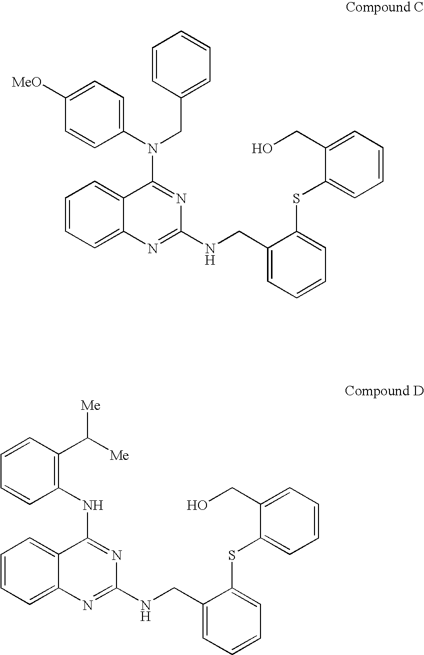

To prepare the compounds shown directly above (compounds C and D) the same approach as shown in Scheme 1 may be taken: compound 2 is reacted N-benzyl-4-methoxyaniline and 2-isopropylaniline, instead of compound 3, and the final step, reaction with reagent 4, is the same as presented in Scheme 1.

Compounds of the invention wherein any R¹ is not hydrogen are prepared from the R¹-substituted reagent 1. The aforementioned synthetic route is merely exemplary of one way in which to prepare the compounds of the invention; alternate procedures will be readily apparent to one of skill in the art.

Moreover, to convert the final product above to another compound of the invention, straightforward reactions may be undertaken. For example, the following scheme (Scheme 2 below) illustrates modification of the terminal hydroxy group into the methyl ether using iodomethane or dimethylsulfate (compound F), and the fluorine-replaced analog using (diethylamino)sulfur trifluoride (DAST) (compound E).

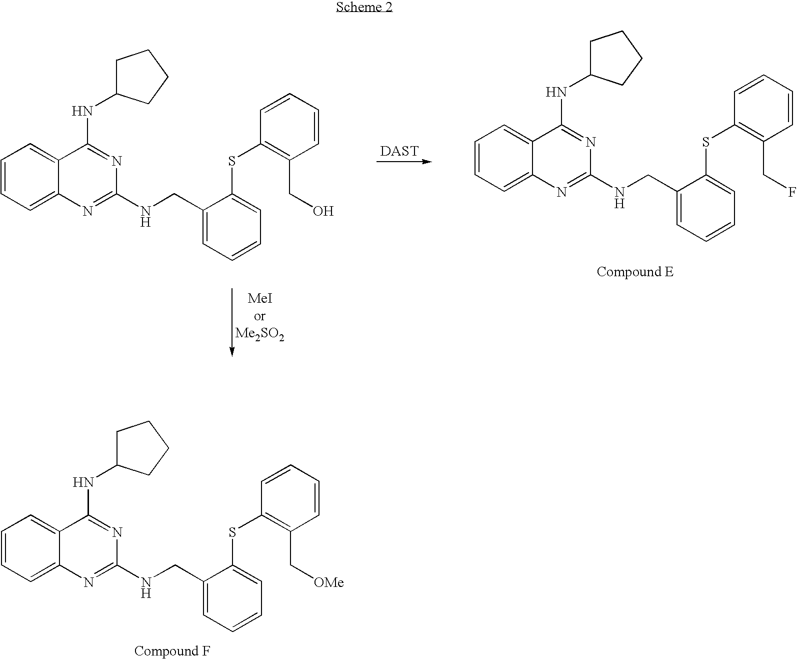

In embodiments of the invention wherein analogs of compounds of Formula II are provided, and in particular wherein at least one of R³, R⁴, R⁴, R⁵ and R⁶ is not hydrogen, the following synthetic route may be used.

In one example, compounds of Formula II, wherein X¹ is cyclopentyl and X² is hydrogen, R² is —SR^{R}, and R^{R} is 2-hydroxymethyl, are depicted as Formula III:

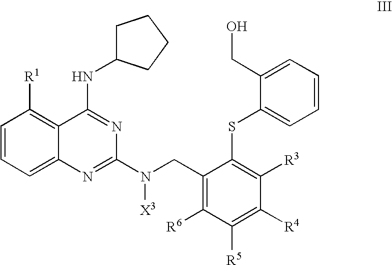

In certain embodiments, compounds of formula III may be prepared in accordance with the following three steps, shown in the schemes 3-5 below.

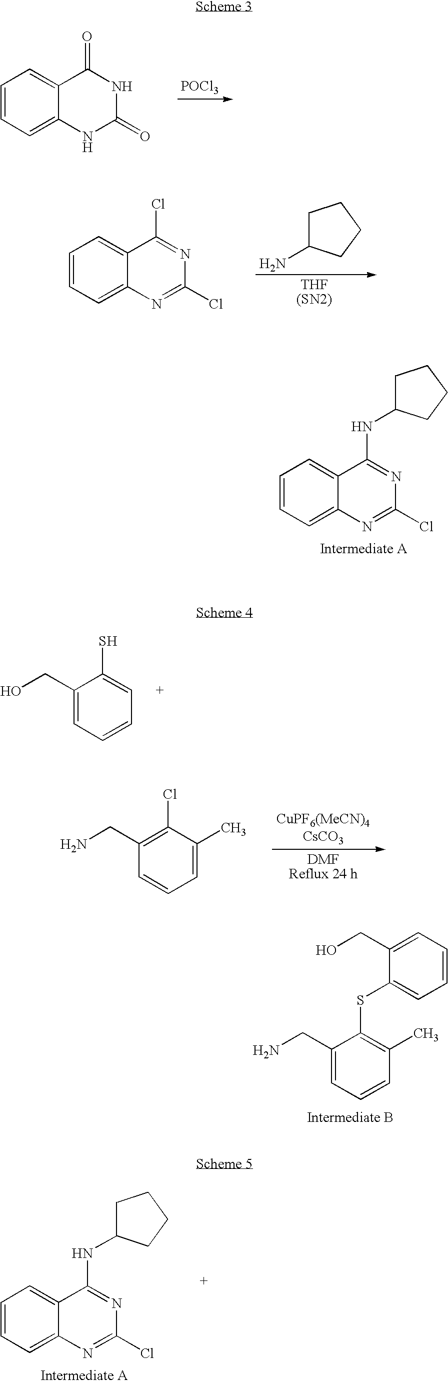

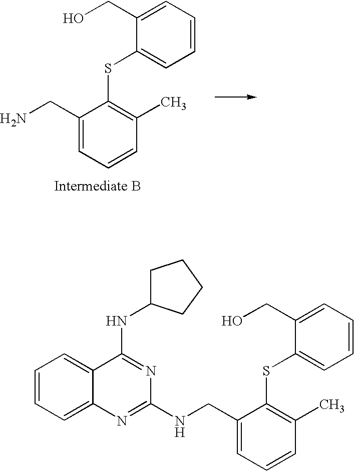

In an analogous fashion, by using the corresponding intermediates, compound of formula III, with the following selections of R³, R⁴, R⁵ and R⁶, can be prepared from the corresponding starting compound 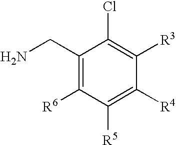
wherein R³ is H, R⁴ and R⁵ taken together are —OCH₂O—, and R⁶ is H; or R³ is OH, R⁴ is H, R⁵ is H, and R⁶ is H; or R³ is H, R⁴ is OH, R⁵ is H, and R⁶ is H; or R³ is H, R⁴ is OH, R⁵ is H, and R⁶ is H; or R³ is H, R⁴ is H, R⁵ is H, and R⁶ is OH; or R³ is Cl, R⁴ is H, R⁵ is H, and R⁶ is H; or R³ is H, R⁴ is Cl, R⁵ is H, and R⁶ is H; or R³ is H, R⁴ is H, R⁵ is Cl, and R⁶ is H; or R³ is H, R⁴ is H, R⁵ is H, and R⁶ is Cl; or R³ is H, R⁴ and R⁵ taken together are —SCH₂S—, and R⁶ is H; or R³ is H, R⁴ is OH, R⁵ is OH, and R⁶ is H. The foregoing compounds are merely exemplary and other combinations and positions of substituents, which are fully embraced herein.

In another route for preparing compounds of the invention, alternate Intermediate B reactants can be prepared as shown below in Scheme 6.

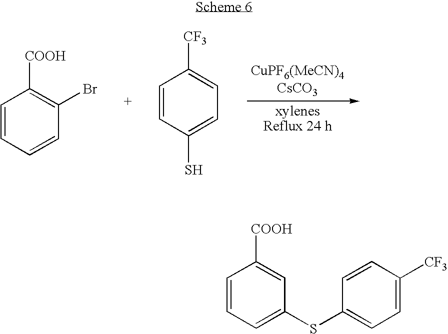

In certain embodiments, the compound of formula I, wherein X¹ is cyclopentyl and X² is hydrogen, can be prepared as shown in the following schemes (Schemes 7-10). 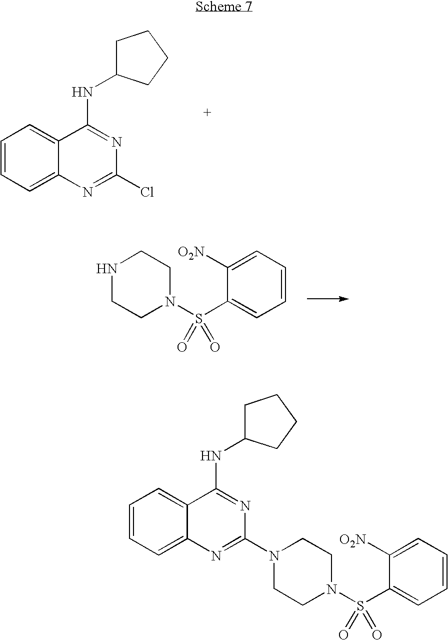 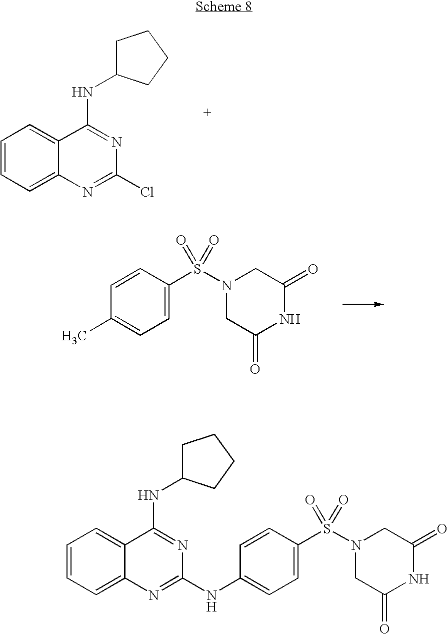 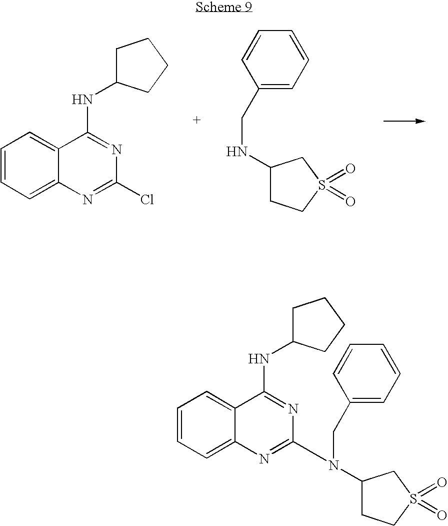 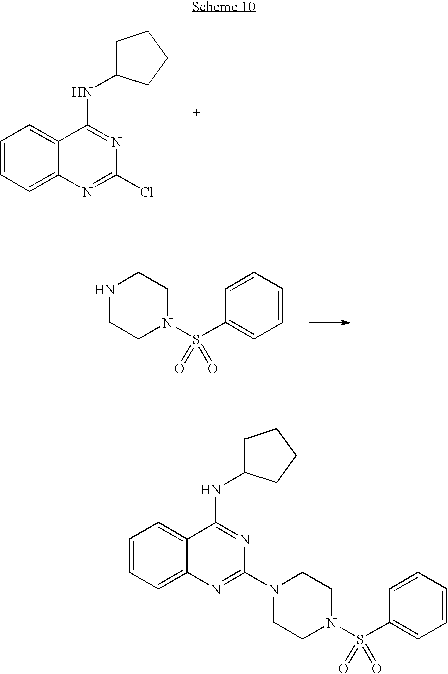
Compounds of the Invention

The compounds of the invention can be prepared by following the teachings described above. Variations on the synthetic schemes herein will be readily apparent to the skilled artisan for generating corresponding analogs of the various compounds of the invention.

One aspect of the invention relates to compounds represented by formula I:

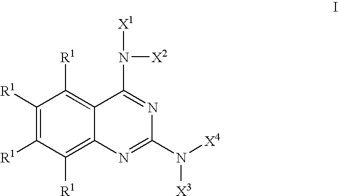

or a pharmaceutically acceptable salt thereof,

wherein, independently for each occurrence,

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

X¹, X², X³ and X⁴ are hydrogen or an optionally substituted aliphatic, alicyclic, heteroaliphatic, heterocyclic, aromatic, heteroaromatic or acyl group; or X¹ and X² taken together with the nitrogen to which they are bonded, or X³ and X⁴ taken together with the nitrogen to which they are bonded, are independently an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned compound wherein R¹ is hydrogen; halogen; a saturated or unsaturated, branched or straight-chain C₁₋₆ alkyl; aryl-C₁₋₆ alkyl; mono- or polyfluorinated C₁₋₆ alkyl; C₁₋₆ alkoxy; C₁₋₆ alkylamino; di(C₁₋₆ alkyl)amino; C₁₋₈ alkylamino-C₁₋₈ alkyl; di(C₁₋₆ alkyl)amino-C₁₋₈ alkyl; cyclo(C₃₋₆)alkyl; aryl, wherein the aryl comprises a six membered aromatic carbocycle (such as phenyl) or a polycyclic aromatic hydrocarbon (such as naphthyl, phenanthracenyl, indanyl); a heterocycle, wherein the heterocycle comprises six membered aromatic heterocycles (such as pyridyl, diazinyl, pyrimidinyl, pyrrolidinyl, piperazinyl, thiazinyl), five membered aromatic heterocycles (such as pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thienyl) or bicyclic systems (such as indolyl, benzothienyl, benzofuranyl, isoindolyl, isobenzothienyl, isobenzofuranyl); wherein any of wherein one or more of the foregoing aliphatic, cyclic, aromatic or heteroaromatic substituents optionally may be further substituted with a C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, cyano, hydroxy, carboxy, carboxy ester, amine (optionally substituted with C₁₋₆ straight chain alkyl), C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned compound wherein R¹ represents two non-hydrogen substituents which combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned compound, wherein R², R³, R⁴, R⁵ and R⁶, and the carbons to which they are bonded, may combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned compound wherein X¹, X², X³, and X⁴ are independently selected from the group consisting of hydrogen, a C₁₋₆ straight chain saturated or unsaturated alkyl group, a C₃₋₆ branched saturated or unsaturated chain alkyl group, a C₃₋₆ cycloalkyl group; and any of the foregoing are optionally substituted with one or more halo, nitro, cyano, hydroxy, carboxy, carboxy ester, amine (optionally substituted with C₁₋₆ straight chain alkyl), C₃₋₆ branched chain alkyl, C₃₋₆ cycloalkyl, aromatic group or aralkyl group (such as phenyl, benzyl or naphthyl, optionally further substituted as described above), fused alkyl or aromatic ring, or heteroaromatic or heterocyclic ring, which may be a saturated or unsaturated ring containing 4-10 ring members and 0-3 heteroatoms selected from the group consisting of O, N and S, the heteroaromatic or heterocyclic ring optionally substituted with one or more halo, C₁₋₆ straight chain alkyl, C₃₋₆ branched chain alkyl, C₃₋₆ cycloalkyl, C₁₋₆ alkyloxy, nitro, cyano, hydroxy, carboxyl, ester, amine (optionally substituted with C₁₋₆ straight chain alkyl), C₃₋₆ branched chain alkyl or C₃₋₆ cycloalkyl, trifluoroxy, trifluoromethyl, difluoromethyl, aryl, the same or different heterocyclic ring, or a fused aromatic, heteroaromatic or heterocyclic ring. The alkyl group of alkyloxy may be a C₁₋₆ straight chain, C₃₋₆ branched or C₃₋₆ cycloalkyl; and any of the alkyl groups herein may be saturated or contain one or more degrees of unsaturation; or X¹ and X² or X³ and X⁴ together with the nitrogen to which they are bonded is an optionally substituted heteroaromatic or heterocyclic ring comprising in addition to the aforementioned nitrogen, 4-10 ring members and 0-3 additional heteroatoms selected from the group consisting of O, N and S, the heteroaromatic or heterocyclic ring optionally further substituted with one or more aliphatic, aromatic, —SR^{R}, —OR^{R}, heteroaromatic or fused rings which may be further substituted as described herein.

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned compound wherein X¹ and X² taken together with the nitrogen to which they are bonded are an optionally substituted heterocyclic group comprising 4-10 ring members and 0-3 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned compound wherein X¹ and X² are hydrogen or an optionally substituted aliphatic, alicyclic, or aromatic group.

In certain embodiments, the present invention relates to the aforementioned compound wherein X¹ and X² are hydrogen, cyclopentyl, benzyl, 4-methoxyphenyl or 2-isopropylphenyl.

In certain embodiments, the present invention relates to the aforementioned compound wherein X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned compound wherein R¹ is hydrogen, halogen, C₁₋₆ alkyl, aryl-C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, aryl, or heterocycle; wherein one or more of the foregoing aliphatic, cyclic, aromatic or heteroaromatic substituents optionally may be further substituted with C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, fluoro, cyano, hydroxy, carboxy, carboxy ester, amine, C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned compound wherein R¹ is hydrogen, halogen, C₁₋₆ alkyl or C₁₋₆ alkoxy.

In certain embodiments, the present invention relates to the aforementioned compound wherein R¹ is hydrogen.

In the non-limiting, exemplary compounds of the invention described herein, any of the —NX¹X² substituents present amongst the various compounds described may be utilized as the —NX³X⁴ substituents, and vice versa. For example, in certain embodiments, the present invention relates to the aforementioned compound wherein —NX¹X² and/or —NX³X⁴ are independently selected from the group consisting of 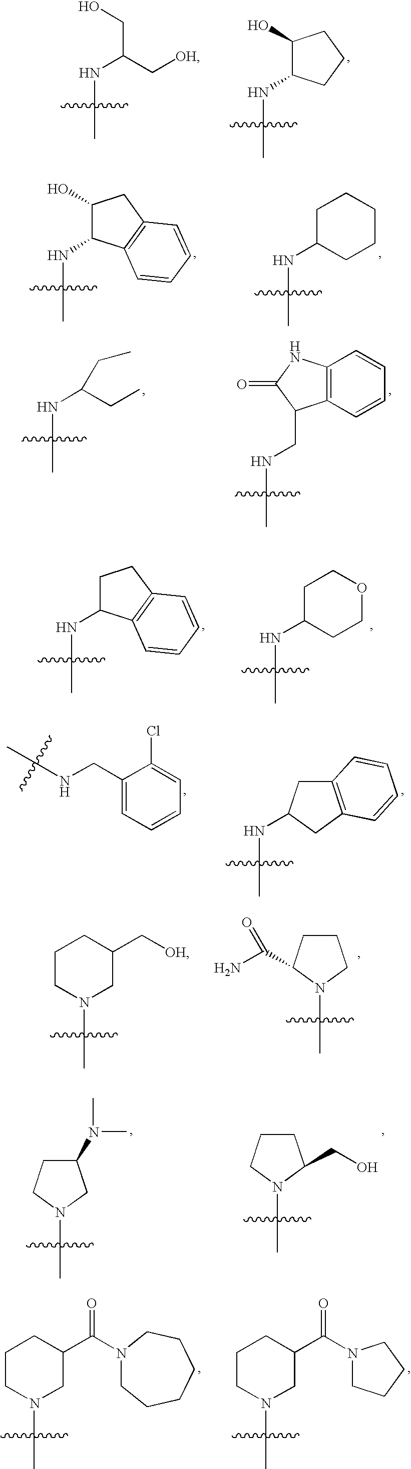 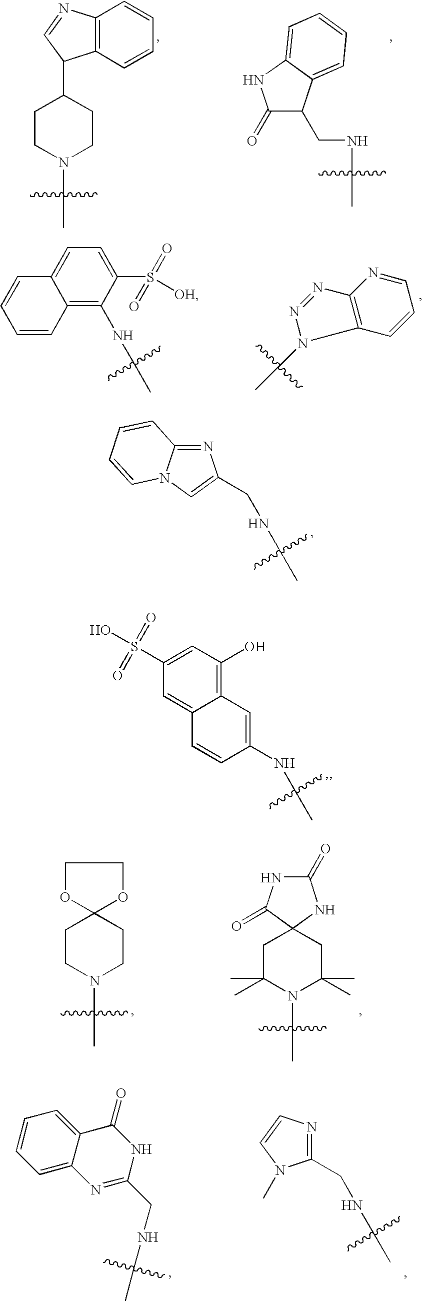 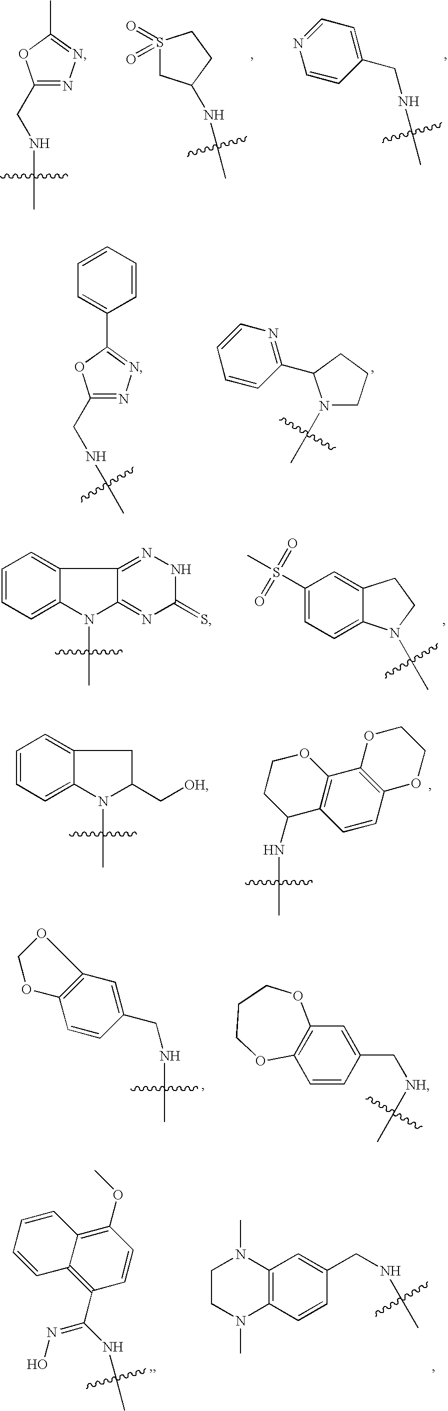 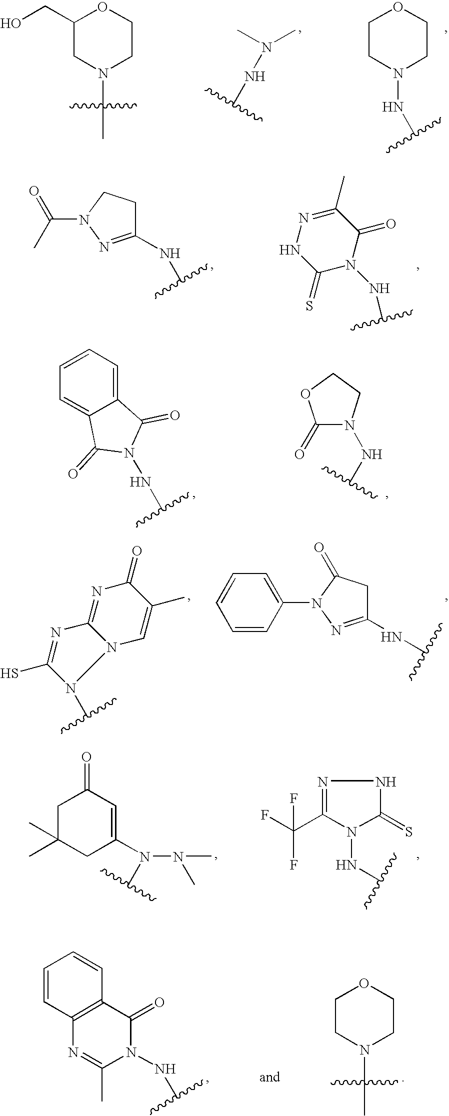
In addition, in certain embodiments, the present invention relates to the aforementioned compound wherein —NX¹X² and/or —NX³X⁴ are independently selected from the group consisting of

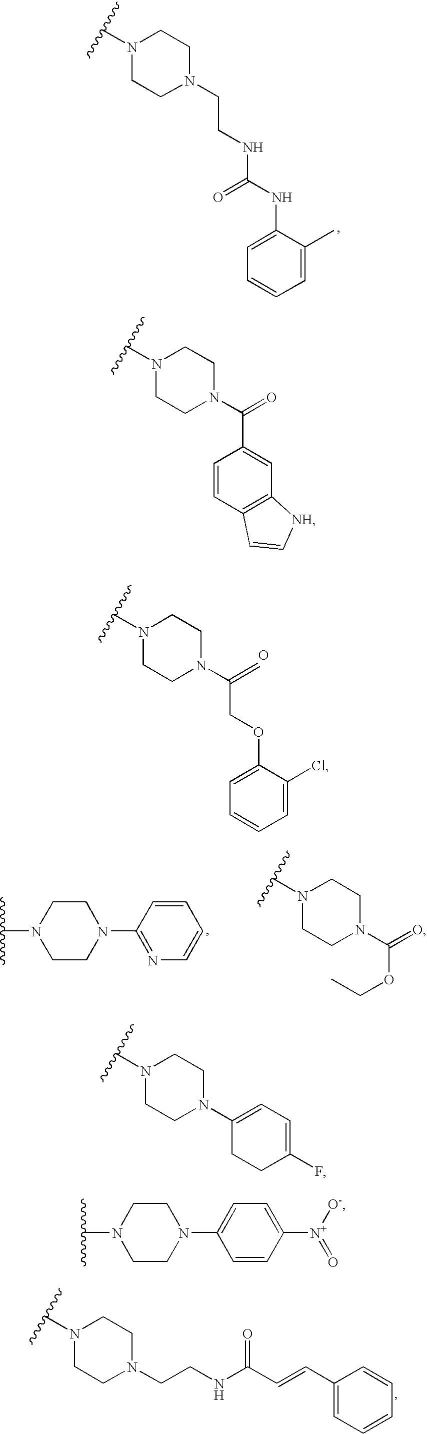

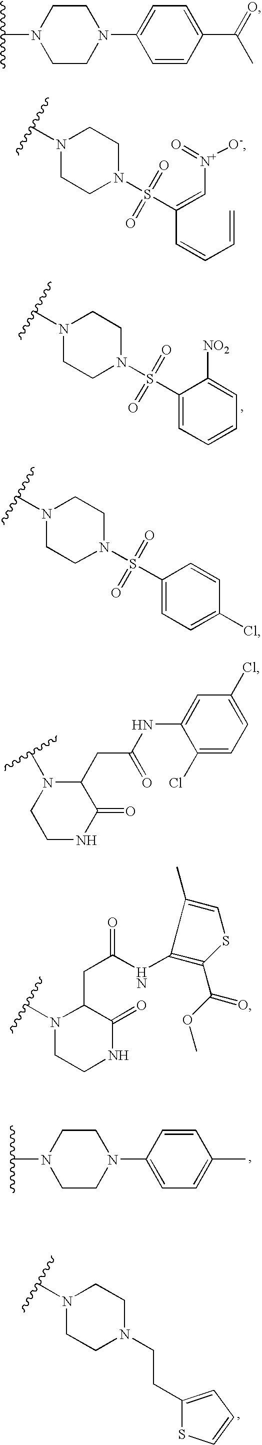

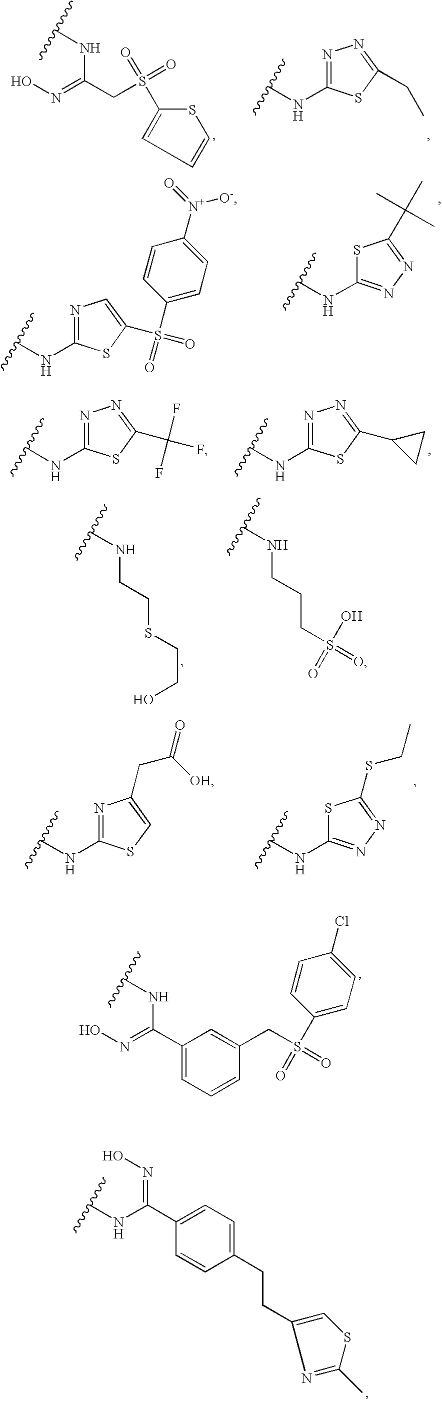

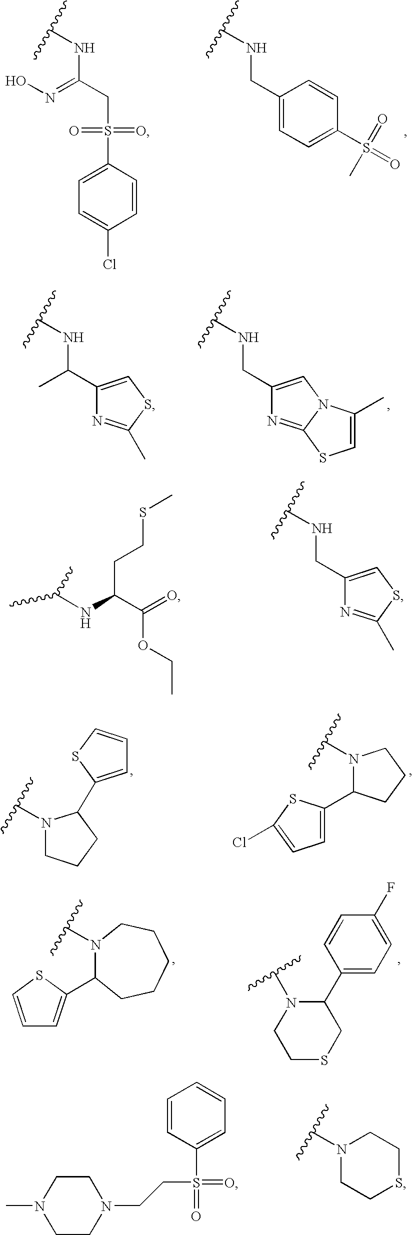

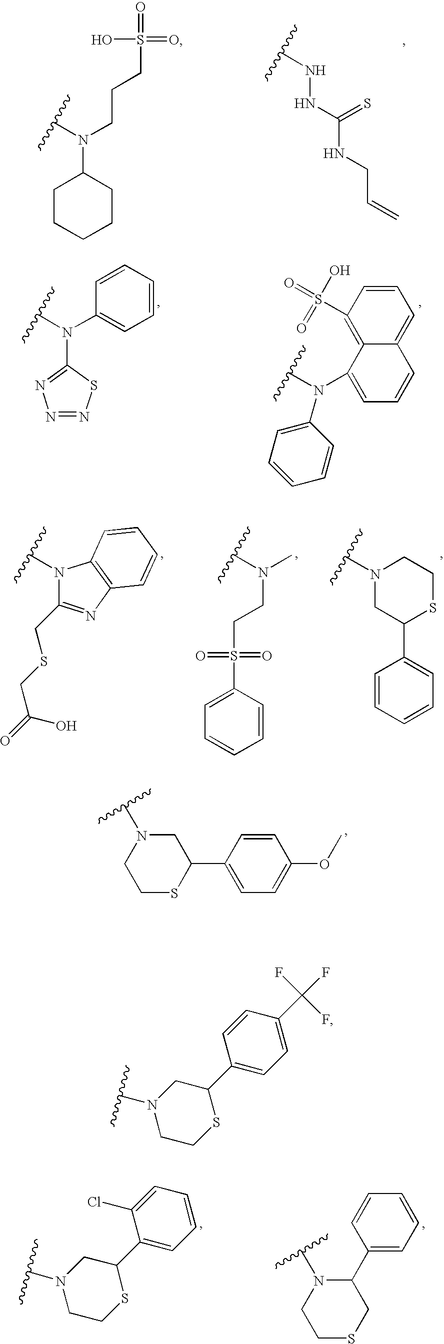

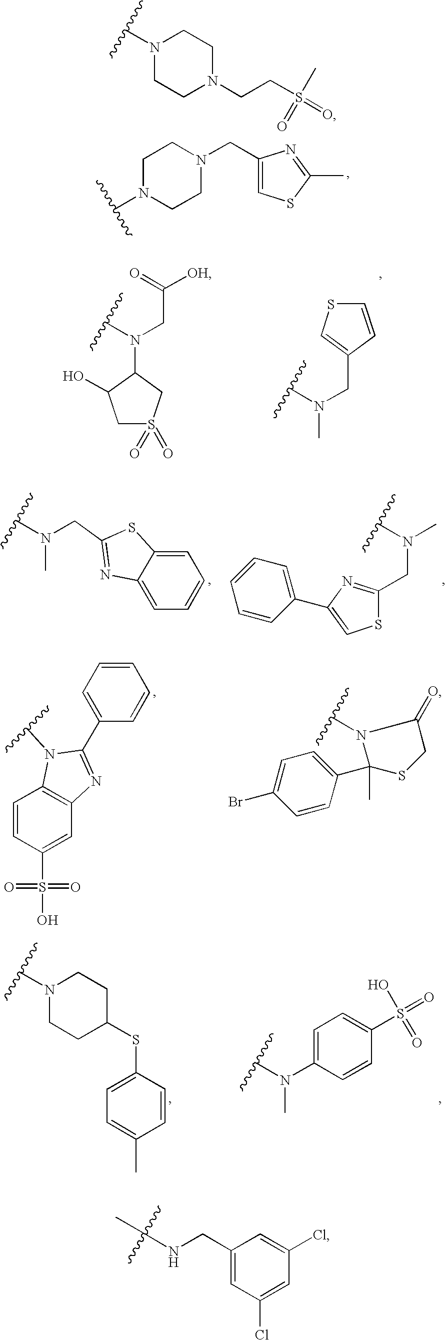

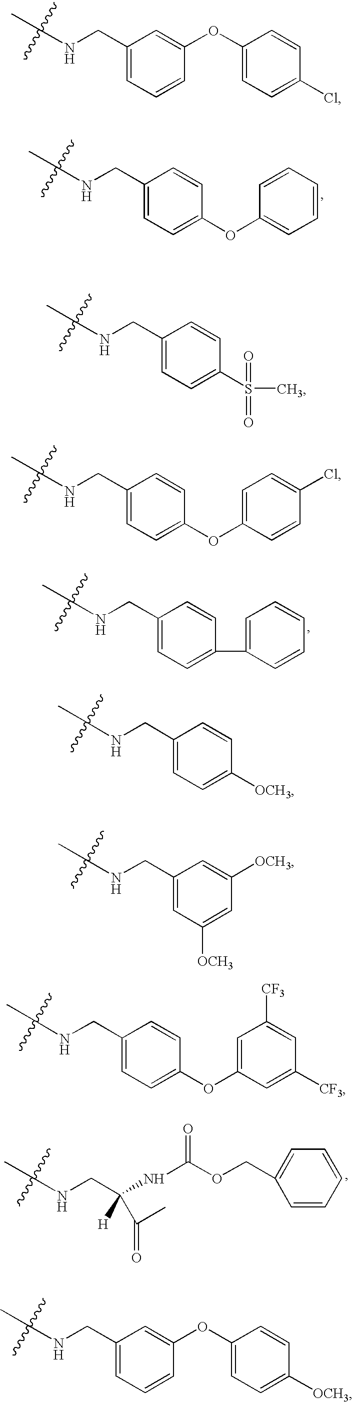

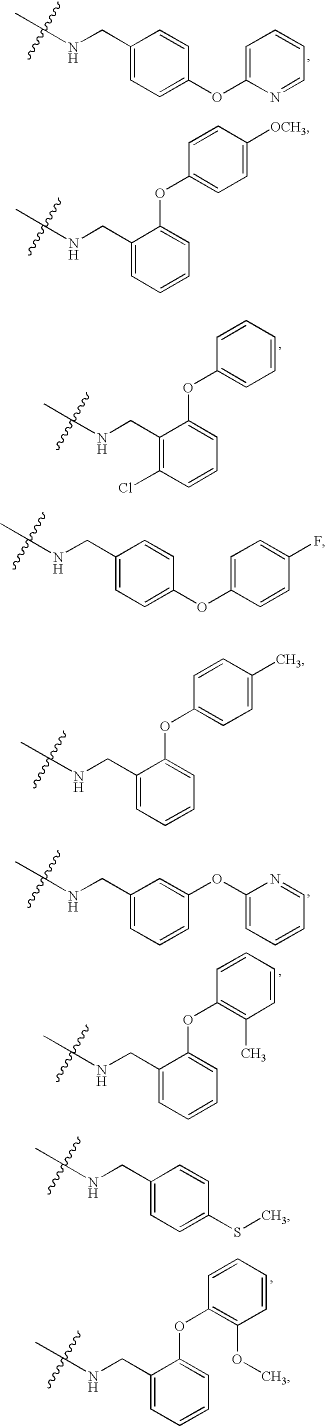

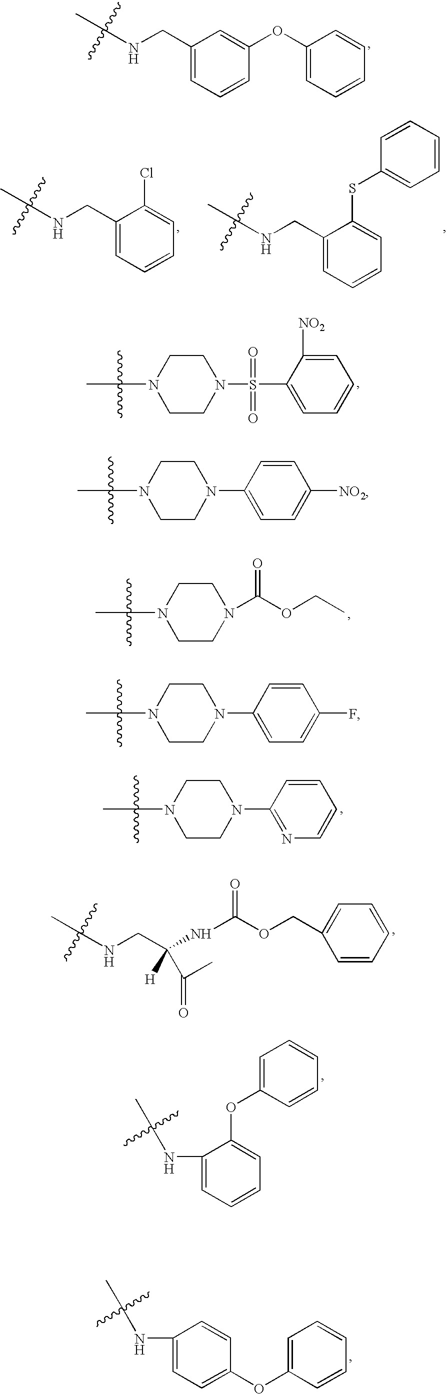

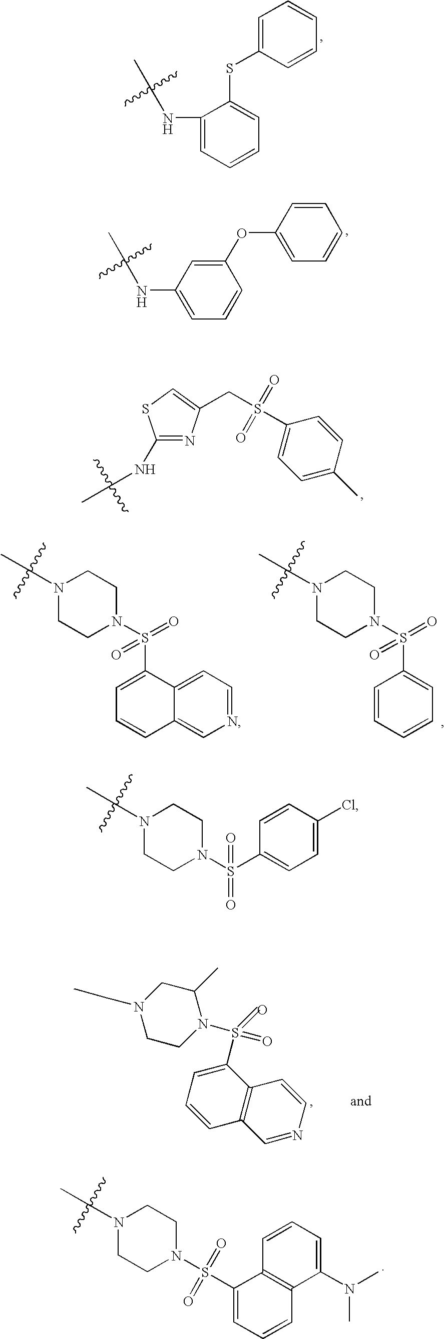

In certain embodiments, the present invention relates to the aforementioned compound wherein X¹ is cyclopentyl an X² is hydrogen. For example, one aspect of the invention relates to compounds selected from the group consisting of the compounds shown below.

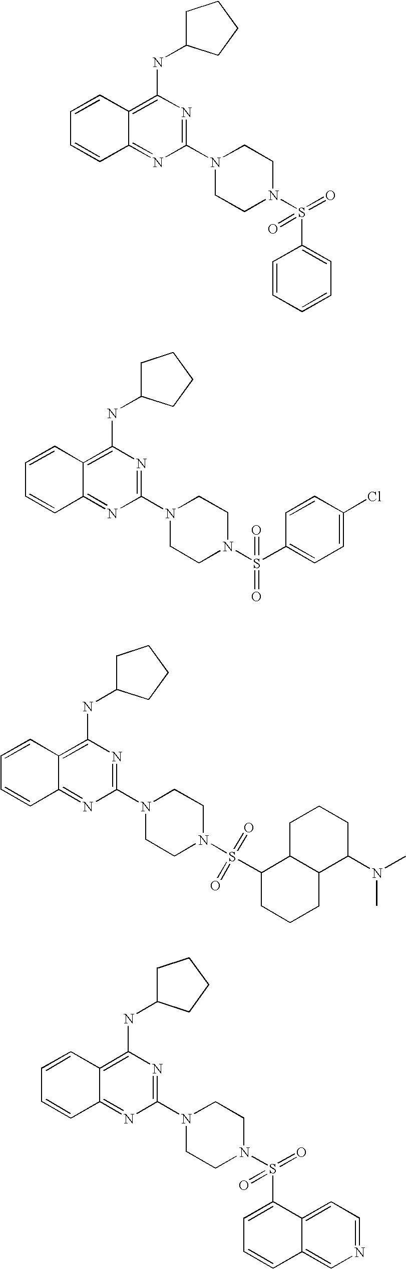

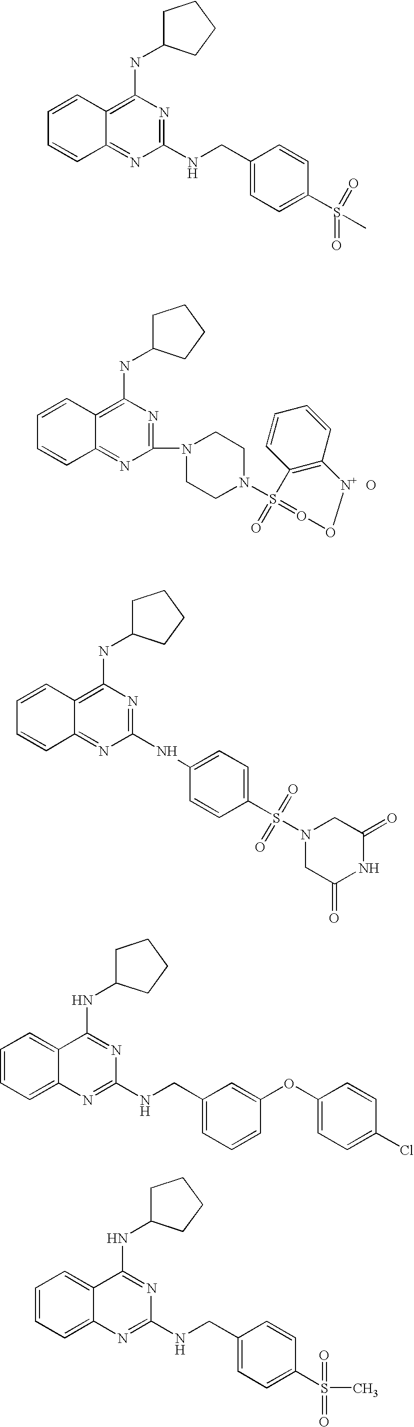

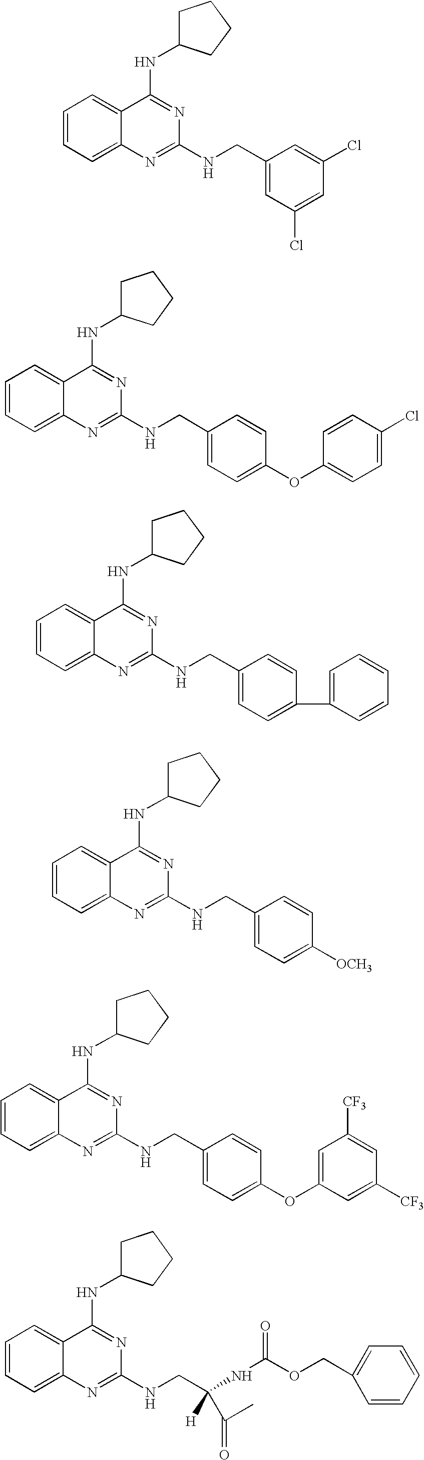

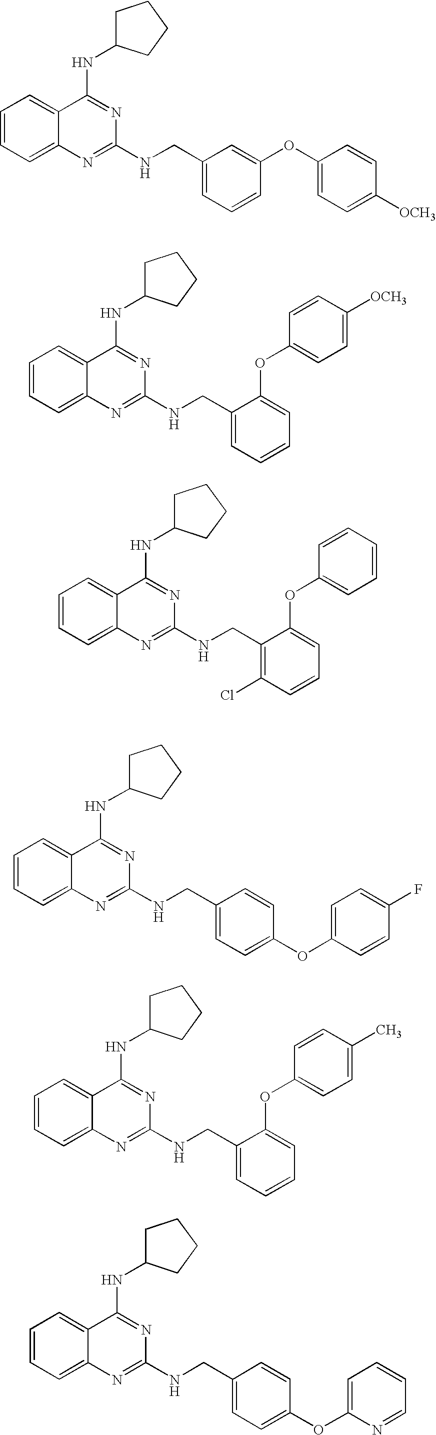

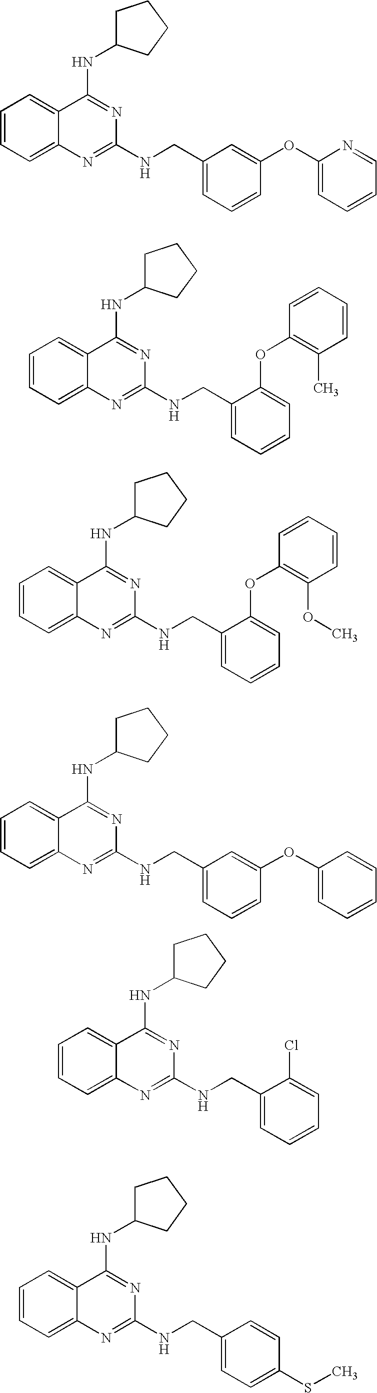

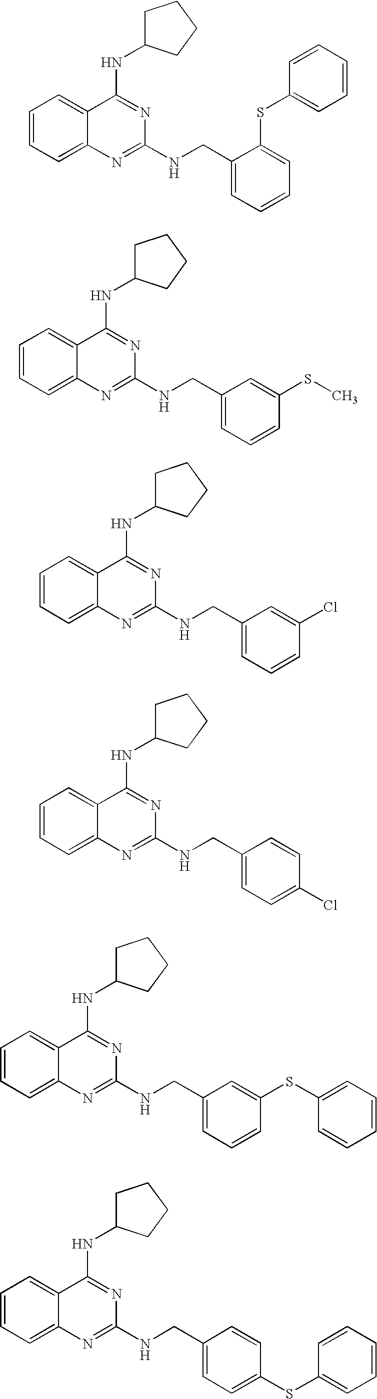

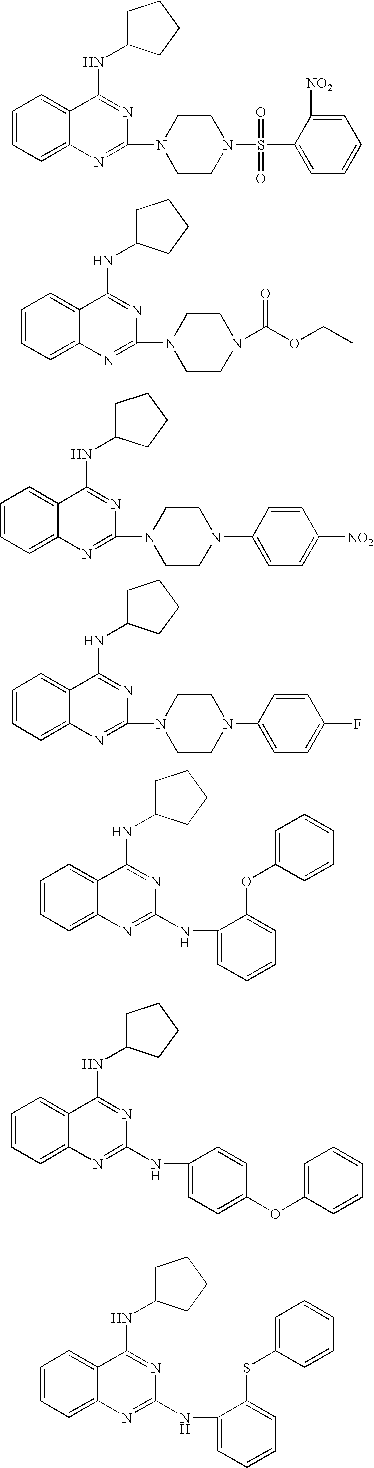

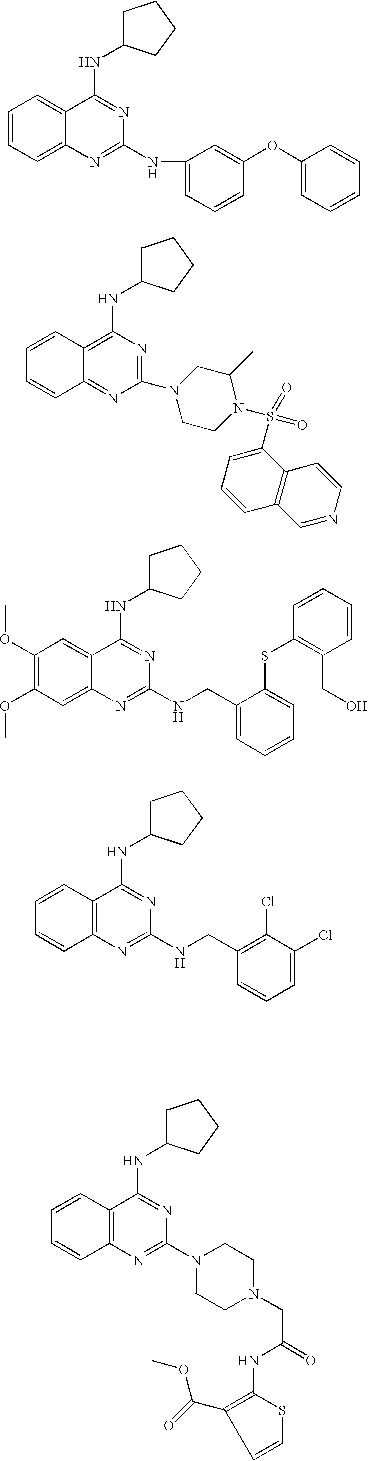

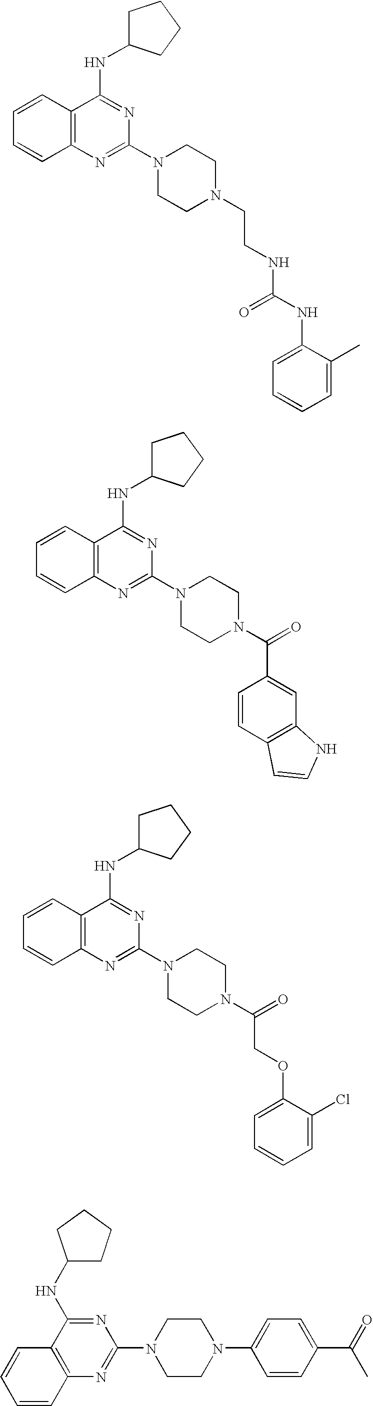

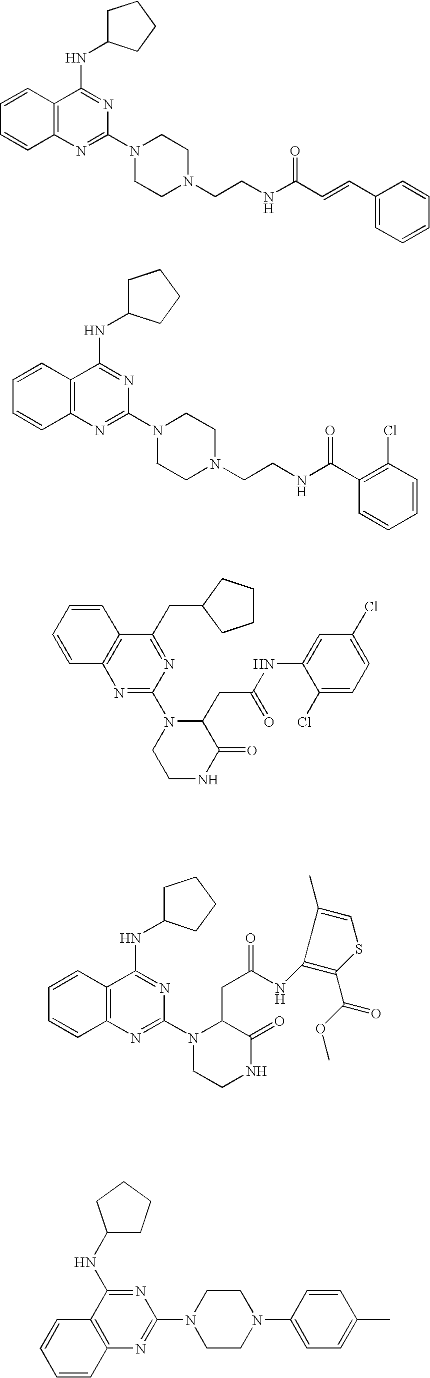

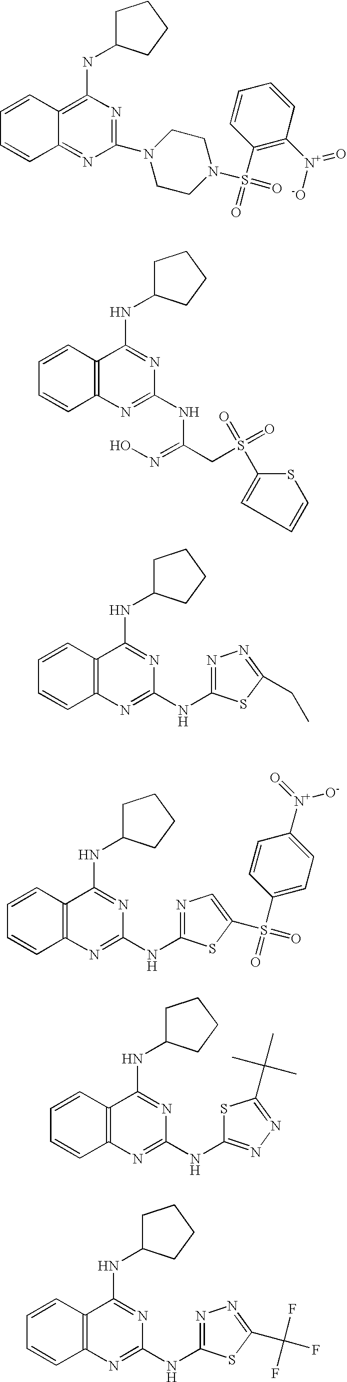

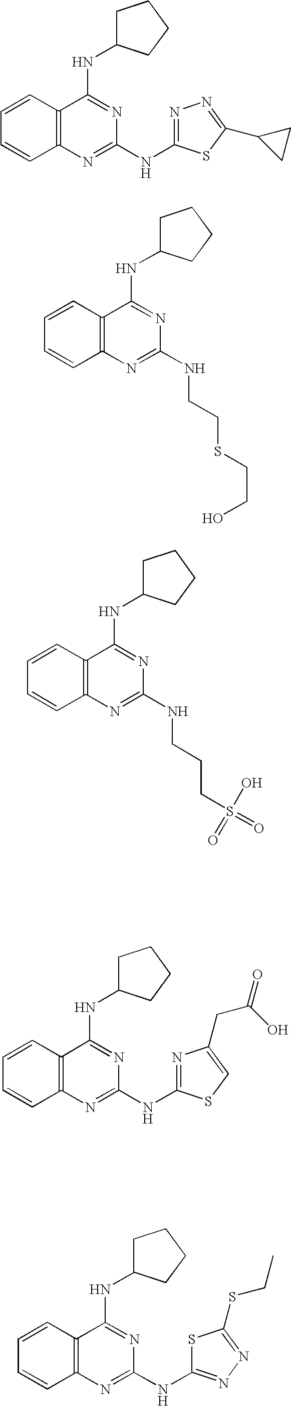

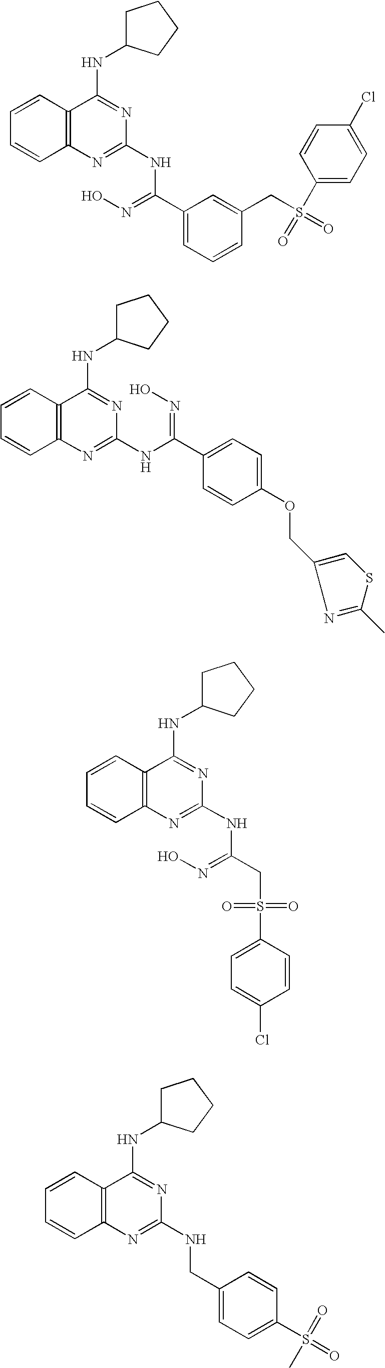

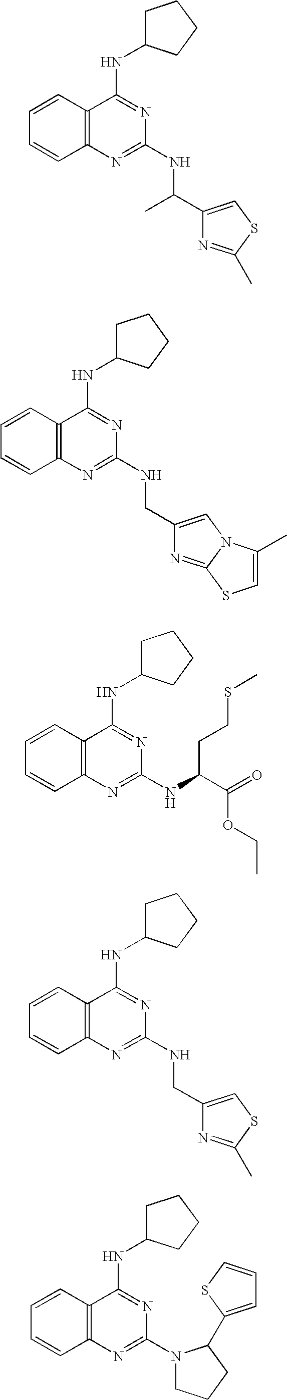

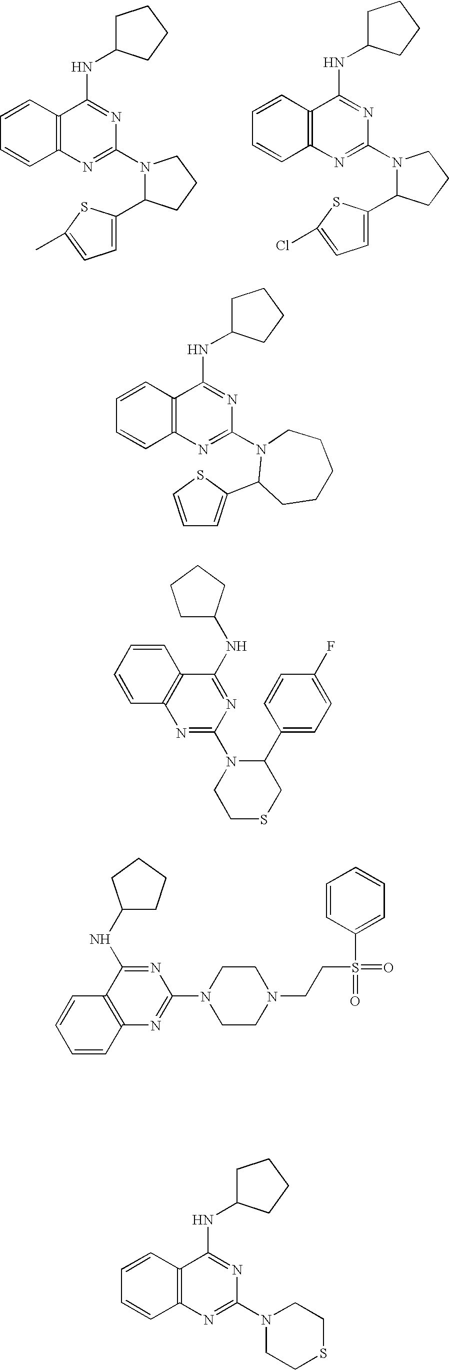

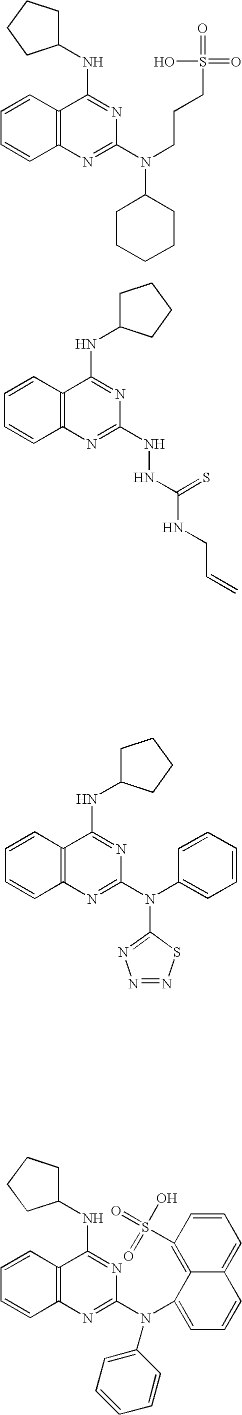

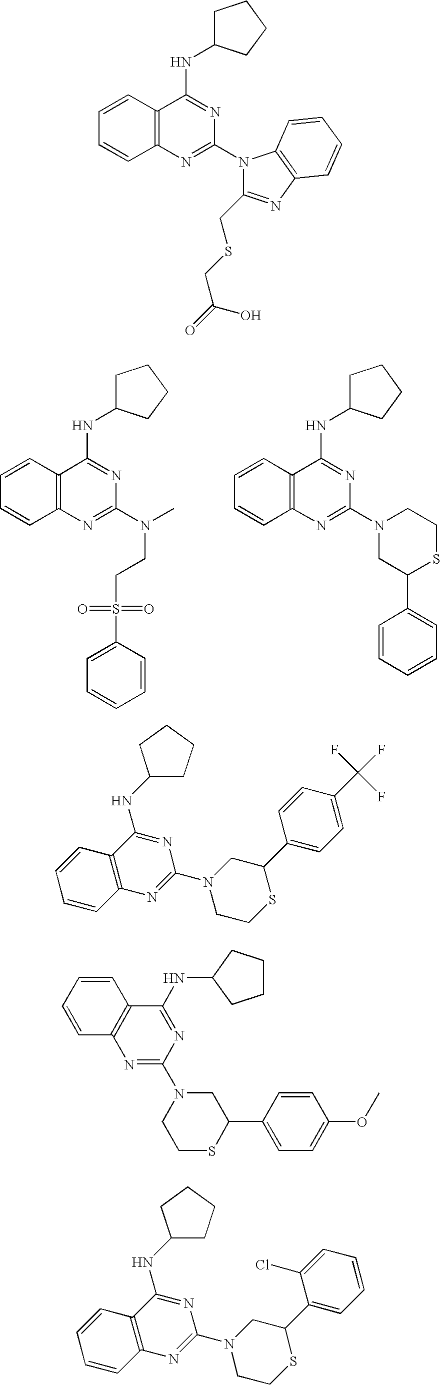

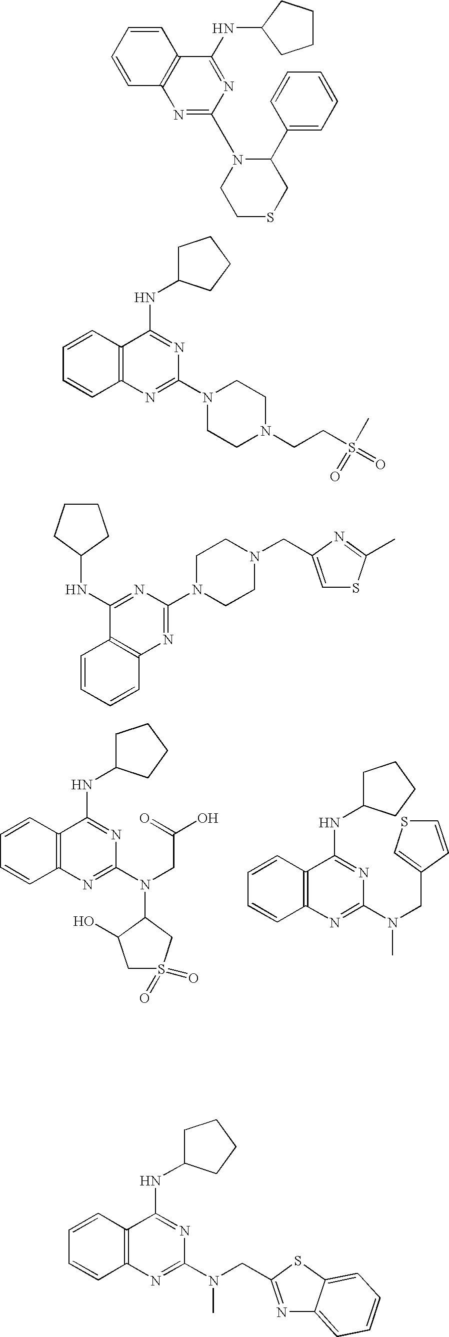

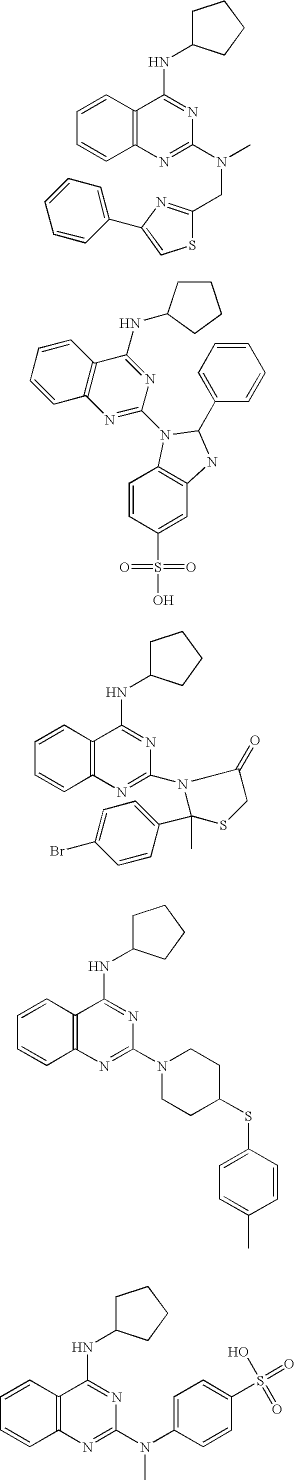

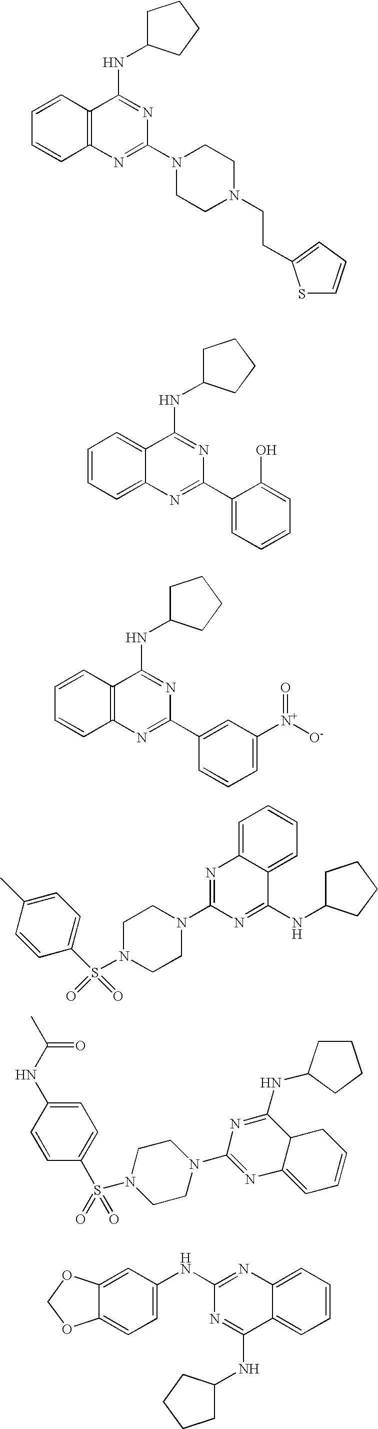

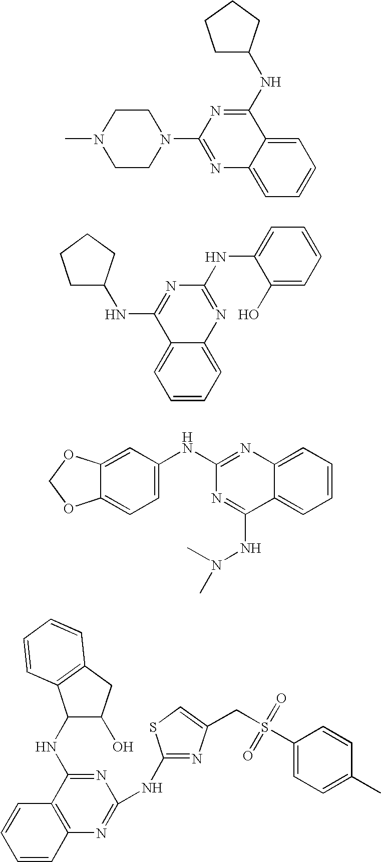

Another aspect of the present invention relates to a compound of formula II:

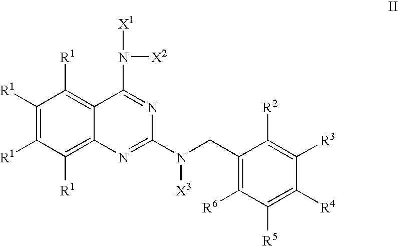

or a pharmaceutically acceptable salt thereof,

wherein, independently for each occurrence:

R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R²R³, R⁴, R⁵, and R⁶ are hydrogen, —F, —Cl, —Br, —I, —OH, —SH, —NO₂, —CN, —OR^{R}, —SR^{R}, S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹, X² and X³ are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned compound provided that when R¹ is hydrogen; R² is —SR^{R}; R³ is hydrogen; R⁴ is hydrogen; R⁵ is hydrogen; R⁶ is hydrogen; R^{R} is 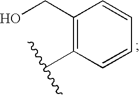
and —NX¹X² is 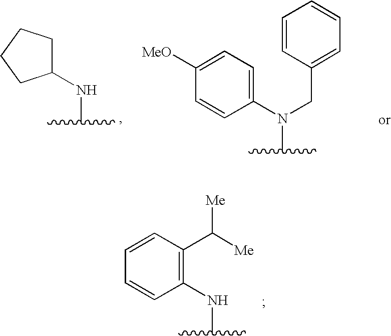
X³ is not hydrogen.

In certain embodiments, the present invention relates to the aforementioned compound wherein R¹ is hydrogen; halogen; a saturated or unsaturated, branched or straight-chain C₁₋₆ alkyl; aryl-C₁₋₆ alkyl; mono- or polyfluorinated C₁₋₆ alkyl; C₁₋₆ alkoxy; C₁₋₆ alkylamino; di(C₁₋₆ alkyl)amino; C₁₋₈ alkylamino-C₁₋₈ alkyl; di(C₁₋₆ alkyl)amino-C₁₋₈ alkyl; cyclo(C₃₋₆)alkyl; aryl, wherein the aryl comprises a six membered aromatic carbocycle (such as phenyl) or a polycyclic aromatic hydrocarbon (such as naphthyl, phenanthracenyl, indanyl); a heterocycle, wherein the heterocycle comprises six membered aromatic heterocycles (such as pyridyl, diazinyl, pyrimidinyl, pyrrolidinyl, piperazinyl, thiazinyl), five membered aromatic heterocycles (such as pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thienyl) or bicyclic systems (such as indolyl, benzothienyl, benzofuranyl, isoindolyl, isobenzothienyl, isobenzofuranyl); wherein any of wherein one or more of the foregoing aliphatic, cyclic, aromatic or heteroaromatic substituents optionally may be further substituted with a C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, cyano, hydroxy, carboxy, carboxy ester, amine (optionally substituted with C₁₋₆ straight chain alkyl), C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned compound wherein R¹ represents two non-hydrogen substituents which combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned compound, wherein R², R³, R⁴, R⁵ and R⁶, and the carbons to which they are bonded, may combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned compound wherein X¹, X² and X³ are independently selected from the group consisting of hydrogen, a C₁₋₆ straight chain saturated or unsaturated alkyl group, a C₃₋₆ branched saturated or unsaturated chain alkyl group, a C₃₋₆ cycloalkyl group; and any of the foregoing are optionally substituted with one or more halo, nitro, cyano, hydroxy, carboxy, carboxy ester, amine (optionally substituted with C₁₋₆ straight chain alkyl), C₃₋₆ branched chain alkyl, C₃₋₆ cycloalkyl, aromatic group or aralkyl group (such as phenyl, benzyl or naphthyl, optionally further substituted as described above), fused alkyl or aromatic ring, or heteroaromatic or heterocyclic ring, which may be a saturated or unsaturated ring containing 4-10 ring members and 0-3 heteroatoms selected from the group consisting of O, N and S, the heteroaromatic or heterocyclic ring optionally substituted with one or more halo, C₁₋₆ straight chain alkyl, C₃₋₆ branched chain alkyl, C₃₋₆ cycloalkyl, C₁₋₆ alkyloxy, nitro, cyano, hydroxy, carboxyl, ester, amine (optionally substituted with C₁₋₆ straight chain alkyl), C₃₋₆ branched chain alkyl or C₃₋₆ cycloalkyl, trifluoroxy, trifluoromethyl, difluoromethyl, aryl, the same or different heterocyclic ring, or a fused aromatic, heteroaromatic or heterocyclic ring. The alkyl group of alkyloxy may be a C₁₋₆ straight chain, C₃₋₆ branched or C₃₋₆ cycloalkyl; and any of the alkyl groups herein may be saturated or contain one or more degrees of unsaturation; or X¹ and X² together with the nitrogen to which they are bonded is an optionally substituted heteroaromatic or heterocyclic ring comprising in addition to the aforementioned nitrogen, 4-10 ring members and 0-3 additional heteroatoms selected from the group consisting of O, N and S, the heteroaromatic or heterocyclic ring optionally further substituted with one or more aliphatic, aromatic, —SR^{R}, —OR^{R}, heteroaromatic or fused rings which may be further substituted as described herein.

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned compound wherein X¹ and X² taken together with the nitrogen to which they are bonded are an optionally substituted heterocyclic group comprising 4-10 ring members and 0-3 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned compound wherein R¹, R³, R⁴, R⁵ and R⁶ are hydrogen; R² is —SR^{R}; and R^{R} is an optionally substituted phenyl group. Examples of substitutions of said phenyl group include a hydroxyalkyl group (such as hydroxymethyl and hydroxyethyl); a haloalkyl group (such as fluoromethyl, difluoromethyl and trifluoromethyl); an alkoxyalkyl group (such as ethoxymethyl and methoxymethyl); a carboxyalkyl group (such as carboxymethyl and carboxyethyl); a —COOH; a C₁₋₆ alkylidene-O(C═O)-alkyl or C₁₋₆ alkylidene-(C═O)-alkoxy group (such as —CH₂—OC(═O)—CH₃ and —CH₂CH₂—C(═O)—OCH₃); an amide, alkylamide or dialkylamide; and an alkylaminocarboxy moiety (such as —OC(═O)NHEt).

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned compound wherein X¹ and X² are hydrogen or an optionally substituted aliphatic, alicyclic, or aromatic group.

In certain embodiments, the present invention relates to the aforementioned compound wherein X¹ and X² are hydrogen, cyclopentyl, benzyl, 4-methoxyphenyl or 2-isopropylphenyl.

In certain embodiments, the present invention relates to the aforementioned compound wherein X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned compound wherein —NX¹X² is selected from the two sets of —NX¹X² moieties described for formula I above.

In certain embodiments, the present invention relates to the aforementioned compound wherein R² is —SR^{R}.

In certain embodiments, the present invention relates to the aforementioned compound wherein R² is —SR^{R}; and R³, R⁴, R⁵ and R⁶ are hydrogen.

In certain embodiments, the present invention relates to the aforementioned compound wherein R² is —SR^{R}; R³, R⁴, R⁵ and R⁶ are hydrogen; and R^{R} is an optionally substituted phenyl.

In certain embodiments, the present invention relates to the aforementioned compound wherein R² is —SR^{R}; R³, R⁴, R⁵ and R⁶ are hydrogen; R^{R} is 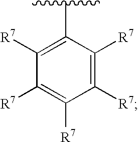
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned compound wherein R² is —SR^{R}; R³, R⁴, R⁵ and R⁶ are hydrogen; R^{R} is 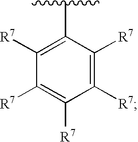
and R⁷ is, independently for each occurrence, hydrogen, hydroxymethyl, hydroxyethyl, fluoromethyl, difluoromethyl, trifluoromethyl, ethoxymethyl, methoxymethyl, carboxymethyl, carboxyethyl, —COOH, —CH₂—OC(═O)—CH₃, —CH₂CH₂—C(═O)—OCH₃ or —O(CO)NHEt.

In certain embodiments, the present invention relates to the aforementioned compound wherein R² is —SR^{R}; R³, R⁴, R⁵ and R⁶ are hydrogen; and R^{R} is

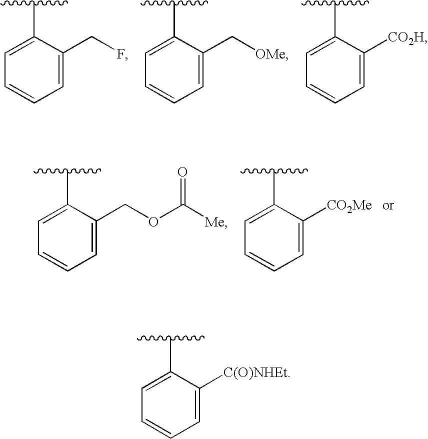

In certain embodiments, the present invention relates to the aforementioned compound wherein R¹ is hydrogen, halogen, C₁₋₆ alkyl, aryl-C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, aryl, or heterocycle; wherein one or more of the foregoing aliphatic, cyclic, aromatic or heteroaromatic substituents optionally may be further substituted with C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, fluoro, cyano, hydroxy, carboxy, carboxy ester, amine, C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned compound wherein R¹ is hydrogen, halogen, C₁₋₆ alkyl or C₁₋₆ alkoxy.

In certain embodiments, the present invention relates to the aforementioned compound wherein R¹ is hydrogen.

In certain embodiments, the present invention relates to the aforementioned compound wherein X³ is hydrogen, aliphatic or alicyclic.

In certain embodiments, the present invention relates to the aforementioned compound wherein X³ is hydrogen or C₁₋₆ alkyl.

In certain embodiments, the present invention relates to the aforementioned compound wherein X³ is hydrogen.

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups; and R² is —SR^{R}.

In certain embodiments, the present invention relates to the aforementioned compound wherein X¹ and X² are hydrogen or an optionally substituted aliphatic, alicyclic, heteroaliphatic, heterocyclic, aromatic, heteroaromatic or acyl group; R² is —SR^{R}; R³, R⁴, R⁵ and R⁶ are hydrogen; and R^{R} is an optionally substituted phenyl.

In certain embodiments, the present invention relates to the aforementioned compound wherein X¹ and X² are hydrogen or an optionally substituted aliphatic, alicyclic, or aromatic group; and R² is —SR^{R}; R³, R⁴, R⁵ and R⁶ are hydrogen; R^{R} is 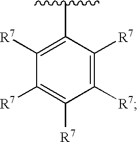
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned compound wherein X¹ and X² are hydrogen or an optionally substituted aliphatic, alicyclic, or aromatic group; and R² is —SR^{R}; R³, R⁴, R⁵ and R⁶ are hydrogen; R^{R} is 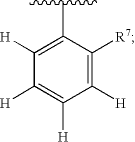
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned compound wherein X¹ and X² are hydrogen, cyclopentyl, benzyl, 4-methoxyphenyl or 2-isopropylphenyl; R² is —SR^{R}; R³, R⁴, R⁵ and R⁶ are hydrogen; and R^{R} is

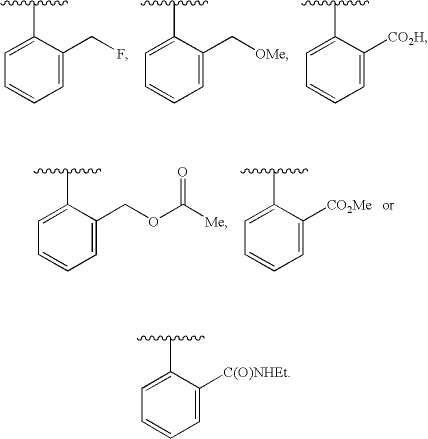

In certain embodiments, the present invention relates to the aforementioned compound wherein X¹ and X² are hydrogen, cyclopentyl, benzyl, 4-methoxyphenyl or 2-isopropylphenyl; R² is —SR^{R}; R³, R⁴, R⁵ and R⁶ are hydrogen; R^{R} is 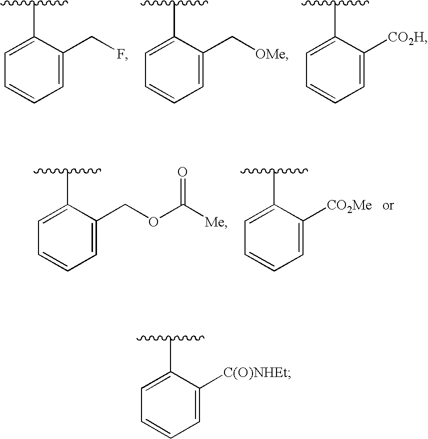
and R¹ is hydrogen.

Another aspect of the invention relates to compound selected from the group consisting of the compounds shown below.

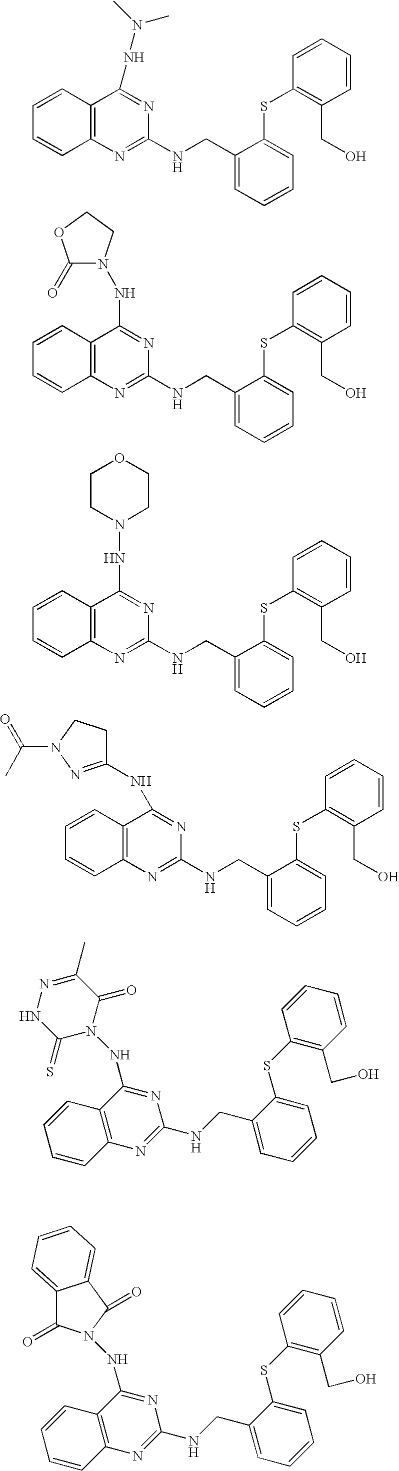

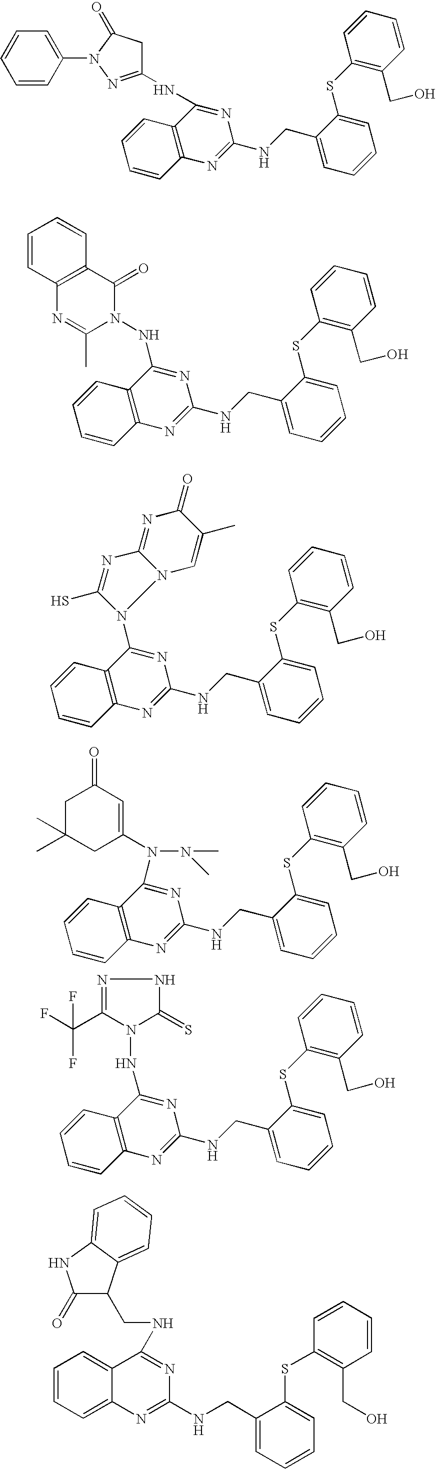

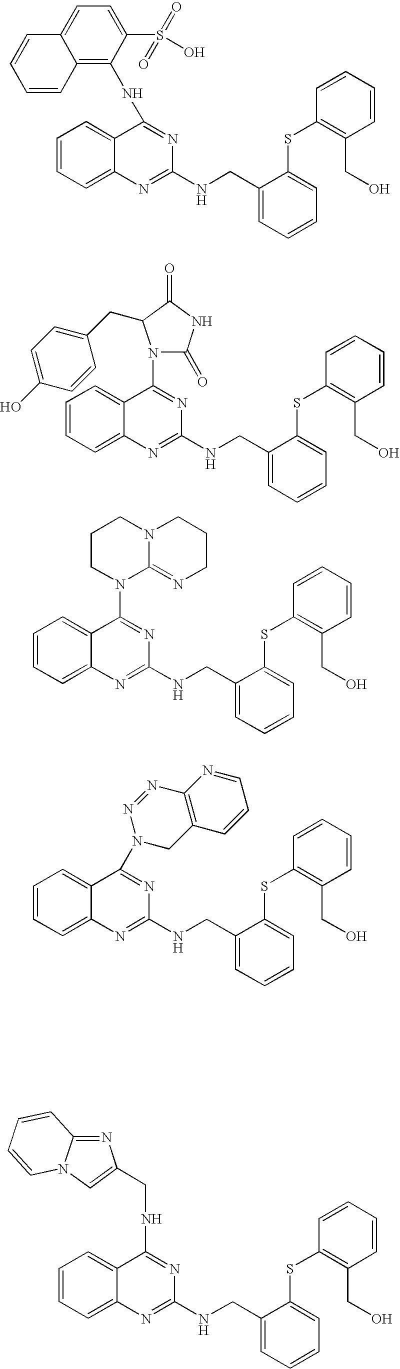

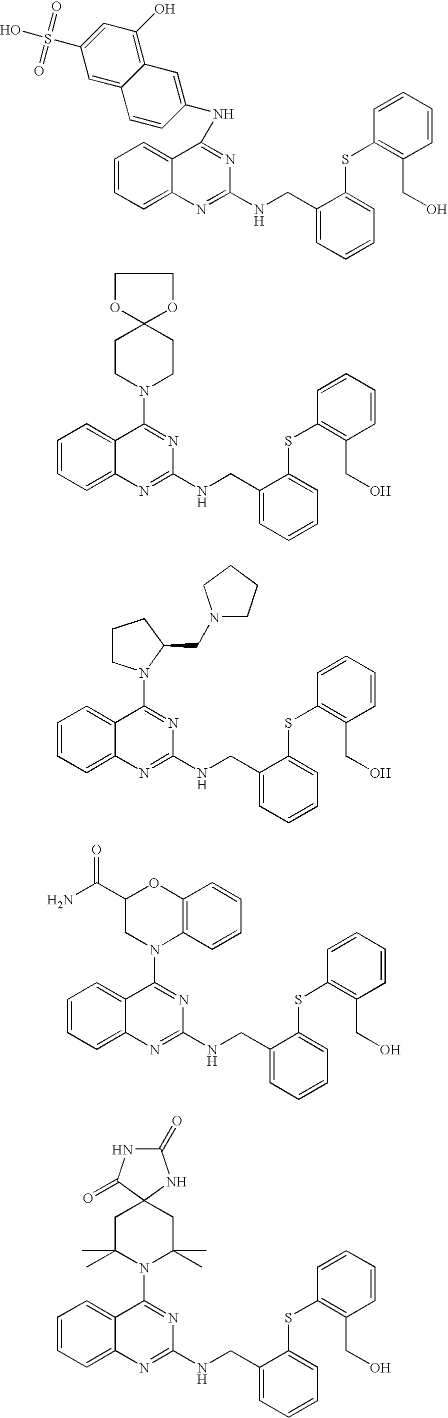

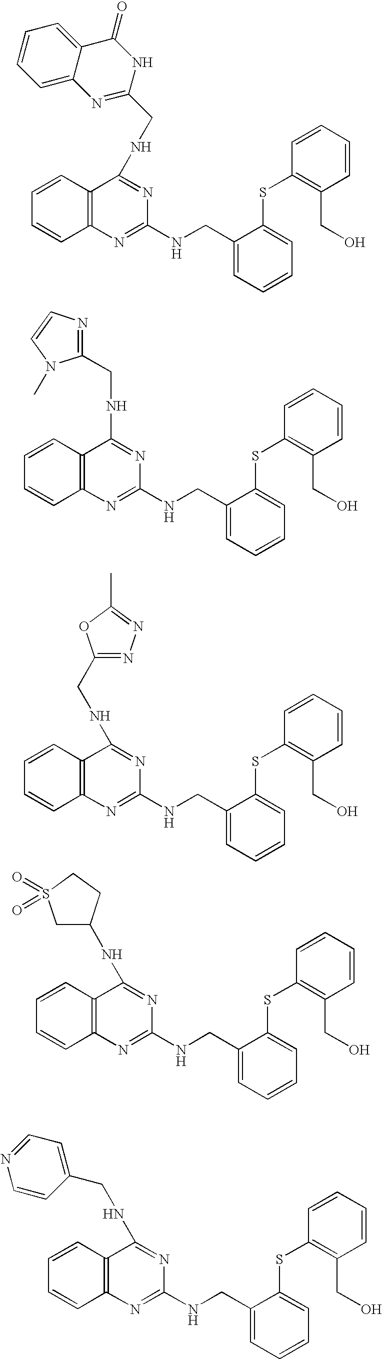

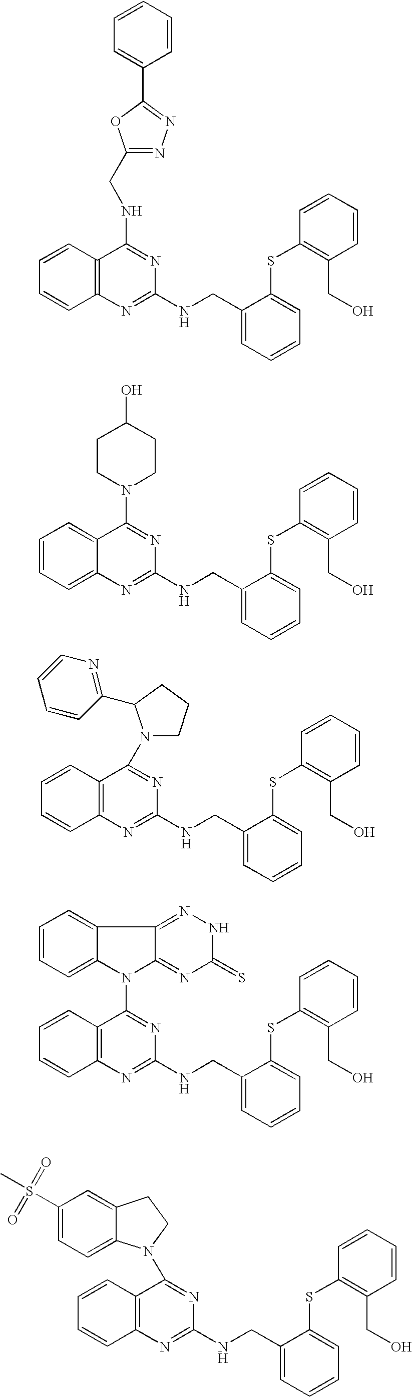

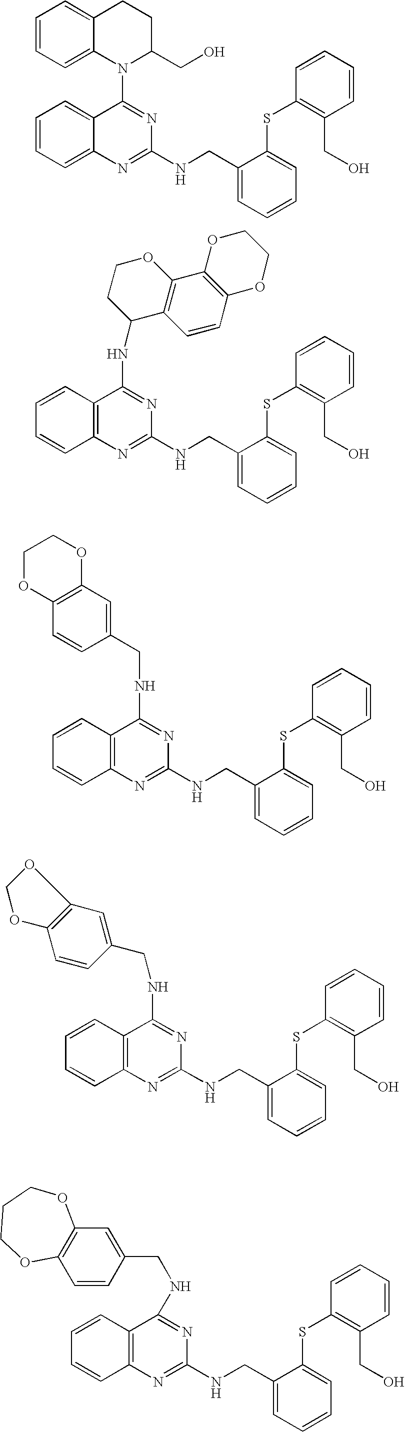

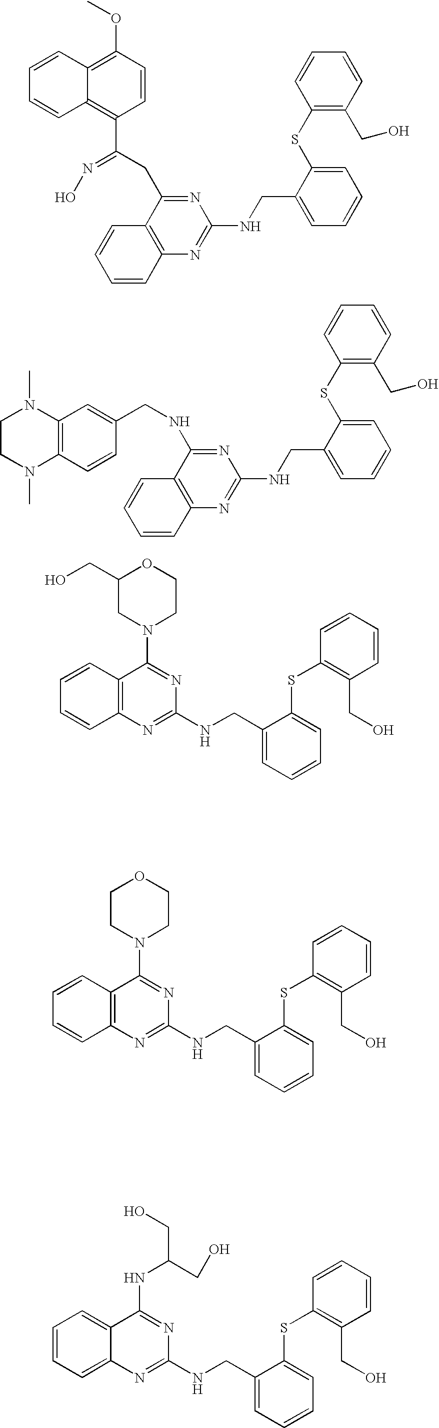

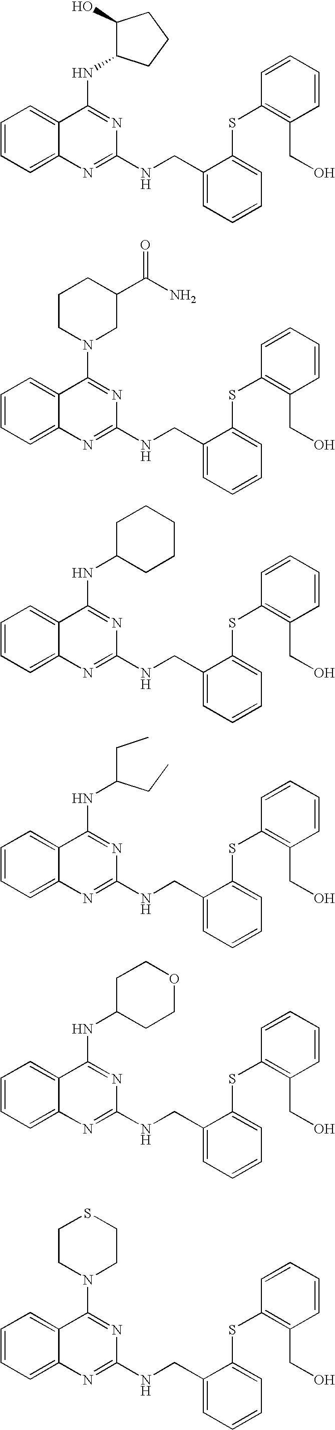

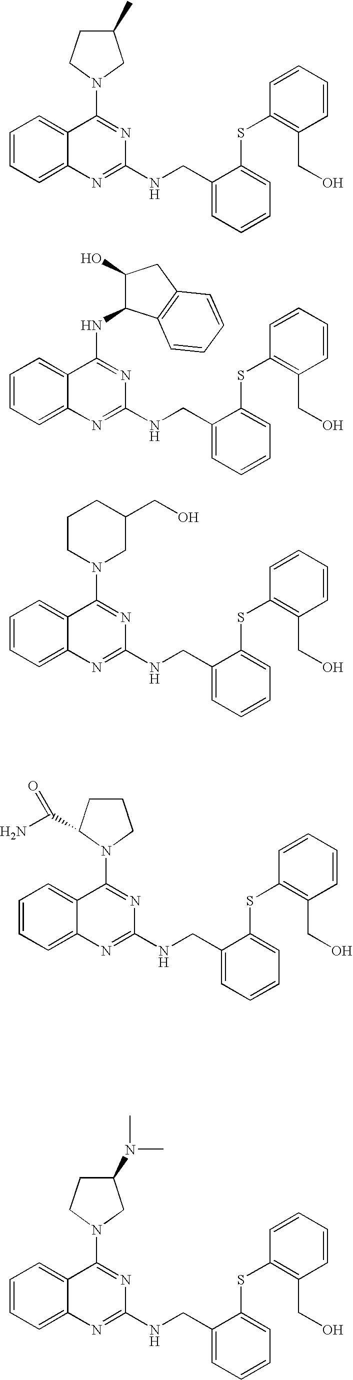

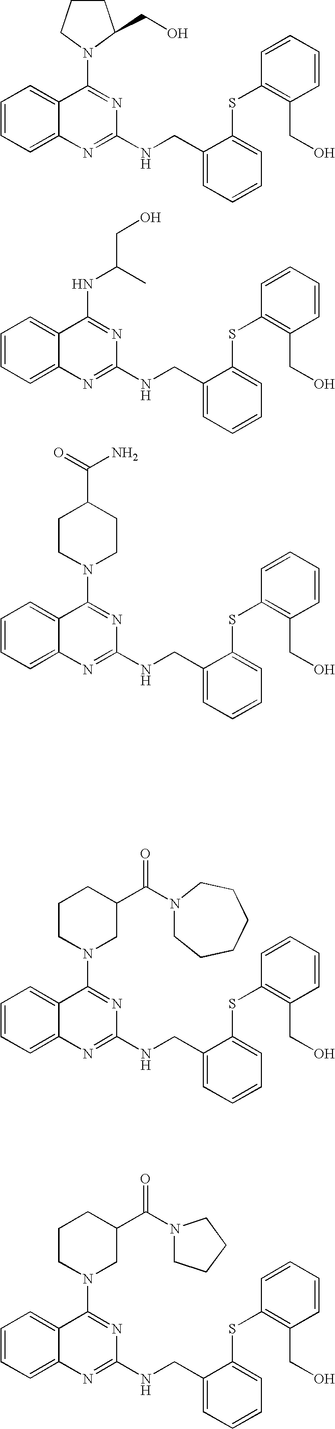

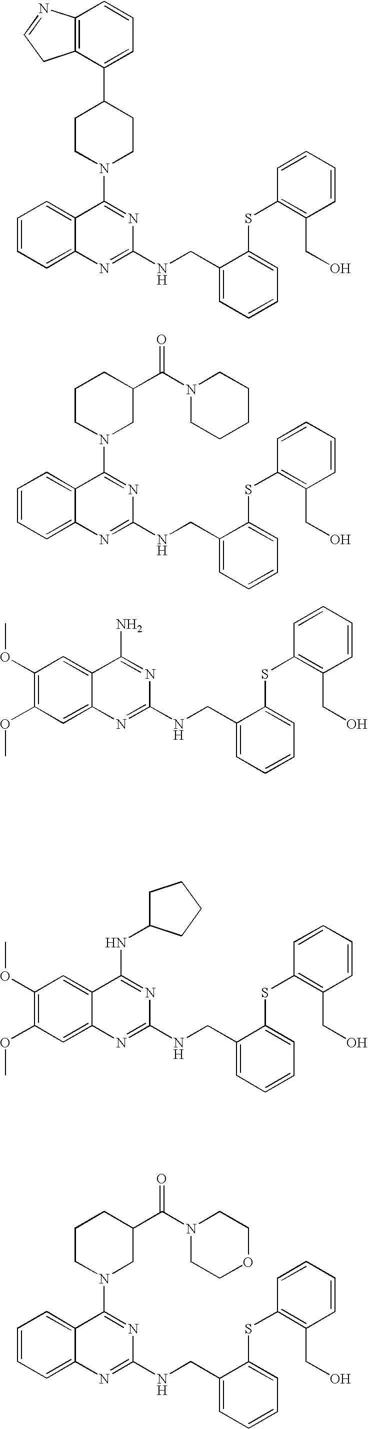

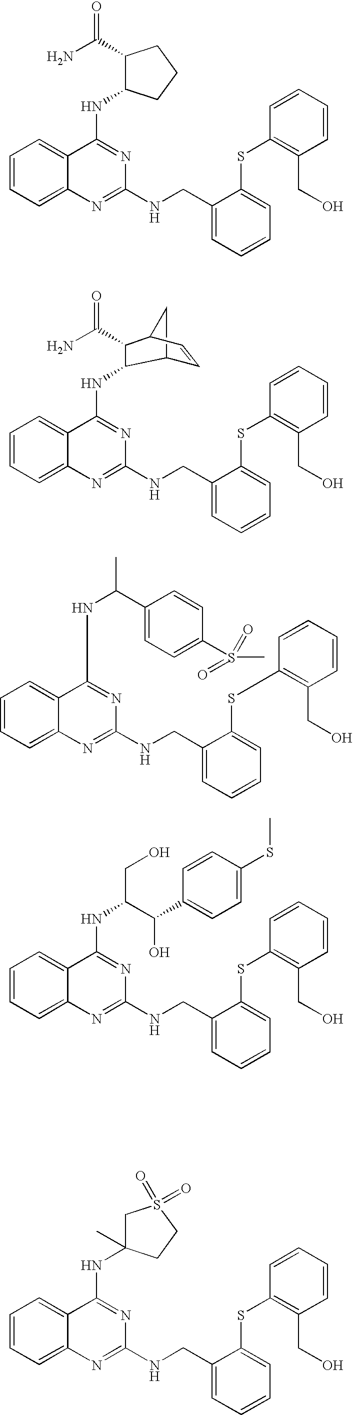

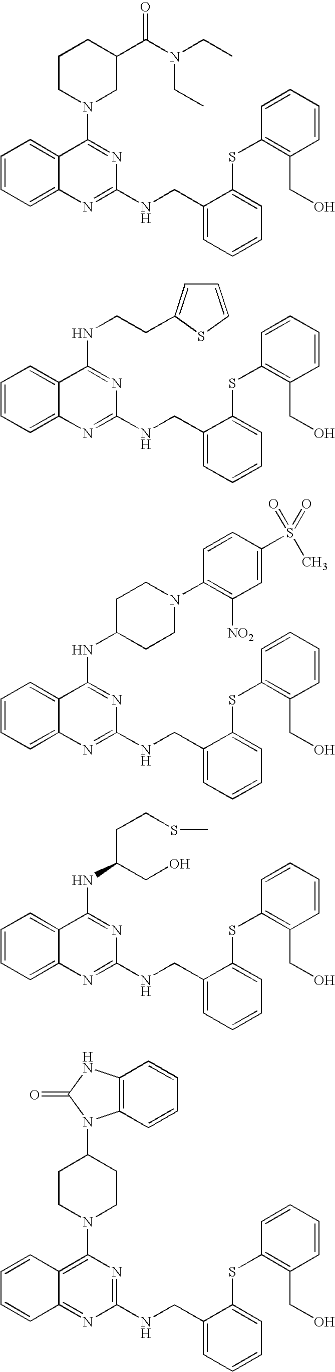

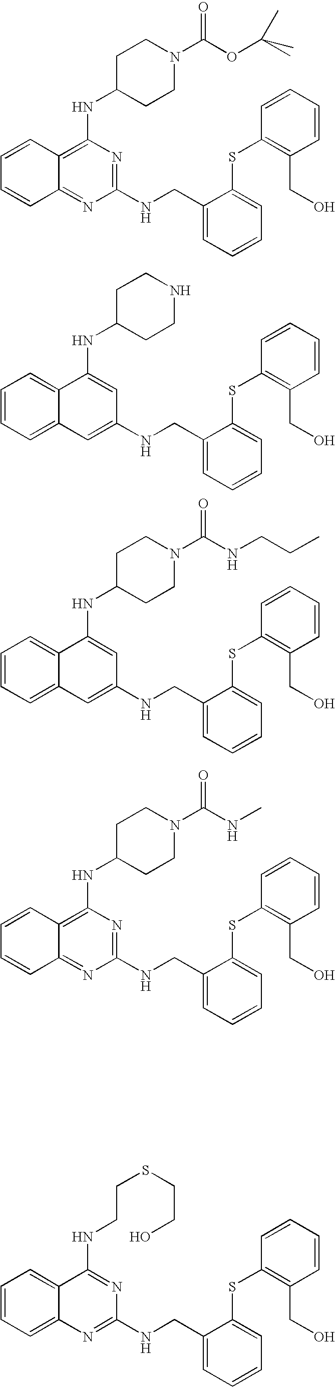

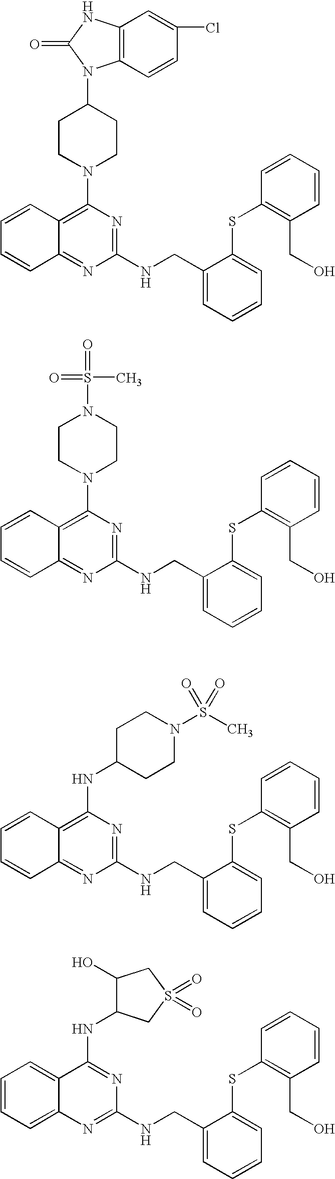

In another embodiment, the present invention relates to a compound of formula IV:

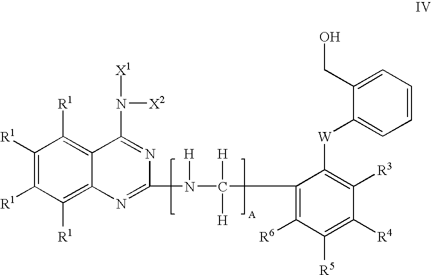

or a pharmaceutically acceptable salt thereof,

wherein, independently for each occurrence:

A is 0 or 1;

W is S, S(O) or S(O)₂;

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned compound provided that when W is S; R¹ is hydrogen; R³ is hydrogen; R⁴ is hydrogen; R⁵ is hydrogen; R⁶ is hydrogen; and —NX¹X² is 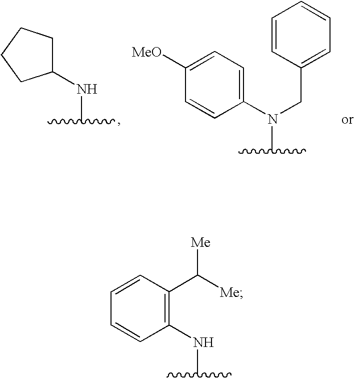
A is not 1.

In certain embodiments, the present invention relates to the aforementioned compound wherein A is 0. In certain embodiments, the present invention relates to the aforementioned compound wherein A is 1.

In certain embodiments, the present invention relates to the aforementioned compound wherein W is S. In certain embodiments, the present invention relates to the aforementioned compound wherein W is S(O). In certain embodiments, the present invention relates to the aforementioned compound wherein W is S(O)₂. In certain embodiments, the present invention relates to the aforementioned compound wherein W is S or S(O)₂.

In certain embodiments, the present invention relates to the aforementioned compound wherein A is 0; R³ is H; R⁴ is H; R⁵ is H; R⁶ is H; and W is S. In certain embodiments, the present invention relates to the aforementioned compound wherein A is 0; R³ is H; R⁴ is H; R⁵ is OH; R⁶ is H; and W is S. In certain embodiments, the present invention relates to the aforementioned compound wherein A is 0; R³ is H; R⁴ is H; R⁵ is H; R⁶ is H; and W is S(O)₂. In certain embodiments, the present invention relates to the aforementioned compound wherein A is 0; R³ is H; R⁴ and R⁵ taken together are —OCH₂O—; R⁶ is H; and W is S(O)₂. In certain embodiments, the present invention relates to the aforementioned compound wherein A is 0; R³ is OH; R⁴ is H; R⁵ is H; R⁶ is H; and W is S(O)₂. In certain embodiments, the present invention relates to the aforementioned compound wherein A is 0; R³ is H; R⁴ is OH; R⁵ is H; R⁶ is H; and W is S(O)₂. In certain embodiments, the present invention relates to the aforementioned compound wherein A is 0; R³ is H; R⁴ is H; R⁵ is OH; R⁶ is H; and W is S(O)₂. In certain embodiments, the present invention relates to the aforementioned compound wherein A is 0; R³ is H; R⁴ is H; R⁵ is H; R⁶ is OH; and W is S(O)₂. In certain embodiments, the present invention relates to the aforementioned compound wherein A is 1; R³ is H; R⁴ is Cl; R⁵ is H; R⁶ is H; and W is S(O)₂. In certain embodiments, the present invention relates to the aforementioned compound wherein A is 0; R³ is Cl; R⁴ is H; R⁵ is H; R⁶ is H; and W is S. In certain embodiments, the present invention relates to the aforementioned compound wherein A is 0; R³ is H; R⁴ is H; R⁵ is Cl; R⁶ is H; and W is S. In certain embodiments, the present invention relates to the aforementioned compound wherein A is 0; R³ is H; R⁴ is H; R⁵ is H; R⁶ is Cl; and W is S. In certain embodiments, the present invention relates to the aforementioned compound wherein A is 1; R³ is Cl; R⁴ is H; R⁵ is H; R⁶ is H; and W is S(O)₂. In certain embodiments, the present invention relates to the aforementioned compound wherein A is 1; R³ is H; R⁴ is Cl; R⁵ is H; R⁶ is H; and W is S(O)₂. In certain embodiments, the present invention relates to the aforementioned compound wherein A is 0; R³ is H; R⁴ is OH; R⁵ is OH; R⁶ is H; and W is S(O)₂. In certain embodiments, the present invention relates to the aforementioned compound wherein A is 0; R³ is Cl; R⁴ is H; R⁵ is H; R⁶ is H; and W is S(O)₂. In certain embodiments, the present invention relates to the aforementioned compound wherein A is 0; R³ is H; R⁴ is Cl; R⁵ is H; R⁶ is H; and W is S(O)₂.

In certain embodiments, the present invention relates to the aforementioned compound wherein X¹ is cyclopentyl; and X² is hydrogen.

Another aspect of the invention relates to compound selected from the group consisting of the compounds shown below.

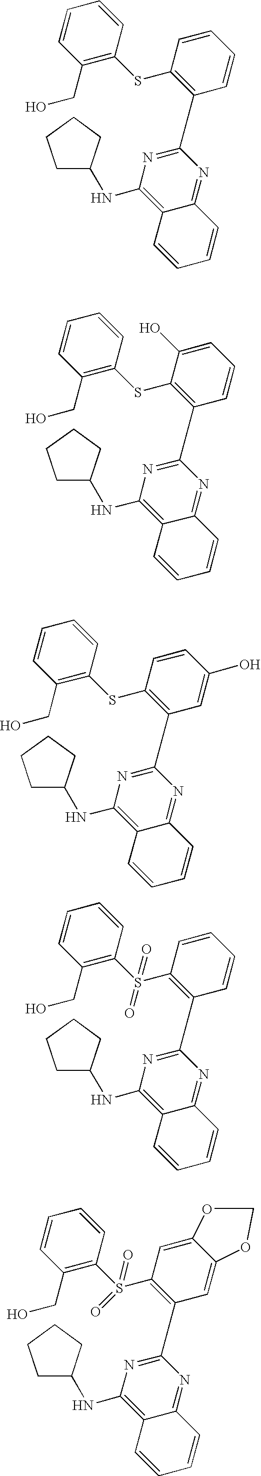

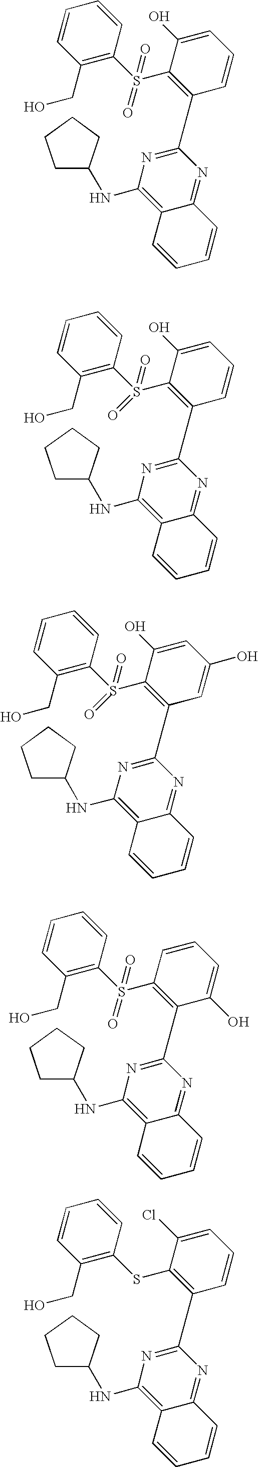

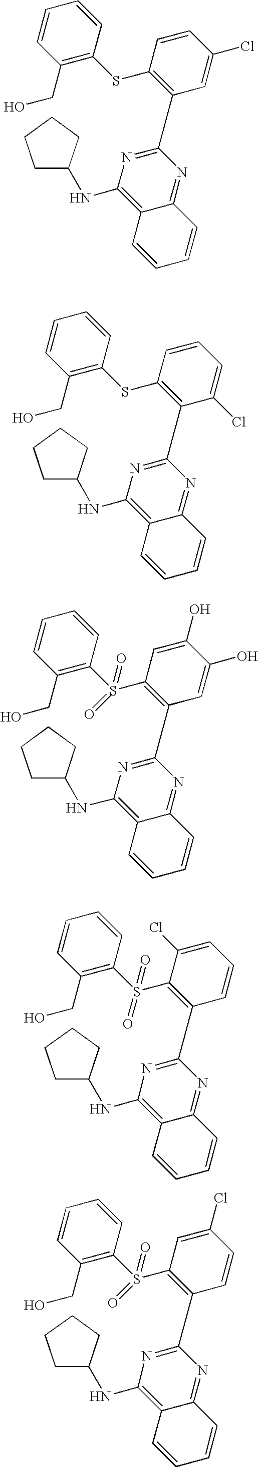

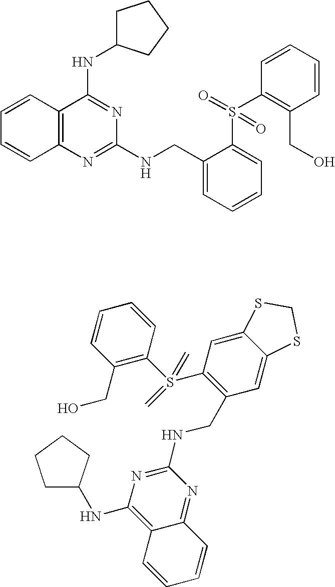

As mentioned before, the foregoing examples are merely illustrative and compounds comprising variants of the substituents as well as their positions are fully embraced by the teachings herein.

In another embodiment, the present invention relates to a compound of formula V:

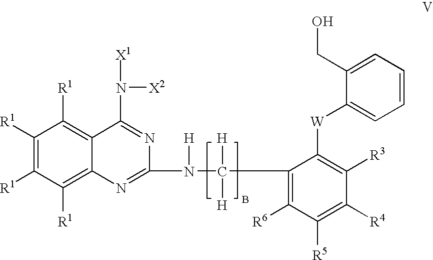

or a pharmaceutically acceptable salt thereof,

wherein, independently for each occurrence:

B is 0, 1, 2, 3, 4, or 5;

W is S, S(O) or S(O)₂;

R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned compound provided that when W is S; R¹ is hydrogen; R³ is hydrogen; R⁴ is hydrogen; R⁵ is hydrogen; R⁶ is hydrogen; and —NX¹X² is 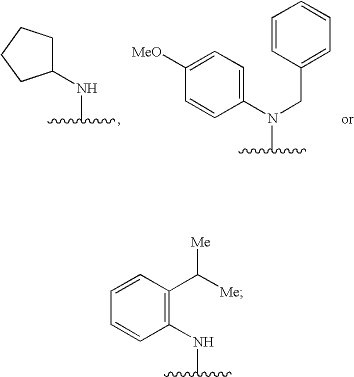
B is not 1.

In certain embodiments, the present invention relates to the aforementioned compound wherein B is 0. In certain embodiments, the present invention relates to the aforementioned compound wherein B is 1. In certain embodiments, the present invention relates to the aforementioned compound wherein B is 2. In certain embodiments, the present invention relates to the aforementioned compound wherein B is 3. In certain embodiments, the present invention relates to the aforementioned compound wherein B is 4. In certain embodiments, the present invention relates to the aforementioned compound wherein B is 5.

In certain embodiments, the present invention relates to the aforementioned compound wherein W is S. In certain embodiments, the present invention relates to the aforementioned compound wherein W is S(O). In certain embodiments, the present invention relates to the aforementioned compound wherein W is S(O)₂. In certain embodiments, the present invention relates to the aforementioned compound wherein W is S or S(O)₂.

In certain embodiments, the present invention relates to the aforementioned compound wherein X¹ is cyclopentyl; R¹, X² and X³ are hydrogen; W is S; and at least one of R³, R⁴, R⁵ and R⁶ is not hydrogen.

In another embodiment, the present invention relates to a compound of formula VI:

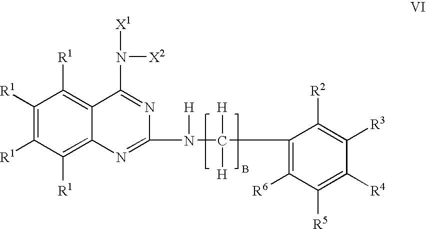

or a pharmaceutically acceptable salt thereof,

wherein, independently for each occurrence:

B is 0, 1, 2, 3, 4, or 5;

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R²R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned compound provided that when R¹ is hydrogen; R² is —SR^{R}; R³ is hydrogen; R⁴ is hydrogen; R⁵ is hydrogen; R⁶ is hydrogen; R^{R} is 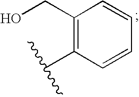
and —NX¹X² is 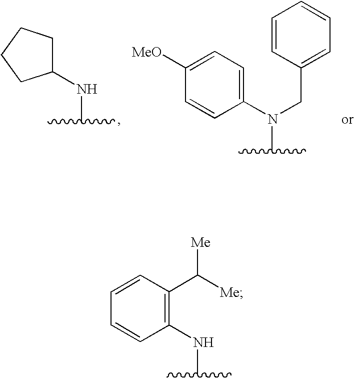
B is not 1.

Importantly, some of the compounds of the invention can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Compounds of the invention may be prepared by crystallization of compounds of formula I-VI under different conditions and may exist as one or a combination of polymorphs of compounds of general formula I-VI forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric forms, their stereoisomers, positional isomer, their polymorphs, their pharmaceutically acceptable salts, their pharmaceutically acceptable solvates and pharmaceutically acceptable compositions containing them.

In addition to those compounds described above, the following compounds and pharmaceutical compositions comprising such compounds are also embraced by the present invention. Furthermore, methods for treating the various conditions and diseases described herein, in particular dysproliferative diseases, also benefit from the administration of the following compounds and pharmaceutical compositions comprising them. Both pharmaceutical compositions and methods are discussed further below.

Research Uses

According to the present invention, the inventive compounds may be assayed in any of the available assays known in the art for identifying compounds having the ability to modulate c-Met activity or Tie-2 activity and in particular to antagonize or block the activities of c-Met or Tie-2 (see “Hyperproliferative Diseases” below). For example, the assay may be cellular or non-cellular, in vivo or in vitro, high- or low-throughput format, etc.

Certain compounds of the invention of particular interest include those with c-Met antagonistic activity, which modulate, for example, inhibit, HGF/SF activity; inhibit HGF/SF-induced phosphorylation of c-Met; inhibit c-Met tyrosine kinase activity; exhibit the ability to antagonize HGF/SF; inhibit cell proliferation; inhibit invasion; exhibit apoptotic activity; exhibit anti-angiogenic activity; and/or are useful for the treatment of HGF/SF-induced disorders.

Such assays for the above activities are, for example: inhibition of endothelial cell proliferation, such as by using human umbilical vein endothelial cells or aortic rings, such as described in the examples below; inhibition of dysproliferative cell growth stimulated by HGF/SF, for example, using U87MG glioma cells, GLT-16 human gastric carcinoma cells, or A549 lung cancer cells as described in the examples below; inhibition of epithelial cell proliferation in response to HGF/SF, such as by using 4 MBr-5 cells, a monkey lung epithelial cell line, as described in the examples below; inhibition of scatter or metastasis, using a matrix-based assay, as described in the examples below; and inhibition of HGF/SF-induced phosphorylation of c-Met, using a reporter cell line assay such as CELLSENSOR™ AP-1-bla HEK 293T Cell Line (Invitrogen), which contains a beta-lactamase reporter gene under control of the AP-1 response element stably integrated into HEK 293T cells. The AP-1-bla HEK 293T cell line responds to agonist treatment as expected from literature and can be adapted for high throughput screening for agonists or antagonists of the AP-1 pathway. These are merely exemplary of assays useful in identifying compounds of the invention.

Assays for Tie-2 inhibition or antagonization can be used as well. Such assays include in vitro protein tyrosine kinase assays that measure incorporation of ³²P-ATP into a kinase specific substrate in the presence of Tie-2; cellular phosphorylation assays using HUVECs stimulated with Ang1 in the presence or absence of compound to assess inhibition of Ang1 signal transduction in endothelial cells; and inhibition of Ang1-induced HUVEC migration.

Pharmaceutical Uses and Methods of Treatment

As discussed above, certain of the compounds as described herein exhibit activity generally as modulators of HGF/SF activity. More specifically, compounds of the invention demonstrate the ability to antagonize HGF/SF activity. Thus, in certain embodiments, compounds of the invention are useful for the treatment of any of a number of conditions or diseases in which HGF/SF or the activities thereof have a pathophysiologically relevant, adverse role or where inhibition or blocking c-Met or HGF/SF signaling inhibition is beneficial (see “Hyperproliferative Diseases” below).

In addition, compounds of the invention have been found to also inhibit or antagonize Tie-2, a receptor tyrosine kinase involved in angiogenesis. The Tie-2 receptor is exclusively expressed on endothelial cells and plays an important role in the regulation of vascular remodeling. Inhibiting Tie-2 by compounds of the invention is another means for preventing the growth of tumors, by impairing formation of tumor vasculature and destabilizing existing vessels. It is a further aspect of the present invention to provide compounds and pharmaceutical compositions that inhibit or antagonize Tie-2, and methods of using such compounds and pharmaceutical compositions for the treatment of dysproliferative diseases such as cancer.

Accordingly, in another aspect of the invention, methods for the treatment of HGF/SF activity related disorders are provided comprising administering a therapeutically effective amount of a compound of formula I or II as described herein, to a subject in need thereof. In certain embodiments, a method for the treatment of undesirable HGF/SF activity related disorders is provided comprising administering a therapeutically effective amount of an inventive compound, or a pharmaceutical composition comprising an inventive compound to a subject in need thereof, in such amounts and for such time as is necessary to achieve the desired result.

Further experiments on the mechanism of action of compounds of the invention revealed that certain compounds of the invention are dual function antagonists of c-Met and Tie-2. As will be shown in the examples below, a tyrosine kinase ELISA assay was performed to determine the effects of compound on four receptor tyrosine kinases (c-Met, PDGFR, IGFR1, and FGFR1). Results show that compounds of the invention inhibit c-Met kinase activity with an IC₅₀ of less than 1 μM, and in some cases less than 0.1 uM, whereas the highest concentrations tested (10 μM) did not affect the kinase activities of PDGFR, IGFR1 or FGFR3.

Further studies showed that inventive compounds inhibit Ang1-induced Tie-2 and downstream effector activation in HUVECs. Tie-2 is an endothelial-cell-restricted receptor. To determine the activity, HUVECs were stimulated with Ang-1 (300 ng/ml) in the presence or absence of a series of concentrations of compound. Cells were lysed and 16 micrograms of protein were loaded on SDS-PAGE for Western blot analysis. Membranes were incubated with primary antibodies. The results show that inventive compound suppressed Ang1-induced activity of Tie-2 and downstream effectors (Akt and Erk) in a dose-dependent manner.

In another assay, human epidermal cancer cells A431 are cultured with RPMI medium containing 0.1% fetal calf serum in 96-well plate in an amount of 3×10⁴ per well overnight. A solution of the test compound in DMSO is added to each well, and the cells are incubated at 37 C for additional one hour, and then hHGF is added to a final concentration of 50 ng/ml for 5 min. The medium is removed and the cells are washed with PBS, and 50 ul of lysis buffer is then added. The mixture is shaken at 4 C for 2 hr to prepare a cell extract. The Human Phospho-c-Met ELISA kit (R&D, DYC2480-2) is used to measure phosphorylated c-Met in these cell lysates. The Met-phosphorylation inhibitory activity for each well is determined by presuming the absorbance with the addition of HGF and the vehicle to compounds to be 0% Met-phosphorylation inhibitory activity and the absorbance with the addition of the vehicle to compounds and without HGF to be 100% Met phosphorylation inhibitory activity. The concentration of the test compound is varied on several levels, the inhibition (%) of Met-phosphorylation is determined for each case, and the concentration of the test compound necessary for inhibiting 50% of met phosphorylation (IC50) is calculated for comparing their inhibitory activity.

Pharmaceutical Compositions

As discussed above this invention is directed in part to compounds that have biological properties useful for the treatment of any of a number of conditions or diseases in which inhibition of HGF/SF or the activities thereof have a therapeutically useful role, such as those described above. Accordingly, in another aspect of the present invention, pharmaceutical compositions are provided, which comprise any one or more of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. The invention is also directed to new uses of known compounds heretofore unrecognized as having the activities described above, and in particular having such activities without co-administration of another compound, more particularly another compound that is not an anti-cancer agent. Thus, the compounds of the invention exhibit anti-cancer and other beneficial activities directly, without the necessity to co-administer with them a compound that is not an anti-cancer compound but whose purpose is to produce or increase the activity of the compounds of the invention. As discussed above, compound embodied herein also have utility in treatment of various dysproliferative orders in addition to cancer, such as inflammatory diseases including inappropriate angiogenesis.

Alternatively, a compound of this invention may be administered to a patient in need thereof in combination with the administration of one or more other therapeutic agents (see discussion of synergism and combination therapy below). For example, additional therapeutic agents for conjoint administration or inclusion in a pharmaceutical composition with a compound of this invention may be an approved agent to treat the same or related indication, or it may be any one of a number of agents undergoing approval in the Food and Drug Administration that ultimately obtain approval for the treatment of any disorder related to HGF/SF activity. Such compounds include, by way of non-limiting examples, small molecule tyrosine kinase inhibitors targeting EGFR (e.g., erlotinib (TARCEVA) or gefitinib (IRESSA)) and c-Kit (e.g., imatinib (GLEEVEC)) and antibodies targeting EGFR (e.g., cetuximab (ERBITUX)) and VEGFR (e.g., bevacizumab (AVASTIN)). Also included are anticancer chemotherapeutic agents such as, for example, aldesleukin (PROLEUKIN); alemtuzumab (CAMPATH); alitretinoin (PANRETIN); allopurinol (ZYLOPRIM); altretamine (HEXALEN); amifostine (ETHYOL); anastrozole (ARIMIDEX); arsenic trioxide (TRISENOX); asparaginase (ELSPAR); BCG Live (TICE BCG); bexarotene capsules or gel (TARGRETIN); bleomycin (BLENOXANE); busulfan intravenous (BUSULFEX); busulfan oral (MYLERAN); calusterone (METHOSARB); capecitabine (XELODA); carboplatin (PARAPLATIN); carmustine (BCNU, BICNU); carmustine with Polifeprosan 20 Implant (GLIADEL WAFER); celecoxib (CELEBREX); chlorambucil (LEUKERAN); cisplatin (PLATINOL); cladribine (LEUSTATIN, 2-CDA); cyclophosphamide (CYTOXAN, NEOSAR); cytarabine (CYTOSAR-U); cytarabine liposomal (DEPOCYT); dacarbazine (DTIC-DOME); dactinomycin, actinomycin D (COSMEGEN); darbepoetin alfa (ARANESP); daunorubicin liposomal (DANUOXOME); daunorubicin, daunomycin (DAUNORUBICIN or CERUBIDINE); denileukin diftitox (ONTAK); dexrazoxane (ZINECARD); docetaxel (TAXOTERE); doxorubicin (ADRIAMYCIN, RUBEX); doxorubicin liposomal (DOXIL); dromostanolone propionate (DROMOSTANOLONE or MASTERONE INJECTION); Elliott's B solution (ELLIOTT'S B SOLUTION); epirubicin (ELLENCE); Epoetin alfa (EPOGEN); estramustine (EMCYT); etoposide phosphate (ETOPOPHOS); etoposide, VP-16 (VEPESID); exemestane (AROMASIN); filgrastim (NEUPOGEN); floxuridine (intraarterial) (FUDR); fludarabine (FLUDARA); fluorouracil, 5-FU (ADRUCIL); fulvestrant (FASLODEX); gemcitabine (GEMZAR); gemtuzumab ozogamicin (MYLOTARG); goserelin acetate (ZOLADEX); hydroxyurea (HYDREA); ibritumomab Tiuxetan (ZEVALIN); idarubicin (IDAMYCIN); ifosfamide (IFEX); interferon alfa-2a (ROFERON-A or INTRON A); irinotecan (CAMPTOSAR); letrozole (FEMARA); leucovorin (WELLCOVORIN or LEUCOVORIN); levamisole (ERGAMISOL); lomustine, CCNU (CEEBU); meclorethamine, nitrogen mustard (MUSTARGEN); megestrol acetate (MEGACE); melphalan, L-PAM (ALKERAN); mercaptopurine, 6-MP (PURINETHOL); mesna (MESNEX); methotrexate (METHOTREXATE); methoxsalen (UVADEX); mitomycin C (MUTAMYCIN or MITOZYTREX); mitotane (LYSODREN); mitoxantrone (NOVANTRONE); nandrolone phenpropionate (DURABOLIN-50); nofetumomab (VERLUMA); oprelvekin (NEUMEGA); oxaliplatin (ELOXATIN); paclitaxel (PAXENE or TAXOL); pamidronate (AREDIA); pegademase (ADAGEN; PEGADEMASE BOVINE); pegaspargase (ONCASPAR); pegfilgrastim (NEULASTA); pentostatin (NIPENT); pipobroman (VERCYTE); plicamycin, mithramycin (MITHRACIN); porfimer sodium (PHOTOFRIN); procarbazine (MATULANE); quinacrine (ATABRINE); rasburicase (ELITEK); rituximab (RITUXAN); sargramostim (PROKINE); streptozocin (ZANOSAR); talc (SCLEROSOL); tamoxifen (NOLVADEX); temozolomide (TEMODAR); teniposide, VM-26 (VUMON); testolactone (TESLAC); thioguanine, 6-TG (THIOGUANINE); thiotepa (THIOPLEX); topotecan (HYCAMTIN); toremifene (FARESTON); tositumomab (BEXXAR); trastuzumab (HERCEPTIN); tretinoin, ATRA (VESANOID); uracil mustard (URACIL MUSTARD CAPSULES); valrubicin (VALSTAR); vinblastine (VELBAN); vincristine (ONCOVIN); vinorelbine (NAVELBINE); and zoledronate (ZOMETA).

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describes pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation,” as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allantoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methylpyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Formulations for intraocular administration are also included. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

In certain embodiments, the pharmaceutical compositions of the present invention further comprise one or more additional therapeutically active ingredients (e.g., anti-inflammatory and/or palliative). For purposes of the invention, the term “palliative” refers to treatment that is focused on the relief of symptoms of a disease and/or side effects of a therapeutic regimen, but is not curative. For example, palliative treatment encompasses painkillers, antinausea medications and anti-sickness drugs.

The terms “co-administration” and “co-administering” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent at the same time.

The term “synergistic” refers to a combination which is more effective than the additive effects of any two or more single agents. A synergistic effect permits the effective treatment of a disease using lower amounts (doses) of either individual therapy. The lower doses result in lower toxicity without reduced efficacy. In addition, a synergistic effect can result in improved efficacy, e.g., improved anticancer activity. Finally, synergy may result in an improved avoidance or reduction of disease as compared to any single therapy.

Combination therapy often allows for the use of lower doses of the first therapeutic or the second therapeutic agent (referred to as “apparent one-way synergy”), or lower doses of both therapeutic agents (referred to as “two-way synergy”) than would normally be required when either drug is used alone. By using lower amounts of either or both drugs, the side effects associated with them are reduced.

In certain embodiments, the synergism exhibited between the second therapeutic agent and the first therapeutic agent is such that the dosage of the first therapeutic agent would be sub-therapeutic if administered without the dosage of the second therapeutic agent. In other embodiments, the present invention relates to a pharmaceutical composition comprising a therapeutically effective dose of a first therapeutic agent together with a dose of a second therapeutic agent effective to augment the therapeutic effect of the first therapeutic agent. Alternatively, the synergism exhibited between the second therapeutic agent and the first therapeutic agent is such that the dosage of the second therapeutic agent would be sub-therapeutic if administered without the dosage of the first therapeutic agent. In other embodiments, the present invention relates to a pharmaceutical composition comprising a therapeutically effective dose of a second therapeutic agent together with a dose of a first therapeutic agent effective to augment the therapeutic effect of the second therapeutic agent.

In certain preferred embodiments, the invention is directed in part to synergistic combinations of the first therapeutic agent in an amount sufficient to render a therapeutic effect together with a second therapeutic agent. For example, in certain embodiments a therapeutic effect is attained which is at least about 2 (or at least about 4, 6, 8, or 10) times greater than that obtained with the dose of the first therapeutic agent alone. In certain embodiments, the synergistic combination provides a therapeutic effect which is up to about 20, 30 or 40 times greater than that obtained with the dose of first therapeutic agent alone. In such embodiments, the synergistic combinations display what is referred to herein as an “apparent one-way synergy”, meaning that the dose of second therapeutic agent synergistically potentiates the effect of the first therapeutic agent, but the dose of first therapeutic agent does not appear to significantly potentiate the effect of the second therapeutic agent.

In certain embodiments, the combination of active agents exhibit two-way synergism, meaning that the second therapeutic agent potentiates the effect of the first therapeutic agent, and the first therapeutic agent potentiates the effect of the second therapeutic agent. Thus, other embodiments of the invention relate to combinations of a second therapeutic agent and a first therapeutic agent where the dose of each drug is reduced due to the synergism between the drugs, and the therapeutic effect derived from the combination of drugs in reduced doses is enhanced. The two-way synergism is not always readily apparent in actual dosages due to the potency ratio of the first therapeutic agent to the second therapeutic agent. For instance, two-way synergism can be difficult to detect when one therapeutic agent displays much greater therapeutic potency relative to the other therapeutic agent.

The synergistic effects of combination therapy may be evaluated by biological activity assays. For example, the therapeutic agents are be mixed at molar ratios designed to give approximately equipotent therapeutic effects based on the EC90 values. Then, three different molar ratios are used for each combination to allow for variability in the estimates of relative potency. These molar ratios are maintained throughout the dilution series. The corresponding monotherapies are also evaluated in parallel to the combination treatments using the standard primary assay format. A comparison of the therapeutic effect of the combination treatment to the therapeutic effect of the monotherapy gives a measure of the synergistic effect.

Compositions of the invention present the opportunity for obtaining relief from moderate to severe cases of disease. Due to the synergistic and/or additive effects provided by the inventive combination of the first and second therapeutic agent, it may be possible to use reduced dosages of each of therapeutic agent. By using lesser amounts of other or both drugs, the side effects associated with each may be reduced in number and degree. Moreover, the inventive combination avoids side effects to which some patients are particularly sensitive.

Descriptions are provided herein of various, non-limiting conditions, diseases and disorders that are amenable to prophylaxis or treatment by the compounds of the invention. One of skill in the art and understanding the role of HGF/SF in the pathophysiology of various diseases as well as the utility of modulators of HGF/SF activity will be cognizant of the myriad conditions, diseases and disorders for which the compounds of the invention are useful.

One aspect of the invention relates to pharmaceutical compositions comprising compounds represented by formula I:

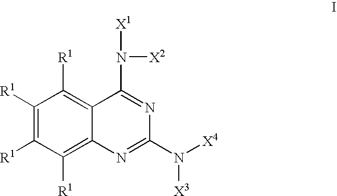

or a pharmaceutically acceptable salt thereof,

wherein, independently for each occurrence,

R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

X¹, X², X³ and X⁴ are hydrogen or an optionally substituted aliphatic, alicyclic, heteroaliphatic, heterocyclic, aromatic, heteroaromatic or acyl group; or X¹ and X² taken together with the nitrogen to which they are bonded, or X³ and X⁴ taken together with the nitrogen to which they are bonded, are independently an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned pharmaceutical composition wherein R¹ is hydrogen; halogen; a saturated or unsaturated, branched or straight-chain C₁₋₆ alkyl; aryl-C₁₋₆ alkyl; mono- or polyfluorinated C₁₋₆ alkyl; C₁₋₆ alkoxy; C₁₋₆ alkylamino; di(C₁₋₆ alkyl)amino; C₁₋₈ alkylamino-C₁₋₈ alkyl; di(C₁₋₆ alkyl)amino-C₁₋₈ alkyl; cyclo(C₃₋₆)alkyl; aryl, wherein the aryl comprises a six membered aromatic carbocycle (such as phenyl) or a polycyclic aromatic hydrocarbon (such as naphthyl, phenanthracenyl, indanyl); a heterocycle, wherein the heterocycle comprises six membered aromatic heterocycles (such as pyridyl, diazinyl, pyrimidinyl, pyrrolidinyl, piperazinyl, thiazinyl), five membered aromatic heterocycles (such as pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thienyl) or bicyclic systems (such as indolyl, benzothienyl, benzofuranyl, isoindolyl, isobenzothienyl, isobenzofuranyl); wherein any of wherein one or more of the foregoing aliphatic, cyclic, aromatic or heteroaromatic substituents optionally may be further substituted with a C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, cyano, hydroxy, carboxy, carboxy ester, amine (optionally substituted with C₁₋₆ straight chain alkyl), C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned pharmaceutical composition wherein R¹ represents two non-hydrogen substituents which combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned pharmaceutical composition wherein R², R³, R⁴, R⁵ and R⁶, and the carbons to which they are bonded, may combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned pharmaceutical composition wherein X¹, X², X³, and X⁴ are independently selected from the group consisting of hydrogen, a C₁₋₆ straight chain saturated or unsaturated alkyl group, a C₃₋₆ branched saturated or unsaturated chain alkyl group, a C₃₋₆ cycloalkyl group; and any of the foregoing are optionally substituted with one or more halo, nitro, cyano, hydroxy, carboxy, carboxy ester, amine (optionally substituted with C₁₋₆ straight chain alkyl), C₃₋₆ branched chain alkyl, C₃₋₆ cycloalkyl, aromatic group or aralkyl group (such as phenyl, benzyl or naphthyl, optionally further substituted as described above), fused alkyl or aromatic ring, or heteroaromatic or heterocyclic ring, which may be a saturated or unsaturated ring containing 4-10 ring members and 0-3 heteroatoms selected from the group consisting of O, N and S, the heteroaromatic or heterocyclic ring optionally substituted with one or more halo, C₁₋₆ straight chain alkyl, C₃₋₆ branched chain alkyl, C₃₋₆ cycloalkyl, C₁₋₆ alkyloxy, nitro, cyano, hydroxy, carboxyl, ester, amine (optionally substituted with C₁₋₆ straight chain alkyl), C₃₋₆ branched chain alkyl or C₃₋₆ cycloalkyl, trifluoroxy, trifluoromethyl, difluoromethyl, aryl, the same or different heterocyclic ring, or a fused aromatic, heteroaromatic or heterocyclic ring. The alkyl group of alkyloxy may be a C₁₋₆ straight chain, C₃₋₆ branched or C₃₋₆ cycloalkyl; and any of the alkyl groups herein may be saturated or contain one or more degrees of unsaturation; or X¹ and X² or X³ and X⁴ together with the nitrogen to which they are bonded is an optionally substituted heteroaromatic or heterocyclic ring comprising in addition to the aforementioned nitrogen, 4-10 ring members and 0-3 additional heteroatoms selected from the group consisting of O, N and S, the heteroaromatic or heterocyclic ring optionally further substituted with one or more aliphatic, aromatic, —SR^{R}, —OR^{R}, heteroaromatic or fused rings which may be further substituted as described herein.

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned pharmaceutical composition wherein X¹ and X² taken together with the nitrogen to which they are bonded are an optionally substituted heterocyclic group comprising 4-10 ring members and 0-3 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned pharmaceutical composition wherein X¹ and X² are hydrogen or an optionally substituted aliphatic, alicyclic, or aromatic group.

In certain embodiments, the present invention relates to the aforementioned pharmaceutical composition wherein X¹ and X² are hydrogen, cyclopentyl, benzyl, 4-methoxyphenyl or 2-isopropylphenyl.

In certain embodiments, the present invention relates to the aforementioned pharmaceutical composition wherein X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned pharmaceutical composition wherein R¹ is hydrogen, halogen, C₁₋₆ alkyl, aryl-C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, aryl, or heterocycle; wherein one or more of the foregoing aliphatic, cyclic, aromatic or heteroaromatic substituents optionally may be further substituted with C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, fluoro, cyano, hydroxy, carboxy, carboxy ester, amine, C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned pharmaceutical composition wherein R¹ is hydrogen, halogen, C₁₋₆ alkyl or C₁₋₆ alkoxy.

In certain embodiments, the present invention relates to the aforementioned pharmaceutical composition wherein R¹ is hydrogen.

In the non-limiting, exemplary pharmaceutical composition of the invention described herein, any of the —NX¹X² substituents present amongst the various compounds described may be utilized as the —NX³X⁴ substituents, and vice versa. In certain embodiments, the present invention relates to the aforementioned pharmaceutical composition wherein —NX¹X² and/or —NX³X⁴ are selected from the two sets of —NX¹X²/—NX³X⁴ moieties described for formula I above.

In certain embodiments, the present invention relates to the aforementioned pharmaceutical composition wherein X¹ is cyclopentyl; and X² is hydrogen. For example, one aspect of the invention relates to pharmaceutical composition comprising a compound selected from the group consisting of the compounds shown below.

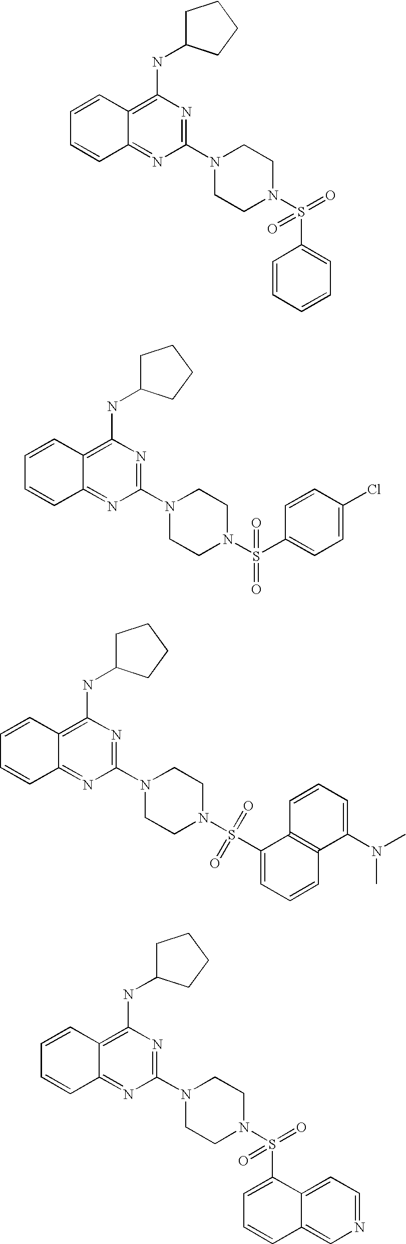

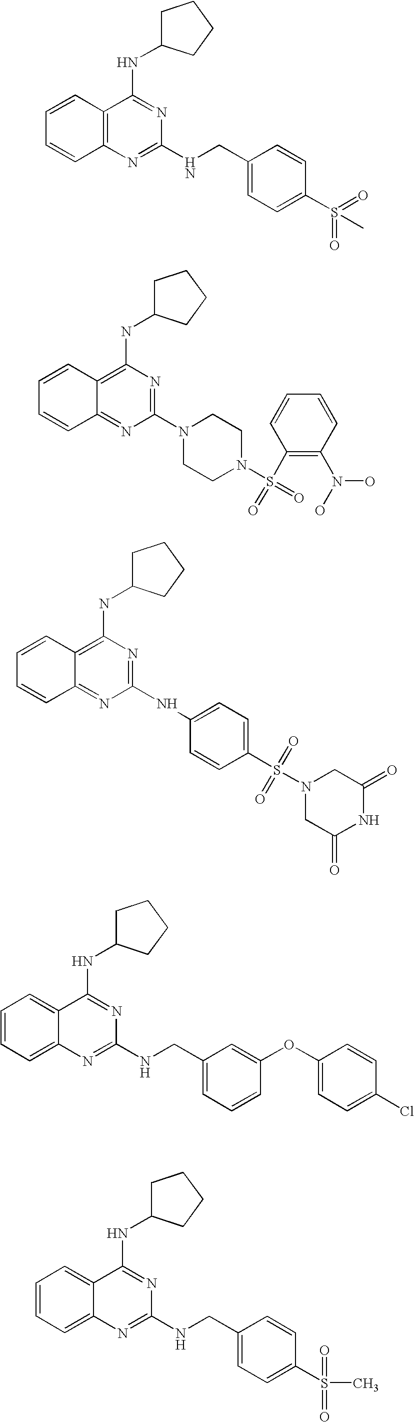

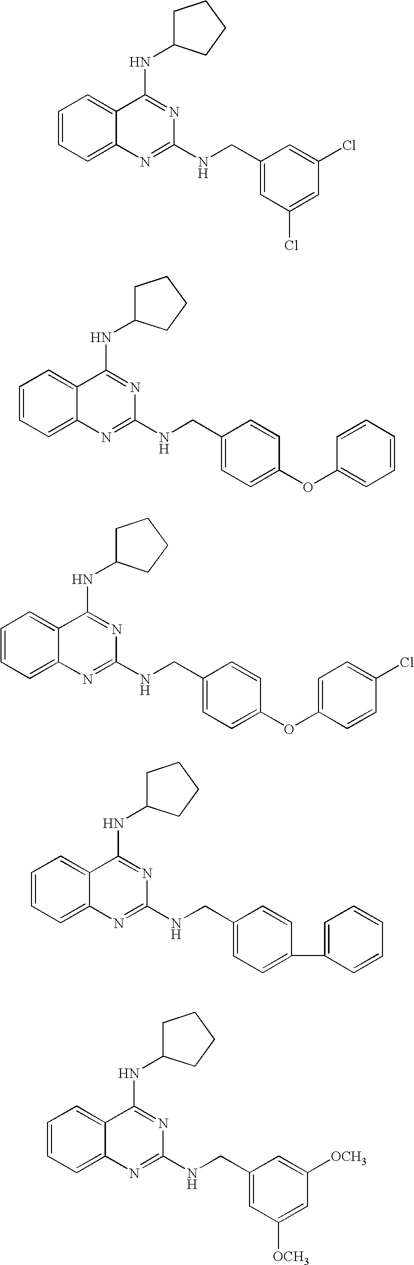

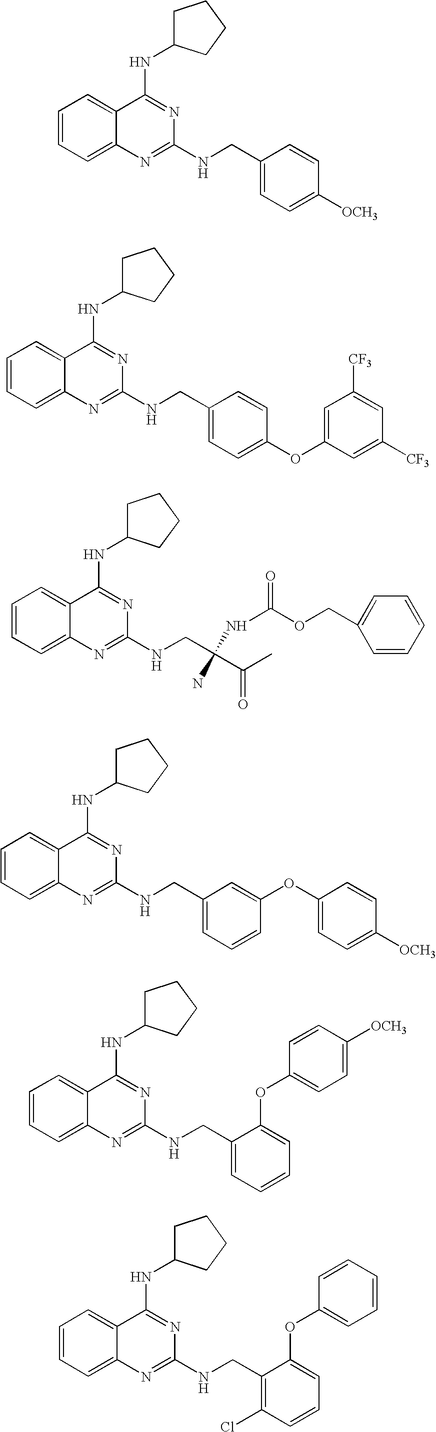

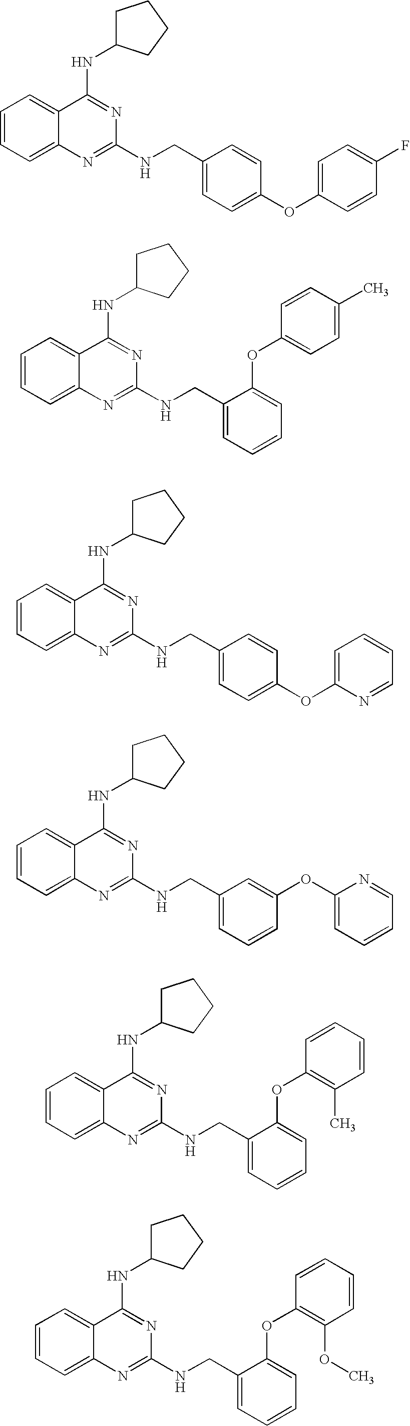

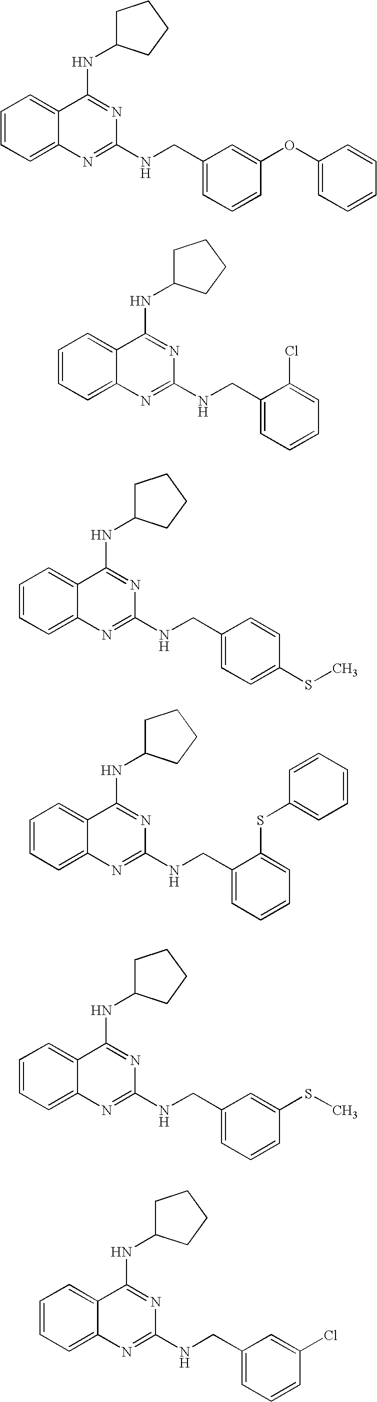

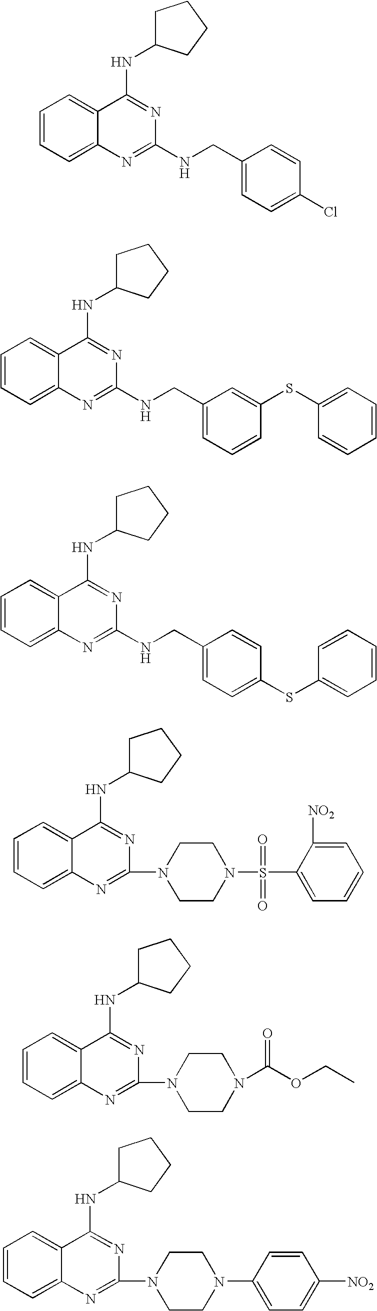

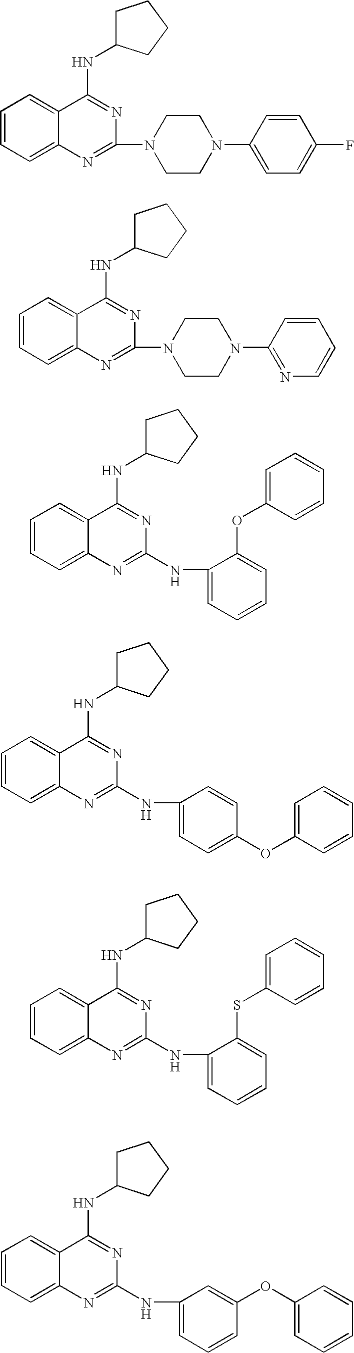

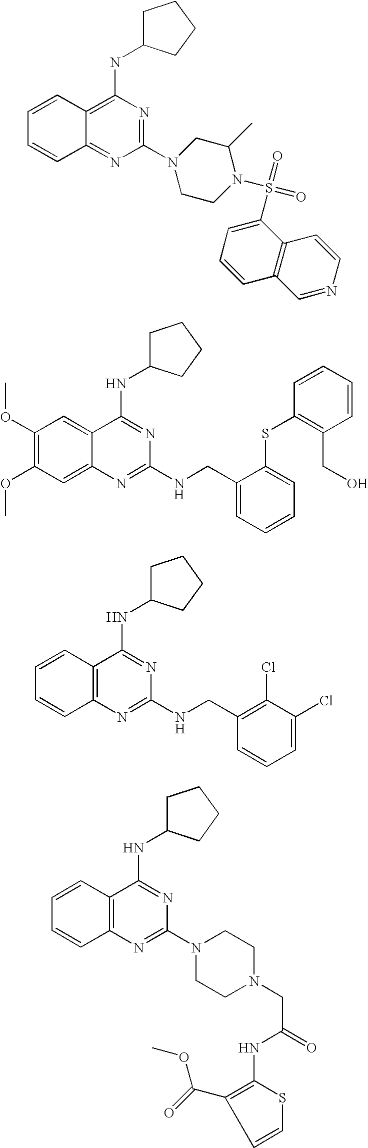

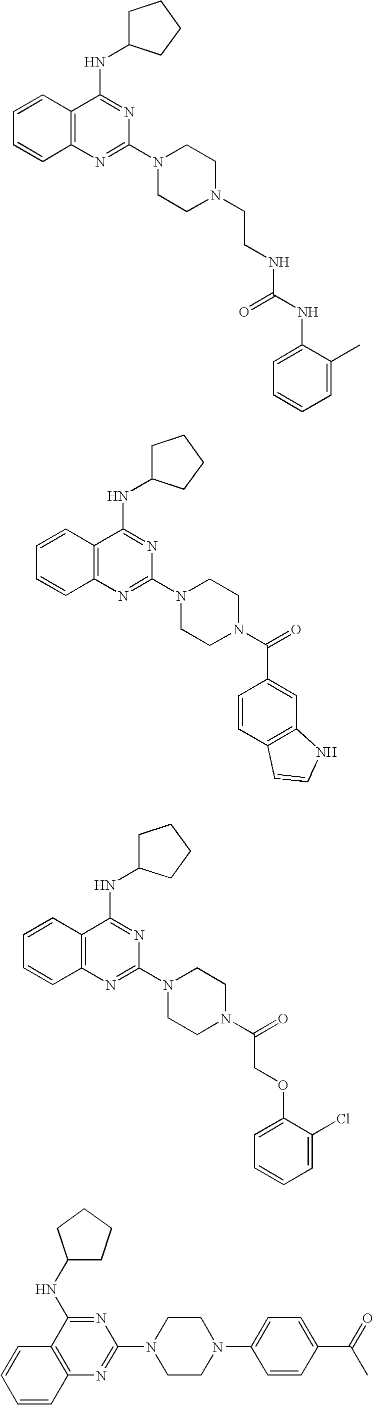

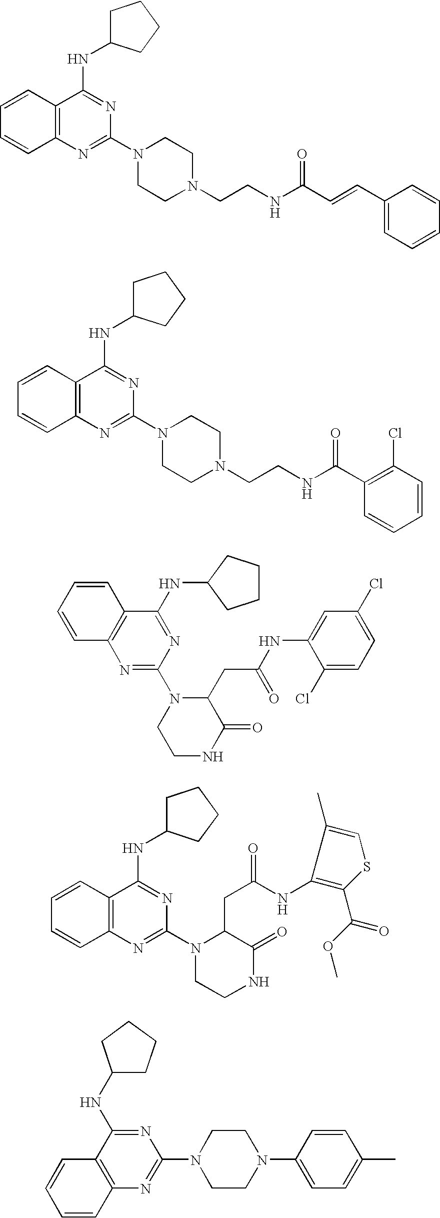

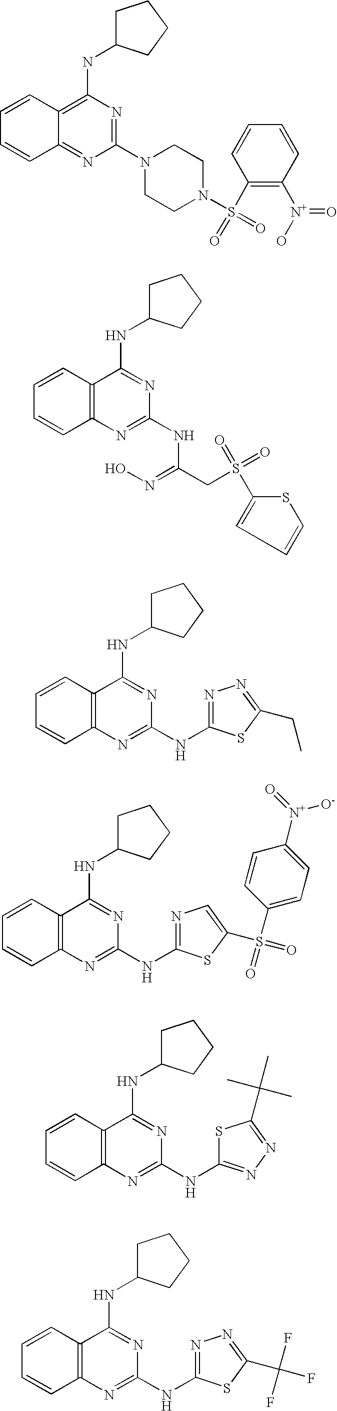

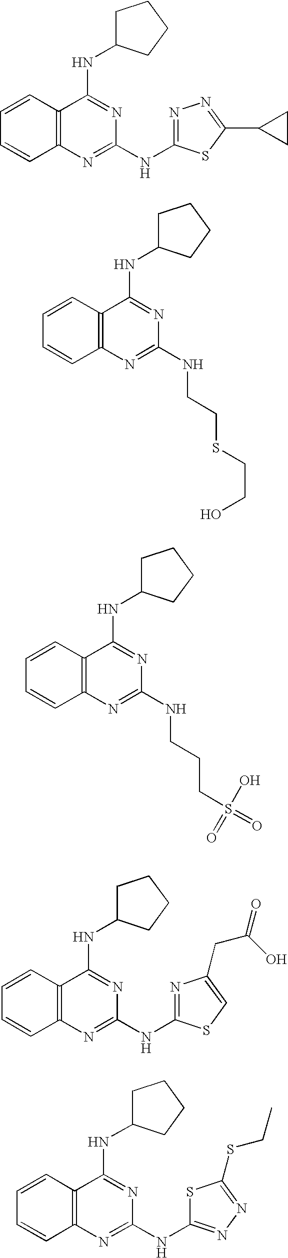

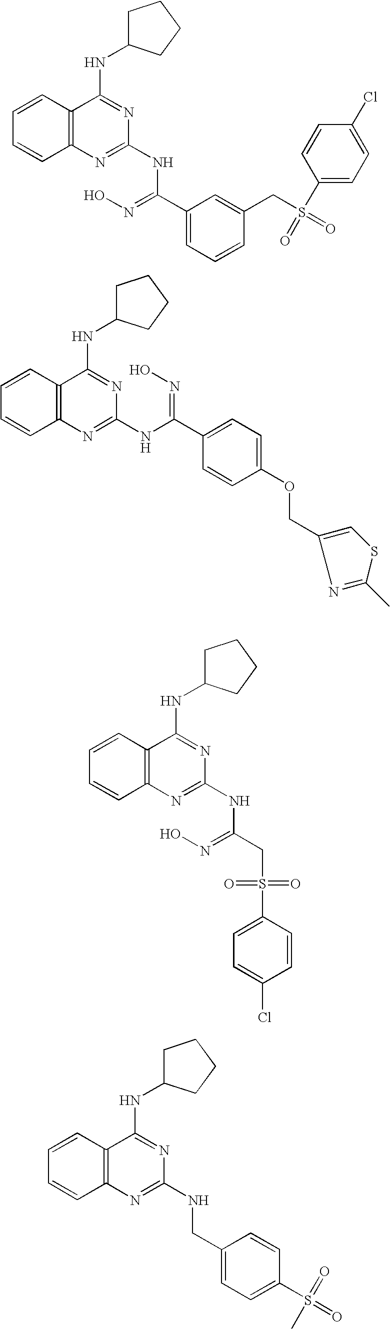

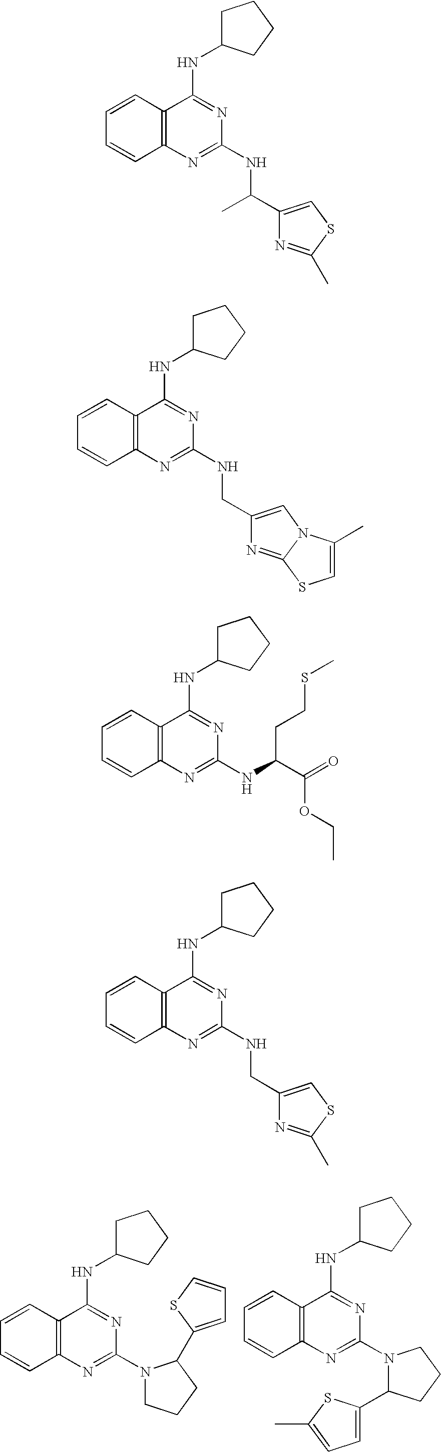

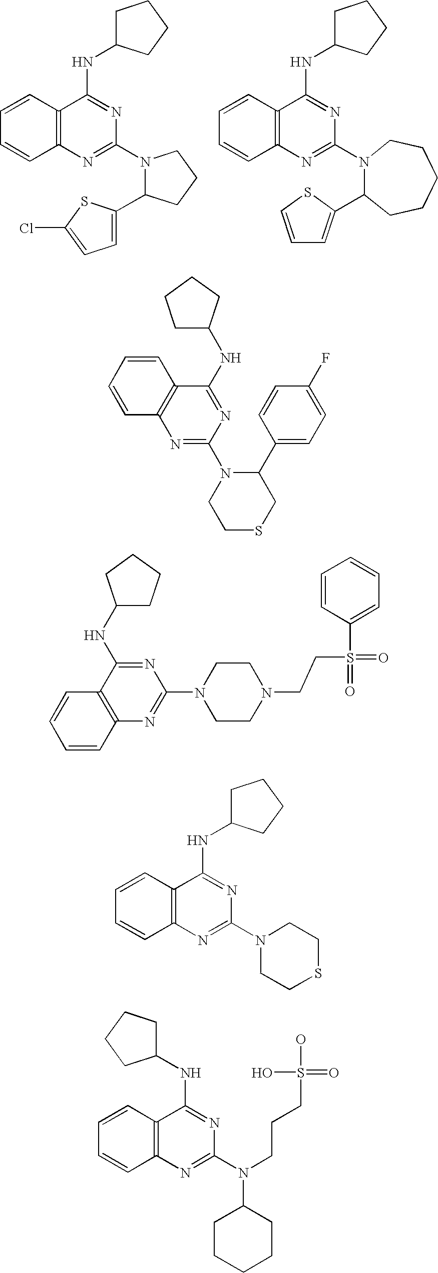

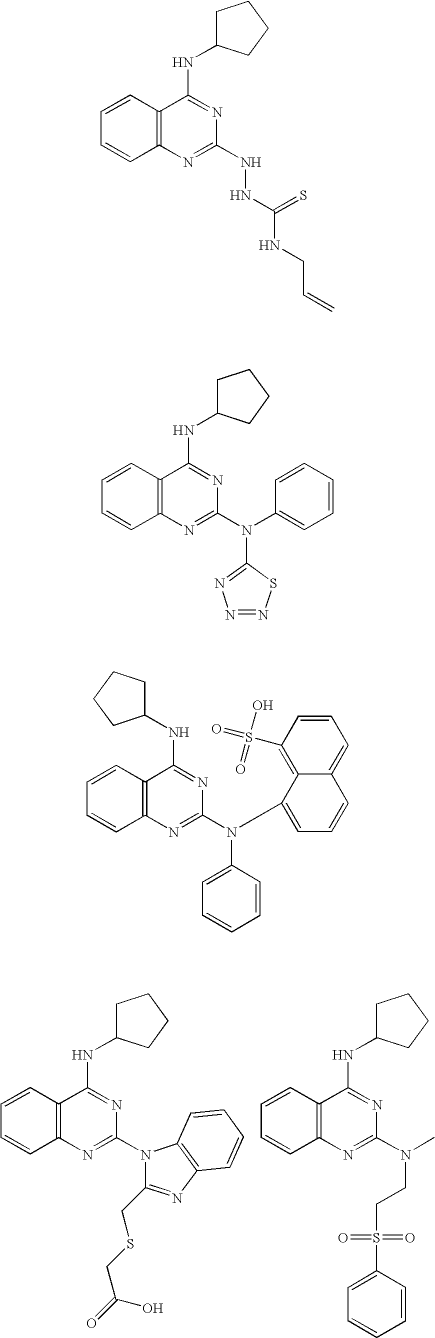

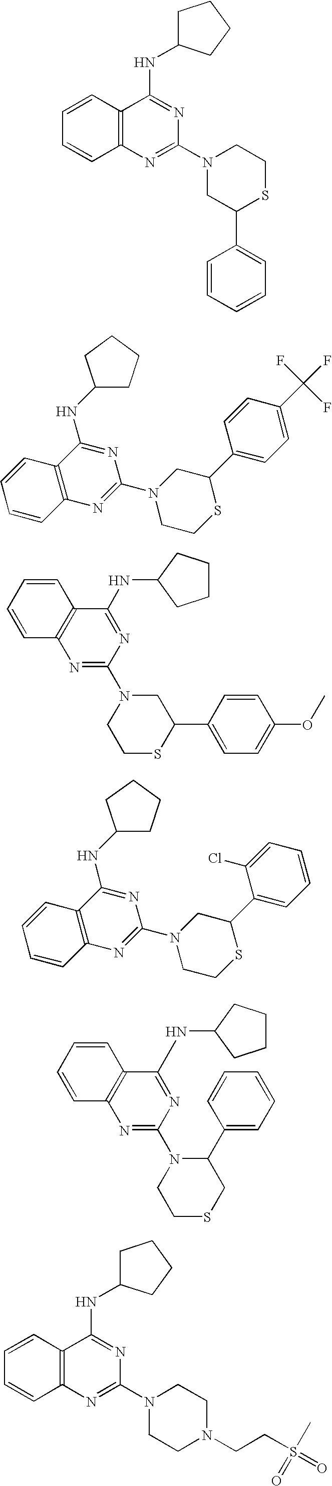

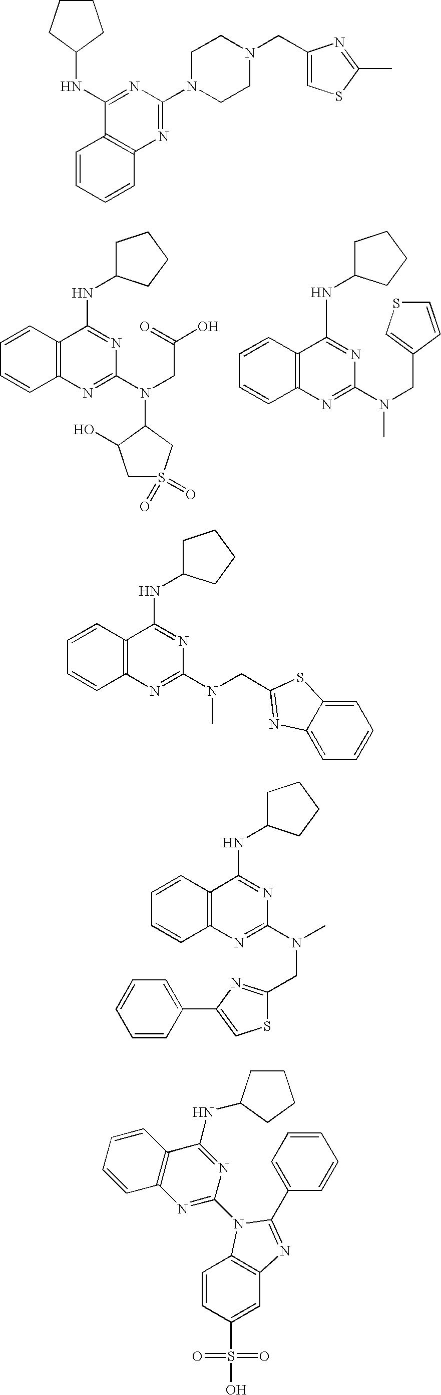

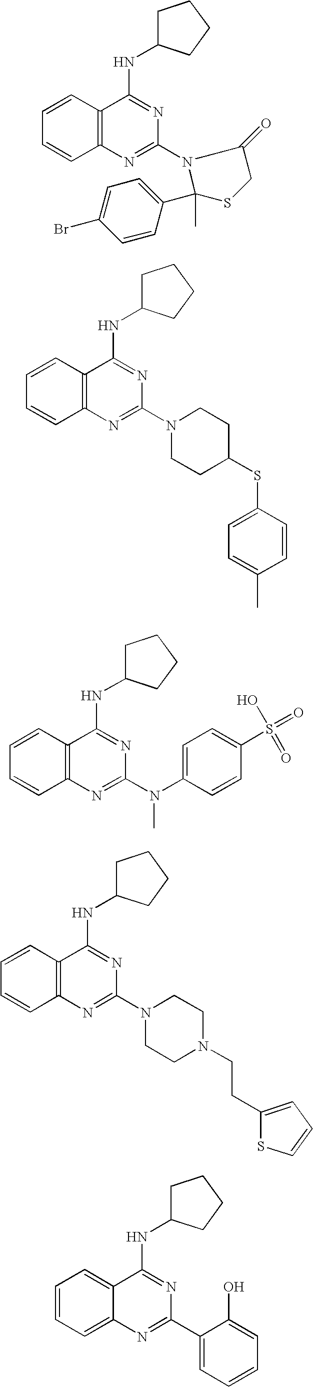

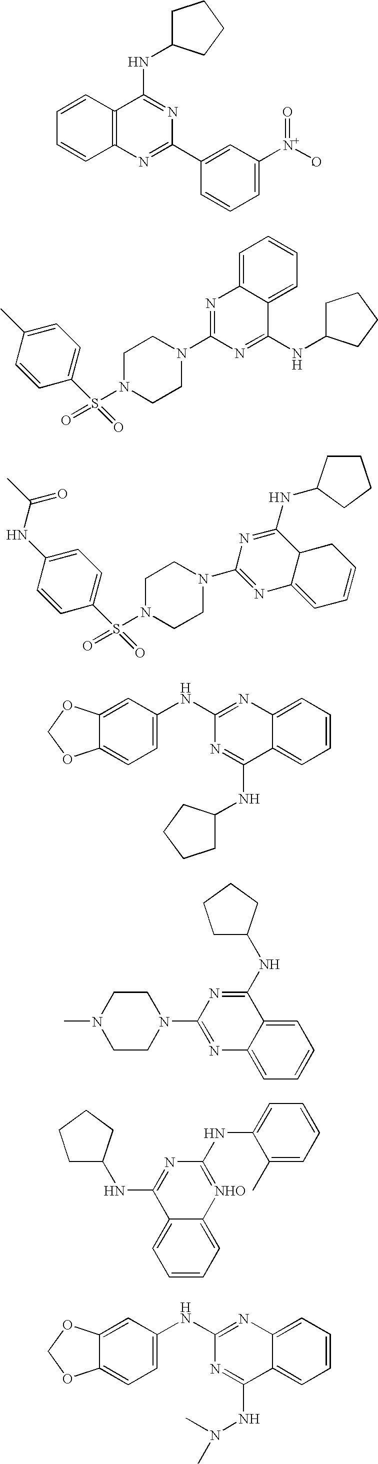

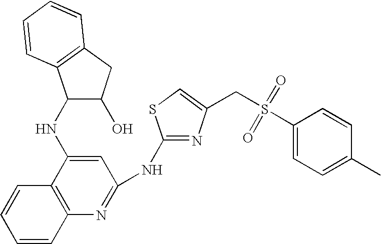

Another aspect of the present invention relates to a pharmaceutical composition comprising a compound of formula II:

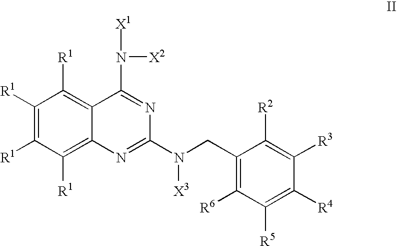

or a pharmaceutically acceptable salt thereof,

wherein, independently for each occurrence:

R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R²R³, R⁴, R⁵, and R⁶ are hydrogen, —F, —Cl, —Br, —I, —OH, —SH, —NO₂, —CN, —OR^{R}, —SR^{R}, S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹, X² and X³ are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned pharmaceutical composition, provided that when R¹ is hydrogen; R² is —SR^{R}; R³ is hydrogen; R⁴ is hydrogen; R⁵ is hydrogen; R⁶ is hydrogen; R^{R} is 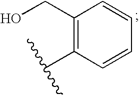
and —NX¹X² is 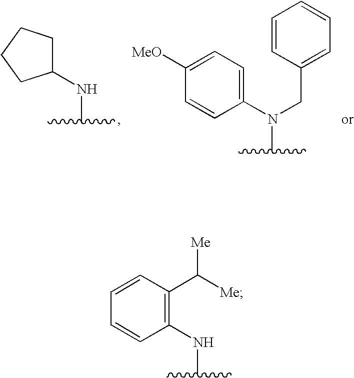
X³ is not hydrogen.

In certain embodiments, the present invention relates to the aforementioned pharmaceutical composition wherein R¹ is hydrogen; halogen; a saturated or unsaturated, branched or straight-chain C₁₋₆ alkyl; aryl-C₁₋₆ alkyl; mono- or polyfluorinated C₁₋₆ alkyl; C₁₋₆ alkoxy; C₁₋₆ alkylamino; di(C₁₋₆ alkyl)amino; C₁₋₈ alkylamino-C₁₋₈ alkyl; di(C₁₋₆ alkyl)amino-C₁₋₈ alkyl; cyclo(C₃₋₆)alkyl; aryl, wherein the aryl comprises a six membered aromatic carbocycle (such as phenyl) or a polycyclic aromatic hydrocarbon (such as naphthyl, phenanthracenyl, indanyl); a heterocycle, wherein the heterocycle comprises six membered aromatic heterocycles (such as pyridyl, diazinyl, pyrimidinyl, pyrrolidinyl, piperazinyl, thiazinyl), five membered aromatic heterocycles (such as pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thienyl) or bicyclic systems (such as indolyl, benzothienyl, benzofuranyl, isoindolyl, isobenzothienyl, isobenzofuranyl); wherein any of wherein one or more of the foregoing aliphatic, cyclic, aromatic or heteroaromatic substituents optionally may be further substituted with a C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, cyano, hydroxy, carboxy, carboxy ester, amine (optionally substituted with C₁₋₆ straight chain alkyl), C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned pharmaceutical composition wherein R¹ represents two non-hydrogen substituents which combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned pharmaceutical composition, wherein R², R³, R⁴, R⁵ and R⁶, and the carbons to which they are bonded, may combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned pharmaceutical composition wherein X¹, X² and X³ are independently selected from the group consisting of hydrogen, a C₁₋₆ straight chain saturated or unsaturated alkyl group, a C₃₋₆ branched saturated or unsaturated chain alkyl group, a C₃₋₆ cycloalkyl group; and any of the foregoing are optionally substituted with one or more halo, nitro, cyano, hydroxy, carboxy, carboxy ester, amine (optionally substituted with C₁₋₆ straight chain alkyl), C₃₋₆ branched chain alkyl, C₃₋₆ cycloalkyl, aromatic group or aralkyl group (such as phenyl, benzyl or naphthyl, optionally further substituted as described above), fused alkyl or aromatic ring, or heteroaromatic or heterocyclic ring, which may be a saturated or unsaturated ring containing 4-10 ring members and 0-3 heteroatoms selected from the group consisting of O, N and S, the heteroaromatic or heterocyclic ring optionally substituted with one or more halo, C₁₋₆ straight chain alkyl, C₃₋₆ branched chain alkyl, C₃₋₆ cycloalkyl, C₁₋₆ alkyloxy, nitro, cyano, hydroxy, carboxyl, ester, amine (optionally substituted with C₁₋₆ straight chain alkyl), C₃₋₆ branched chain alkyl or C₃₋₆ cycloalkyl, trifluoroxy, trifluoromethyl, difluoromethyl, aryl, the same or different heterocyclic ring, or a fused aromatic, heteroaromatic or heterocyclic ring. The alkyl group of alkyloxy may be a C₁₋₆ straight chain, C₃₋₆ branched or C₃₋₆ cycloalkyl; and any of the alkyl groups herein may be saturated or contain one or more degrees of unsaturation; or X¹ and X² together with the nitrogen to which they are bonded is an optionally substituted heteroaromatic or heterocyclic ring comprising in addition to the aforementioned nitrogen, 4-10 ring members and 0-3 additional heteroatoms selected from the group consisting of O, N and S, the heteroaromatic or heterocyclic ring optionally further substituted with one or more aliphatic, aromatic, —SR^{R}, —OR^{R}, heteroaromatic or fused rings which may be further substituted as described herein.

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned pharmaceutical composition wherein X¹ and X² taken together with the nitrogen to which they are bonded are an optionally substituted heterocyclic group comprising 4-10 ring members and 0-3 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned pharmaceutical composition wherein R¹, R³, R⁴, R⁵ and R⁶ are hydrogen; R² is —SR^{R}; and R^{R} is an optionally substituted phenyl group. Examples of substitutions of said phenyl group include a hydroxyalkyl group (such as hydroxymethyl and hydroxyethyl); a haloalkyl group (such as fluoromethyl, difluoromethyl and trifluoromethyl); an alkoxyalkyl group (such as ethoxymethyl and methoxymethyl); a carboxyalkyl group (such as carboxymethyl and carboxyethyl); a —COOH; a C₁₋₆ alkylidene-O(C═O)-alkyl or C₁₋₆ alkylidene-(C═O)-alkoxy group (such as —CH₂—OC(═O)—CH₃ and —CH₂CH₂—C(═O)—OCH₃); an amide, alkylamide or dialkylamide; and an alkylaminocarboxy moiety (such as —OC(═O)NHEt).

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned pharmaceutical composition wherein X¹ and X² are hydrogen or an optionally substituted aliphatic, alicyclic, or aromatic group.

In certain embodiments, the present invention relates to the aforementioned pharmaceutical composition wherein X¹ and X² are hydrogen, cyclopentyl, benzyl, 4-methoxyphenyl or 2-isopropylphenyl.

In certain embodiments, the present invention relates to the aforementioned pharmaceutical composition wherein X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned pharmaceutical composition wherein —NX¹X² is selected from the two sets of —NX¹X² moieties described for formula I above.

In certain embodiments, the present invention relates to the aforementioned pharmaceutical composition wherein R² is —SR^{R}.

In certain embodiments, the present invention relates to the aforementioned pharmaceutical composition wherein R² is —SR^{R}; and R³, R⁴, R⁵ and R⁶ are hydrogen.

In certain embodiments, the present invention relates to the aforementioned pharmaceutical composition wherein R² is —SR^{R}; R³, R⁴, R⁵ and R⁶ are hydrogen; and R^{R} is an optionally substituted phenyl.

In certain embodiments, the present invention relates to the aforementioned pharmaceutical composition wherein R² is —SR^{R}; R³, R⁴, R⁵ and R⁶ are hydrogen; R^{R} is 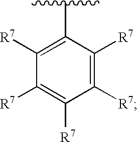
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned pharmaceutical composition wherein R² is —SR^{R}; R³, R⁴, R⁵ and R⁶ are hydrogen; R^{R} is 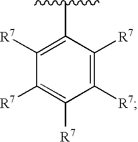
and R⁷ is, independently for each occurrence, hydrogen, hydroxymethyl, hydroxyethyl, fluoromethyl, difluoromethyl, trifluoromethyl, ethoxymethyl, methoxymethyl, carboxymethyl, carboxyethyl, —COOH, —CH₂—OC(═O)—CH₃, —CH₂CH₂—C(═O)—OCH₃ or —O(CO)NHEt.

In certain embodiments, the present invention relates to the aforementioned pharmaceutical composition wherein R² is —SR^{R}; R³, R⁴, R⁵ and R⁶ are hydrogen; and R^{R} is

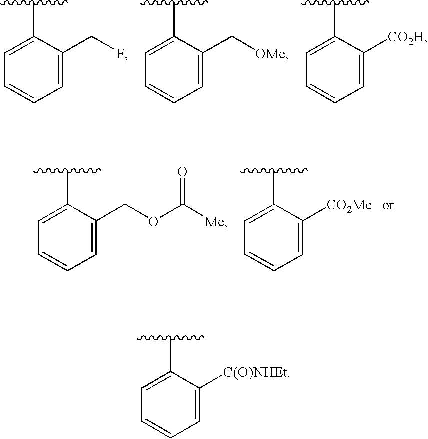

In certain embodiments, the present invention relates to the aforementioned pharmaceutical composition wherein R¹ is hydrogen, halogen, C₁₋₆ alkyl, aryl-C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, aryl, or heterocycle; wherein one or more of the foregoing aliphatic, cyclic, aromatic or heteroaromatic substituents optionally may be further substituted with C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, fluoro, cyano, hydroxy, carboxy, carboxy ester, amine, C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned pharmaceutical composition wherein R¹ is hydrogen, halogen, C₁₋₆ alkyl or C₁₋₆ alkoxy.

In certain embodiments, the present invention relates to the aforementioned pharmaceutical composition wherein R¹ is hydrogen.

In certain embodiments, the present invention relates to the aforementioned pharmaceutical composition wherein X³ is hydrogen, aliphatic or alicyclic.

In certain embodiments, the present invention relates to the aforementioned pharmaceutical composition wherein X³ is hydrogen or C₁₋₆ alkyl.

In certain embodiments, the present invention relates to the aforementioned pharmaceutical composition wherein X³ is hydrogen.

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups; and R² is —SR^{R}.

In certain embodiments, the present invention relates to the aforementioned pharmaceutical composition wherein X¹ and X² are hydrogen or an optionally substituted aliphatic, alicyclic, heteroaliphatic, heterocyclic, aromatic, heteroaromatic or acyl group; R² is —SR^{R}; R³, R⁴, R⁵ and R⁶ are hydrogen; and R^{R} is an optionally substituted phenyl.

In certain embodiments, the present invention relates to the aforementioned pharmaceutical composition wherein X¹ and X² are hydrogen or an optionally substituted aliphatic, alicyclic, or aromatic group; and R² is —SR^{R}; R³, R⁴, R⁵ and R⁶ are hydrogen; R^{R} is 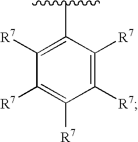
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned pharmaceutical composition wherein X¹ and X² are hydrogen or an optionally substituted aliphatic, alicyclic, or aromatic group; and R² is —SR^{R}; R³, R⁴, R⁵ and R⁶ are hydrogen; R^{R} is 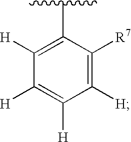
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned pharmaceutical composition wherein X¹ and X² are hydrogen, cyclopentyl, benzyl, 4-methoxyphenyl or 2-isopropylphenyl; R² is —SR^{R}; R³, R⁴, R⁵ and R⁶ are hydrogen; and R^{R} is

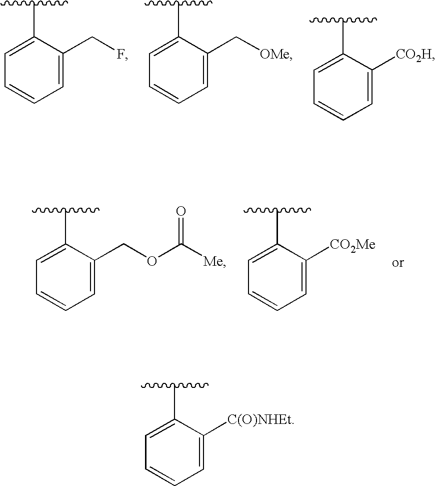

In certain embodiments, the present invention relates to the aforementioned pharmaceutical composition wherein X¹ and X² are hydrogen, cyclopentyl, benzyl, 4-methoxyphenyl or 2-isopropylphenyl; R² is —SR^{R}; R³, R⁴, R⁵ and R⁶ are hydrogen; R^{R} is 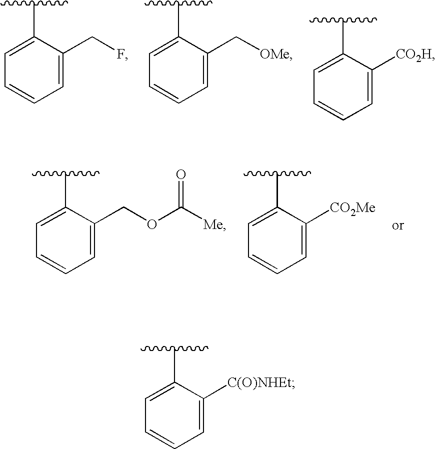
and R¹ is hydrogen.

Another aspect of the invention relates to pharmaceutical composition comprising a compound selected from the group consisting of the compounds shown below.

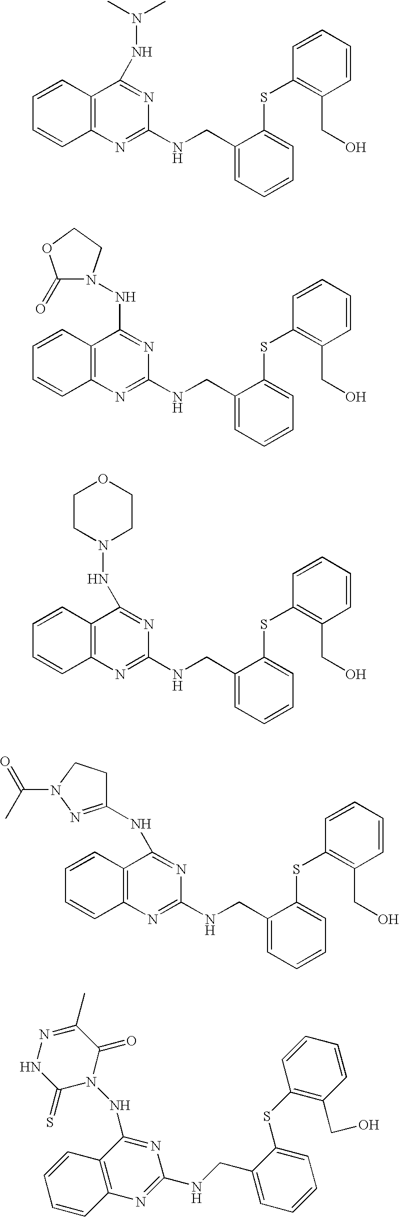

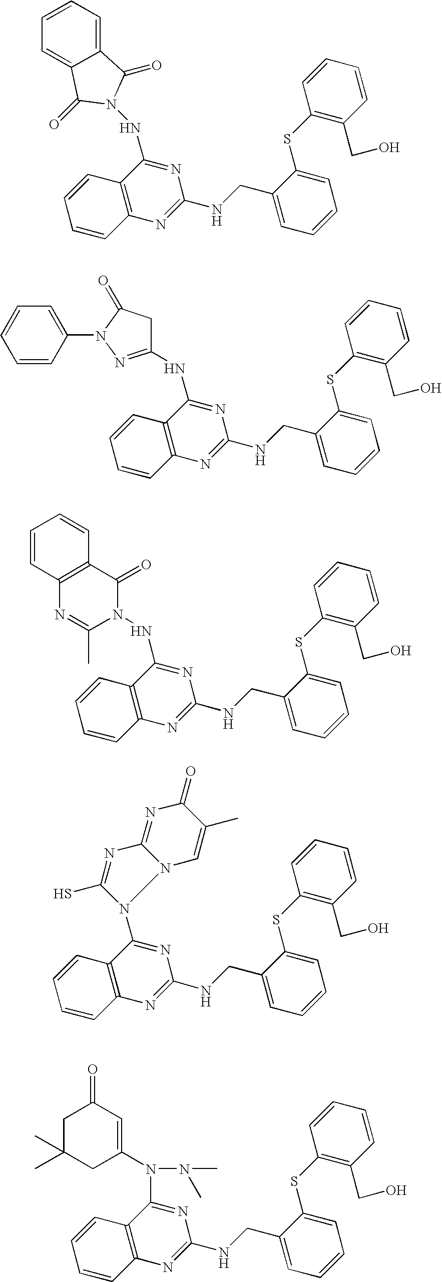

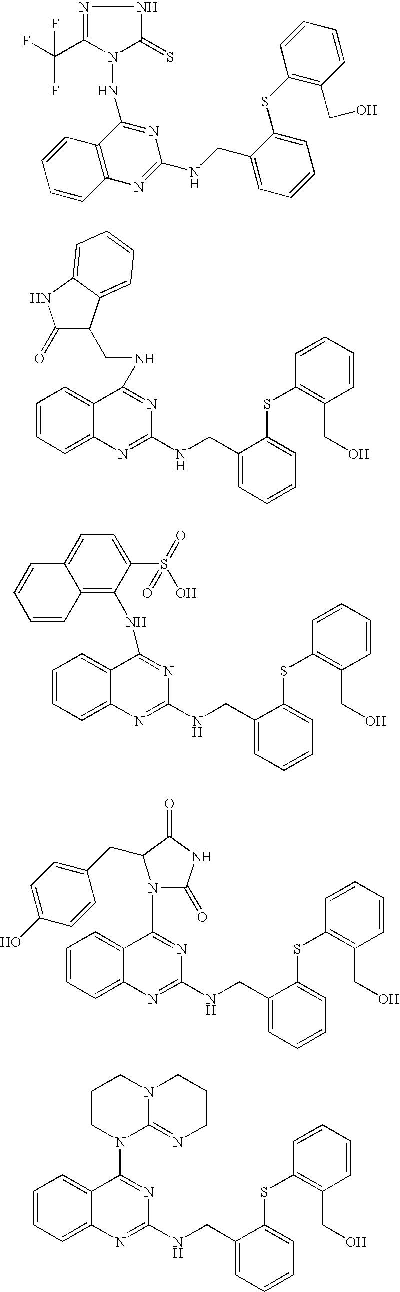

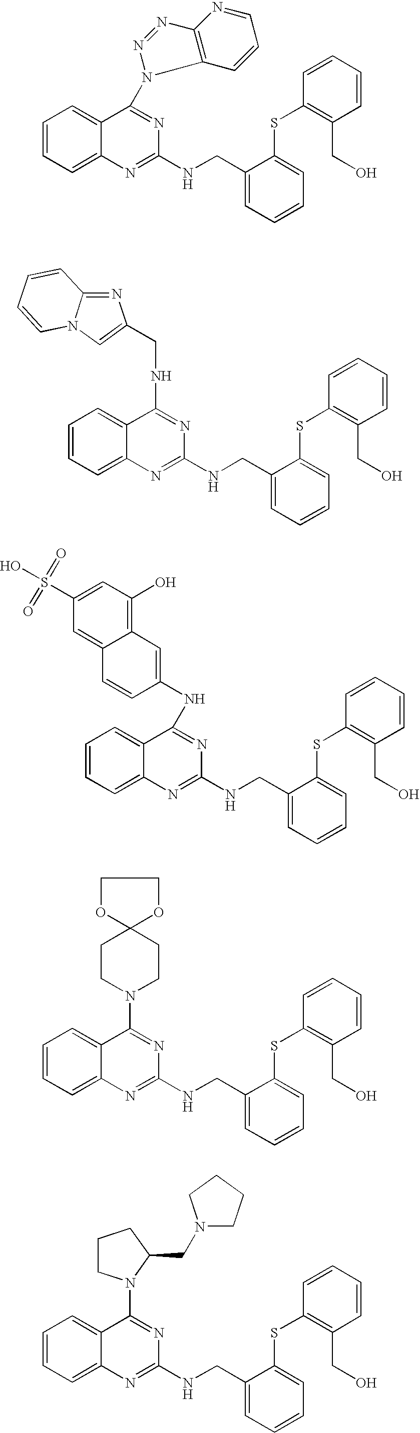

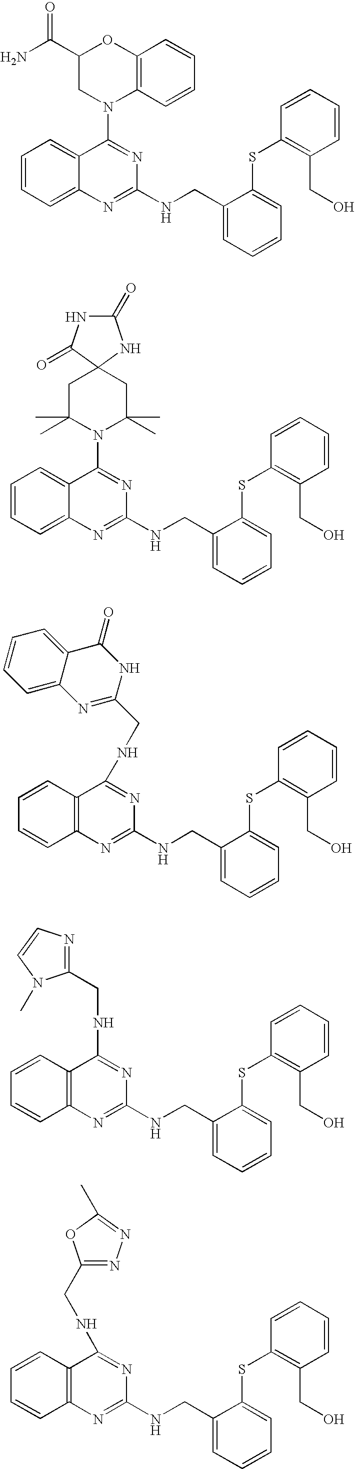

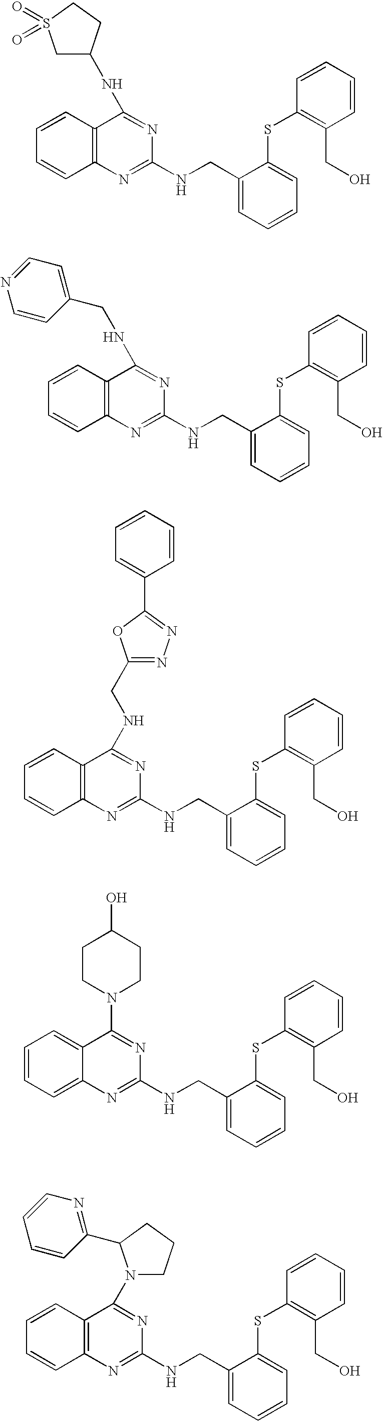

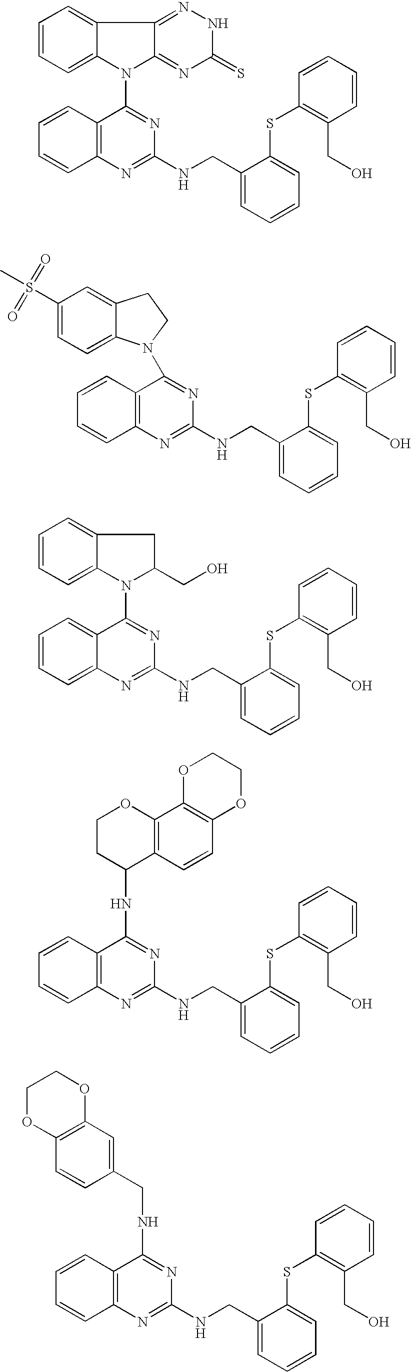

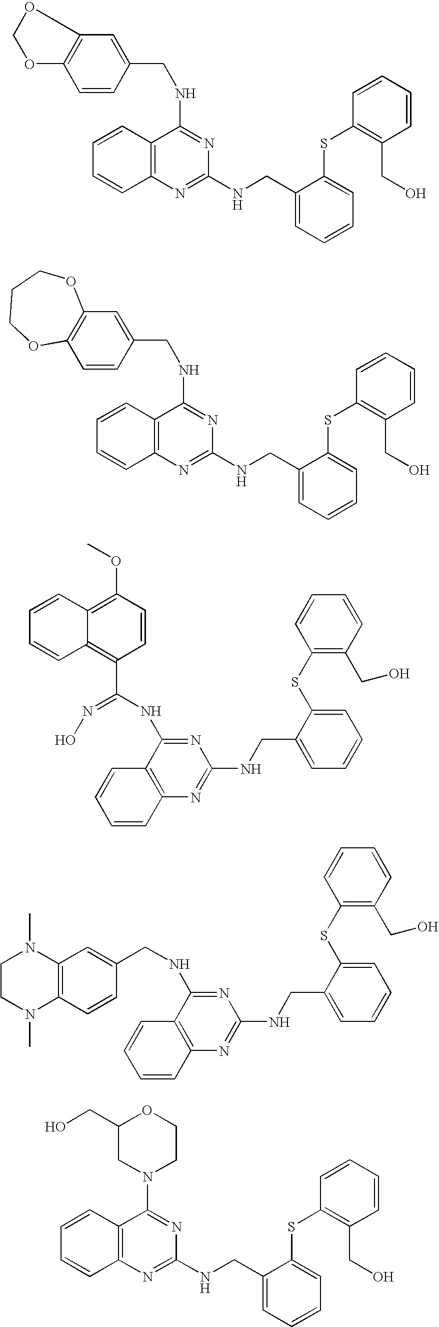

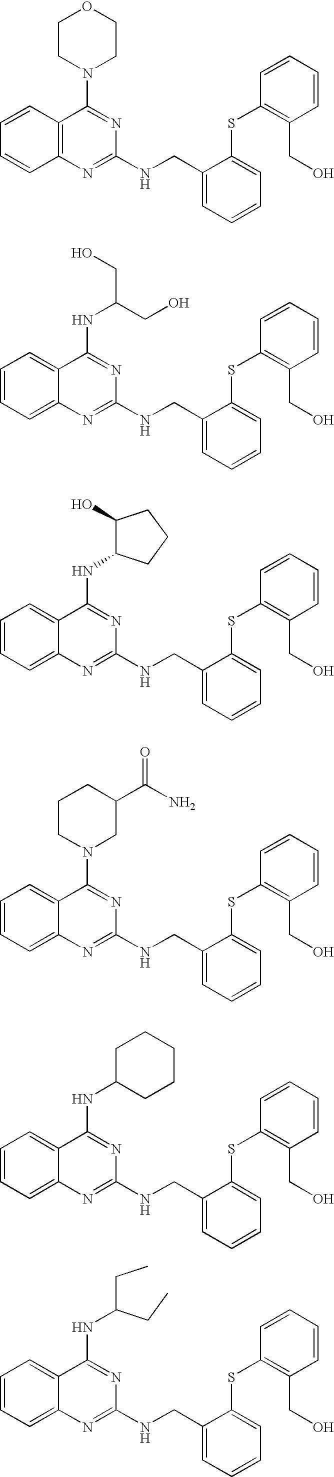

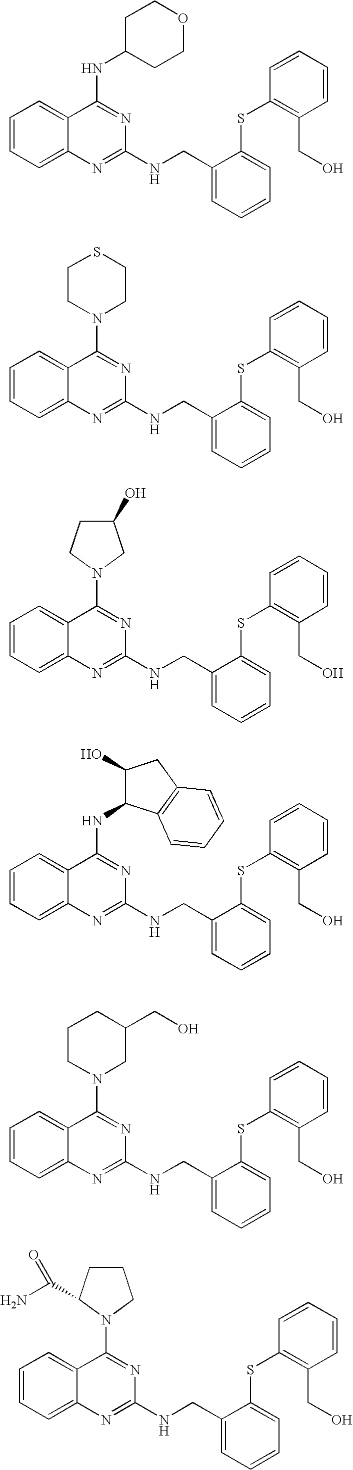

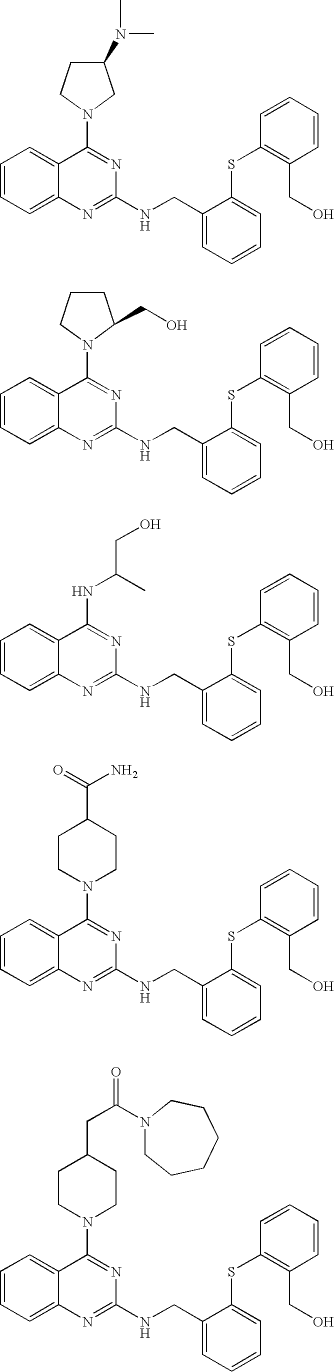

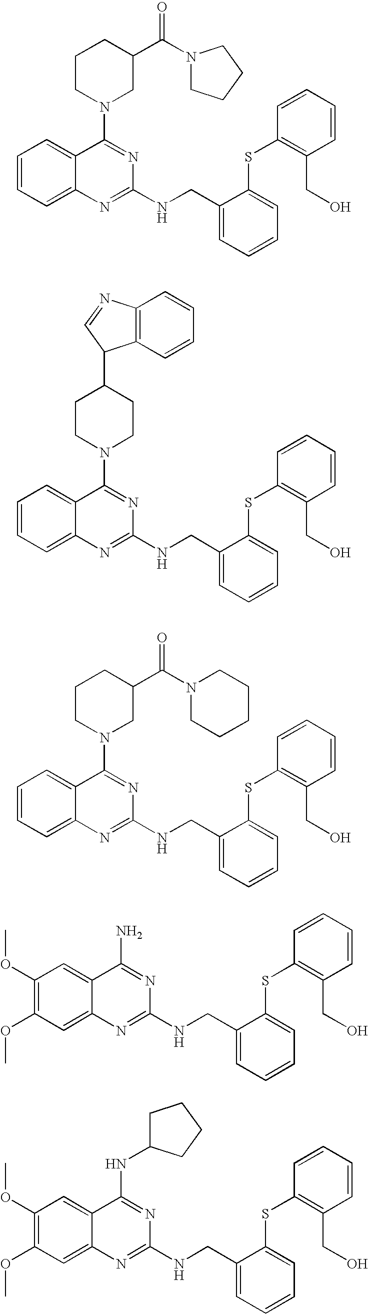

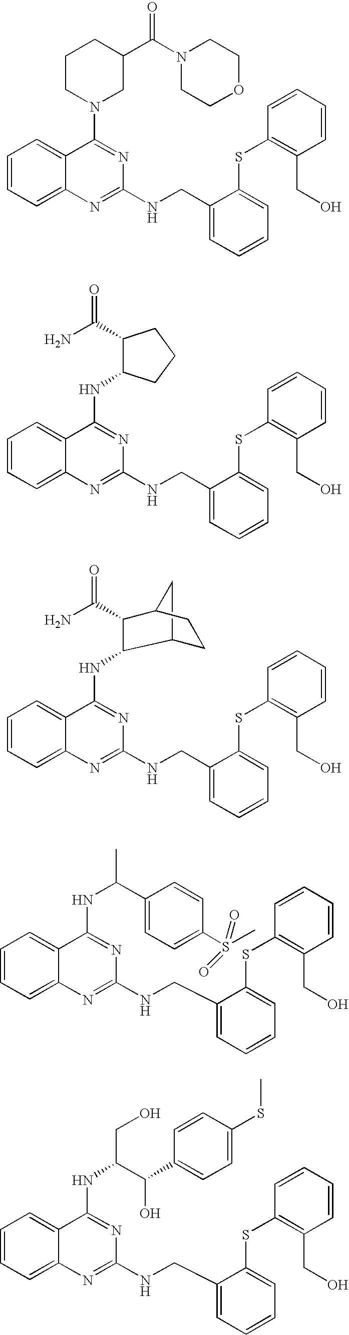

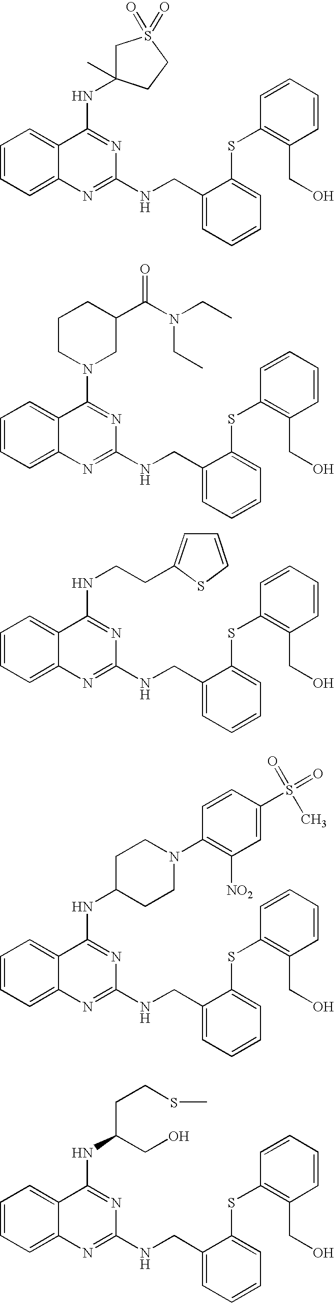

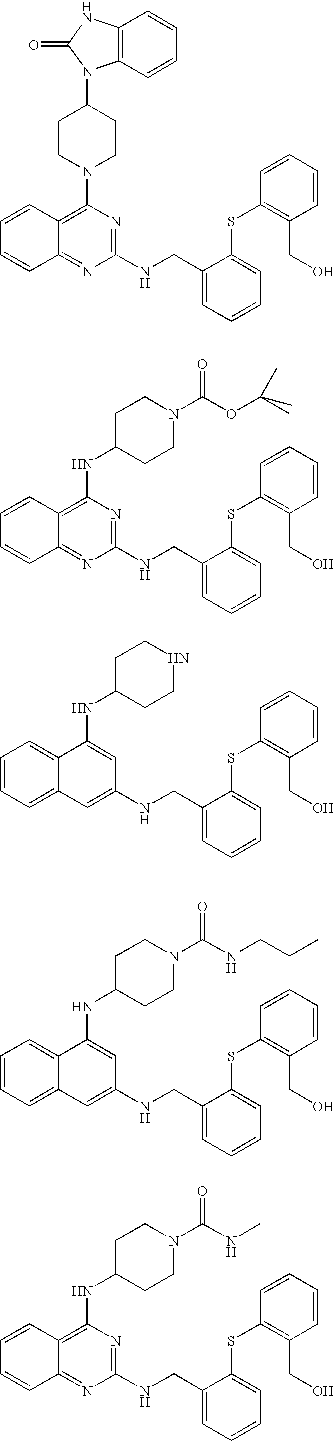

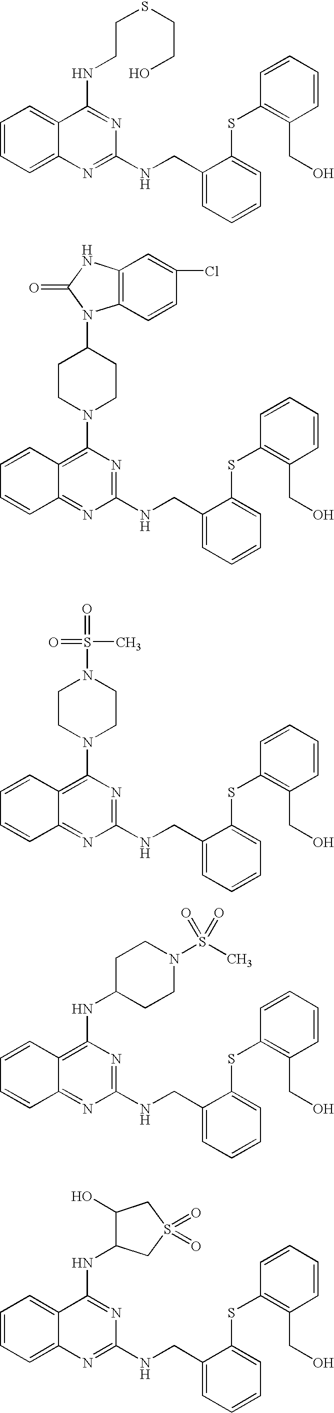

In another embodiment, the present invention relates to a pharmaceutical composition comprising a compound of formula IV:

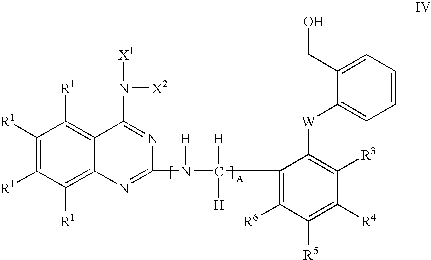

or a pharmaceutically acceptable salt thereof,

wherein, independently for each occurrence:

A is 0 or 1;

W is S, S(O) or S(O)₂;

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned pharmaceutical composition provided that when W is S; R¹ is hydrogen; R³ is hydrogen; R⁴ is hydrogen; R⁵ is hydrogen; R⁶ is hydrogen; and —NX¹X² is 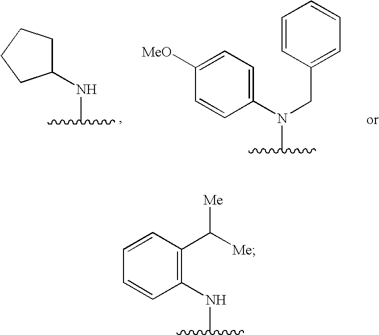
A is not 1.

In certain embodiments, the present invention relates to the aforementioned pharmaceutical composition wherein A is 0. In certain embodiments, the present invention relates to the aforementioned compound wherein A is 1.

In certain embodiments, the present invention relates to the aforementioned pharmaceutical composition wherein W is S. In certain embodiments, the present invention relates to the aforementioned pharmaceutical composition wherein W is S(O). In certain embodiments, the present invention relates to the aforementioned pharmaceutical composition wherein W is S(O)₂. In certain embodiments, the present invention relates to the aforementioned pharmaceutical composition wherein W is S or S(O)₂.

In certain embodiments, the present invention relates to the aforementioned pharmaceutical composition wherein A is 0; R³ is H; R⁴ is H; R⁵ is H; R⁶ is H; and W is S. In certain embodiments, the present invention relates to the aforementioned pharmaceutical composition wherein A is 0; R³ is H; R⁴ is H; R⁵ is OH; R⁶ is H; and W is S. In certain embodiments, the present invention relates to the aforementioned pharmaceutical composition wherein A is 0; R³ is H; R⁴ is H; R⁵ is H; R⁶ is H; and W is S(O)₂. In certain embodiments, the present invention relates to the aforementioned pharmaceutical composition wherein A is 0; R³ is H; R⁴ and R⁵ taken together are —OCH₂O—; R⁶ is H; and W is S(O)₂. In certain embodiments, the present invention relates to the aforementioned pharmaceutical composition wherein A is 0; R³ is OH; R⁴ is H; R⁵ is H; R⁶ is H; and W is S(O)₂. In certain embodiments, the present invention relates to the aforementioned pharmaceutical composition wherein A is 0; R³ is H; R⁴ is OH; R⁵ is H; R⁶ is H; and W is S(O)₂. In certain embodiments, the present invention relates to the aforementioned pharmaceutical composition wherein A is 0; R³ is H; R⁴ is H; R⁵ is OH; R⁶ is H; and W is S(O)₂. In certain embodiments, the present invention relates to the aforementioned pharmaceutical composition wherein A is 0; R³ is H; R⁴ is H; R⁵ is H; R⁶ is OH; and W is S(O)₂. In certain embodiments, the present invention relates to the aforementioned pharmaceutical composition wherein A is 1; R³ is H; R⁴ is Cl; R⁵ is H; R⁶ is H; and W is S(O)₂. In certain embodiments, the present invention relates to the aforementioned pharmaceutical composition wherein A is 0; R³ is Cl; R⁴ is H; R⁵ is H; R⁶ is H; and W is S. In certain embodiments, the present invention relates to the aforementioned pharmaceutical composition wherein A is 0; R³ is H; R⁴ is H; R⁵ is Cl; R⁶ is H; and W is S. In certain embodiments, the present invention relates to the aforementioned pharmaceutical composition wherein A is 0; R³ is H; R⁴ is H; R⁵ is H; R⁶ is Cl; and W is S. In certain embodiments, the present invention relates to the aforementioned pharmaceutical composition wherein A is 1; R³ is Cl; R⁴ is H; R⁵ is H; R⁶ is H; and W is S(O)₂. In certain embodiments, the present invention relates to the aforementioned pharmaceutical composition wherein A is 1; R³ is H; R⁴ is Cl; R⁵ is H; R⁶ is H; and W is S(O)₂. In certain embodiments, the present invention relates to the aforementioned pharmaceutical composition wherein A is 0; R³ is H; R⁴ is OH; R⁵ is OH; R⁶ is H; and W is S(O)₂. In certain embodiments, the present invention relates to the aforementioned pharmaceutical composition wherein A is 0; R³ is Cl; R⁴ is H; R⁵ is H; R⁶ is H; and W is S(O)₂. In certain embodiments, the present invention relates to the aforementioned pharmaceutical composition wherein A is 0; R³ is H; R⁴ is Cl; R⁵ is H; R⁶ is H; and W is S(O)₂.

In certain embodiments, the present invention relates to the aforementioned pharmaceutical composition wherein X¹ is cyclopentyl; and X² is hydrogen.

Another aspect of the invention relates to a pharmaceutical composition comprising one a compound selected from the group consisting of the compounds shown below.

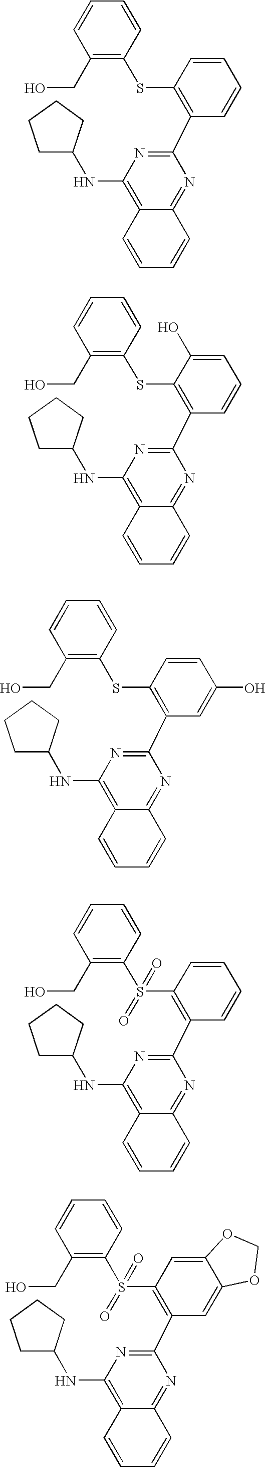

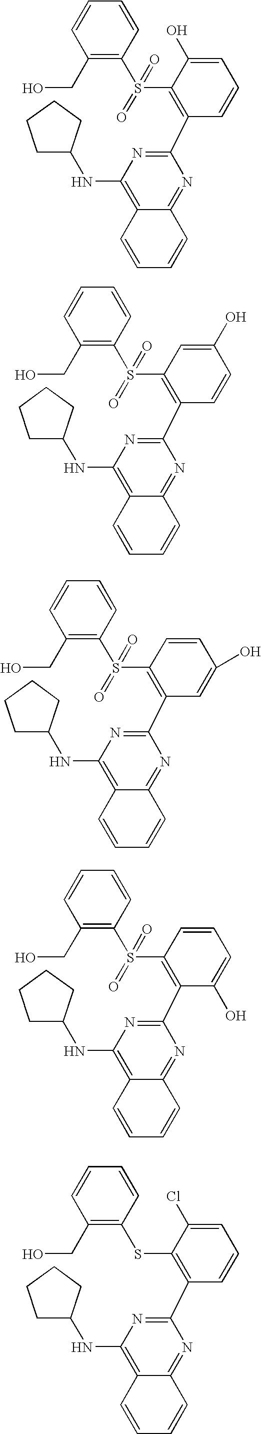

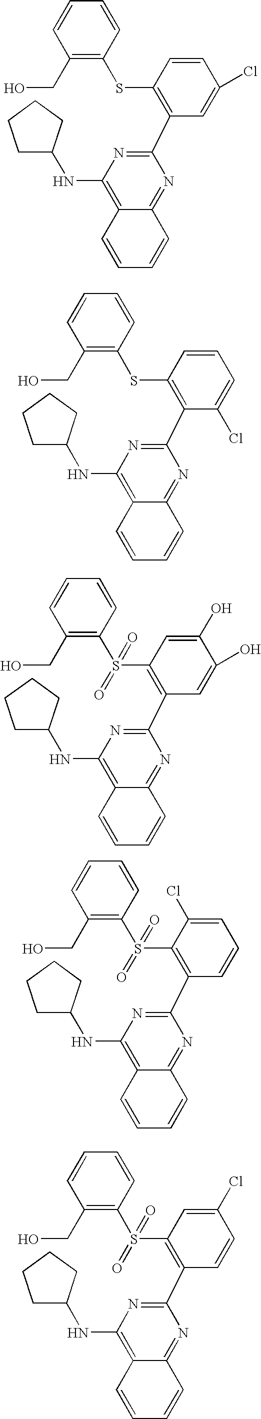

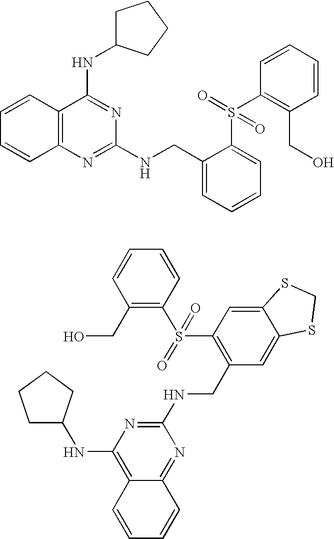

As mentioned before, the foregoing examples are merely illustrative and pharmaceutical composition comprising compounds comprising variants of the substituents as well as their positions are fully embraced by the teachings herein.

In another embodiment, the present invention relates to a pharmaceutical composition comprising a compound of formula V:

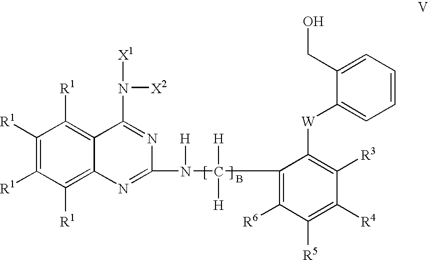

or a pharmaceutically acceptable salt thereof,

wherein, independently for each occurrence:

B is 0, 1, 2, 3, 4, or 5;

W is S, S(O) or S(O)₂;

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶ together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned pharmaceutical composition provided that when W is S; R¹ is hydrogen; R³ is hydrogen; R⁴ is hydrogen; R⁵ is hydrogen; R⁶ is hydrogen; and —NX¹X² is 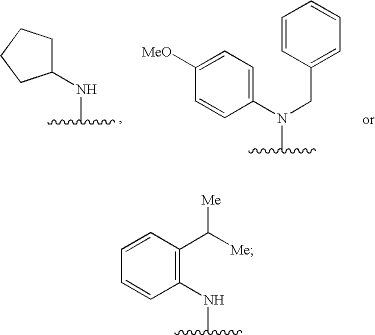
B is not 1.

In certain embodiments, the present invention relates to the aforementioned pharmaceutical composition wherein B is 0. In certain embodiments, the present invention relates to the aforementioned pharmaceutical composition wherein B is 1. In certain embodiments, the present invention relates to the aforementioned pharmaceutical composition wherein B is 2. In certain embodiments, the present invention relates to the aforementioned pharmaceutical composition wherein B is 3. In certain embodiments, the present invention relates to the aforementioned pharmaceutical composition wherein B is 4. In certain embodiments, the present invention relates to the aforementioned pharmaceutical composition wherein B is 5.

In certain embodiments, the present invention relates to the aforementioned pharmaceutical composition wherein W is S. In certain embodiments, the present invention relates to the aforementioned pharmaceutical composition wherein W is S(O). In certain embodiments, the present invention relates to the aforementioned pharmaceutical composition wherein W is S(O)₂. In certain embodiments, the present invention relates to the aforementioned pharmaceutical composition wherein W is S or S(O)₂.

In certain embodiments, the present invention relates to the aforementioned pharmaceutical composition wherein X¹ is cyclopentyl; R¹, X² and X³ are hydrogen; W is S; and at least one of R³, R⁴, R⁵ and R⁶ is not hydrogen.

In another embodiment, the present invention relates to a pharmaceutical composition comprising a compound of formula VI:

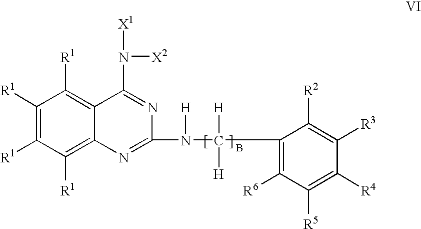

or a pharmaceutically acceptable salt thereof,

wherein, independently for each occurrence:

B is 0, 1, 2, 3, 4, or 5;

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R²R³, R⁴, R⁵, and R⁶ are hydrogen, —F, —Cl, —Br, —I, —OH, —SH, —NO₂, —CN, —OR^{R}, —SR^{R}, S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned pharmaceutical composition provided that when R¹ is hydrogen; R² is —SR^{R}; R³ is hydrogen; R⁴ is hydrogen; R⁵ is hydrogen; R⁶ is hydrogen; R^{R} is 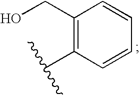
and —NX¹X² is 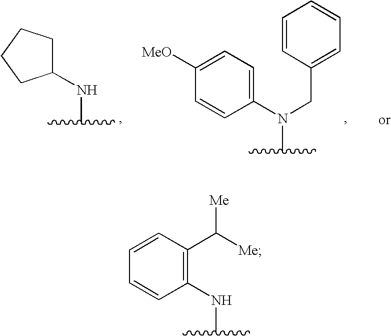
B is not 1.

Importantly, some of the compounds of the invention can comprise one or more asymmetric centers, and thus can exist in various isomeric forms, e.g., stereoisomers and/or diastereomers. Thus, the compounds in the pharmaceutical compositions described above may be in the form of an individual enantiomer, diastereomer or geometric isomer, or may be in the form of a mixture of stereoisomers. In certain embodiments, the compounds of the aforementioned pharmaceutical composition are enantiopure compounds. In certain other embodiments, mixtures of stereoisomers or diastereomers are provided in the pharmaceutical compositions.

Methods Using Compounds of the Invention

Methods for treating the various conditions and diseases described herein, in particular dysproliferative and inflammatory diseases, also benefit from the administration of the following compounds and pharmaceutical compositions comprising them.

In certain embodiments, the method involves the administration of a therapeutically effective amount of the compound or a pharmaceutically acceptable derivative thereof to a subject (including, but not limited to a human or animal) in need of it. Subjects for which the benefits of the compounds of the invention are intended for administration include, in addition to humans, livestock, domesticated, zoo and companion animals.

It will be appreciated that the compounds and compositions, according to the method of the present invention, may be administered using any amount and any route of administration effective for the treatment of conditions or diseases in which inhibiting c-Met or the activities thereof have a therapeutically useful role, and likewise for Tie-2. Thus, the expression “effective amount” as used herein, refers to a sufficient amount of agent to modulate c-Met activity (e.g., partially inhibit or block HGF/SF activity) or signaling or phosphorylation of c-Met or downstream signaling molecules, and to exhibit a therapeutic effect. The exact amount required will vary from subject to subject, depending on the species, age, and general condition of the subject, the severity of the infection, the particular therapeutic agent, its mode and route of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

Dysproliferative disorders refers in one embodiment to abnormal proliferation of cells, including hyperproliferative disorders, hyperplasia, metaplasia, dysplasia, by way of non-limiting examples, as described below.

Hyperproliferative Disorders. In certain embodiments, compounds and compositions of the invention can be used to treat or detect hyperproliferative disorders, including neoplasms. Compounds and compositions of the invention may inhibit the proliferation associated with the disorder through direct or indirect interactions. Alternatively, compounds and compositions of the invention may proliferate other cells which can inhibit the hyperproliferative disorder.

Examples of hyperproliferative disorders that can be treated or detected by compounds and compositions of the invention include, but are not limited to neoplasms located in the: colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvis, skin, soft tissue, spleen, thorax, and urogenital tract.

Similarly, other hyperproliferative disorders can also be treated or detected by compounds and compositions of the invention. Examples of such hyperproliferative disorders include, but are not limited to: acute childhood lymphoblastic leukemia, acute lymphoblastic leukemia, acute lymphocytic leukemia, acute myeloid leukemia, adrenocortical carcinoma, adult (primary) hepatocellular cancer, adult (primary) liver cancer, adult acute lymphocytic leukemia, adult acute myeloid leukemia, adult Hodgkin's disease, adult Hodgkin's lymphoma, adult lymphocytic leukemia, adult non-Hodgkin's lymphoma, adult primary liver cancer, adult soft tissue sarcoma, AIDS-related lymphoma, AIDS-related malignancies, anal cancer, astrocytoma, bile duct cancer, bladder cancer, bone cancer, brain stem glioma, brain tumors, breast cancer, cancer of the renal pelvis and ureter, central nervous system (primary) lymphoma, central nervous system lymphoma, cerebellar astrocytoma, cerebral astrocytoma, cervical cancer, childhood (primary) hepatocellular cancer, childhood (primary) liver cancer, childhood acute lymphoblastic leukemia, childhood acute myeloid leukemia, childhood brain stem glioma, childhood cerebellar astrocytoma, childhood cerebral astrocytoma, childhood extracranial germ cell tumors, childhood Hodgkin's disease, childhood Hodgkin's lymphoma, childhood hypothalamic and visual pathway glioma, childhood lymphoblastic leukemia, childhood medulloblastoma, childhood non-Hodgkin's lymphoma, childhood pineal and supratentorial primitive neuroectodermal tumors, childhood primary liver cancer, childhood rhabdomyosarcoma, childhood soft tissue sarcoma, childhood visual pathway and hypothalamic glioma, chronic lymphocytic leukemia, chronic myelogenous leukemia, colon cancer, cutaneous T-cell lymphoma, endocrine pancreas islet cell carcinoma, endometrial cancer, ependymoma, epithelial cancer, esophageal cancer, Ewing's sarcoma and related tumors, exocrine pancreatic cancer, extracranial germ cell tumor, extragonadal germ cell tumor, extrahepatic bile duct cancer, eye cancer, female breast cancer, Gaucher's disease, gallbladder cancer, gastric cancer, gastrointestinal carcinoid tumor, gastrointestinal tumors, germ cell tumors, gestational trophoblastic tumor, hairy cell leukemia, head and neck cancer, hepatocellular cancer, Hodgkin's disease, Hodgkin's lymphoma, hypergammaglobulinemia, hypopharyngeal cancer, intestinal cancers, intraocular melanoma, islet cell carcinoma, islet cell pancreatic cancer, Kaposi's sarcoma, kidney cancer, laryngeal cancer, lip and oral cavity cancer, liver cancer, lung cancer, lymphoproliferative disorders, macroglobulinemia, male breast cancer, malignant mesothelioma, malignant thymoma, medulloblastoma, melanoma, mesothelioma, metastatic occult primary squamous neck cancer, metastatic primary squamous neck cancer, metastatic squamous neck cancer, multiple myeloma, multiple myeloma/plasma cell neoplasm, myelodysplastic syndrome, myelogenous leukemia, myeloid leukemia, myeloproliferative disorders, nasal cavity and paranasal sinus cancer, nasopharyngeal cancer, neuroblastoma, non-Hodgkin's lymphoma during pregnancy, nonmelanoma skin cancer, non-small cell lung cancer, occult primary metastatic squamous neck cancer, oropharyngeal cancer, osteo-/malignant fibrous sarcoma, osteosarcoma/malignant fibrous histiocytoma, osteosarcoma/malignant fibrous histiocytoma of bone, ovarian epithelial cancer, ovarian germ cell tumor, ovarian low malignant potential tumor, pancreatic cancer, paraproteinemias, purpura, parathyroid cancer, penile cancer, pheochromocytoma, pituitary tumor, plasma cell neoplasm/multiple myeloma, primary central nervous system lymphoma, primary liver cancer, prostate cancer, rectal cancer, renal cell cancer, renal pelvis and ureter cancer, retinoblastoma, rhabdomyosarcoma, salivary gland cancer, sarcoidosis sarcomas, Sezary syndrome, skin cancer, small cell lung cancer, small intestine cancer, soft tissue sarcoma, squamous neck cancer, stomach cancer, supratentorial primitive neuroectodermal and pineal tumors, T-cell lymphoma, testicular cancer, thymoma, thyroid cancer, transitional cell cancer of the renal pelvis and ureter, transitional renal pelvis and ureter cancer, trophoblastic tumors, ureter and renal pelvis cell cancer, urethral cancer, uterine cancer, uterine Sarcoma, vaginal Cancer, visual pathway and hypothalamic glioma, vulvar cancer, Waldenstrom's macroglobulinemia, Wilms' tumor, and any other hyperproliferative disease, located in an organ system listed above.

In another preferred embodiment, the compounds and compositions of the invention are used to diagnose, prognose, prevent, and/or treat premalignant conditions and to prevent progression to a neoplastic or malignant state, including but not limited to those disorders described above. Such uses are indicated in conditions known to precede or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79).

Hyperplasia is a form of controlled cell proliferation, involving an increase in cell number in a tissue or organ, without significant alteration in structure or function. Hyperplastic disorders which can be diagnosed, prognosed, prevented, and/or treated with compounds and compositions of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be diagnosed, prognosed, prevented, and/or treated with compounds and compositions of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be diagnosed, prognosed, prevented, and/or treated with compounds and compositions of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be diagnosed, prognosed, prevented, and/or treated with compounds and compositions of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In certain embodiments, the present invention relates to the aforementioned method, wherein said cancer or other dysproliferative disease is selected from the group consisting of leukemias, myeloid leukemias, lymphocytic leukemias, lymphomas, myeloproliferative diseases, solid tumors, sarcomas, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In certain embodiments, the present invention relates to the aforementioned method, wherein said cancer or other dysproliferative disease is selected from the group consisting of brain tumors, glioma, diabetic retinopathy, and pancreatic cancers.

In certain embodiments, the present invention relates to the aforementioned method, wherein said cancer or other dysproliferative disease is selected from the group consisting of arteriovenous (AV) malformations, psoriasis, benign prostatic hypertrophy, cutaneous fungal infections, warts, birthmarks, moles, nevi, skin tags, lipomas, angiomas hemangiomas, and cutaneous lesions.

Another aspect of the present invention relates to a method of intentional ablation or destruction of tissues or organs in a human or animal by administering to a patient in need thereof an effective amount of a compound of the invention or pharmaceutical composition of the invention.

Inflammatory Disorders. Another aspect of the invention related to a method for treating an inflammatory disease or disorder such as rheumatoid arthritis, in which, for example, inappropriate angiogenesis leads to the formation of pannus and associated pathology in a joint, or neovascularization from the retina in diabetic patients leading to blindness.

Rheumatoid arthritis (RA) is a chronic disease, characterized mainly by inflammation of the lining, or synovium, of the joints. It can lead to long-term joint damage, resulting in chronic pain, loss of function and disability. The disease progresses in three stages. The first stage is the swelling of the synovial lining, causing pain, warmth, stiffness, redness and swelling around the joint. Second is the rapid division and growth of cells, or pannus, which causes the synovium to thicken. In the third stage, the inflamed cells release enzymes that may digest bone and cartilage, often causing the involved joint to lose its shape and alignment, additional pain, and loss of movement. Because it is a chronic disease, RA continues indefinitely and may never resolve. Frequent flares in disease activity can occur. RA is also a systemic disease, with the potential to affect other organs in the body.

Approximately 2.1 million people in the United States, or 1% of the population, have R^{A}. It can affect anyone, including children, but 70% of individuals with R^{A} are women. Onset usually occurs between 30 and 50 years of age. R^{A} often goes into remission in pregnant women, although symptoms tend to increase in intensity after delivery. R^{A} develops more often than expected the year after giving birth. While women are two to three times more likely to get R^{A} than men, men tend to be more severely affected when afflicted.

Inflammation and angiogenesis are two of the fundamental processes that underlie pathologic disorders. Tissue injury induces inflammation, and inflammation triggers angiogenesis, which in turn, initiates tissue repair and tissue growth. RA is an inflammatory disease as well as an angiogenic disease. The joint in RA contains a massive proliferating synovium, which forms an invading tissue, termed pannus. The formation of pannus is central to joint erosion and results in the destruction of cartilage and bone. Angiogenesis is an important component of most inflammatory reactions and subsequent repair/growth processes. Persistent angiogenesis is critical both to maintaining the chronic architectural changes in the RA synovium via delivery of nutrients and inflammatory cells, and to providing an important source of cytokines and protease activity.

As mentioned above, Met and Tie2 are two receptor tyrosine kinases have been identified as therapeutic targets against angiogenesis. Studies described here have identified active kinase inhibitors that competes with ATP for the ATP-binding site of Met. Results indicate that compounds of the invention significantly and selectively inhibits Met activation and inhibits downstream signaling events initiated by its ligand, hepatocyte growth factor/scatter factor (HGF/SF). Moreover, compounds of the invention also inhibit activity of Tie-2. In vitro, compounds inhibits endothelial and tumor cell growth and, most relevant here, HGF/SF-induced angiogenesis. In a rat model of R^{A}, inventive compound ameliorates the disease course and reduces tissue damage.

Methods Using New Compounds and Pharmaceutical Compositions. In one embodiment, the invention embraces a method of treating a dysproliferative or inflammatory disease such as cancer or rheumatoid arthritis comprising administering to a subject in need thereof a therapeutically effective amount of a compound or pharmaceutical composition comprising a compound of formula I:

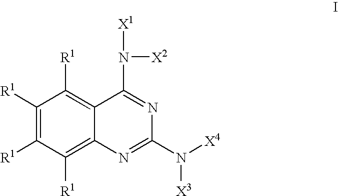

or a pharmaceutically acceptable salt thereof,

wherein, independently for each occurrence,

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

X¹, X², X³ and X⁴ are hydrogen or an optionally substituted aliphatic, alicyclic, heteroaliphatic, heterocyclic, aromatic, heteroaromatic or acyl group; or X¹ and X² taken together with the nitrogen to which they are bonded, or X³ and X⁴ taken together with the nitrogen to which they are bonded, are independently an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned method wherein R¹ is hydrogen; halogen; a saturated or unsaturated, branched or straight-chain C₁₋₆ alkyl; aryl-C₁₋₆ alkyl; mono- or polyfluorinated C₁₋₆ alkyl; C₁₋₆ alkoxy; C₁₋₆ alkylamino; di(C₁₋₆ alkyl)amino; C₁₋₈ alkylamino-C₁₋₈ alkyl; di(C₁₋₆ alkyl)amino-C₁₋₈ alkyl; cyclo(C₃₋₆)alkyl; aryl, wherein the aryl comprises a six membered aromatic carbocycle (such as phenyl) or a polycyclic aromatic hydrocarbon (such as naphthyl, phenanthracenyl, indanyl); a heterocycle, wherein the heterocycle comprises six membered aromatic heterocycles (such as pyridyl, diazinyl, pyrimidinyl, pyrrolidinyl, piperazinyl, thiazinyl), five membered aromatic heterocycles (such as pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thienyl) or bicyclic systems (such as indolyl, benzothienyl, benzofuranyl, isoindolyl, isobenzothienyl, isobenzofuranyl); wherein any of wherein one or more of the foregoing aliphatic, cyclic, aromatic or heteroaromatic substituents optionally may be further substituted with a C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, cyano, hydroxy, carboxy, carboxy ester, amine (optionally substituted with C₁₋₆ straight chain alkyl), C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned method wherein R¹ represents two non-hydrogen substituents which combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned method wherein R², R³, R⁴, R⁵ and R⁶, and the carbons to which they are bonded, may combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned method wherein X¹, X², X³, and X⁴ are independently selected from the group consisting of hydrogen, a C₁₋₆ straight chain saturated or unsaturated alkyl group, a C₃₋₆ branched saturated or unsaturated chain alkyl group, a C₃₋₆ cycloalkyl group; and any of the foregoing are optionally substituted with one or more halo, nitro, cyano, hydroxy, carboxy, carboxy ester, amine (optionally substituted with C₁₋₆ straight chain alkyl), C₃₋₆ branched chain alkyl, C₃₋₆ cycloalkyl, aromatic group or aralkyl group (such as phenyl, benzyl or naphthyl, optionally further substituted as described above), fused alkyl or aromatic ring, or heteroaromatic or heterocyclic ring, which may be a saturated or unsaturated ring containing 4-10 ring members and 0-3 heteroatoms selected from the group consisting of O, N and S, the heteroaromatic or heterocyclic ring optionally substituted with one or more halo, C₁₋₆ straight chain alkyl, C₃₋₆ branched chain alkyl, C₃₋₆ cycloalkyl, C₁₋₆ alkyloxy, nitro, cyano, hydroxy, carboxyl, ester, amine (optionally substituted with C₁₋₆ straight chain alkyl), C₃₋₆ branched chain alkyl or C₃₋₆ cycloalkyl, trifluoroxy, trifluoromethyl, difluoromethyl, aryl, the same or different heterocyclic ring, or a fused aromatic, heteroaromatic or heterocyclic ring. The alkyl group of alkyloxy may be a C₁₋₆ straight chain, C₃₋₆ branched or C₃₋₆ cycloalkyl; and any of the alkyl groups herein may be saturated or contain one or more degrees of unsaturation; or X¹ and X² or X³ and X⁴ together with the nitrogen to which they are bonded is an optionally substituted heteroaromatic or heterocyclic ring comprising in addition to the aforementioned nitrogen, 4-10 ring members and 0-3 additional heteroatoms selected from the group consisting of O, N and S, the heteroaromatic or heterocyclic ring optionally further substituted with one or more aliphatic, aromatic, —SR^{R}, —OR^{R}, heteroaromatic or fused rings which may be further substituted as described herein.

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned method wherein X¹ and X² taken together with the nitrogen to which they are bonded are an optionally substituted heterocyclic group comprising 4-10 ring members and 0-3 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned method wherein X¹ and X² are hydrogen or an optionally substituted aliphatic, alicyclic, or aromatic group.

In certain embodiments, the present invention relates to the aforementioned method wherein X¹ and X² are hydrogen, cyclopentyl, benzyl, 4-methoxyphenyl or 2-isopropylphenyl.

In certain embodiments, the present invention relates to the aforementioned method wherein X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned method wherein R¹ is hydrogen, halogen, C₁₋₆ alkyl, aryl-C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, aryl, or heterocycle; wherein one or more of the foregoing aliphatic, cyclic, aromatic or heteroaromatic substituents optionally may be further substituted with C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, fluoro, cyano, hydroxy, carboxy, carboxy ester, amine, C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned method wherein R¹ is hydrogen, halogen, C₁₋₆ alkyl or C₁₋₆ alkoxy.

In certain embodiments, the present invention relates to the aforementioned method wherein R¹ is hydrogen.

In the non-limiting, exemplary method of the invention described herein, any of the —NX¹X² substituents present amongst the various compounds described may be utilized as the —NX³X⁴ substituents, and vice versa. In certain embodiments, the present invention relates to the aforementioned method wherein —NX¹X² and/or —NX³X⁴ are selected from the two sets of —NX¹X²/—NX³X⁴ moieties described for formula I above.

In certain embodiments, the present invention relates to the aforementioned method wherein X¹ is cyclopentyl; and X² is hydrogen. For example, one aspect of the invention relates to method comprising a compound selected from the group consisting of the compounds shown below.

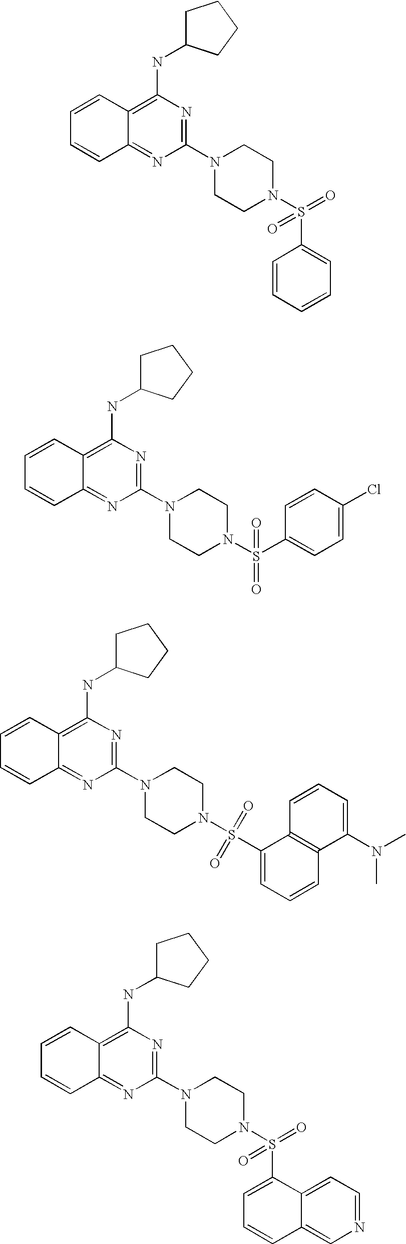

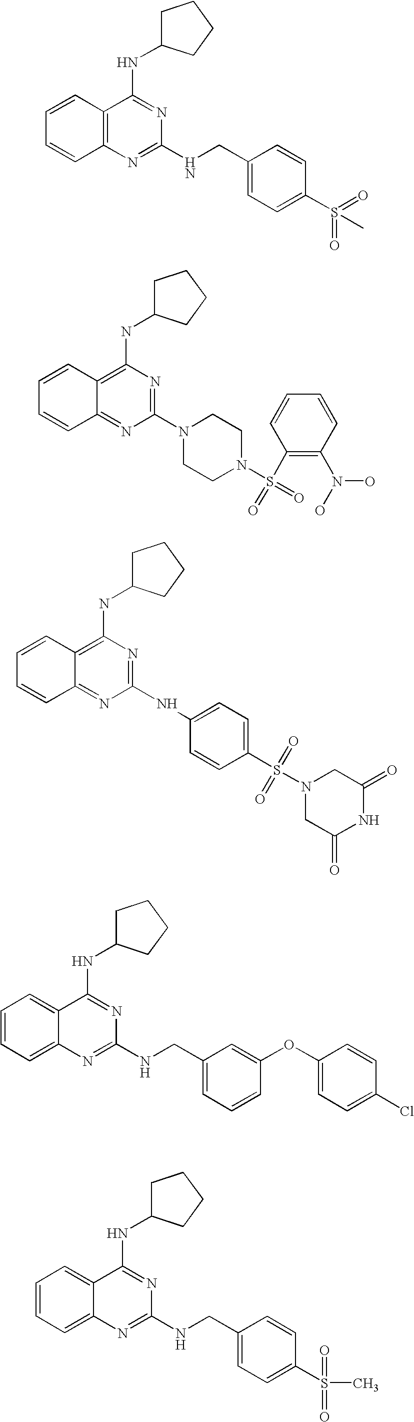

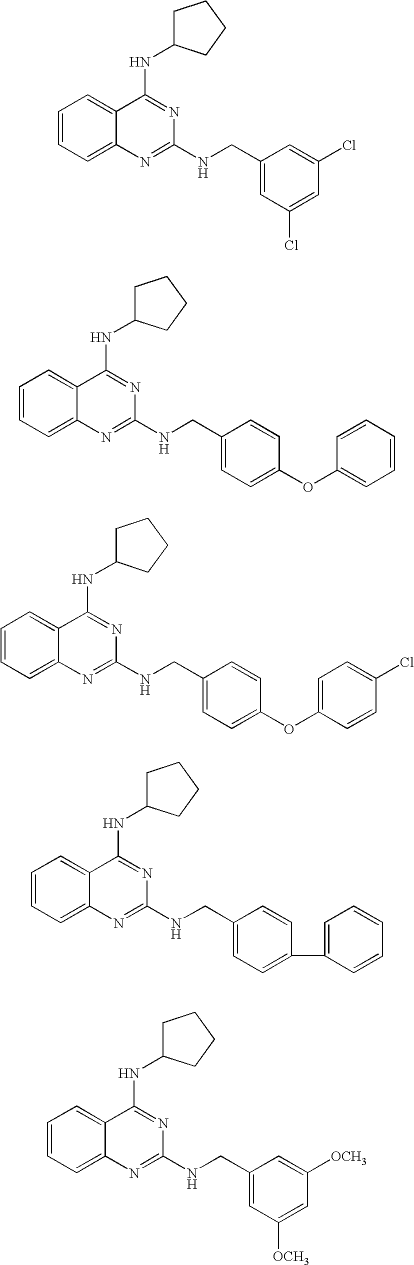

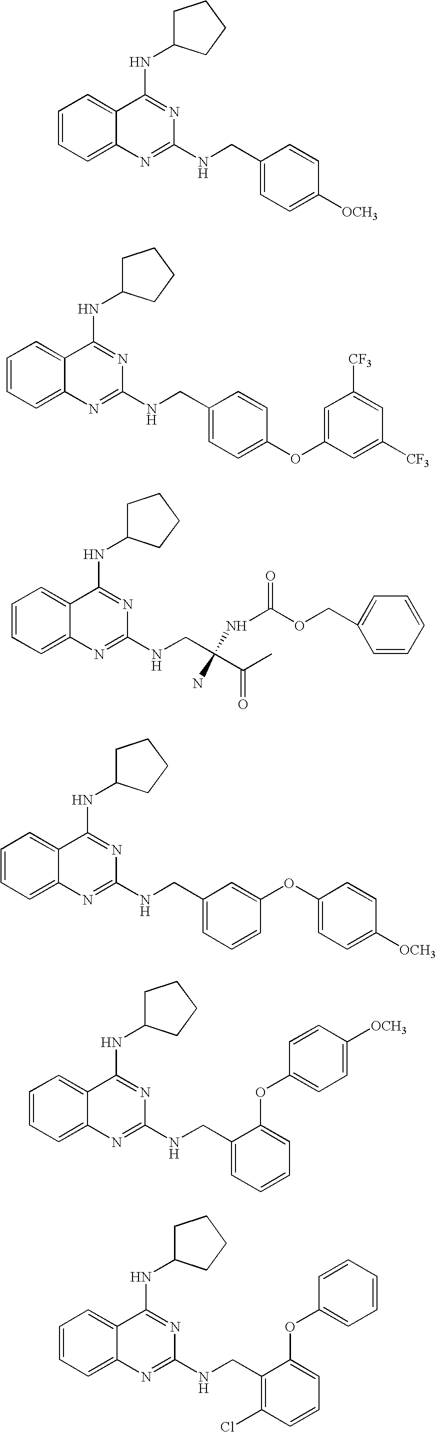

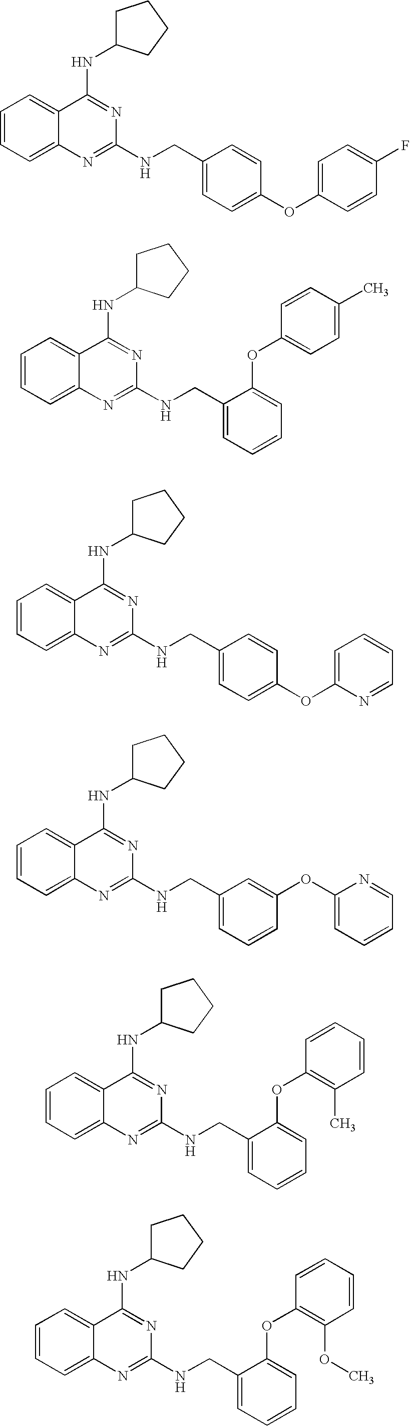

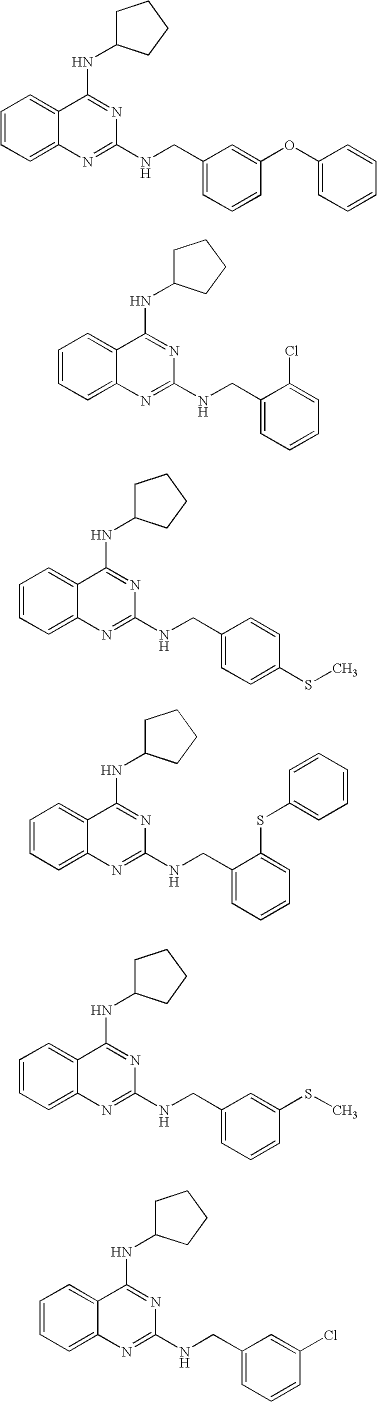

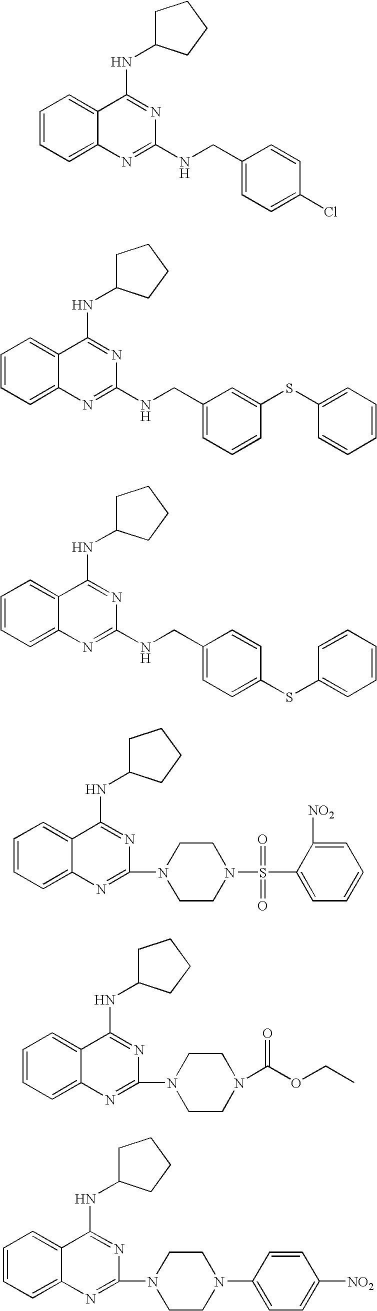

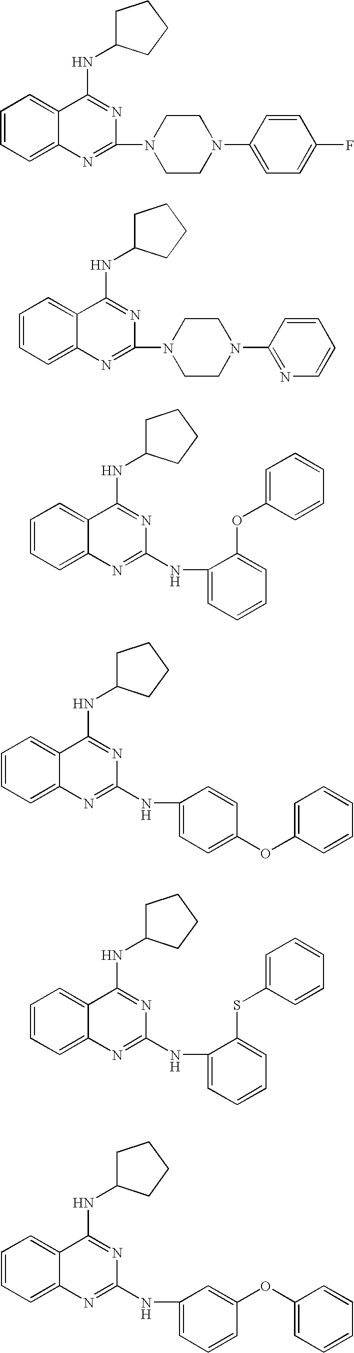

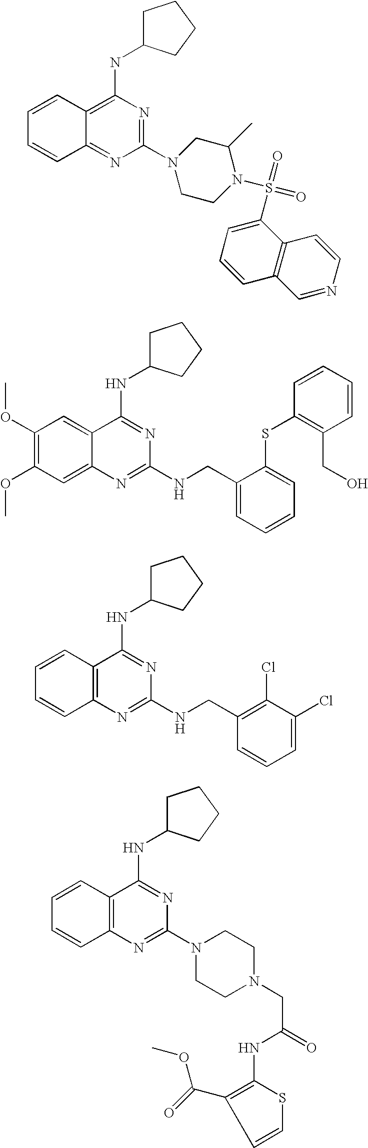

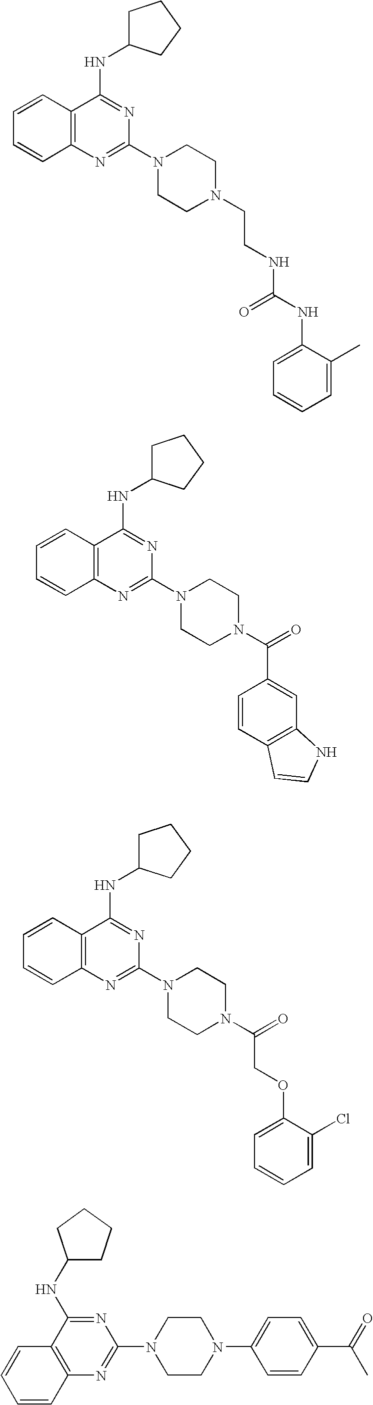

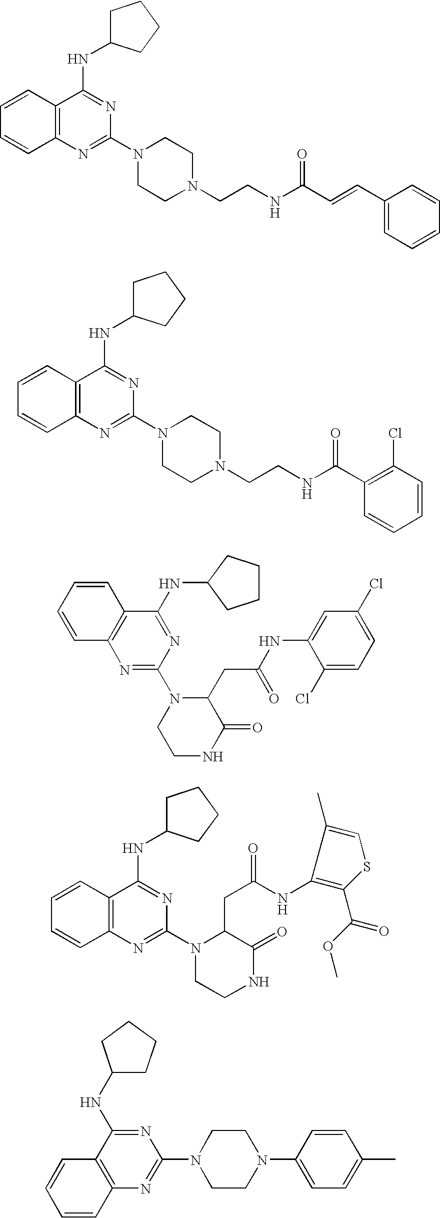

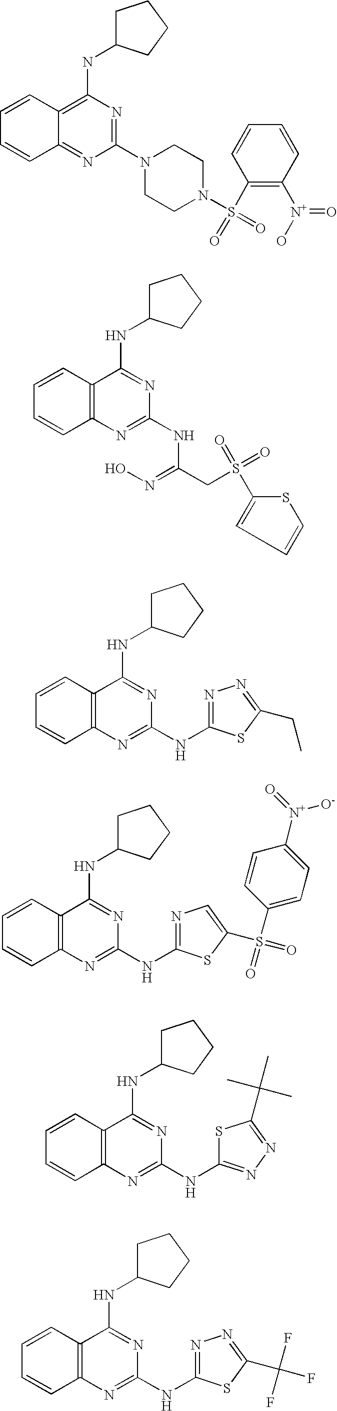

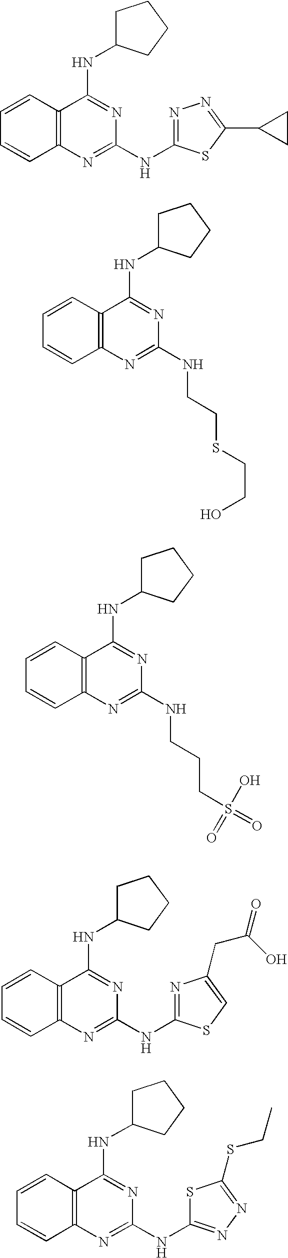

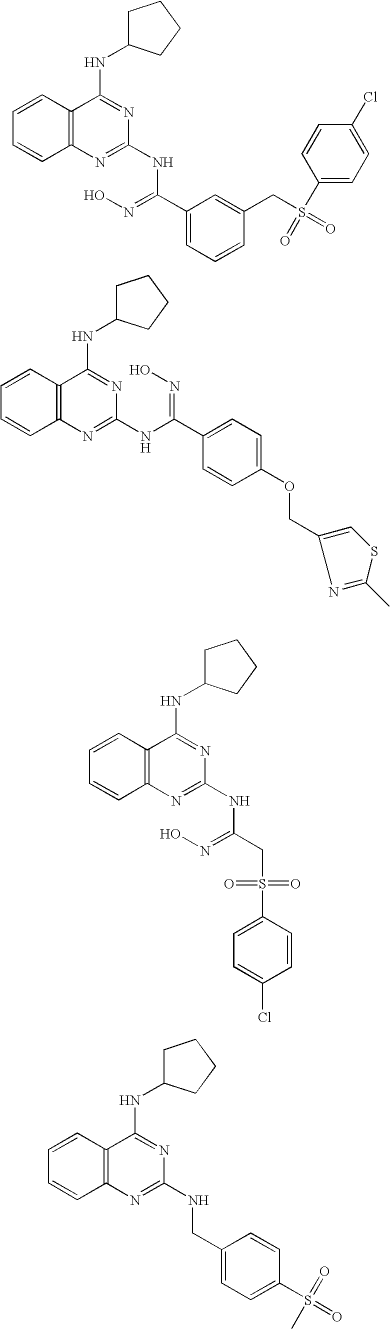

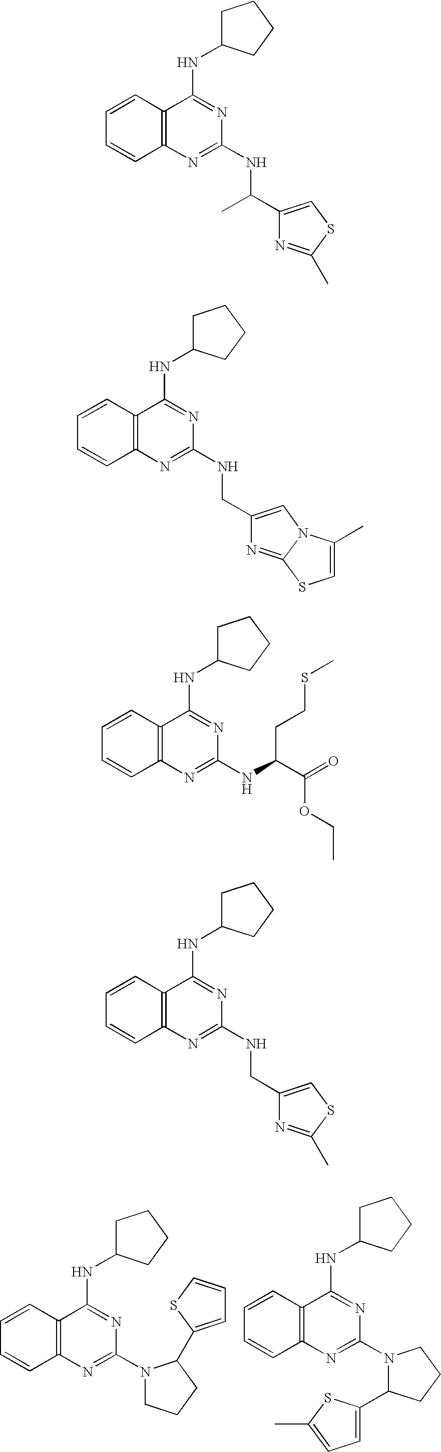

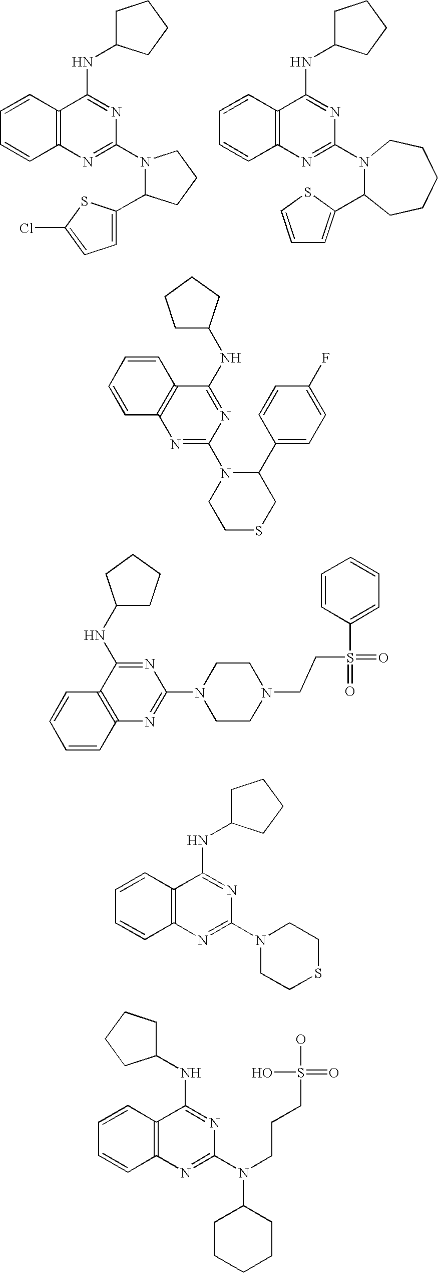

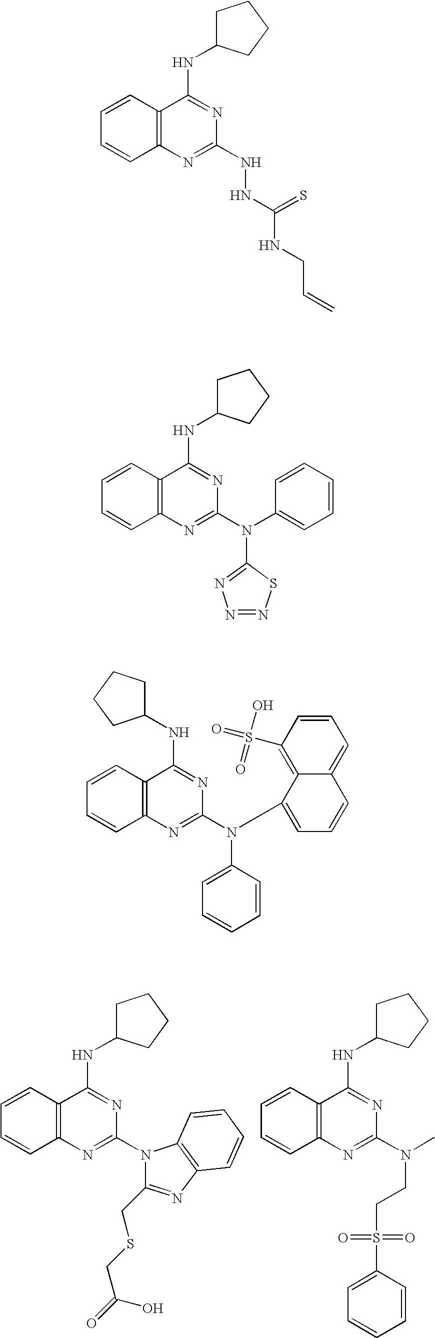

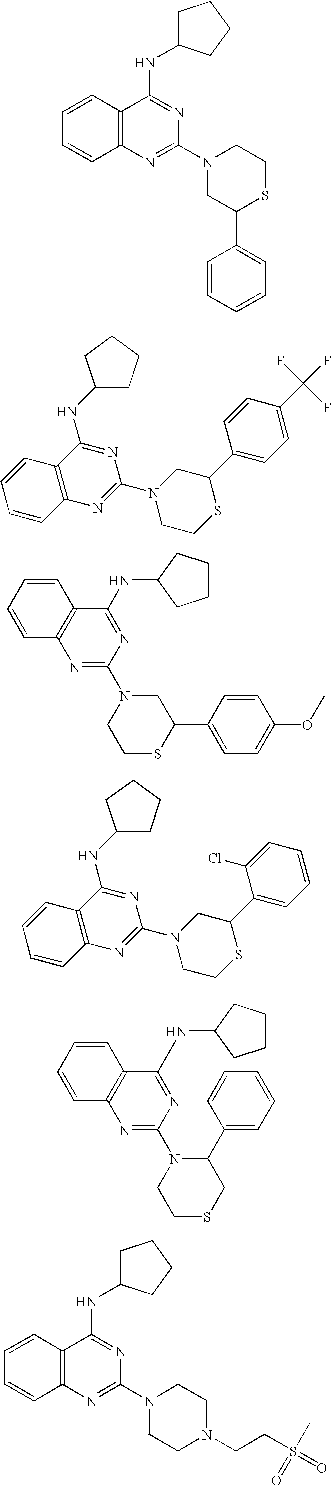

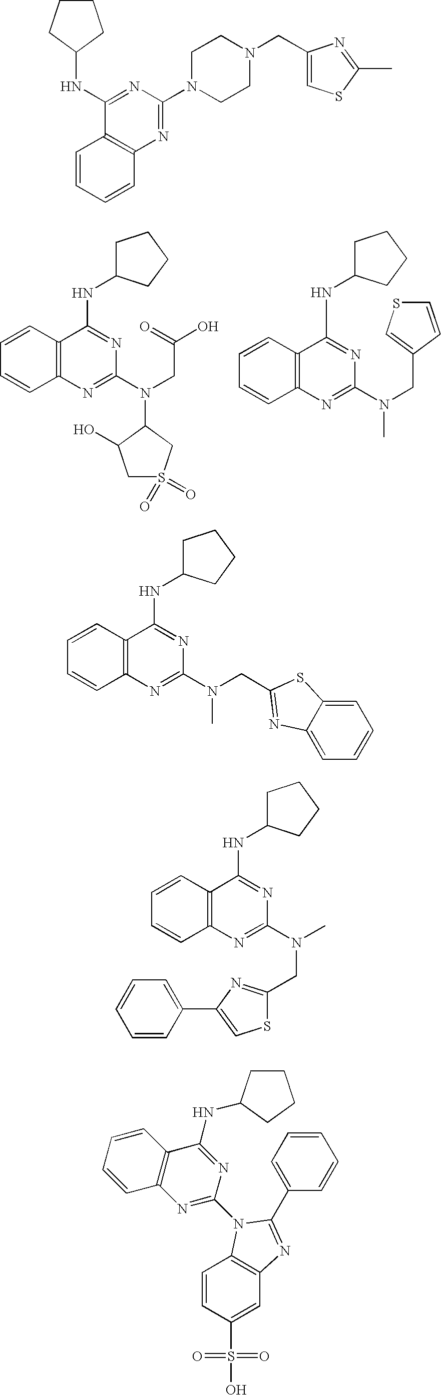

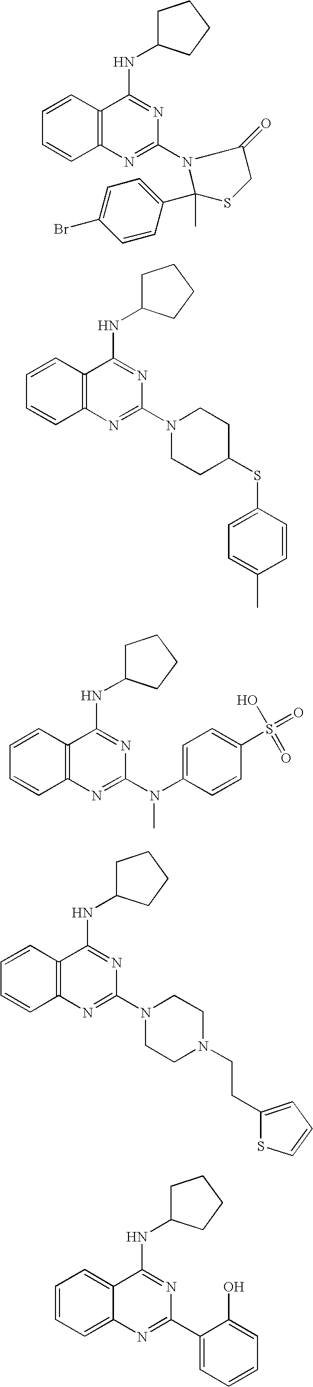

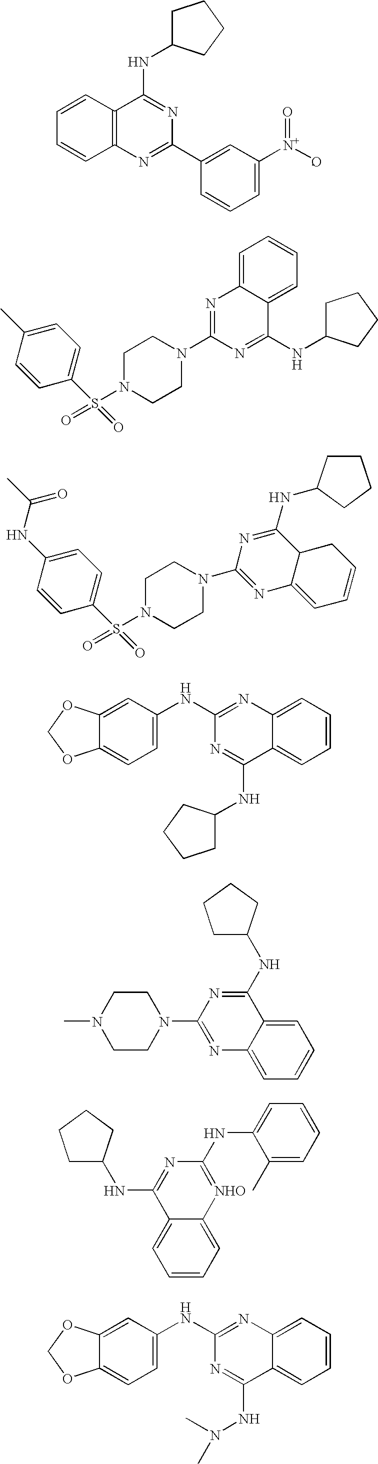

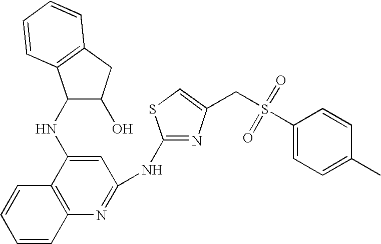

Another aspect of the present invention relates to a pharmaceutical composition comprising a compound of formula II:

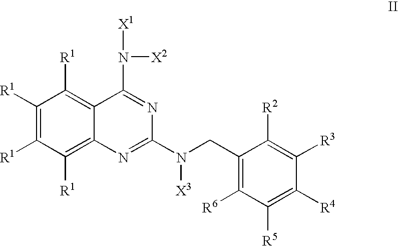

or a pharmaceutically acceptable salt thereof,

wherein, independently for each occurrence:

R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R², R³, R⁴, R⁵, and R⁶ are hydrogen, —F, —Cl, —Br, —I, —OH, —SH, —NO₂, —CN, —OR^{R}, —SR^{R}, S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹, X² and X³ are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned method, provided that when R¹ is hydrogen; R² is —SR^{R}; R³ is hydrogen; R⁴ is hydrogen; R⁵ is hydrogen; R⁶ is hydrogen; R^{R} is 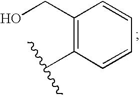
and —NX¹X² is 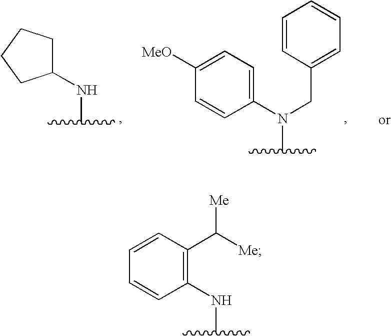
X³ is not hydrogen.

In certain embodiments, the present invention relates to the aforementioned method wherein R¹ is hydrogen; halogen; a saturated or unsaturated, branched or straight-chain C₁₋₆ alkyl; aryl-C₁₋₆ alkyl; mono- or polyfluorinated C₁₋₆ alkyl; C₁₋₆ alkoxy; C₁₋₆ alkylamino; di(C₁₋₆ alkyl)amino; C₁₋₈ alkylamino-C₁₋₈ alkyl; di(C₁₋₆ alkyl)amino-C₁₋₈ alkyl; cyclo(C₃₋₆)alkyl; aryl, wherein the aryl comprises a six membered aromatic carbocycle (such as phenyl) or a polycyclic aromatic hydrocarbon (such as naphthyl, phenanthracenyl, indanyl); a heterocycle, wherein the heterocycle comprises six membered aromatic heterocycles (such as pyridyl, diazinyl, pyrimidinyl, pyrrolidinyl, piperazinyl, thiazinyl), five membered aromatic heterocycles (such as pyrrolyl, pyrazole, imidazolyl, imidazolidinyl, imidazolenyl, oxazolyl, isoxazolyl, thiazolyl, thiazolidinyl, thiazolinyl, isothiazolyl, isothiazolidinyl, isothiazolinyl, furanyl, thienyl) or bicyclic systems (such as indolyl, benzothienyl, benzofuranyl, isoindolyl, isobenzothienyl, isobenzofuranyl); wherein any of wherein one or more of the foregoing aliphatic, cyclic, aromatic or heteroaromatic substituents optionally may be further substituted with a C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, cyano, hydroxy, carboxy, carboxy ester, amine (optionally substituted with C₁₋₆ straight chain alkyl), C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned method wherein R¹ represents two non-hydrogen substituents which combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned method, wherein R², R³, R⁴, R⁵ and R⁶, and the carbons to which they are bonded, may combine to form a ring ranging in total ring size from five to nine, wherein one or more of the methylene hydrogen atoms may be replaced with halogen, C₁₋₆ alkyl, aryl-C₁₋₆ alkyl, mono- or polyfluorinated C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, or aryl; wherein the aryl comprises any six membered aromatic carbocycle, heterocycle, bicyclic systems such as described herein and is optionally further substituted as described above.

In certain embodiments, the present invention relates to the aforementioned method wherein X¹, X² and X³ are independently selected from the group consisting of hydrogen, a C₁₋₆ straight chain saturated or unsaturated alkyl group, a C₃₋₆ branched saturated or unsaturated chain alkyl group, a C₃₋₆ cycloalkyl group; and any of the foregoing are optionally substituted with one or more halo, nitro, cyano, hydroxy, carboxy, carboxy ester, amine (optionally substituted with C₁₋₆ straight chain alkyl), C₃₋₆ branched chain alkyl, C₃₋₆ cycloalkyl, aromatic group or aralkyl group (such as phenyl, benzyl or naphthyl, optionally further substituted as described above), fused alkyl or aromatic ring, or heteroaromatic or heterocyclic ring, which may be a saturated or unsaturated ring containing 4-10 ring members and 0-3 heteroatoms selected from the group consisting of O, N and S, the heteroaromatic or heterocyclic ring optionally substituted with one or more halo, C₁₋₆ straight chain alkyl, C₃₋₆ branched chain alkyl, C₃₋₆ cycloalkyl, C₁₋₆ alkyloxy, nitro, cyano, hydroxy, carboxyl, ester, amine (optionally substituted with C₁₋₆ straight chain alkyl), C₃₋₆ branched chain alkyl or C₃₋₆ cycloalkyl, trifluoroxy, trifluoromethyl, difluoromethyl, aryl, the same or different heterocyclic ring, or a fused aromatic, heteroaromatic or heterocyclic ring. The alkyl group of alkyloxy may be a C₁₋₆ straight chain, C₃₋₆ branched or C₃₋₆ cycloalkyl; and any of the alkyl groups herein may be saturated or contain one or more degrees of unsaturation; or X¹ and X² together with the nitrogen to which they are bonded is an optionally substituted heteroaromatic or heterocyclic ring comprising in addition to the aforementioned nitrogen, 4-10 ring members and 0-3 additional heteroatoms selected from the group consisting of O, N and S, the heteroaromatic or heterocyclic ring optionally further substituted with one or more aliphatic, aromatic, —SR^{R}, —OR^{R}, heteroaromatic or fused rings which may be further substituted as described herein.

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned method wherein X¹ and X² taken together with the nitrogen to which they are bonded are an optionally substituted heterocyclic group comprising 4-10 ring members and 0-3 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned method wherein R¹, R³, R⁴, R⁵ and R⁶ are hydrogen; R² is —SR^{R}; and R^{R} is an optionally substituted phenyl group. Examples of substitutions of said phenyl group include a hydroxyalkyl group (such as hydroxymethyl and hydroxyethyl); a haloalkyl group (such as fluoromethyl, difluoromethyl and trifluoromethyl); an alkoxyalkyl group (such as ethoxymethyl and methoxymethyl); a carboxyalkyl group (such as carboxymethyl and carboxyethyl); a —COOH; a C₁₋₆ alkylidene-O(C═O)-alkyl or C₁₋₆ alkylidene-(C═O)-alkoxy group (such as —CH₂—OC(═O)—CH₃ and —CH₂CH₂—C(═O)—OCH₃); an amide, alkylamide or dialkylamide; and an alkylaminocarboxy moiety (such as —OC(═O)NHEt).

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group.

In certain embodiments, the present invention relates to the aforementioned method wherein X¹ and X² are hydrogen or an optionally substituted aliphatic, alicyclic, or aromatic group.

In certain embodiments, the present invention relates to the aforementioned method wherein X¹ and X² are hydrogen, cyclopentyl, benzyl, 4-methoxyphenyl or 2-isopropylphenyl.

In certain embodiments, the present invention relates to the aforementioned method wherein X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups.

In certain embodiments, the present invention relates to the aforementioned method wherein —NX¹X² is selected from the two sets of —NX¹X² moieties described for formula I above.

In certain embodiments, the present invention relates to the aforementioned method wherein R² is —SR^{R}.

In certain embodiments, the present invention relates to the aforementioned method wherein R² is —SR^{R}; and R³, R⁴, R⁵ and R⁶ are hydrogen.

In certain embodiments, the present invention relates to the aforementioned method wherein R² is —SR^{R}; R³, R⁴, R⁵ and R⁶ are hydrogen; and R^{R} is an optionally substituted phenyl.

In certain embodiments, the present invention relates to the aforementioned method wherein R² is —SR^{R}; R³, R⁴, R⁵ and R⁶ are hydrogen; R^{R} is 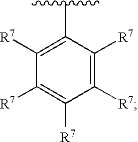
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned method
wherein R² is —SR^{R}; R³, R⁴, R⁵ and R⁶ are hydrogen; R^{R} is 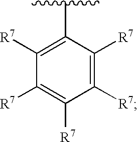
and R⁷ is, independently for each occurrence, hydrogen, hydroxymethyl, hydroxyethyl, fluoromethyl, difluoromethyl, trifluoromethyl, ethoxymethyl, methoxymethyl, carboxymethyl, carboxyethyl, —COOH, —CH₂—OC(═O)—CH₃, —CH₂CH₂—C(═O)—OCH₃ or —O(CO)NHEt.

In certain embodiments, the present invention relates to the aforementioned method wherein R² is —SR^{R}; R³, R⁴, R⁵ and R⁶ are hydrogen; and R^{R} is

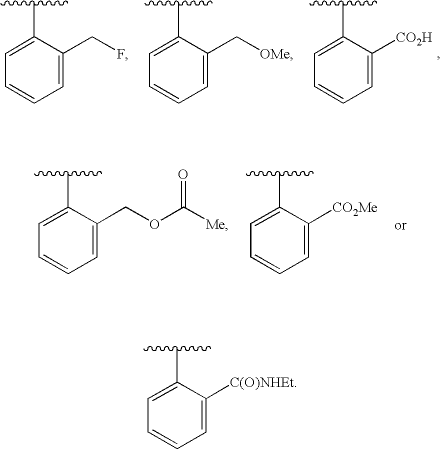

In certain embodiments, the present invention relates to the aforementioned method wherein R¹ is hydrogen, halogen, C₁₋₆ alkyl, aryl-C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, cyclo(C₃₋₆)alkyl, aryl, or heterocycle; wherein one or more of the foregoing aliphatic, cyclic, aromatic or heteroaromatic substituents optionally may be further substituted with C₁₋₆ alkyl, C₁₋₆ alkoxy, C₁₋₆ alkylamino, di(C₁₋₆ alkyl)amino, C₁₋₈ alkylamino-C₁₋₈ alkyl, di(C₁₋₆ alkyl)amino-C₁₋₈ alkyl, nitro, fluoro, cyano, hydroxy, carboxy, carboxy ester, amine, C₃₋₆ branched chain alkyl, C₃₋₆ cycloalkyl, trifluoroxy, trifluoromethyl, difluoromethyl, aryl, heterocyclic ring, or a fused aromatic or heterocyclic ring.

In certain embodiments, the present invention relates to the aforementioned method wherein R¹ is hydrogen, halogen, C₁₋₆ alkyl or C₁₋₆ alkoxy.

In certain embodiments, the present invention relates to the aforementioned method wherein R¹ is hydrogen.

In certain embodiments, the present invention relates to the aforementioned method wherein X³ is hydrogen, aliphatic or alicyclic.

In certain embodiments, the present invention relates to the aforementioned method wherein X³ is hydrogen or C₁₋₆ alkyl.

In certain embodiments, the present invention relates to the aforementioned method wherein X³ is hydrogen.

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heterocyclic group comprising 5-6 ring members and 0-1 additional heteroatoms selected from the group consisting of O, N and S; the heterocyclic group optionally further substituted with one or more optionally substituted aliphatic, alicyclic, heteroaliphatic, heterocyclic, aromatic, heteroaromatic or acyl groups; and R² is —SR^{R}.

In certain embodiments, the present invention relates to the aforementioned method wherein X¹ and X² are hydrogen or an optionally substituted aliphatic, alicyclic, heteroaliphatic, heterocyclic, aromatic, heteroaromatic or acyl group; R² is —SR^{R}; R³, R⁴, R⁵ and R⁶ are hydrogen; and R^{R} is an optionally substituted phenyl.

In certain embodiments, the present invention relates to the aforementioned method wherein X¹ and X² are hydrogen or an optionally substituted aliphatic, alicyclic, or aromatic group; and R² is —SR^{R}; R³, R⁴, R⁵ and R⁶ are hydrogen; R^{R} is 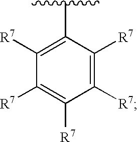
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned method wherein X¹ and X² are hydrogen or an optionally substituted aliphatic, alicyclic, or aromatic group; and R² is —SR^{R}; R³, R⁴, R⁵ and R⁶ are hydrogen; R^{R} is 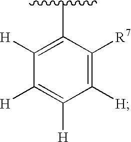
and R⁷ is, independently for each occurrence, hydrogen, hydroxyalkyl, haloalkyl group, alkoxyalkyl, carboxyalkyl, C₁₋₆ alkylidene-O(C═O)-alkyl, C₁₋₆ alkylidene-(C═O)-alkoxy, amide, alkylamide, dialkylamide or a carbamate radical.

In certain embodiments, the present invention relates to the aforementioned method wherein X¹ and X² are hydrogen, cyclopentyl, benzyl, 4-methoxyphenyl or 2-isopropylphenyl; R² is —SR; R³, R⁴, R⁵ and R⁶ are hydrogen; and R^{R} is

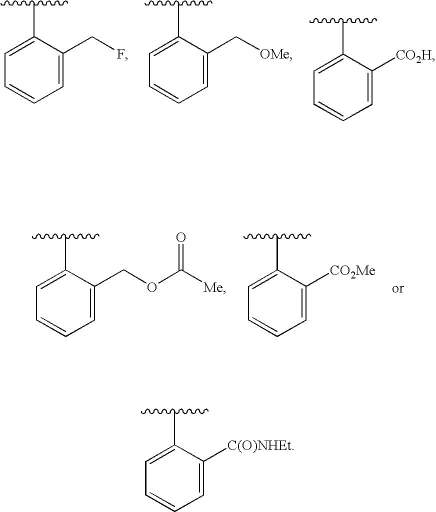

In certain embodiments, the present invention relates to the aforementioned method wherein X¹ and X² are hydrogen, cyclopentyl, benzyl, 4-methoxyphenyl or 2-isopropylphenyl; R² is —SR^{R}; R³, R⁴, R⁵ and R⁶ are hydrogen; R^{R} is 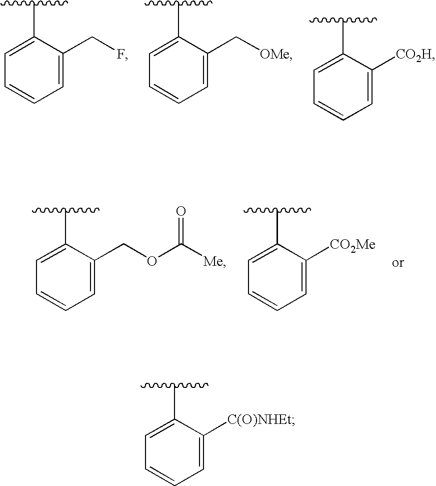
and R¹ is hydrogen.

In another embodiment, the invention embraces a method of treating a dysproliferative or inflammatory disease such as cancer or rheumatoid arthritis comprising administering to a subject in need thereof a therapeutically effective amount of a compound or pharmaceutical composition comprising a compound of selected from the compounds listed below.

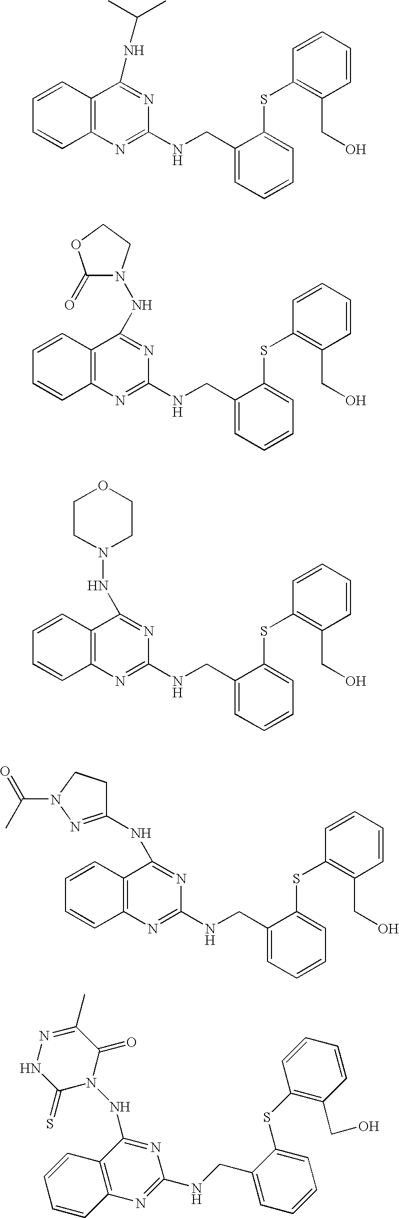

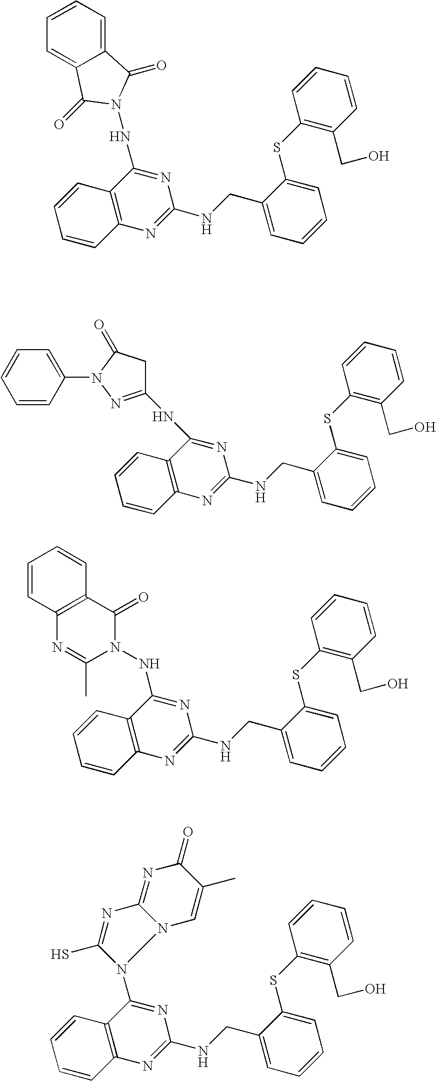

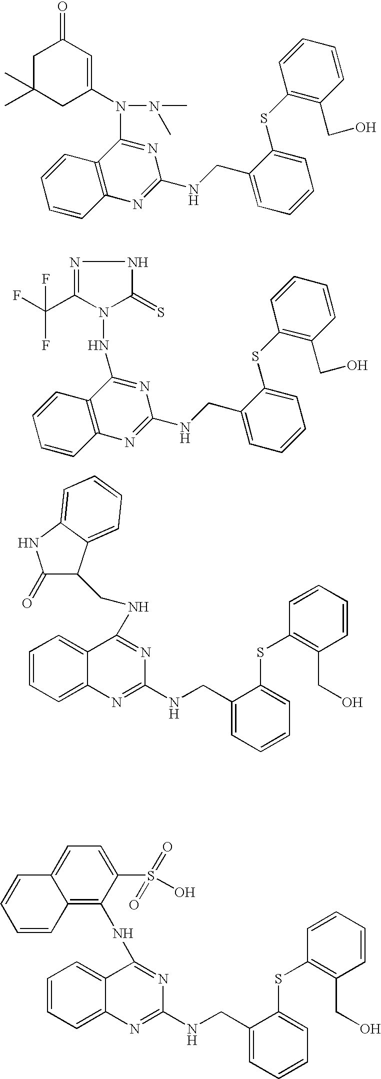

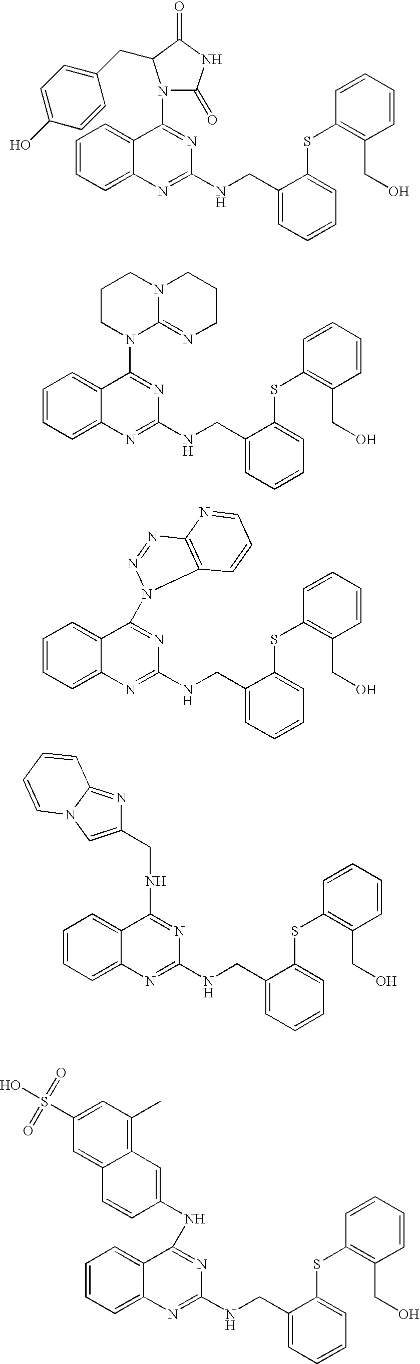

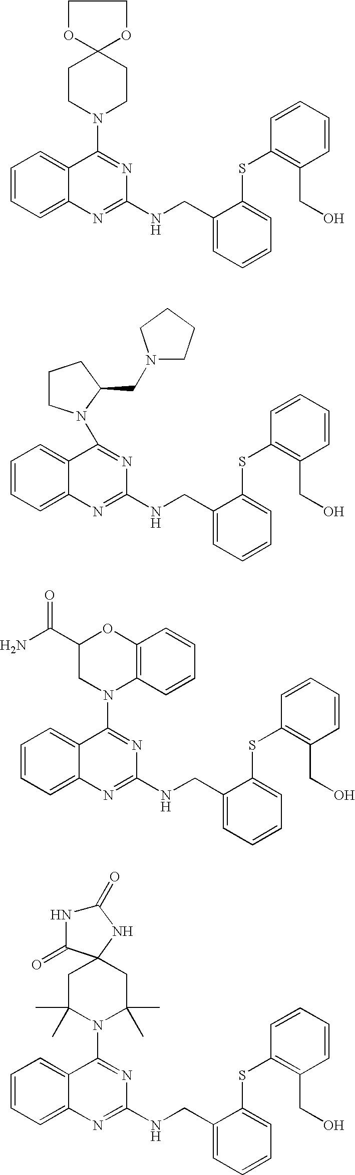

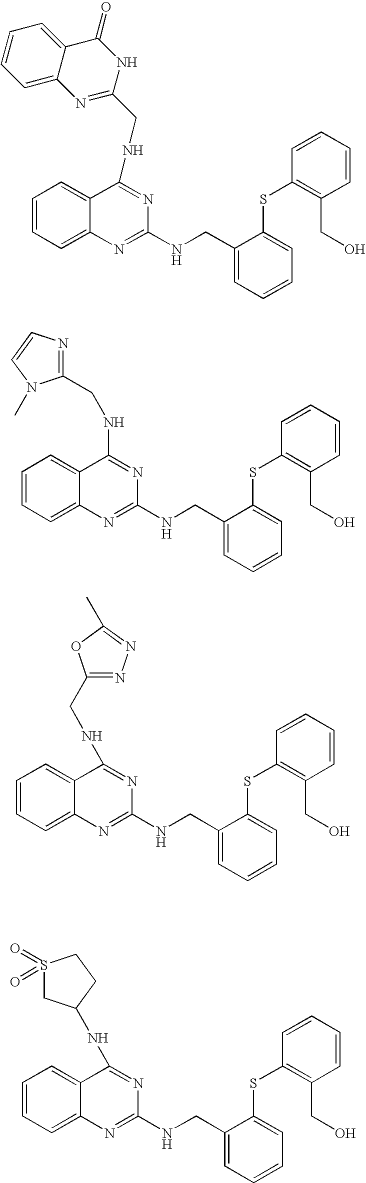

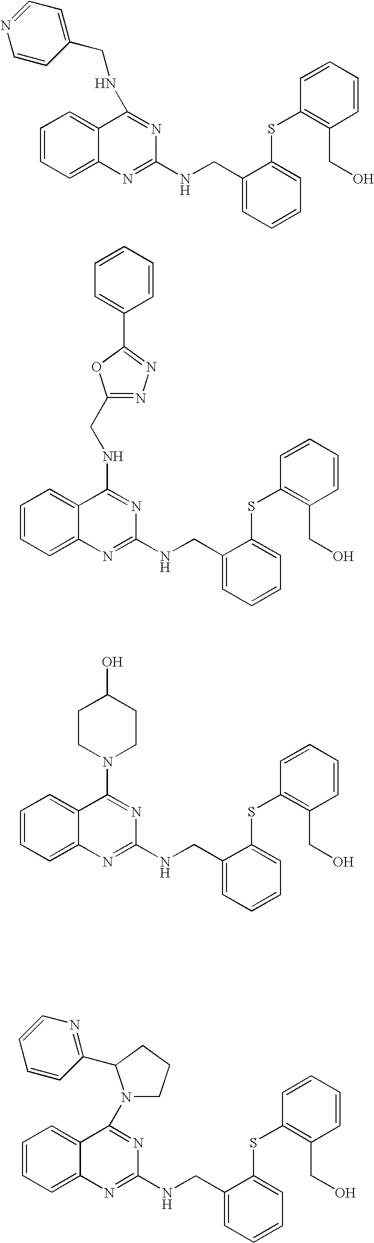

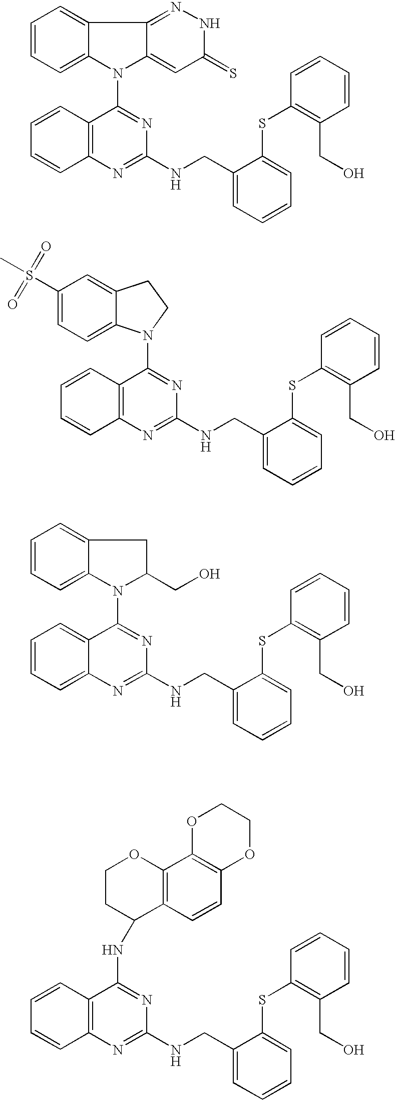

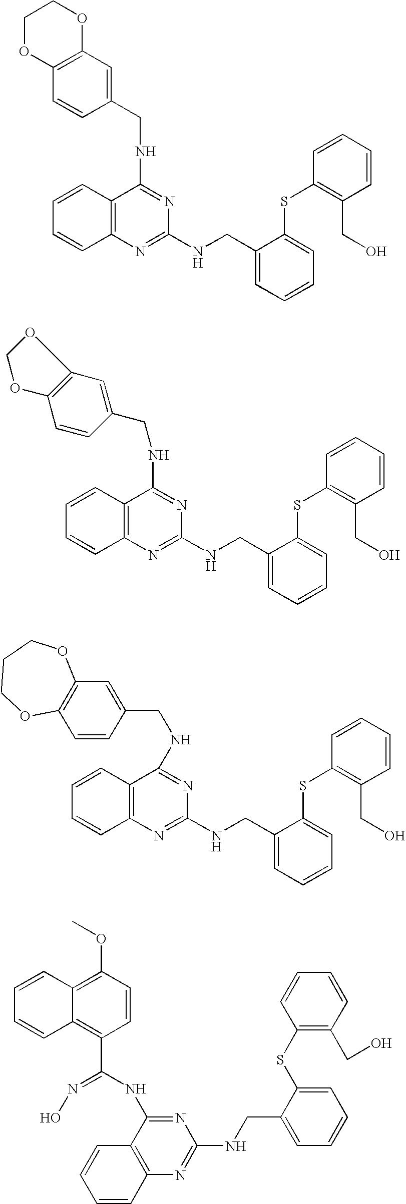

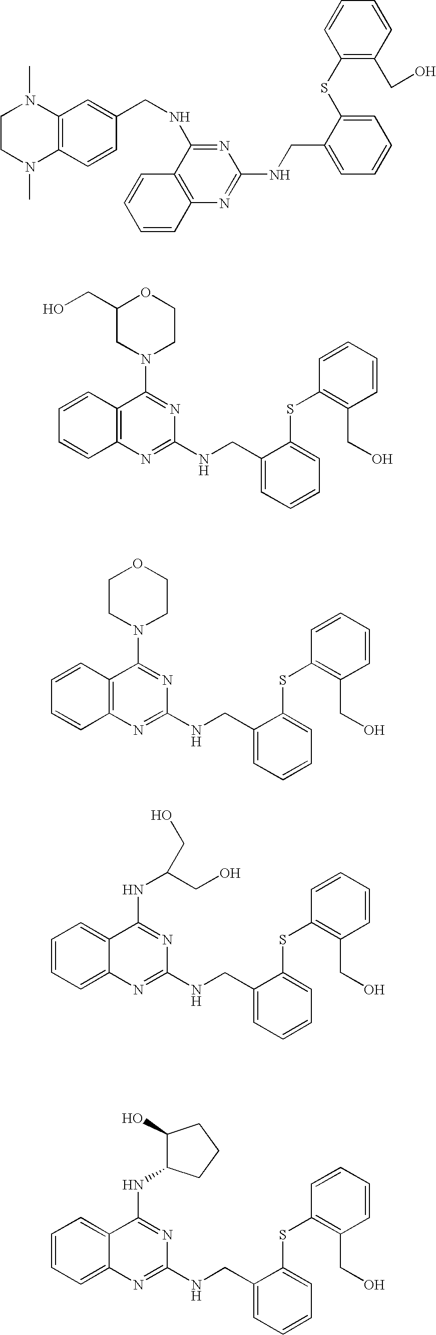

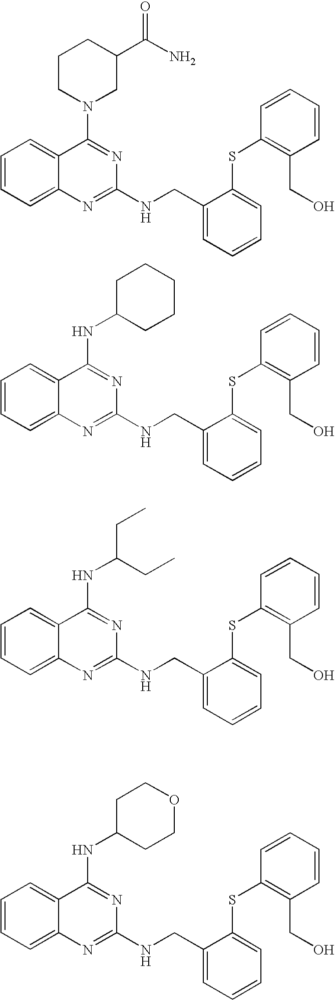

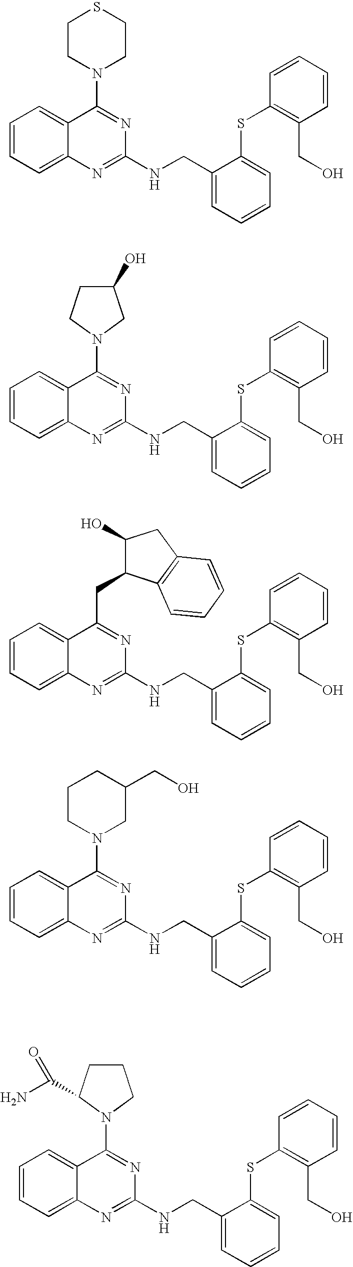

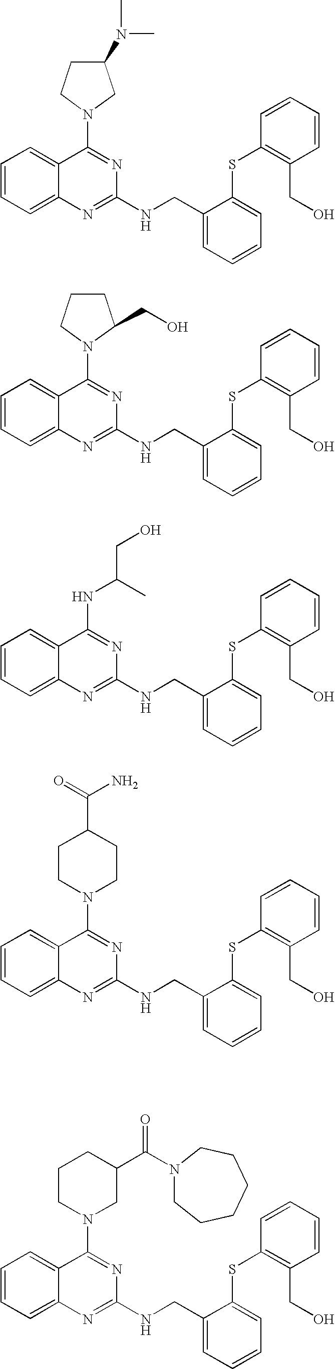

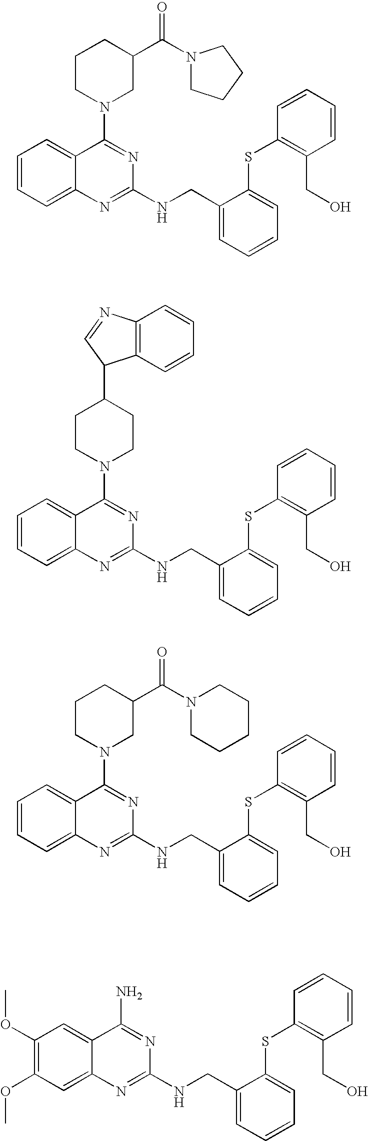

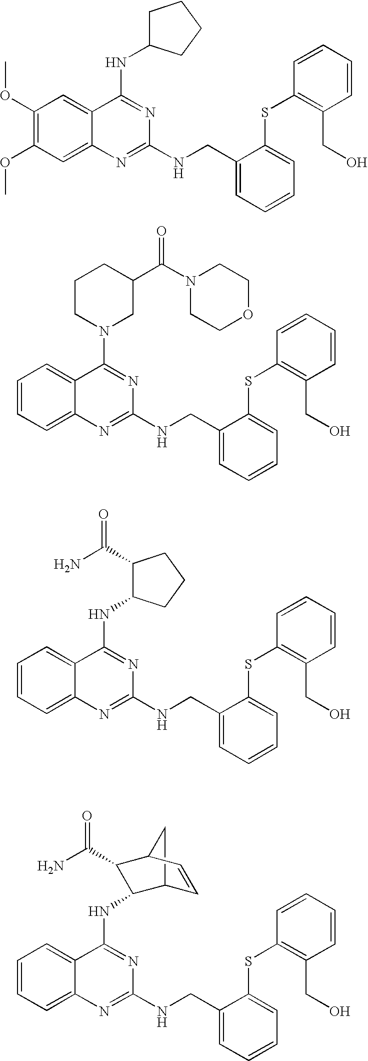

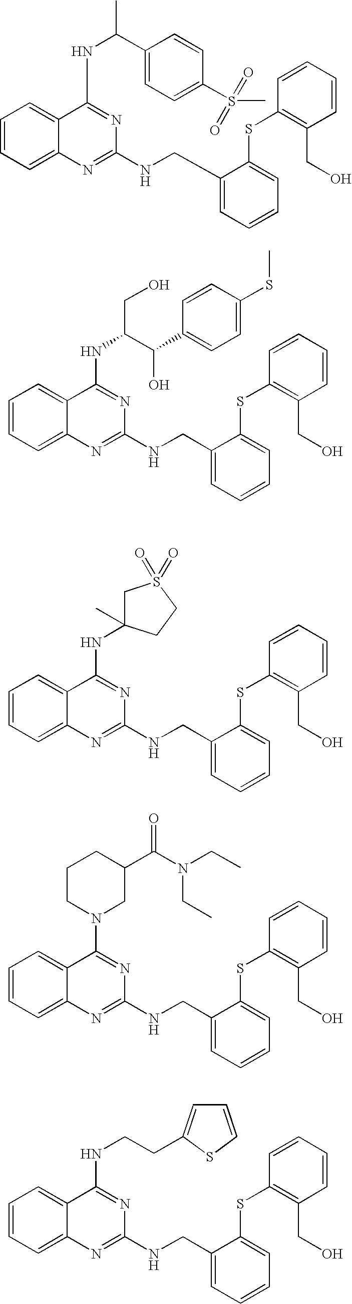

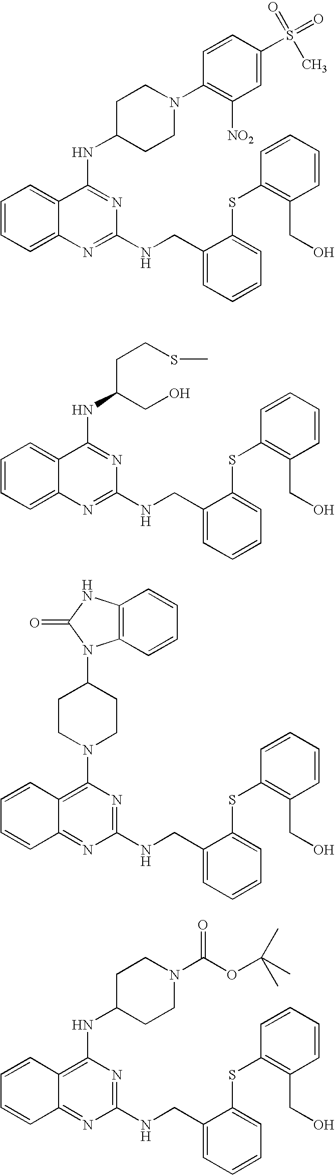

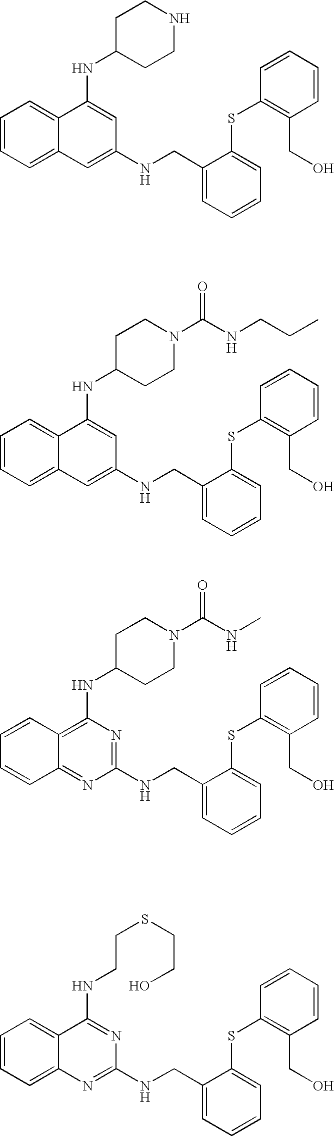

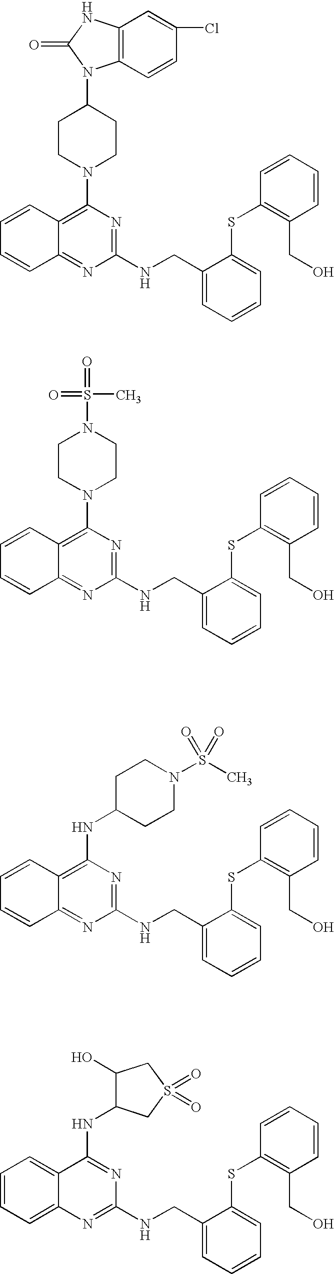

In another embodiment, the present invention relates to a method comprising a compound of formula IV:

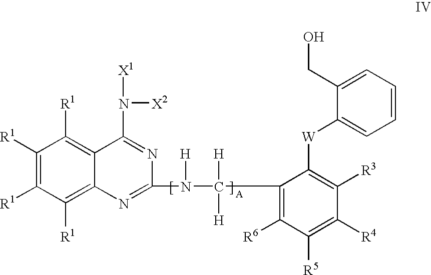

or a pharmaceutically acceptable salt thereof,

wherein, independently for each occurrence:

A is 0 or 1;

W is S, S(O) or S(O)₂;

R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶ together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned method provided that when W is S; R¹ is hydrogen; R³ is hydrogen; R⁴ is hydrogen; R⁵ is hydrogen; R⁶ is hydrogen; and —NX¹X² is 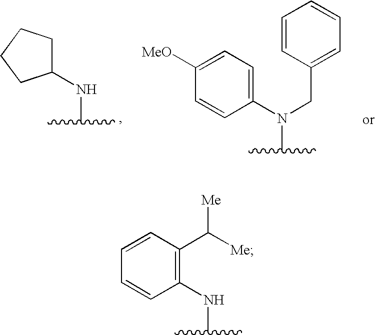
A is not 1.

In certain embodiments, the present invention relates to the aforementioned method wherein A is 0. In certain embodiments, the present invention relates to the aforementioned compound wherein A is 1.

In certain embodiments, the present invention relates to the aforementioned method wherein W is S. In certain embodiments, the present invention relates to the aforementioned method wherein W is S(O). In certain embodiments, the present invention relates to the aforementioned method wherein W is S(O)₂. In certain embodiments, the present invention relates to the aforementioned method wherein W is S or S(O)₂.

In certain embodiments, the present invention relates to the aforementioned method wherein A is 0; R³ is H; R⁴ is H; R⁵ is H; R⁶ is H; and W is S. In certain embodiments, the present invention relates to the aforementioned method wherein A is 0; R³ is H; R⁴ is H; R⁵ is OH; R⁶ is H; and W is S. In certain embodiments, the present invention relates to the aforementioned method wherein A is 0; R³ is H; R⁴ is H; R⁵ is H; R⁶ is H; and W is S(O)₂. In certain embodiments, the present invention relates to the aforementioned method wherein A is 0; R³ is H; R⁴ and R⁵ taken together are —OCH₂O—; R⁶ is H; and W is S(O)₂. In certain embodiments, the present invention relates to the aforementioned method wherein A is 0; R³ is OH; R⁴ is H; R⁵ is H; R⁶ is H; and W is S(O)₂. In certain embodiments, the present invention relates to the aforementioned method wherein A is 0; R³ is H; R⁴ is OH; R⁵ is H; R⁶ is H; and W is S(O)₂. In certain embodiments, the present invention relates to the aforementioned method wherein A is 0; R³ is H; R⁴ is H; R⁵ is OH; R⁶ is H; and W is S(O)₂. In certain embodiments, the present invention relates to the aforementioned method wherein A is 0; R³ is H; R⁴ is H; R⁵ is H; R⁶ is OH; and W is S(O)₂. In certain embodiments, the present invention relates to the aforementioned method wherein A is 1; R³ is H; R⁴ is Cl; R⁵ is H; R⁶ is H; and W is S(O)₂. In certain embodiments, the present invention relates to the aforementioned method wherein A is 0; R³ is Cl; R⁴ is H; R⁵ is H; R⁶ is H; and W is S. In certain embodiments, the present invention relates to the aforementioned method wherein A is 0; R³ is H; R⁴ is H; R⁵ is Cl; R⁶ is H; and W is S. In certain embodiments, the present invention relates to the aforementioned method wherein A is 0; R³ is H; R⁴ is H; R⁵ is H; R⁶ is Cl; and W is S. In certain embodiments, the present invention relates to the aforementioned method wherein A is 1; R³ is Cl; R⁴ is H; R⁵ is H; R⁶ is H; and W is S(O)₂. In certain embodiments, the present invention relates to the aforementioned method wherein A is 1; R³ is H; R⁴ is Cl; R⁵ is H; R⁶ is H; and W is S(O)₂. In certain embodiments, the present invention relates to the aforementioned method wherein A is 0; R³ is H; R⁴ is OH; R⁵ is OH; R⁶ is H; and W is S(O)₂. In certain embodiments, the present invention relates to the aforementioned method wherein A is 0; R³ is Cl; R⁴ is H; R⁵ is H; R⁶ is H; and W is S(O)₂. In certain embodiments, the present invention relates to the aforementioned method wherein A is 0; R³ is H; R⁴ is Cl; R⁵ is H; R⁶ is H; and W is S(O)₂.

In certain embodiments, the present invention relates to the aforementioned method wherein X¹ is cyclopentyl; and X² is hydrogen.

In another embodiment, the invention embraces a method of treating a dysproliferative or inflammatory disease such as cancer or rheumatoid arthritis comprising administering to a subject in need thereof a therapeutically effective amount of a compound or pharmaceutical composition comprising a compound selected from the compounds listed below.

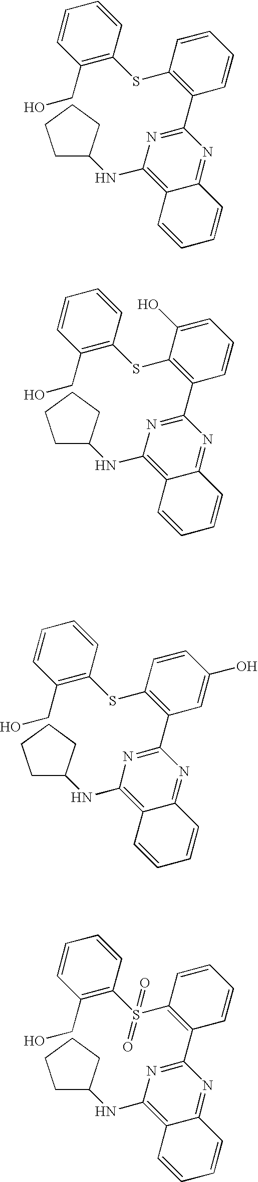

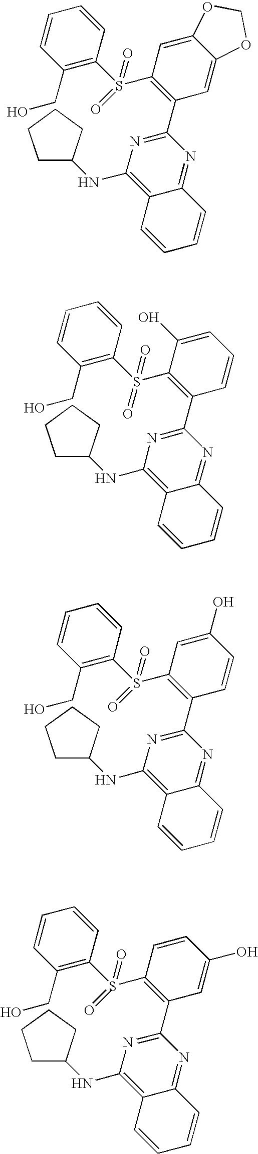

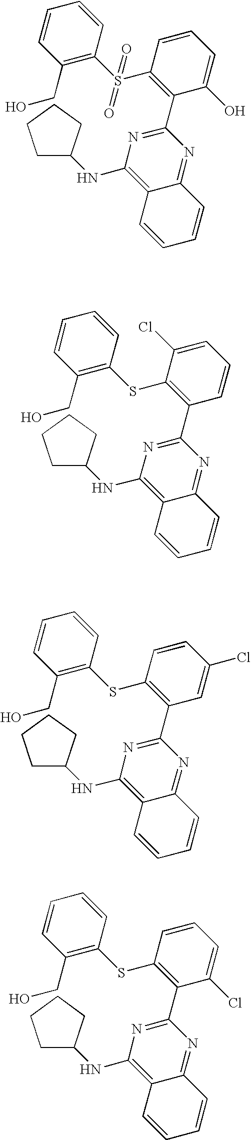

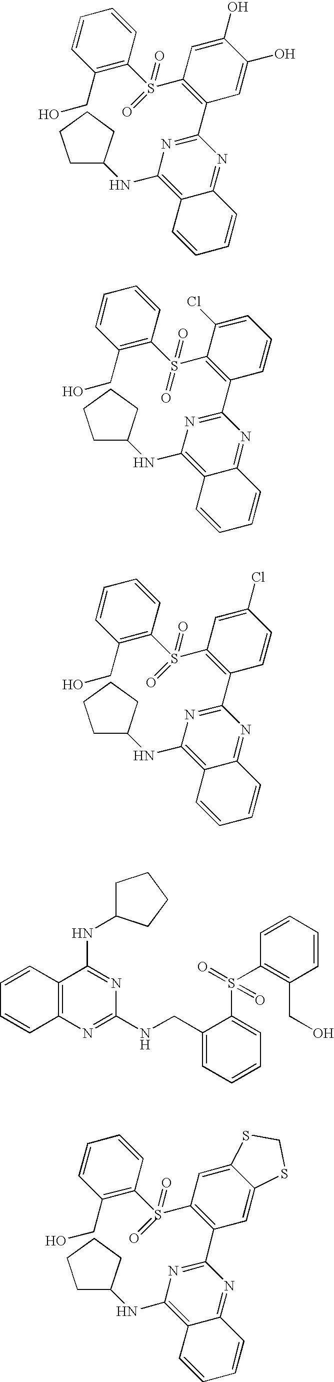

As mentioned before, the foregoing examples are merely illustrative and method comprising compounds comprising variants of the substituents as well as their positions are fully embraced by the teachings herein.

In another embodiment, the invention embraces a method of treating a dysproliferative or inflammatory disease such as cancer or rheumatoid arthritis comprising administering to a subject in need thereof a therapeutically effective amount of a compound or pharmaceutical composition comprising a compound of formula V:

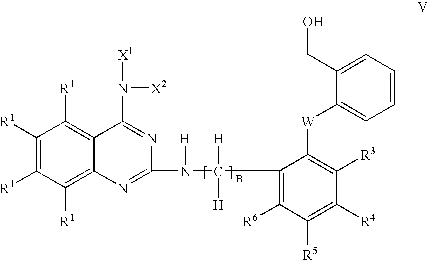

or a pharmaceutically acceptable salt thereof,

wherein, independently for each occurrence:

B is 0, 1, 2, 3, 4, or 5;

W is S, S(O) or S(O)₂;

R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned method provided that when W is S; R¹ is hydrogen; R³ is hydrogen; R⁴ is hydrogen; R⁵ is hydrogen; R⁶ is hydrogen; and —NX¹X² is 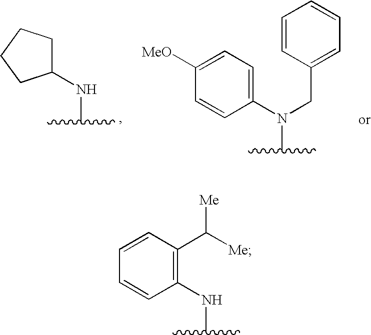
B not 1.

In certain embodiments, the present invention relates to the aforementioned method wherein B is 0. In certain embodiments, the present invention relates to the aforementioned method wherein B is 1. In certain embodiments, the present invention relates to the aforementioned method wherein B is 2. In certain embodiments, the present invention relates to the aforementioned method wherein B is 3. In certain embodiments, the present invention relates to the aforementioned method wherein B is 4. In certain embodiments, the present invention relates to the aforementioned method wherein B is 5.

In certain embodiments, the present invention relates to the aforementioned method wherein W is S. In certain embodiments, the present invention relates to the aforementioned method wherein W is S(O). In certain embodiments, the present invention relates to the aforementioned method wherein W is S(O)₂. In certain embodiments, the present invention relates to the aforementioned method wherein W is S or S(O)₂.

In certain embodiments, the present invention relates to the aforementioned method wherein X¹ is cyclopentyl; R¹, X² and X³ are hydrogen; W is S; and at least one of R³, R⁴, R⁵ and R⁶ is not hydrogen.

In another embodiment, the invention embraces a method of treating a dysproliferative or inflammatory disease such as cancer or rheumatoid arthritis comprising administering to a subject in need thereof a therapeutically effective amount of a compound or pharmaceutical composition comprising a compound of formula VI:

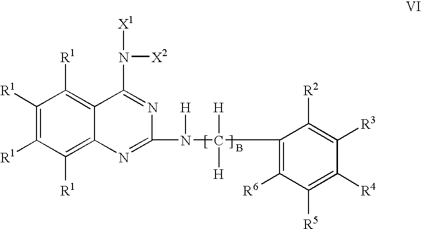

or a pharmaceutically acceptable salt thereof,

wherein, independently for each occurrence:

B is 0, 1, 2, 3, 4, or 5;

R¹ is hydrogen, —F, —Cl, —Br, —I, —OH, —SH, —NO₂, —CN, —OR¹, —SR¹, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;

R², R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};

X¹ and X² are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;

R^{R} is hydrogen or an optionally substituted aliphatic, alicyclic, heteroaliphatic, heterocyclic, aromatic, heteroaromatic or acyl moiety;

R^{A} is hydrogen or an optionally substituted aliphatic, alicyclic, heteroaliphatic, heterocyclic, aromatic or heteroaromatic moiety;

R^{B} is hydrogen, —OH, —SO₂R^{D}, or an optionally substituted aliphatic, alicyclic, heteroaliphatic, heterocyclic, aromatic, heteroaromatic or acyl moiety;

R^{C} is hydrogen, —OH, —SO₂R^{D}, or an optionally substituted aliphatic, alicyclic, heteroaliphatic, heterocyclic, aromatic, heteroaromatic or acyl moiety;

R^{D} is hydrogen, —N(R^{E})₂, or an optionally substituted aliphatic, alicyclic, heteroaliphatic, heterocyclic, aromatic or heteroaromatic moiety; and

R^{E} is hydrogen or an optionally substituted aliphatic moiety.

In certain embodiments, the present invention relates to the aforementioned method provided that when R¹ is hydrogen; R² is —SR^{R}; R³ is hydrogen; R⁴ is hydrogen; R⁵ is hydrogen; R⁶ is hydrogen; R^{R} is 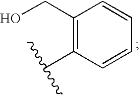
and —NX¹X² is 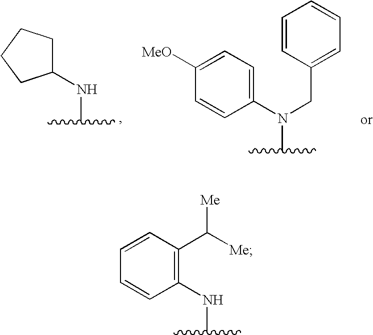
B is not 1.

Importantly, some of the compounds of the invention can comprise one or more asymmetric centers, and thus can exist in various isomeric forms, e.g., stereoisomers and/or diastereomers. Thus, the compounds in the methods described above may be in the form of an individual enantiomer, diastereomer or geometric isomer, or may be in the form of a mixture of stereolsomers. In certain embodiments, the methods of the present invention relates to the aforementioned compounds, and pharmaceutical composition, which are enantiopure compounds. In certain other embodiments, mixtures of stereoisomers or diastereomers are provided in the aforementioned methods.

### EXEMPLIFICATION

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Example 1

Following the synthesis guidance provided above, the following compounds were prepared.

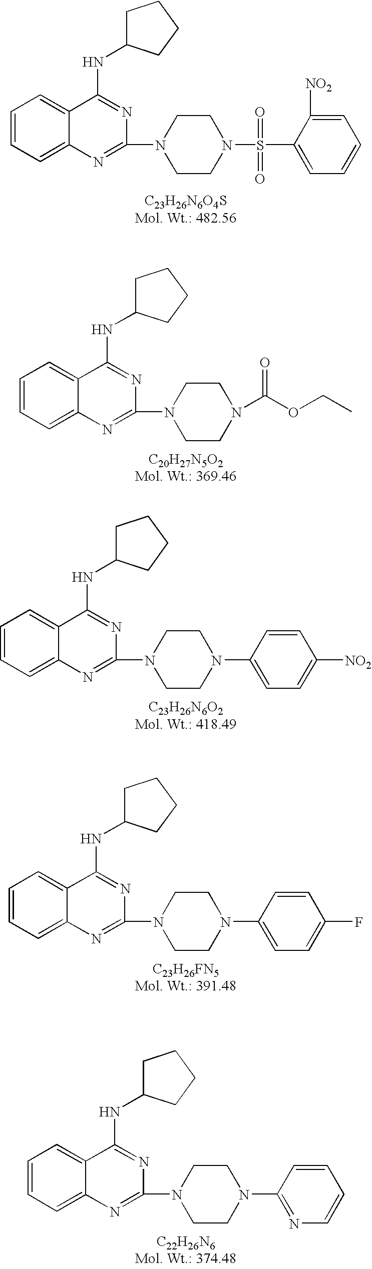

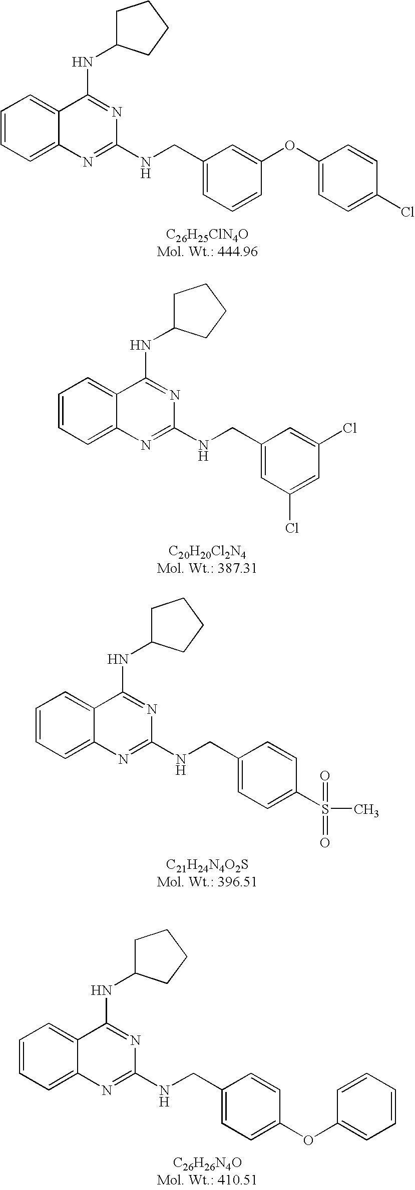

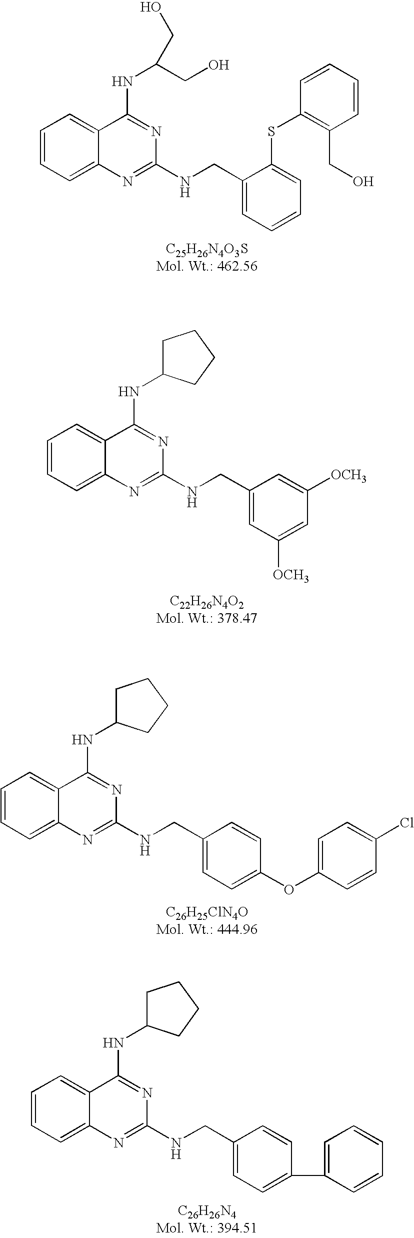

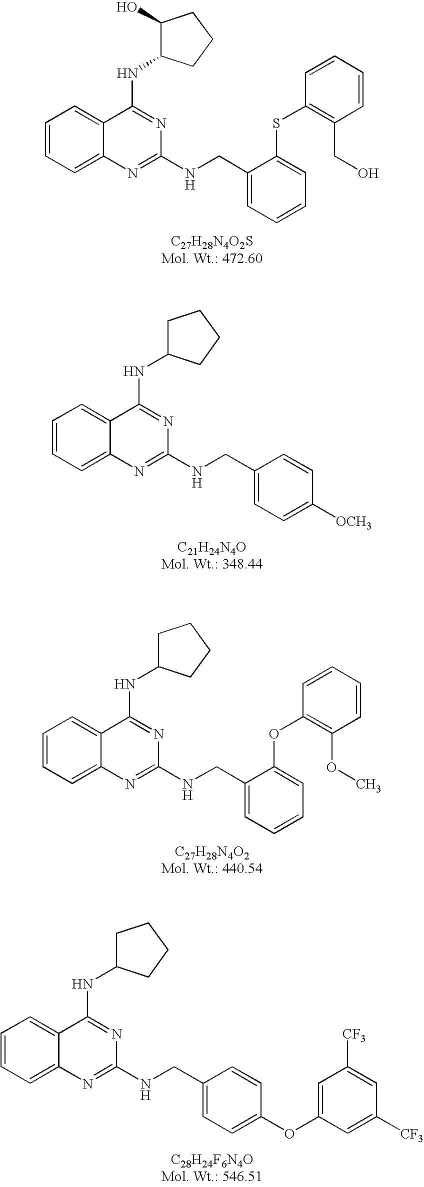

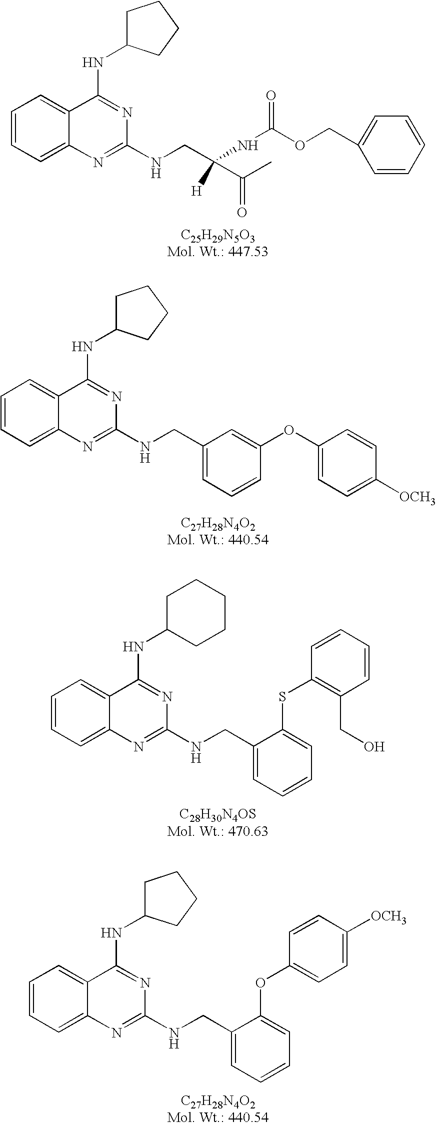

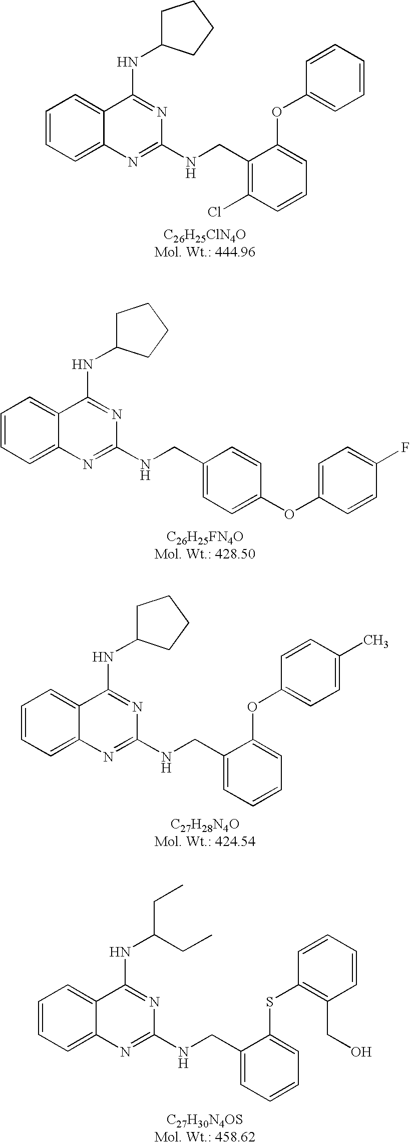

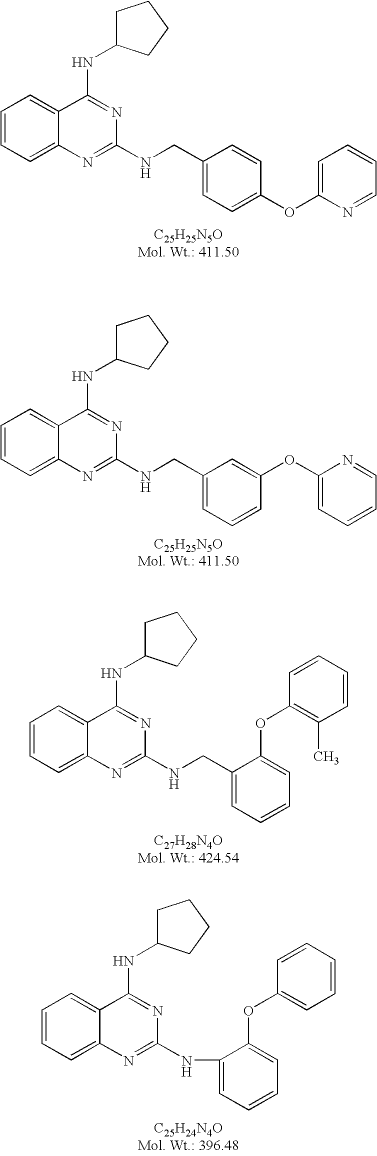

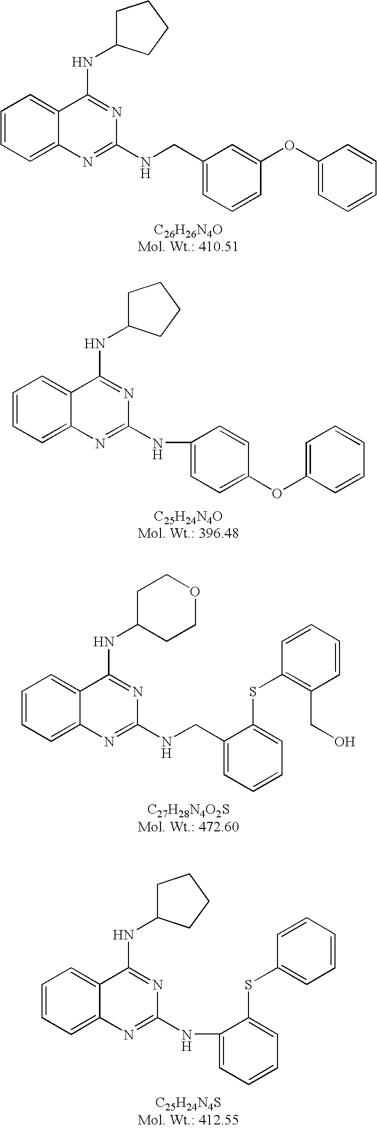

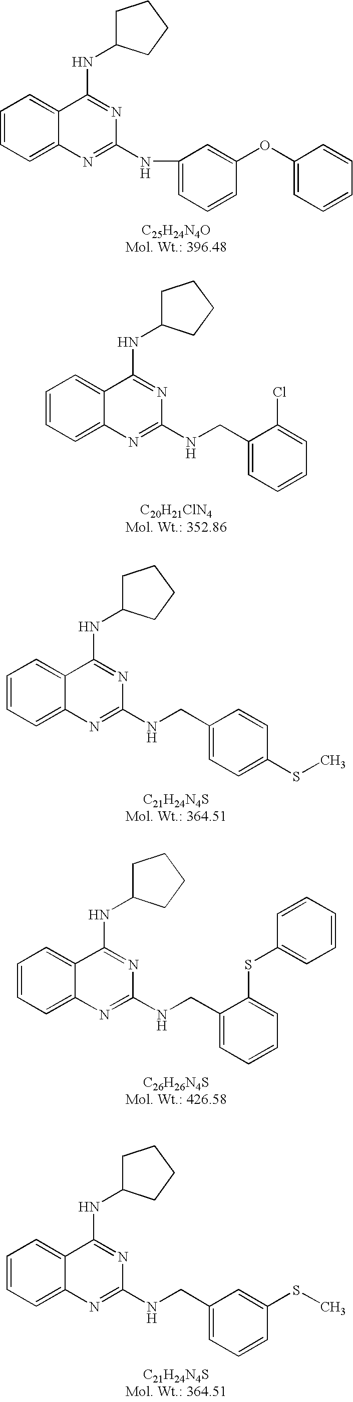

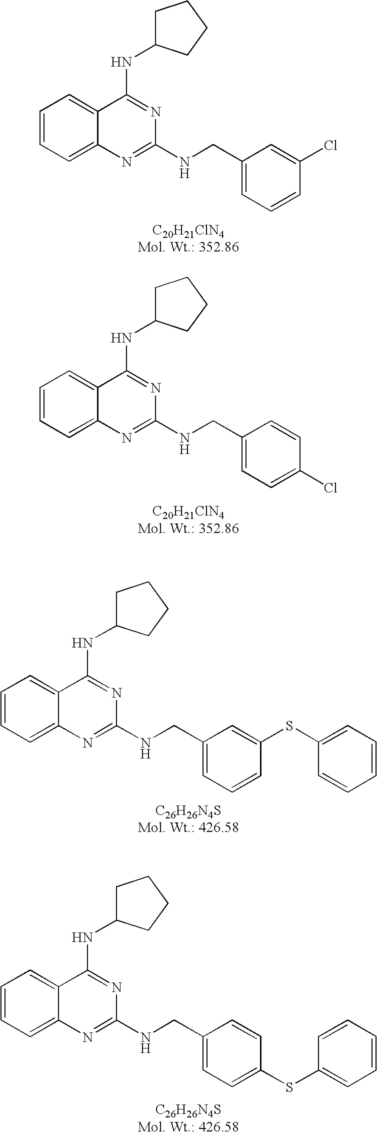

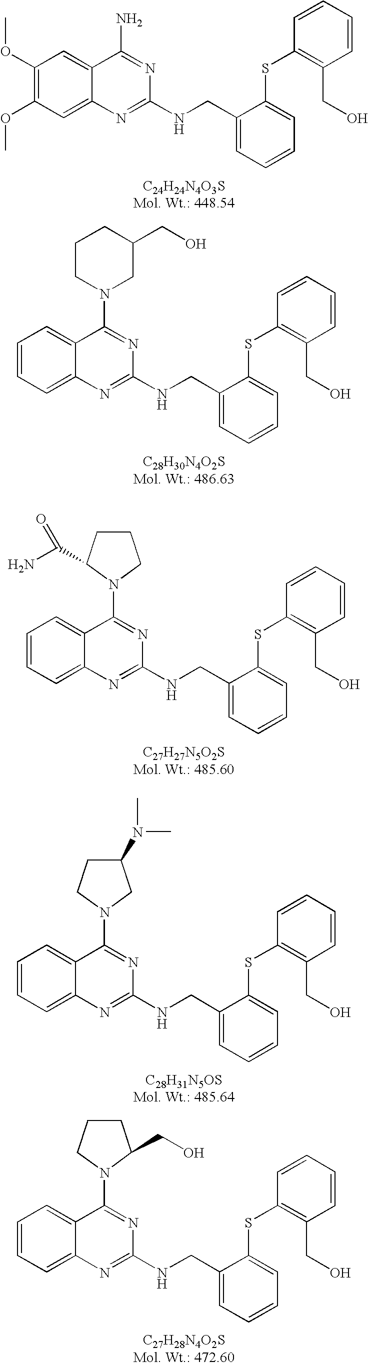

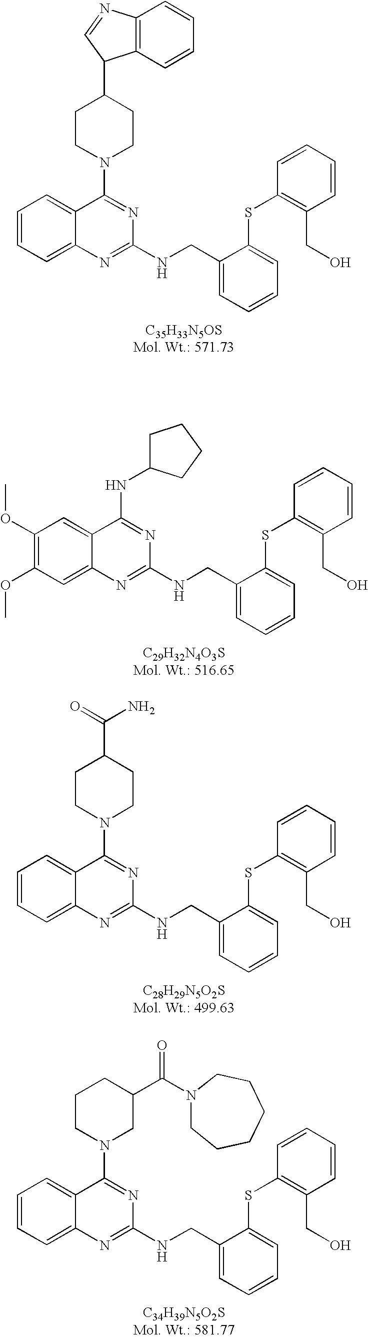

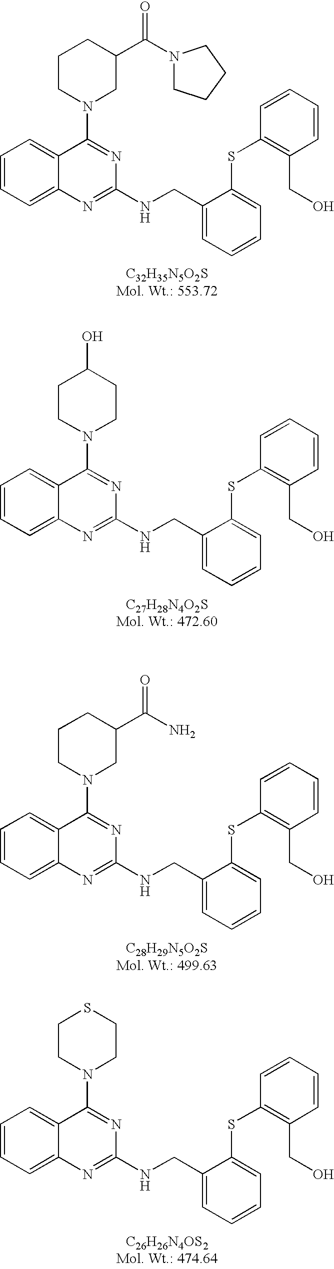

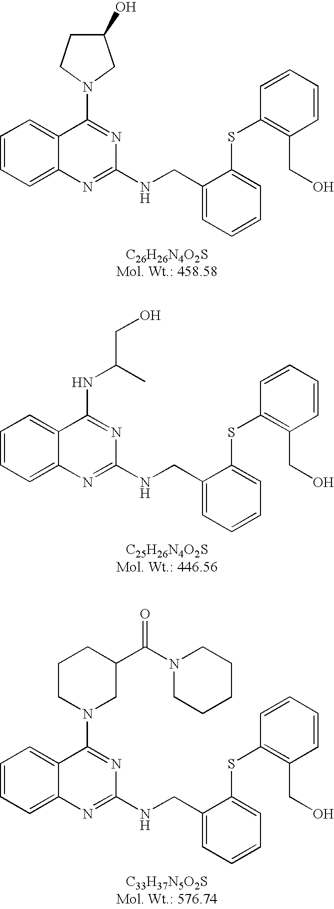

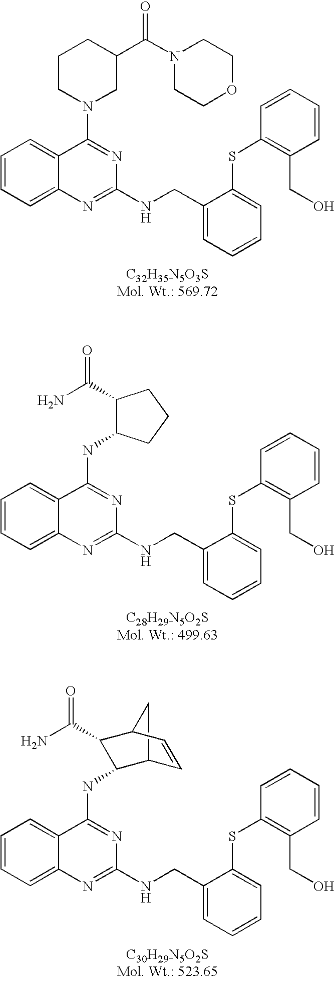

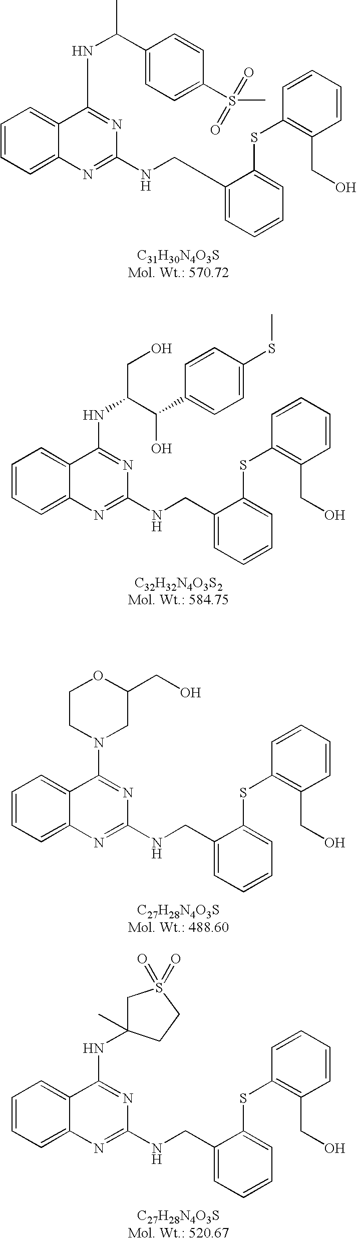

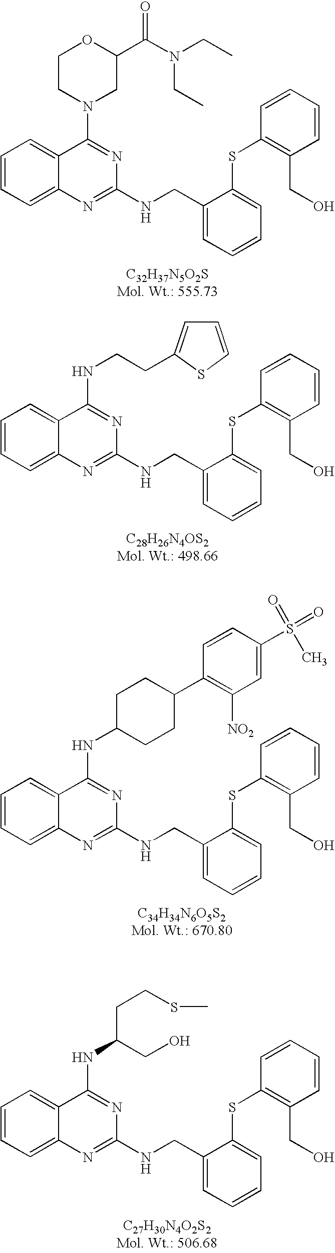

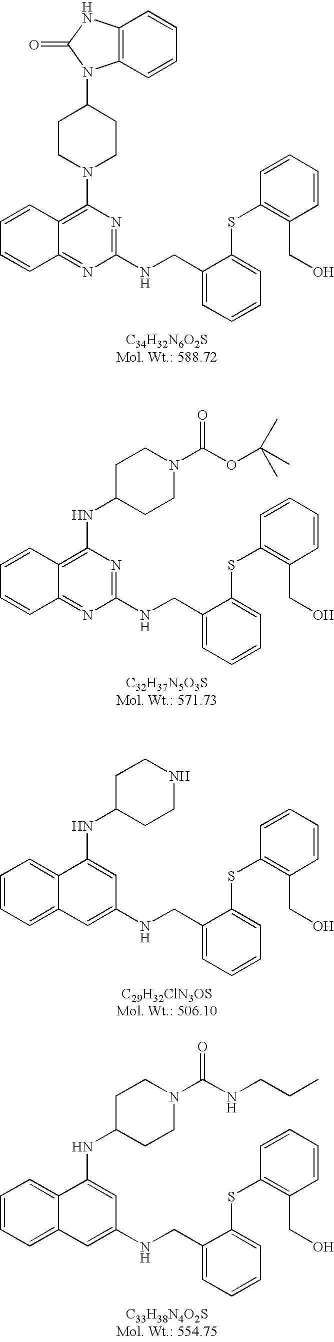

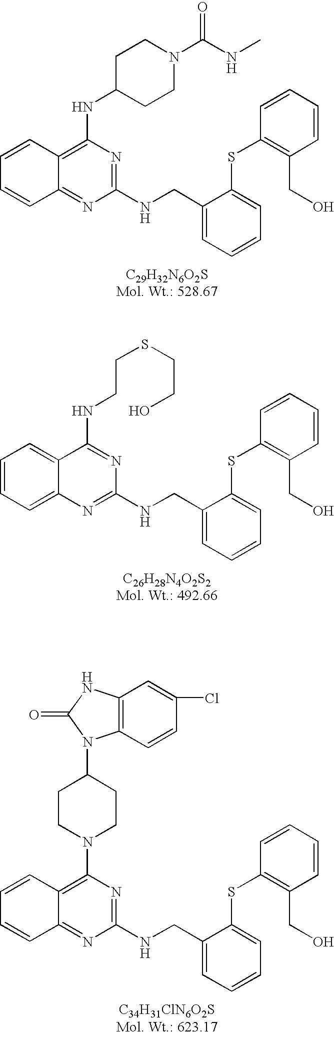

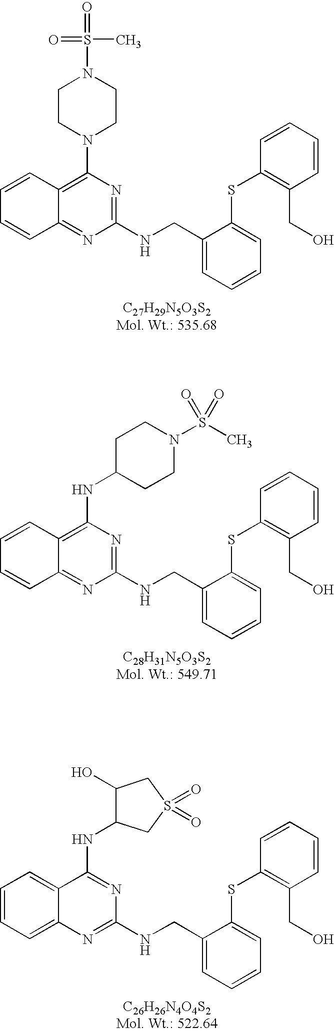

### Example 2

Compounds of the invention were tested for c-Met inhibitory activity in HGF/SF-induced HUVEC cell proliferation in vitro. Briefly, HUVEC cells were seeded into 48-well plates and serum starved for 2 hours in medium containing 1% BSA, and then treated with test compounds in multiple concentrations in the presence or absence of HGF/SF (25 ng/mL, R&D Systems) overnight. This experiment also included negative (vehicle alone) and positive (HGF/SF alone) controls. Cell proliferation was measured by the incorporation of [³H]-thymidine and counted using Beta scintillation counter. Compounds of the invention inhibited HGF/SF stimulation of endothelial cell proliferation.

Compounds were evaluated for biological activity in one or more other in vitro assays. In an assay evaluating inhibition of HGF-induced proliferation of 4MBR-5 monkey epithelial cells expressing the HGF receptor, c-Met, on day one 4MBR-5 cells were seeded and HGF and compounds were added. After 24 hour incubation, ³H-thymidine was added, and 24 hours later, the cells were harvested and thymidine incorporation was measured. In another assay, as described above, a reporter cell line (CELLSENSOR™ AP-1-bla HEK 293T Cell Line (Invitrogen)) was used to detect signaling induced by HGF.

In addition, c-Met kinase and Tie-2 kinase inhibition was measured using an enzymatic assay. Human epidermal cancer cells A431 are cultured with RPMI medium containing 0.1% fetal calf serum in 96-well plate in an amount of 3×10⁴ per well overnight. A solution of the test compound in DMSO is added to each well, and the cells are incubated at 37 C for additional one hour, and then hHGF is added to a final concentration of 50 ng/ml for 5 min. The medium is removed and the cells are washed with PBS, and 50 ul of lysis buffer is then added. The mixture is shaken at 4 C for 2 hr to prepare a cell extract. The Human Phospho-c-Met ELISA kit (R&D, DYC2480-2) is used to measure phosphorylated c-Met in these cell lysates. The Met-phosphorylation inhibitory activity for each well is determined by presuming the absorbance with the addition of HGF and the vehicle to compounds to be 0% Met-phosphorylation inhibitory activity and the absorbance with the addition of the vehicle to compounds and without HGF to be 100% Met phosphorylation inhibitory activity. The concentration of the test compound is varied on several levels, the inhibition (%) of Met-phosphorylation is determined for each case, and the concentration of the test compound necessary for inhibiting 50% of met phosphorylation (IC50) is calculated for comparing their inhibitory activity.

To measure Tie-2 inhibition, HUVECs were stimulated with Ang1 (300 ng/ml) in the presence or absence of a series of concentrations of test compound. Cells were lysed and 16 ug of total protein were loaded on 7.5% SDS-PAGE for Western blot analysis of Tie-2 phosphorylation. Membranes were incubated with primary anti-phosphoro-Tie-2 antibodies. In addition, in vitro protein tyrosine kinase assays were used in a radiometric format (KinaseProfiler™, Upstate). Kinase activity is measured via incorporation of ³²P-ATP into a kinase-specific substrate in the presence of test compounds.

Compounds of the invention generally showed IC50 values for inhibition of c-Met of 1 μM or below, with some compounds inhibiting c-Met with an IC50 below 100 nM. Tie-2 inhibitory activity was likewise generally less than 2 μM IC₅₀, with certain compounds having IC50s less than 200 nM:

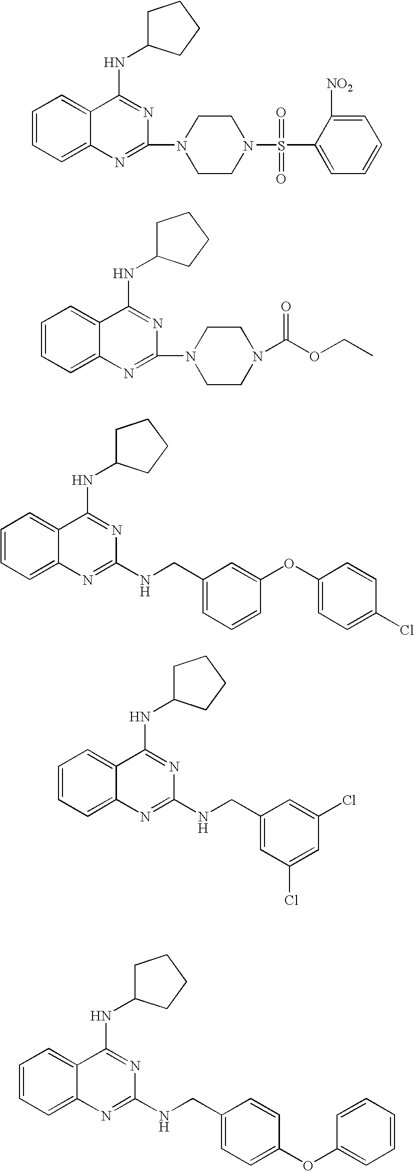

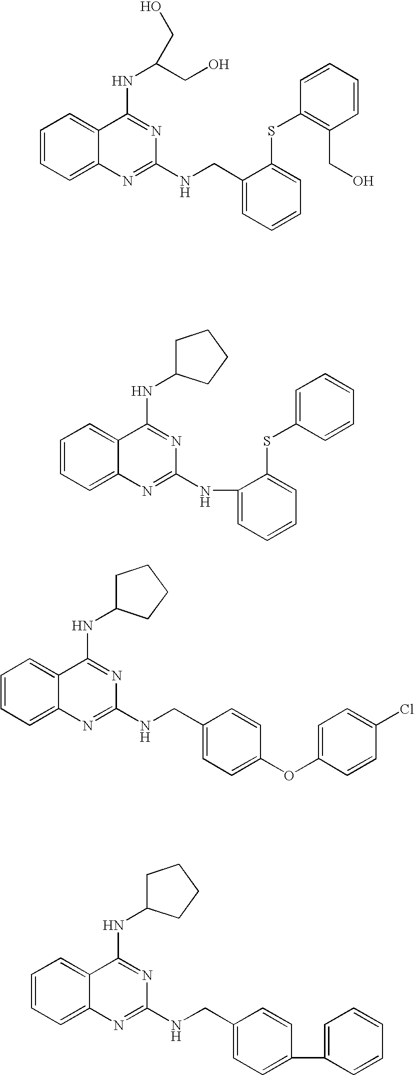

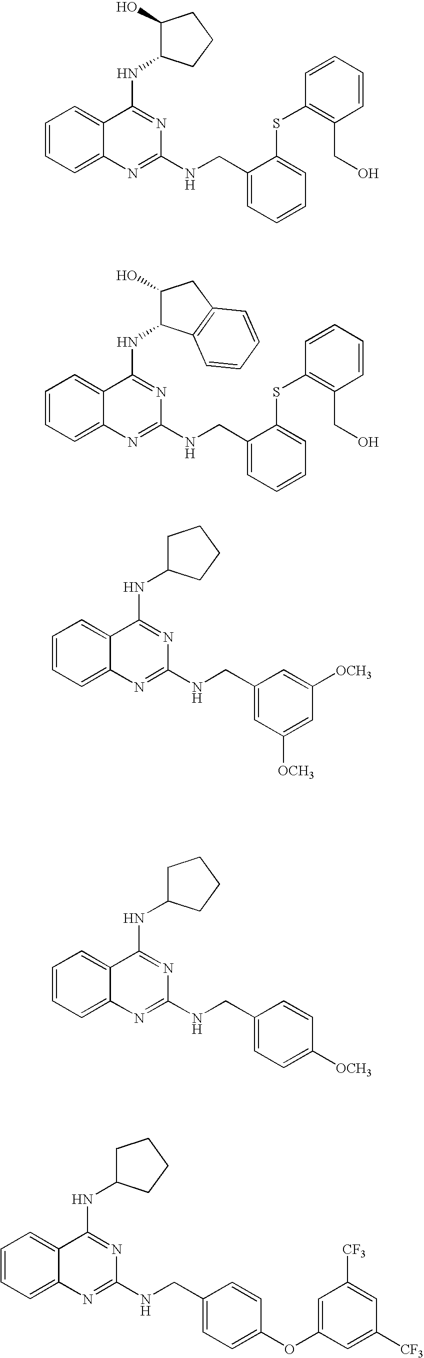

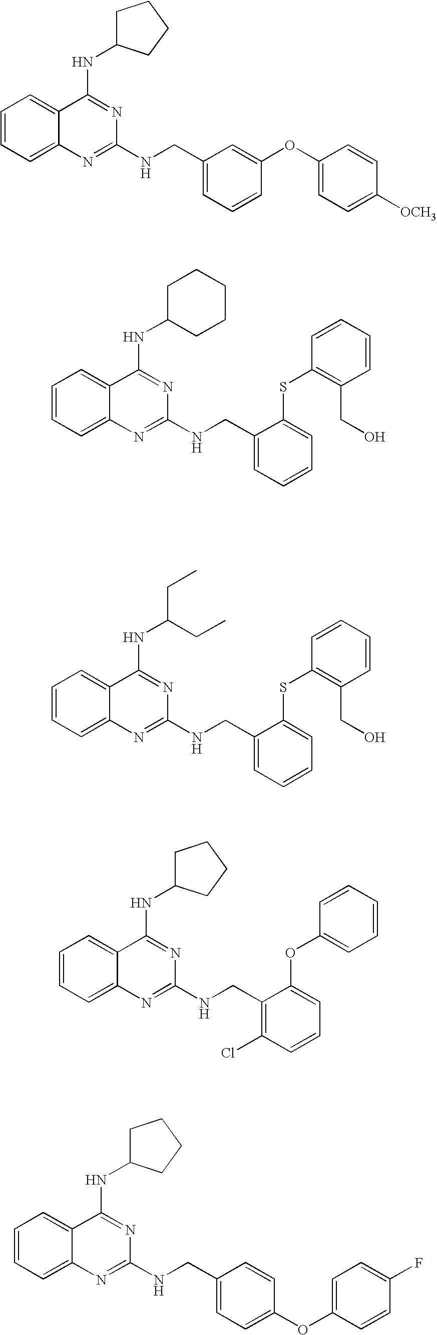

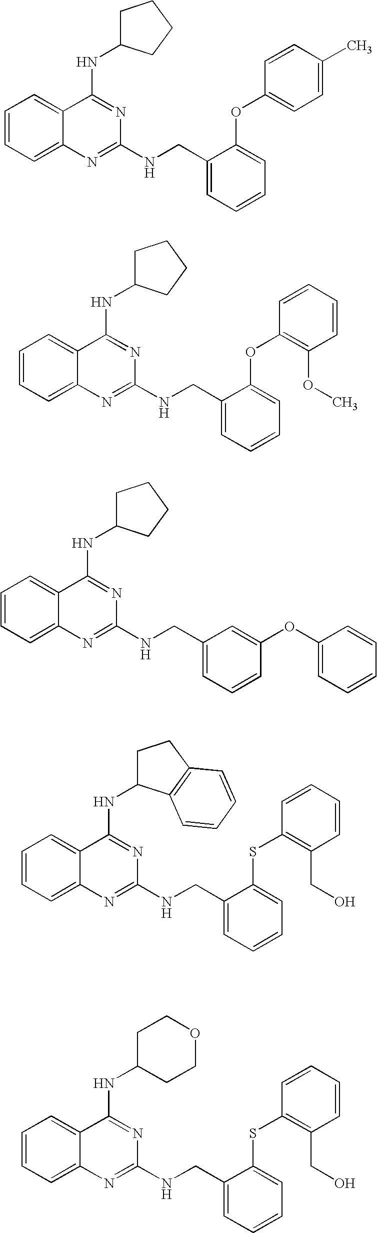

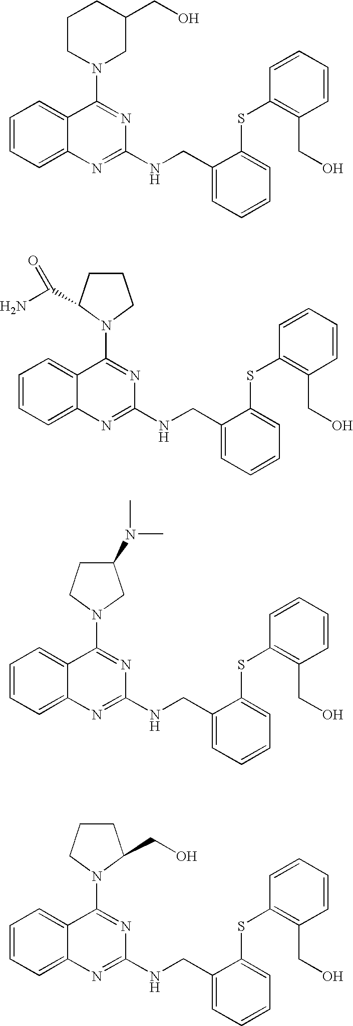

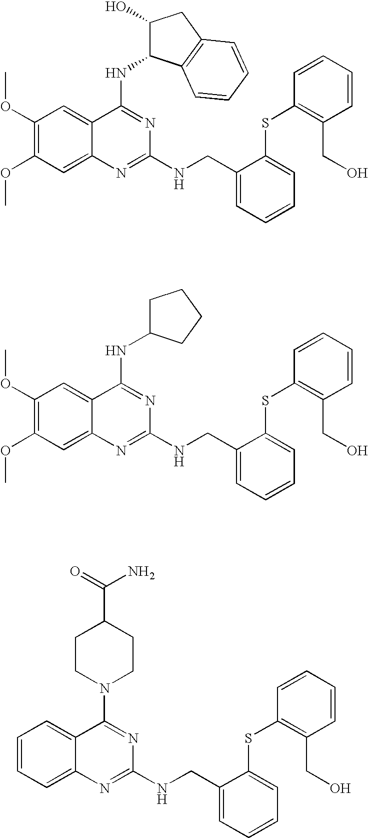

### Example 3

In a pilot study, efficacy of compounds of the invention were evaluated in a standard murine model of tumorigenesis. Human tumor xenografts were generated with A549 lung cancer cells (5×10⁶) and were grown as subcutaneous tumors in nude mice. When tumors reached a volume of ˜40 mm³ on day 5, animals were randomly divided into vehicle control, compound (10 mg/kg, i.p.), administered daily from days 5-33. The long (L) and short (W) axes of the subcutaneous tumors were measured with calipers twice weekly, and the tumor volume (TV) calculated as (L×W²)/2. Compared to vehicle, both compounds markedly reduced TV (p<0.05). More importantly, tumors from two mice in one of the treated groups exhibited regression following drug treatment. The data indicate compounds described here has therapeutic efficacy against human lung cancer xenografts in mice.

### Example 4

Homologous rat type II collagen (RII) (Chrondrex, Inc., CA) was dissolved in 0.1N acetic acid (1 mg/ml at 4 C) and emulsified with an equal volume of cold Freund's incomplete adjuvant (IFA) (Sigma, Mo.). Lewis mail rats (300 grams) received intradermal injection of RII/IFA on the back at dose of 2 mg/kg. A booster injection of the RII/IFA emulsion (100 ug of collagen in 0.1 ml) was injected at the base of the tail on day 7. On day 8, the animals were divided into two groups: Group I received compound (5 mg/kg) and Group 2 vehicle, intraperitoneally, daily for 28 days.

Arthritis Scoring: The rats were examined every second day for clinical arthritis. Each limb was scored on 5-point scale for disease severity as joints and weights the arthritis severity by joint size, as follows: 0, no arthritis; 1, redness or swelling of 1 toe/finger joint; 2, redness and swelling of more than 1 toe/finger joint; 3, ankle and tarsal-metatarsal joint involvement; 4, entire paw red or swollen. The total score was determined by adding the 4 individual leg scores to a maximum score of 16.

All animals in the compound-treated group showed good recovery.

### Incorporation by Reference

All of the U.S. patents, U.S. patent application publications, and published PCT patent applications designating the U.S. that are cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of treating a dysproliferative or inflammatory disease, comprising administering to a subject in need thereof a therapeutically effective amount of a compound of Formula II or pharmaceutical composition comprising a compound of Formula II: 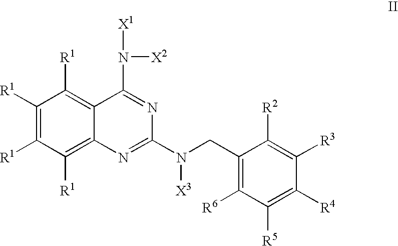
or a pharmaceutically acceptable salt thereof,
wherein, independently for each occurrence:
R¹ is hydrogen, —F, —Cl, —Br, —I, —OH, —SH, —NO₂, —CN, —OR^{R}, —SR^{D}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;
R²R³, R⁴, R⁵, and R⁶ are hydrogen, —F, —Cl, —Br, —I, —OH, —SH, —NO₂, —CN, —OR^{R}, —SR^{R}, —S(═O)R^{D}, —S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or R² and R³, R³ and R⁴, R⁴ and R⁵, or R⁵ and R⁶, together with the carbons to which they are bound, represent a fused 5-9 membered alicyclic, heterocyclic, aromatic or heteroaromatic ring; provided that at least one of R², R³ and R⁴ is —SR^{R};
X¹, X² and X³ are hydrogen or an optionally substituted aliphatic, alicyclic, heteroaliphatic, heterocyclic, aromatic, heteroaromatic or acyl group; or X¹ and X² taken together with the nitrogen to which they are bonded represent an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;
R^{R} is hydrogen or an optionally substituted aliphatic, alicyclic, heteroaliphatic, heterocyclic, aromatic, heteroaromatic or acyl moiety;
R^{A} is hydrogen or an optionally substituted aliphatic, alicyclic, heteroaliphatic, heterocyclic, aromatic or heteroaromatic moiety;
R^{B} is hydrogen, —OH, —SO₂R^{D}, or an optionally substituted aliphatic, alicyclic, heteroaliphatic, heterocyclic, aromatic, heteroaromatic or acyl moiety;
R^{C} is hydrogen, —OH, —SO₂R^{D}, or an optionally substituted aliphatic, alicyclic, heteroaliphatic, heterocyclic, aromatic, heteroaromatic or acyl moiety;
R^{D} is hydrogen, —N(R^{E})₂, or an optionally substituted aliphatic, alicyclic, heteroaliphatic, heterocyclic, aromatic or heteroaromatic moiety; and
R^{E} is hydrogen or an optionally substituted aliphatic moiety.

2. The method of claim 1, wherein the dysproliferative disease is cancer.

3. The method of claim 1, wherein the inflammatory disease is rheumatoid arthritis.

4. The method of claim 1, wherein the compound is

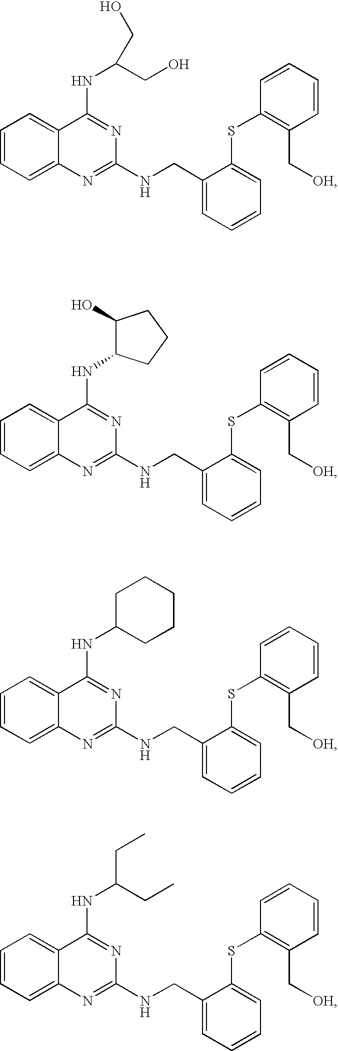

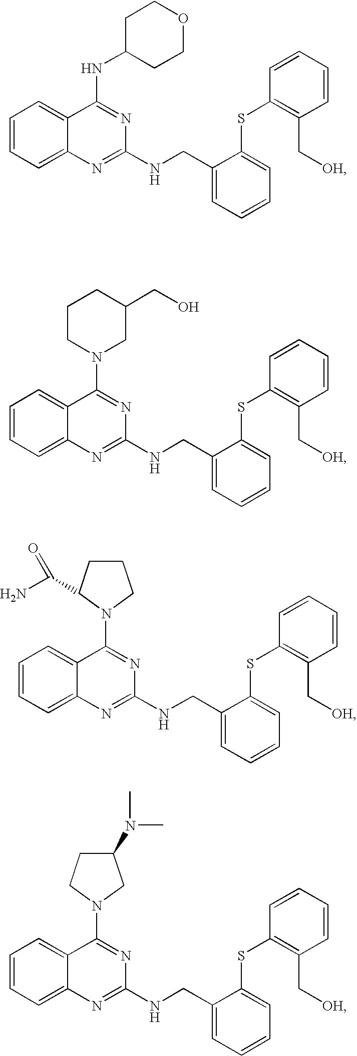

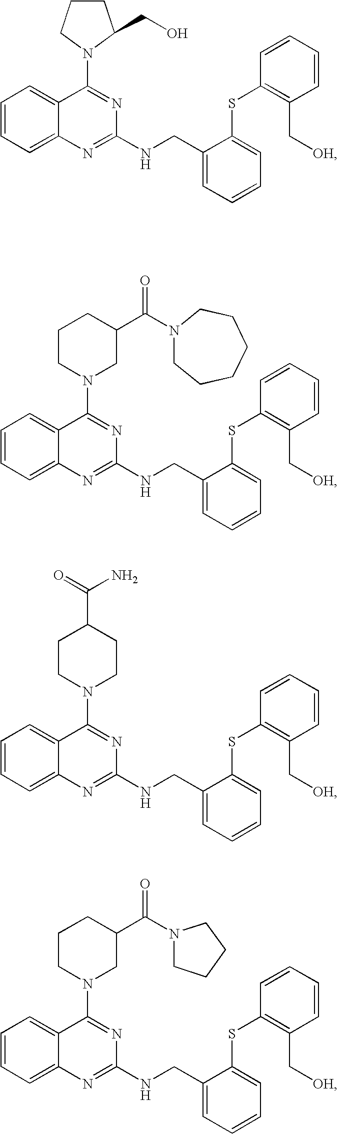

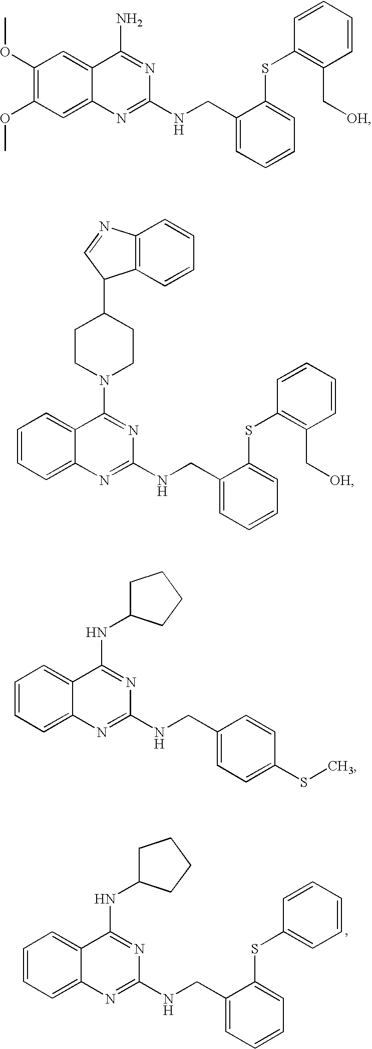

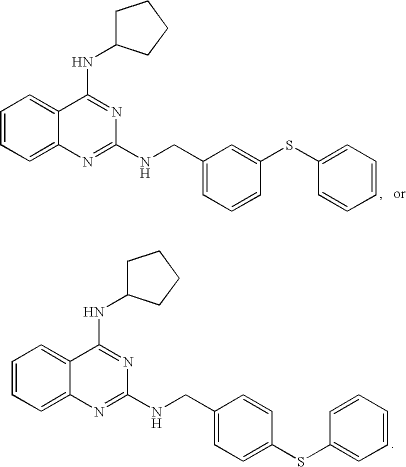

5. A method of treating a dysproliferative or inflammatory disease, comprising administering to a subject in need thereof a therapeutically effective amount of a compound of Formula I or pharmaceutical composition comprising a compound of Formula I: 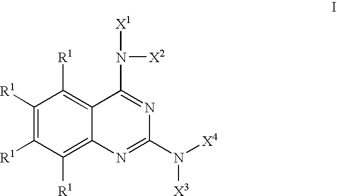
or a pharmaceutically acceptable salt thereof,
wherein, independently for each occurrence,
R¹ is hydrogen, —F, —Cl, —Br, —I, —OH, —SH, —NO₂, —CN, —OR^{R}, —SR^{D}, —S(═O)R^{D}, S(═O)₂R^{D}, —NR^{B}R^{C}, —C(═O)R^{A}, —C(═O)OR^{A} or an optionally substituted aliphatic, alicyclic, heteroaliphatic, heterocyclic, aromatic, heteroaromatic or acyl moiety; or any two R¹, together with the carbons to which they are bound, represent a fused 5-9 membered alicyclic, heterocyclic, aromatic or heteroaromatic ring;
X¹, X², X³ and X⁴ are hydrogen or an optionally substituted aliphatic, alicyclic, heteroaliphatic, heterocyclic, aromatic, heteroaromatic or acyl group; or X¹ and X² taken together with the nitrogen to which they are bonded, or X³ and X⁴ taken together with the nitrogen to which they are bonded, are independently an optionally substituted heteroaromatic or heterocyclic group comprising 4-10 ring members and 0-3 additional heteroatoms selected from the group consisting of O, N and S; the heteroaromatic or heterocyclic group optionally further substituted with one or more optionally substituted aliphatic, alicyclic, heteroaliphatic, heterocyclic, aromatic, heteroaromatic or acyl groups;
R^{R} is an optionally substituted aliphatic, alicyclic, heteroaliphatic, heterocyclic, aromatic, heteroaromatic or acyl moiety;
R^{A} is hydrogen or an optionally substituted aliphatic, alicyclic, heteroaliphatic, heterocyclic, aromatic or heteroaromatic moiety;
R^{B} is hydrogen, —OH, —SO₂R^{D}, or an optionally substituted aliphatic, alicyclic, heteroaliphatic, heterocyclic, aromatic, heteroaromatic or acyl moiety;
R^{C} is hydrogen, —OH, —SO₂R^{D}, or an optionally substituted aliphatic, alicyclic, heteroaliphatic, heterocyclic, aromatic, heteroaromatic or acyl moiety;
R^{D} is hydrogen, —N(R^{E})₂, or an optionally substituted aliphatic, alicyclic, heteroaliphatic, heterocyclic, aromatic or heteroaromatic moiety; and
R^{E} is hydrogen or an optionally substituted aliphatic moiety.

6. The method of claim 5, wherein the dysproliferative disease is cancer.

7. The method of claim 5, wherein the inflammatory disease is rheumatoid arthritis.

8. The method of claim 5, wherein the compound is 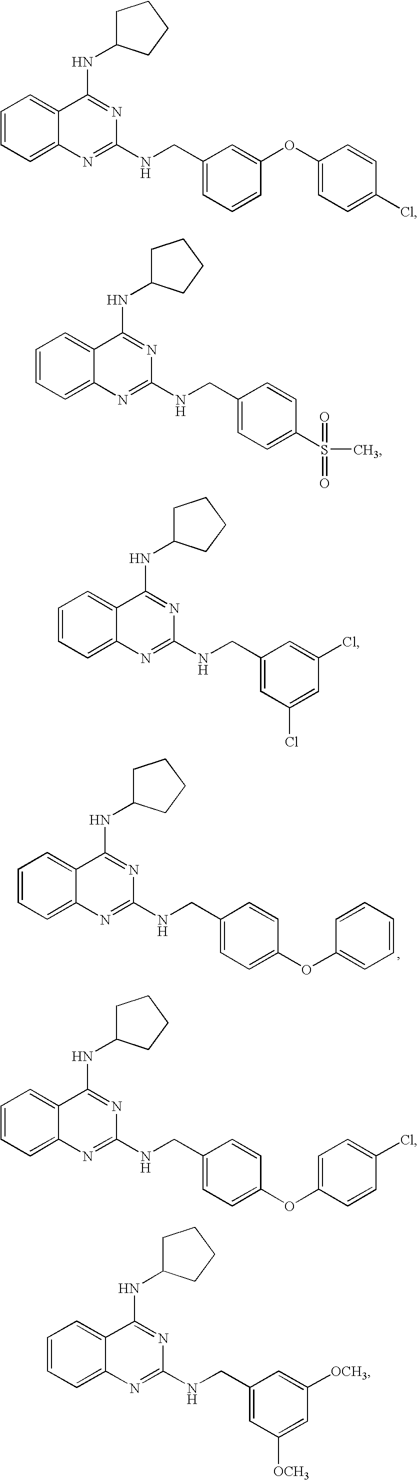 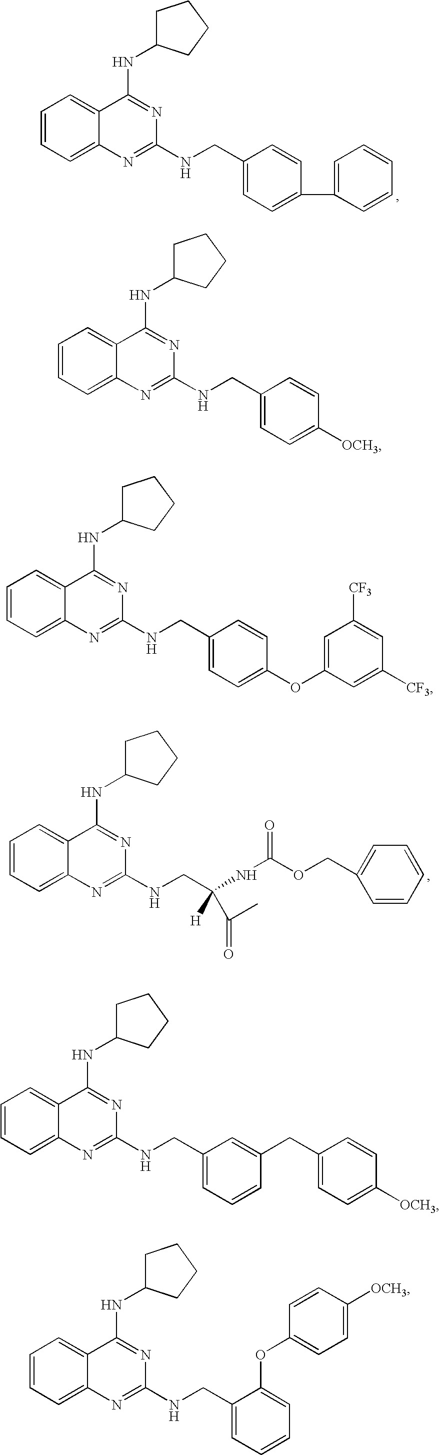 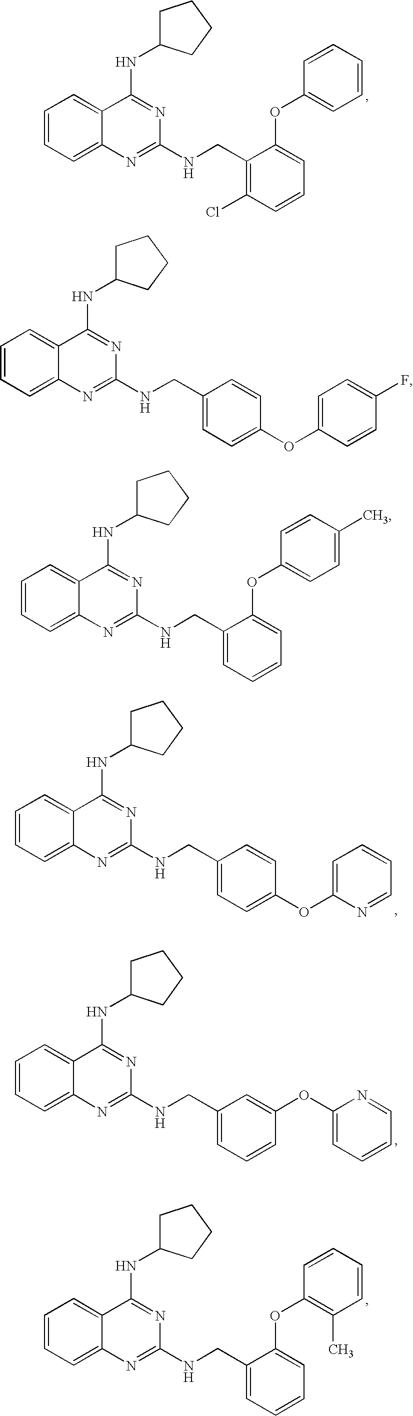 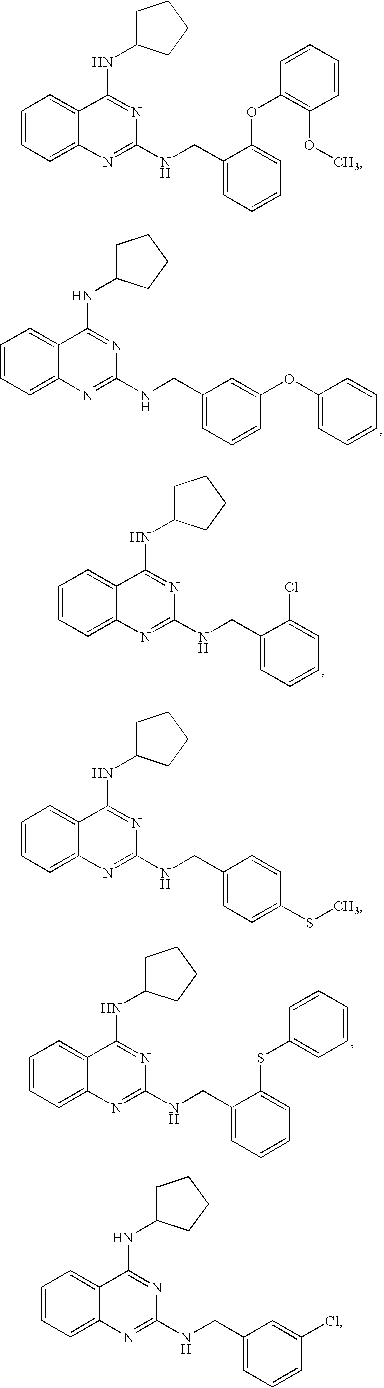 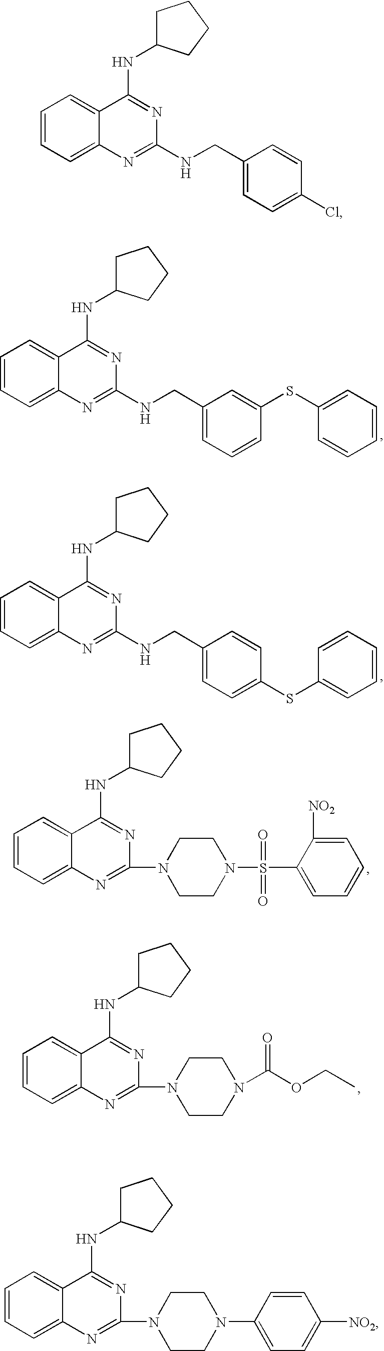 [chemical structure]
